(19) United States
(12) Patent Application Publication
(10) Pub. No.: US 2026/0305166 A1
(43) Pub. Date: Oct. 1, 2026

(54) HETEROCYCLIC COMPOUND AND ORGANIC LIGHT-EMITTING DEVICE COMPRISING SAME

(71) Applicant: LT Materials Co., Ltd., Yongin-si, Gyeonggi-do (KR)

(72) Inventors: Jin Hwan SHIN, Yongin-si, Gyeonggi-do (KR); Seok Hyeon YU, Yongin-si, Gyeonggi-do (KR); Yong Geun JUNG, Yongin-si, Gyeonggi-do (KR); Yong Woo KIM, Yongin-si, Gyeonggi-do (KR)

(21) Appl. No.: 19/475,752

(22) PCT Filed: Nov. 27, 2023
(86) PCT No.: PCT/KR2023/019212

§ 371 Date: Oct. 15, 2025

(30) Foreign Application Priority Data
Apr. 24, 2023 (KR) 10-2023-0053365

(51) Int. Cl. H10K 85/60; C07B 59/00; C07D 209/88; C07D 209/94; C07D 401/14; C07D 413/14; C07D 487/04; C07D 491/048; C07D 495/04; C09K 11/02; H10K 50/11

(52) U.S. Cl. CPC H10K 85/6572; C07B 59/002; C07D 209/88; C07D 209/94; C07D 401/14; C07D 413/14; C07D 487/04; C07D 491/048; C07D 495/04; C09K 11/02; H10K 85/615; H10K 85/657; H10K 50/11

(57) ABSTRACT

Provided is a heterocyclic compound represented by Chemical Formula 1 and an organic light-emitting device including the same. When the heterocyclic compound represented by Chemical Formula 1 is used in a light emitting layer, hole transport and electron transport properties are strengthened, a hole transfer ability is improved through adjusting a band gap and a triplet energy level (T1 level) value, a driving voltage of an organic light-emitting device is lowered and light efficiency thereof is improved by increasing stability of the molecule, and lifetime properties of the organic light-emitting device is improved due to improved thermal stability of the compound.

### TECHNICAL FIELD

This application claims priority to Korean Patent Application No. 10-2023-0053365 filed on Apr. 24, 2023, the disclosure of which is incorporated herein by reference in its entirety.

The present invention relates to a heterocyclic compound and an organic light-emitting device including the same.

### BACKGROUND ART

An organic light-emitting device is one type of self-emissive display devices, and has advantages of having a wide viewing angle and a high response speed as well as having an excellent contrast.

The organic light-emitting device has a structure of disposing an organic thin film between two electrodes. When a voltage is applied to the organic light-emitting device having such a structure, electrons and holes injected from the two electrodes bind and pair in the organic thin film, and then light is emitted as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of serving as a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light-emitting device.

### PRIOR ART DOCUMENTS

### Patent Documents

U.S. Pat. No. 4,356,429

### DISCLOSURE

### Technical Problem

The present invention is directed to providing a heterocyclic compound, and an organic light-emitting device including the same.

### Technical Solution

One embodiment of the present invention provides a heterocyclic compound represented by the following chemical formula 1:

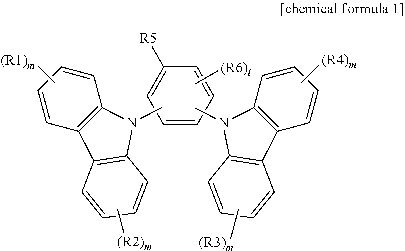

in chemical formula 1,
R1 to R4 are the same or different, and are each independently selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; a substituted or unsubstituted C2-C60 heterocycloalkyl group; and HAr,
the HAr is a substituted or unsubstituted C2-C60 heteroaryl group containing at least one nitrogen,
any one of R1 to R4 is HAr,
R5 is a substituted or unsubstituted C6-C60 aryl group,
R6 is selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; and a substituted or unsubstituted C2-C60 heterocycloalkyl group,
l is an integer of 1-3, and
m is an integer of 1-4.

In addition, one embodiment of the present invention provides an organic light-emitting device including: a first electrode; a second electrode provided opposite to the first electrode; and one or more organic material layers provided between the first electrode and the second electrode, wherein one or more layers of the organic material layers include the heterocyclic compound according to the present invention.

### Advantageous Effects

A heterocyclic compound according to one embodiment can be used as a material of an organic material layer of an organic light-emitting device. The compound is capable of performing roles of a hole injection layer material, an electron blocking layer material, a hole transport layer material, a light emitting layer material, an electron transport layer material, a hole blocking layer material, an electron injection layer material and the like in an organic light-emitting device. Particularly, the compound can be used as a material of a light emitting layer of an organic light-emitting device.

More specifically, when the heterocyclic compound according to one embodiment is used in a light emitting layer, hole transport and electron transport properties are strengthened, a hole transfer ability is improved through adjusting a band gap and a triplet energy level (T1 level) value, a driving voltage of an organic light-emitting device is lowered and light efficiency thereof is improved by increasing stability of the molecule, and lifetime properties of the organic light-emitting device is improved due to improved thermal stability of the compound.

### DESCRIPTION OF DRAWINGS

FIGS. 1 to 4 are diagrams each schematically illustrating a lamination structure of an organic light-emitting device according to one embodiment of the present invention.

### MODE FOR DISCLOSURE

Hereinafter, the present invention will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of: deuterium; halogen; a cyano group; a C1-C60 linear or branched alkyl group; a C2-C60 linear or branched alkenyl group; a C2-C60 linear or branched alkynyl group; a C3-C60 monocyclic or polycyclic cycloalkyl group; a C2-C60 monocyclic or polycyclic heterocycloalkyl group; a C6-C60 monocyclic or polycyclic aryl group; a C2-C60 monocyclic or polycyclic heteroaryl group; —SiRR′R″; —P(═O)RR′; a C1-C20 alkylamine group; a C6-C60 monocyclic or polycyclic arylamine group; and a C2-C60 monocyclic or polycyclic heteroarylamine group or being unsubstituted, or being substituted with a substituent in which two or more substituents selected from among the substituents exemplified above are linked or being unsubstituted.

In the present specification, R, R′ and R″ are the same or different, and may be each independently a substituted or unsubstituted C1-C60 alkyl group; a substituted or unsubstituted C6-C60 aryl group; or a substituted or unsubstituted C2-C60 heteroaryl group.

In the present specification, “the number of protons” means the number of substituents that a specific compound may have, and specifically, the number of protons may mean the number of hydrogens. For example, unsubstituted benzene may be expressed to have the number of protons of 5, an unsubstituted naphthyl group may be expressed to have the number of protons of 7, a naphthyl group substituted with a phenyl group may be expressed to have the number of protons of 6, and an unsubstituted biphenyl group may be expressed to have the number of protons of 9.

In the present specification, the halogen may be fluorine, chlorine, bromine or iodine.

In the present specification, the alkyl group includes a linear or branched form having 1 to 60 carbon atoms, and may be further substituted with other substituents. The number of carbon atoms of the alkyl group may be from 1 to 60, specifically from 1 to 40 and more specifically from 1 to 20. Specific examples of the alkyl group may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes a linear or branched form having 2 to 60 carbon atoms, and may be further substituted with other substituents. The number of carbon atoms of the alkenyl group may be from 2 to 60, specifically from 2 to 40 and more specifically from 2 to 20. Specific examples of the alkenyl group may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group and the like, but are not limited thereto.

In the present specification, the alkynyl group includes a linear or branched form having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples of the alkoxy group may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes a monocyclic or polycyclic group having 3 to 60 carbon atoms, and may be further substituted with other substituents. Herein, the polycyclic group means a group in which the cycloalkyl group is directly linked to or fused with another cyclic group. Herein, the another cyclic group may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples of the cycloalkyl group may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes a monocyclic or polycyclic group having 2 to 60 carbon atoms, and may be further substituted with other substituents. Herein, the polycyclic group means a group in which the heterocycloalkyl group is directly linked to or fused with another cyclic group. Herein, the another cyclic group may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes a monocyclic or polycyclic group having 6 to 60 carbon atoms, and may be further substituted with other substituents. Herein, the polycyclic group means a group in which the aryl group is directly linked to or fused with another cyclic group. Herein, the another cyclic group may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

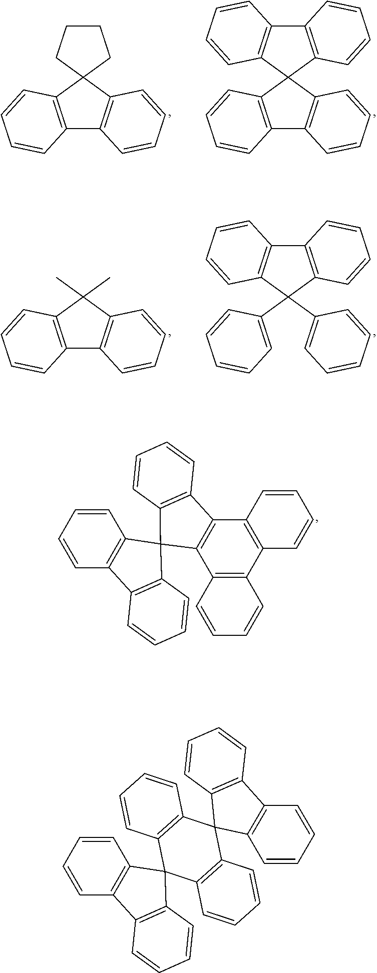

and the like may be included, however, the structure is not limited thereto.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the following spiro group may include any one of groups of the following structural formulae.

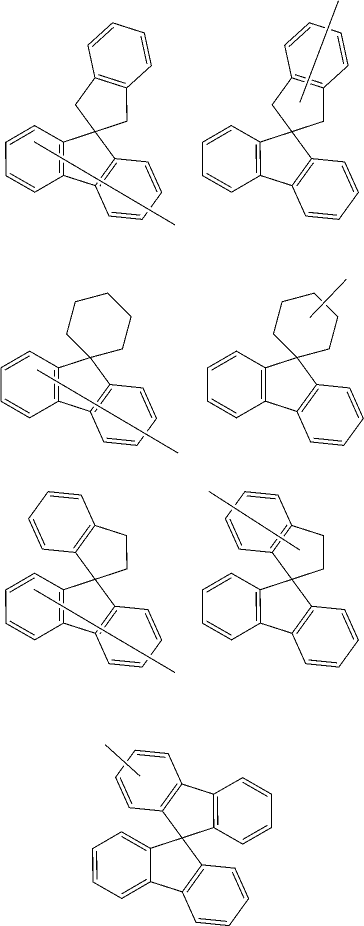

In the present specification, the heteroaryl group includes S, O, Se, N or Si as a heteroatom, includes a monocyclic or polycyclic group having 2 to 60 carbon atoms, and may be further substituted with other substituents. Herein, the polycyclic group means a group in which the heteroaryl group is directly linked to or fused with another cyclic group. Herein, the another cyclic group may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, an imidazopyridinyl group, a diazanaphthalenyl group, a triazaindenyl group, a 2-indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole group, a spirobi (dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for a divalent group. In addition, the heteroarylene group means the heteroaryl group having two bonding sites, that is, a divalent group. The descriptions on the heteroaryl group provided above may be applied thereto except for a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present invention, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present invention, a “case of a substituent being not indicated in a chemical formula or compound structure” may mean that positions to which substituents may come are all hydrogen or deuterium. In other words, since deuterium is an isotope of hydrogen, some hydrogen atoms may be deuterium that is an isotope, and herein, a content of the deuterium may be from 0% to 100%.

In one embodiment of the present invention, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present invention, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present invention, an isotope means an atom with the same atomic number (Z) but with a different mass number (A), and may also be interpreted as an element with the same number of protons but with a different number of neutrons.

In one embodiment of the present invention, a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

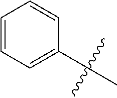 [SMILES: *c1ccccc1]

may mean that the total number of substituents that the phenyl group may have is 5 (T1 in the formula), and the number of deuterium atoms among these is 1 (T2 in the formula). In other words, having a deuterium content of 20% in a phenyl group may be represented by the following structural formulae.

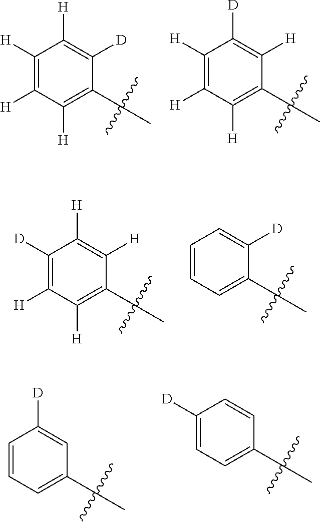 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present invention, “a phenyl group having a deuterium content of 0%” may mean a phenyl group that does not include a deuterium atom, that is, a phenyl group that has 5 hydrogen atoms.

In the present invention, the C6-C60 aromatic hydrocarbon ring means a compound including an aromatic ring formed with C6-C60 carbons and hydrogens. Examples thereof may include benzene, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

One embodiment of the present invention provides a heterocyclic compound represented by the following chemical formula 1:

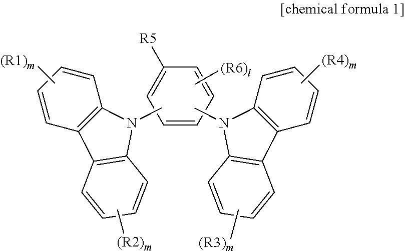

in chemical formula 1,
R1 to R4 are the same or different, and are each independently selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; a substituted or unsubstituted C2-C60 heterocycloalkyl group; and HAr,
the HAr is a substituted or unsubstituted C2-C60 heteroaryl group containing at least one nitrogen,
any one of R1 to R4 is HAr,
R5 is a substituted or unsubstituted C6-C60 aryl group,
R6 is selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; and a substituted or unsubstituted C2-C60 heterocycloalkyl group,
l is an integer of 1-3, and

m is an integer of 1-4.

In one embodiment of the present invention, the compound of chemical formula 1 may be represented by the following chemical formula 1-a:

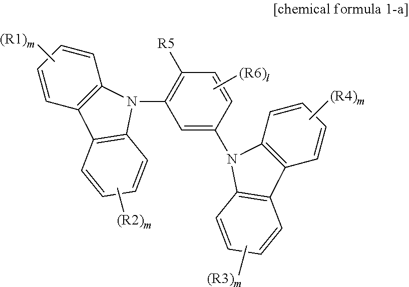

in chemical formula 1-a,
R1 to R6, 1 and m have the same definitions as in chemical formula 1.

In one embodiment of the present invention, R5 may be a substituted or unsubstituted C6-C30 aryl group.

In one embodiment of the present invention, R5 may be a substituted or unsubstituted C6-C20 aryl group.

In one embodiment of the present invention, R5 may be any one selected from among the following structural formulae:

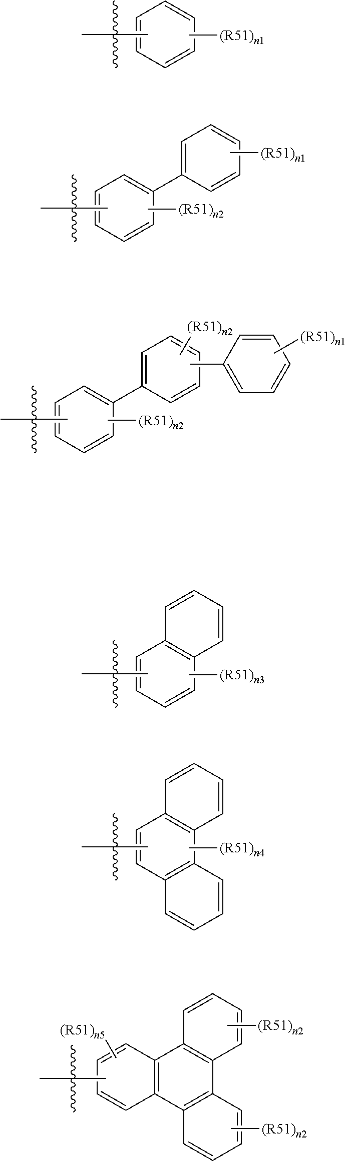

herein,
R51 is selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; and a substituted or unsubstituted C2-C60 heterocycloalkyl group, or two or more groups adjacent to each other bond to each other to form a substituted or unsubstituted C6-C60 aromatic hydrocarbon ring or a substituted or unsubstituted C2-C60 heteroring,
n1 is an integer of 1-5,
n2 is an integer of 1-4,
n3 is an integer of 1-7,
n4 is an integer of 1-9, and
n5 is an integer of 1-3.

In one embodiment of the present invention, R51 is selected from the group consisting of: hydrogen; deuterium; halogen; a cyano group; a substituted or unsubstituted C1-C30 alkyl group; a substituted or unsubstituted C2-C30 alkenyl group; a substituted or unsubstituted C2-C30 alkynyl group; a substituted or unsubstituted C1-C30 alkoxy group; a substituted or unsubstituted C3-C30 cycloalkyl group; and a substituted or unsubstituted C2-C30 heterocycloalkyl group, or two or more groups adjacent to each other may bond to each other to form a substituted or unsubstituted C6-C30 aromatic hydrocarbon ring or a substituted or unsubstituted C2-C30 heteroring.

In one embodiment of the present invention, R51 is selected from the group consisting of: hydrogen; deuterium; halogen; a cyano group; a substituted or unsubstituted C1-C10 alkyl group; a substituted or unsubstituted C2-C10 alkenyl group; a substituted or unsubstituted C2-C10 alkynyl group; a substituted or unsubstituted C1-C10 alkoxy group; a substituted or unsubstituted C3-C10 cycloalkyl group; and a substituted or unsubstituted C2-C10 heterocycloalkyl group, or two or more groups adjacent to each other may bond to each other to form a substituted or unsubstituted C6-C10 aromatic hydrocarbon ring or a substituted or unsubstituted C2-C10 heteroring.

In one embodiment of the present invention, R51 is hydrogen; or deuterium, or two or more groups adjacent to each other may bond to each other to form a substituted or unsubstituted C2-C10 aromatic hydrocarbon ring.

In one embodiment of the present invention, R51 may be hydrogen or deuterium.

In one embodiment of the present invention, R5 may be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, or a substituted or unsubstituted triphenylenyl group.

In one embodiment of the present invention, R5 may be a substituted or unsubstituted phenyl group, a substituted or unsubstituted biphenyl group, or a substituted or unsubstituted naphthyl group.

In the definition of R5, “substituted” in the “substituted or unsubstituted” may be being substituted with hydrogen or deuterium.

In one embodiment of the present invention, R6 may be hydrogen; deuterium; halogen; a cyano group; a substituted or unsubstituted C1-C30 alkyl group; a substituted or unsubstituted C2-C30 alkenyl group; a substituted or unsubstituted C2-C30 alkynyl group; a substituted or unsubstituted C1-C30 alkoxy group; a substituted or unsubstituted C3-C30 cycloalkyl group; or a substituted or unsubstituted C2-C30 heterocycloalkyl group.

In one embodiment of the present invention, R6 may be hydrogen; deuterium; halogen; a cyano group; a substituted or unsubstituted C1-C10 alkyl group; a substituted or unsubstituted C2-C10 alkenyl group; a substituted or unsubstituted C2-C10 alkynyl group; a substituted or unsubstituted C1-C10 alkoxy group; a substituted or unsubstituted C3-C10 cycloalkyl group; or a substituted or unsubstituted C2-C10 heterocycloalkyl group.

In one embodiment of the present invention, R6 may be hydrogen; deuterium; halogen; a cyano group; a substituted or unsubstituted C1-C10 alkyl group; a substituted or unsubstituted C2-C10 alkenyl group; or a substituted or unsubstituted C2-C10 alkynyl group.

In one embodiment of the present invention, R6 may be hydrogen; or deuterium.

In one embodiment of the present invention, I may be an integer of 1, 2 or 3.

In one embodiment of the present invention, R1 to R4 are the same or different, and may be each independently hydrogen; deuterium; halogen; a cyano group; a substituted or unsubstituted C1-C30 alkyl group; a substituted or unsubstituted C2-C30 alkenyl group; a substituted or unsubstituted C2-C30 alkynyl group; a substituted or unsubstituted C1-C30 alkoxy group; a substituted or unsubstituted C3-C30 cycloalkyl group; a substituted or unsubstituted C2-C30 heterocycloalkyl group; or HAr.

In one embodiment of the present invention, R1 to R4 are the same or different, and may be each independently hydrogen; deuterium; halogen; a cyano group; a substituted or unsubstituted C1-C10 alkyl group; a substituted or unsubstituted C2-C10 alkenyl group; a substituted or unsubstituted C2-C10 alkynyl group; a substituted or unsubstituted C1-C10 alkoxy group; a substituted or unsubstituted C3-C10 cycloalkyl group; a substituted or unsubstituted C2-C10 heterocycloalkyl group; or HAr.

In one embodiment of the present invention, R1 to R4 are the same or different, and may be each independently hydrogen; deuterium; halogen; a cyano group; a substituted or unsubstituted C1-C10 alkyl group; a substituted or unsubstituted C2-C10 alkenyl group; a substituted or unsubstituted C2-C10 alkynyl group; or HAr.

In one embodiment of the present invention, R1 to R4 are the same or different, and may be each independently hydrogen; deuterium; or HAr.

In one embodiment of the present invention, m may be an integer of 1, 2, 3 or 4.

In one embodiment of the present invention, HAr may be a substituted or unsubstituted C2-C60 heteroaryl group containing at least one nitrogen.

In one embodiment of the present invention, HAr may be a substituted or unsubstituted C2-C40 heteroaryl group containing at least one nitrogen.

In one embodiment of the present invention, HAr may be a substituted or unsubstituted C2-C30 heteroaryl group containing at least one nitrogen.

In one embodiment of the present invention, HAr may be a substituted or unsubstituted C2-C20 heteroaryl group containing at least one nitrogen.

In one embodiment of the present invention, any one of R1 to R4 may be HAr.

In one embodiment of the present invention, when R1 is HAr, R2 to R4 may be each independently hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; or a substituted or unsubstituted C2-C60 heterocycloalkyl group.

In one embodiment of the present invention, when R2 is HAr, R1, R3 and R4 may be each independently hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; or a substituted or unsubstituted C2-C60 heterocycloalkyl group.

In one embodiment of the present invention, when R3 is HAr, R1, R2 and R4 may be each independently hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; or a substituted or unsubstituted C2-C60 heterocycloalkyl group.

In one embodiment of the present invention, when R4 is HAr, R1 to R3 may be each independently hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; or a substituted or unsubstituted C2-C60 heterocycloalkyl group.

In one embodiment of the present invention, HAr may be represented by the following chemical formula 1-2:

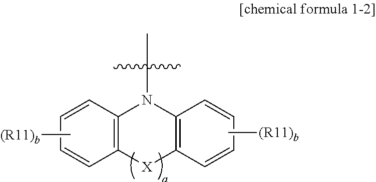

in chemical formula 1-2,
R11s are each independently selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; and a substituted or unsubstituted C2-C60 heterocycloalkyl group, or two or more groups adjacent to each other bond to each other to form a substituted or unsubstituted C6-C60 aromatic hydrocarbon ring or a substituted or unsubstituted C2-C60 heteroring,
X is O or CRaRb,
Ra and Rb are each independently selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; a substituted or unsubstituted C2-C60 heterocycloalkyl group; and a substituted or unsubstituted C6-C60 aryl group,
a is an integer of 0 or 1, and
b is an integer of 1-4.

In one embodiment of the present invention, R11s are each independently selected from the group consisting of: hydrogen; deuterium; halogen; a cyano group; a substituted or unsubstituted C1-C30 alkyl group; a substituted or unsubstituted C2-C30 alkenyl group; a substituted or unsubstituted C2-C30 alkynyl group; a substituted or unsubstituted C1-C30 alkoxy group; a substituted or unsubstituted C3-C30 cycloalkyl group; and a substituted or unsubstituted C2-C30 heterocycloalkyl group, or two or more groups adjacent to each other may bond to each other to form a substituted or unsubstituted C6-C30 aromatic hydrocarbon ring or a substituted or unsubstituted C2-C30 heteroring.

In one embodiment of the present invention, R11s are each independently selected from the group consisting of: hydrogen; deuterium; halogen; a cyano group; a substituted or unsubstituted C1-C10 alkyl group; a substituted or unsubstituted C2-C10 alkenyl group; a substituted or unsubstituted C2-C10 alkynyl group; a substituted or unsubstituted C1-C10 alkoxy group; a substituted or unsubstituted C3-C10 cycloalkyl group; and a substituted or unsubstituted C2-C10 heterocycloalkyl group, or two or more groups adjacent to each other may bond to each other to form a substituted or unsubstituted C6-C20 aromatic hydrocarbon ring or a substituted or unsubstituted C2-C20 heteroring.

In one embodiment of the present invention, R11s are each independently hydrogen; or deuterium, or two or more groups adjacent to each other may bond to each other to form a substituted or unsubstituted C6-C20 aromatic hydrocarbon ring or a substituted or unsubstituted C2-C20 heteroring.

In one embodiment of the present invention, R11s are each independently hydrogen; or deuterium, or two or more groups adjacent to each other may bond to each other to form a substituted or unsubstituted C6-C10 aromatic hydrocarbon ring or a substituted or unsubstituted C2-C10 heteroring.

In one embodiment of the present invention, X may be O or CRaRb.

In one embodiment of the present invention, Ra and Rb may be each independently hydrogen; deuterium; halogen; a cyano group; a substituted or unsubstituted C1-C30 alkyl group; a substituted or unsubstituted C2-C30 alkenyl group; a substituted or unsubstituted C2-C30 alkynyl group; a substituted or unsubstituted C1-C30 alkoxy group; a substituted or unsubstituted C3-C30 cycloalkyl group; a substituted or unsubstituted C2-C30 heterocycloalkyl group; or a substituted or unsubstituted C6-C30 aryl group.

In one embodiment of the present invention, Ra and Rb may be each independently hydrogen; deuterium; halogen; a cyano group; a substituted or unsubstituted C1-C10 alkyl group; a substituted or unsubstituted C2-C10 alkenyl group; a substituted or unsubstituted C2-C10 alkynyl group; a substituted or unsubstituted C1-C10 alkoxy group; a substituted or unsubstituted C3-C10 cycloalkyl group; a substituted or unsubstituted C2-C10 heterocycloalkyl group; or a substituted or unsubstituted C6-C10 aryl group.

In one embodiment of the present invention, Ra and Rb may be each independently hydrogen; deuterium; halogen; a cyano group; a substituted or unsubstituted C1-C10 alkyl group; or a substituted or unsubstituted C6-C10 aryl group.

In one embodiment of the present invention, Ra and Rb may be each independently hydrogen; deuterium; a methyl group; or a phenyl group.

In one embodiment of the present invention, a may be an integer of 0 or 1.

In one embodiment of the present invention, b may be an integer of 1, 2, 3 or 4.

In one embodiment of the present invention, the group represented by chemical formula 1-2 may be represented by any one of the following chemical formula 1-2-a to chemical formula 1-2-e:

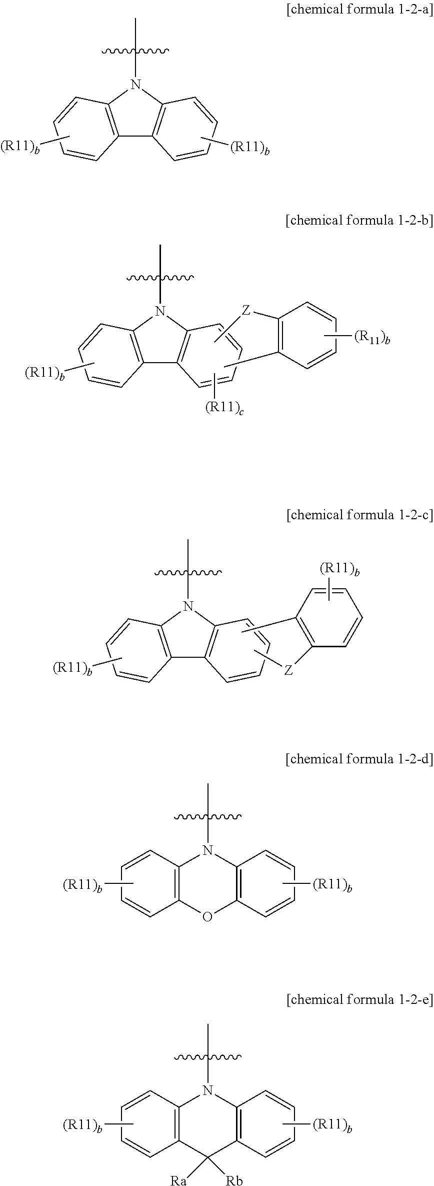

in chemical formulae 1-2-a to 1-2-e,
Z is NRc, O, S or CRdRe,
Rc, Rd and Re are each independently selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; a substituted or unsubstituted C2-C60 heterocycloalkyl group; and a substituted or unsubstituted C6-C60 aryl group, c is an integer of 1 or 2, and
Ra, Rb, R11 and b have the same definitions as in chemical formula 1-2.

In one embodiment of the present invention, Z may be NRc, O, S or CRdRe.

In one embodiment of the present invention, Rc, Rd and Re may be each independently hydrogen; deuterium; halogen; a cyano group; a substituted or unsubstituted C1-C30 alkyl group; a substituted or unsubstituted C2-C30 alkenyl group; a substituted or unsubstituted C2-C30 alkynyl group; a substituted or unsubstituted C1-C30 alkoxy group; a substituted or unsubstituted C3-C30 cycloalkyl group; a substituted or unsubstituted C2-C30 heterocycloalkyl group; or a substituted or unsubstituted C6-C30 aryl group.

In one embodiment of the present invention, Rc, Rd and Re may be each independently hydrogen; deuterium; halogen; a cyano group; a substituted or unsubstituted C1-C10 alkyl group; a substituted or unsubstituted C2-C10 alkenyl group; a substituted or unsubstituted C2-C10 alkynyl group; a substituted or unsubstituted C1-C10 alkoxy group; a substituted or unsubstituted C3-C10 cycloalkyl group; a substituted or unsubstituted C2-C10 heterocycloalkyl group; or a substituted or unsubstituted C6-C10 aryl group.

In one embodiment of the present invention, Rc, Rd and Re may be each independently hydrogen; deuterium; a substituted or unsubstituted C1-C10 alkyl group; or a substituted or unsubstituted C6-C10 aryl group.

In one embodiment of the present invention, Rc, Rd and Re may be each independently hydrogen; deuterium; a methyl group; or a phenyl group.

In one embodiment of the present invention, c may be an integer of 1 or 2.

In one embodiment of the present invention, the groups represented by chemical formulae 1-2-b and 1-2-c may respectively be represented by any one of the following chemical formulae 1-2-b-1 to 1-2-b-3 and the following chemical formulae 1-2-c-1 to 1-2-c-3:

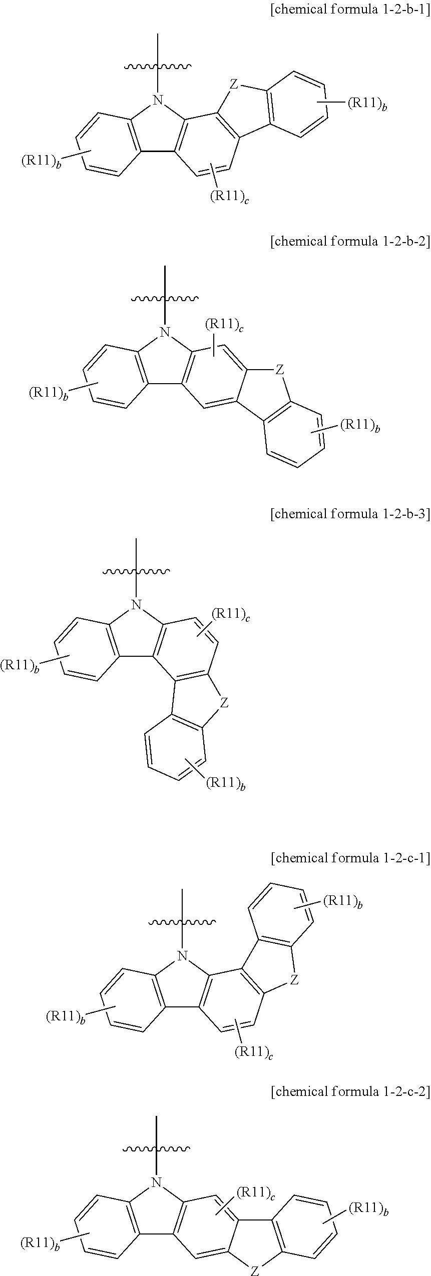

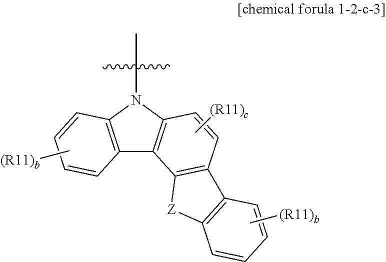

in chemical formulae 1-2-b-1 to 1-2-b-3 and chemical formulae 1-2-c-1 to 1-2-c-3, R11, Z, b and c have the same definitions as in chemical formulae 1-2-b and 1-2-c.

In one embodiment of the present invention, the heterocyclic compound represented by chemical formula 1 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In one embodiment of the present invention, the heterocyclic compound represented by chemical formula 1 may not include deuterium, or may have a deuterium content of 1% to 100% based on the total number of hydrogen atoms and deuterium atoms.

In one embodiment of the present invention, the heterocyclic compound represented by chemical formula 1 may not include deuterium, or may have a deuterium content of 20% to 90% based on the total number of hydrogen atoms and deuterium atoms.

In one embodiment of the present invention, the heterocyclic compound represented by chemical formula 1 may not include deuterium, or may have a deuterium content of 30% to 80% based on the total number of hydrogen atoms and deuterium atoms.

In one embodiment of the present invention, the heterocyclic compound represented by chemical formula 1 may not include deuterium, or may have a deuterium content of 40% to 70% based on the total number of hydrogen atoms and deuterium atoms.

For example, the deuterium content in the heterocyclic compound represented by chemical formula 1 may be 0% or greater, 1% or greater, 5% or greater, 10% or greater, 15% or greater, 20% or greater, 25% or greater, 30% or greater, 35% or greater, 40% or greater, 45% or greater or 50% or greater, and 100% or less, 95% or less, 90% or less, 85% or less, 80% or less, 75% or less, 70% or less, 65% or less or 60% or less based on the total number of hydrogen atoms and deuterium atoms.

In one embodiment of the present invention, the heterocyclic compound represented by chemical formula 1 may be any one selected from the group consisting of the following compounds:

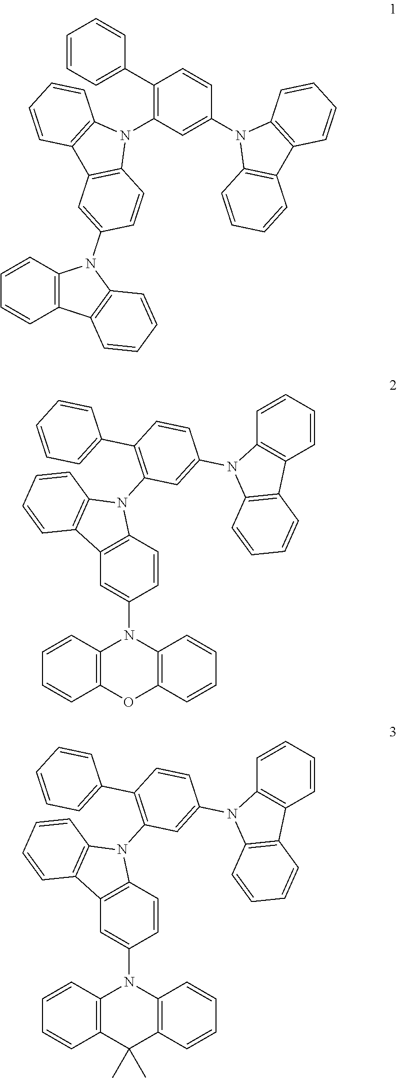 [SMILES: c1ccc(-c2ccc(-n3c4ccccc4c4ccccc43)cc2-n2c3ccccc3c3cc(-n4c5ccccc5c5ccccc54)ccc32)cc1 ; c1ccc(-c2ccc(-n3c4ccccc4c4ccccc43)cc2-n2c3ccccc3c3cc(N4c5ccccc5Oc5ccccc54)ccc32)cc1 ; CC1(C)c2ccccc2N(c2ccc3c(c2)c2ccccc2n3-c2cc(-n3c4ccccc4c4ccccc43)ccc2-c2ccccc2)c2ccccc21]

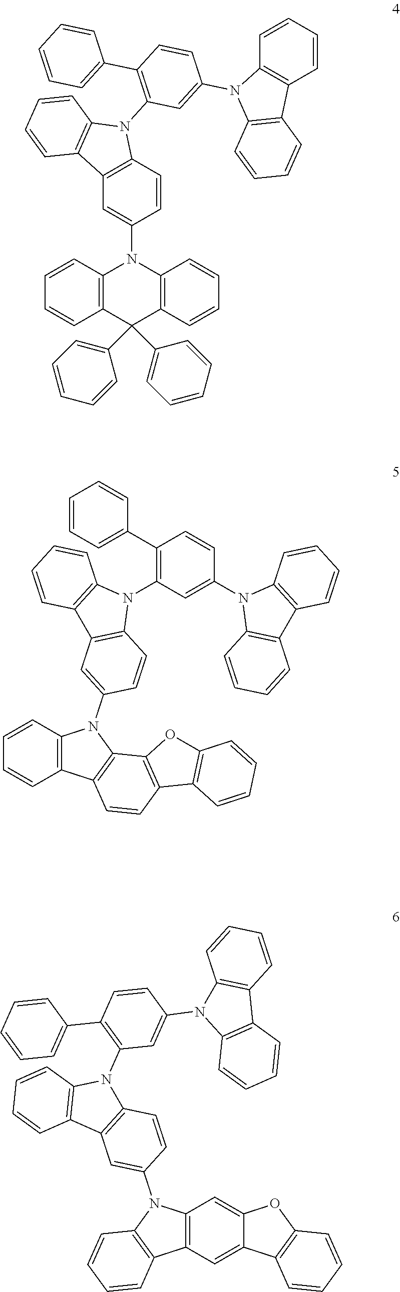 [SMILES: c1ccc(-c2ccc(-n3c4ccccc4c4ccccc43)cc2-n2c3ccccc3c3cc(N4c5ccccc5C(c5ccccc5)(c5ccccc5)c5ccccc54)ccc32)cc1 ; c1ccc(-c2ccc(-n3c4ccccc4c4ccccc43)cc2-n2c3ccccc3c3cc(-n4c5ccccc5c5ccc6c7ccccc7oc6c54)ccc32)cc1 ; c1ccc(-c2ccc(-n3c4ccccc4c4ccccc43)cc2-n2c3ccccc3c3cc(-n4c5ccccc5c5cc6c(cc54)oc4ccccc46)ccc32)cc1]

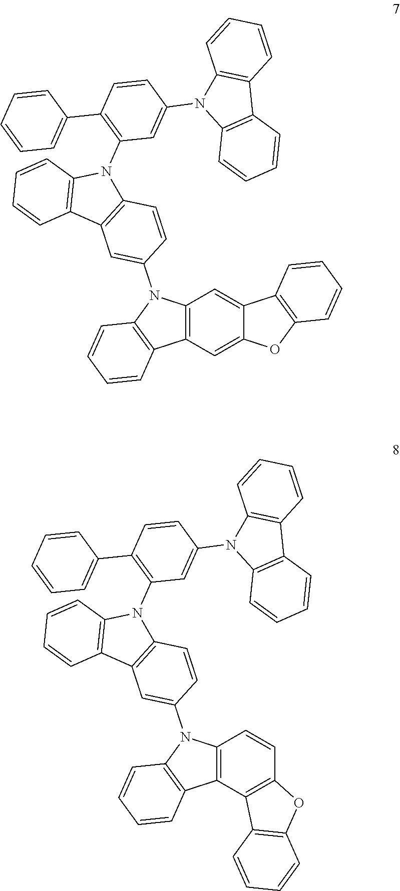 [SMILES: c1ccc(-c2ccc(-n3c4ccccc4c4ccccc43)cc2-n2c3ccccc3c3cc(-n4c5ccccc5c5cc6oc7ccccc7c6cc54)ccc32)cc1 ; c1ccc(-c2ccc(-n3c4ccccc4c4ccccc43)cc2-n2c3ccccc3c3cc(-n4c5ccccc5c5c6c(ccc54)oc4ccccc46)ccc32)cc1]

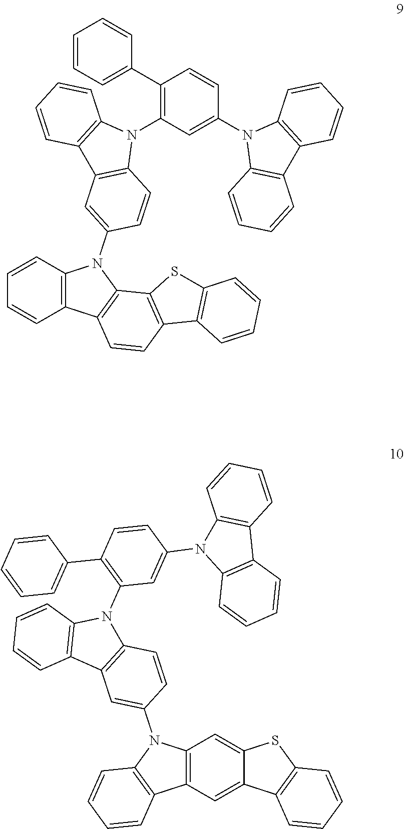 [SMILES: c1ccc(-c2ccc(-n3c4ccccc4c4ccccc43)cc2-n2c3ccccc3c3cc(-n4c5ccccc5c5ccc6c7ccccc7sc6c54)ccc32)cc1 ; c1ccc(-c2ccc(-n3c4ccccc4c4ccccc43)cc2-n2c3ccccc3c3cc(-n4c5ccccc5c5cc6c(cc54)sc4ccccc46)ccc32)cc1]

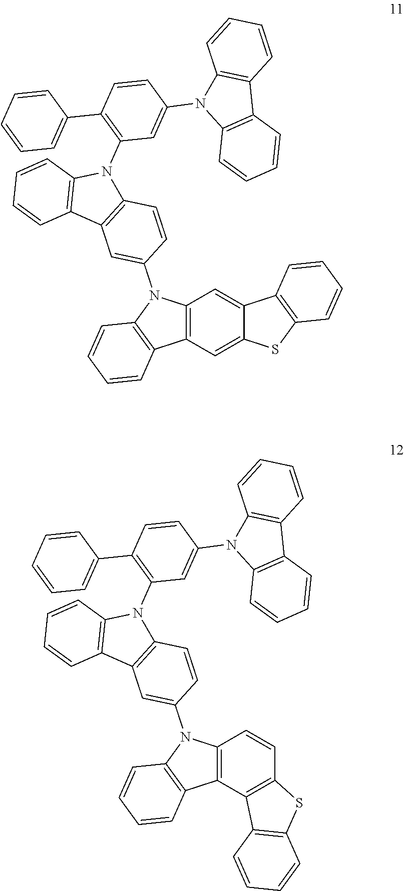 [SMILES: c1ccc(-c2ccc(-n3c4ccccc4c4ccccc43)cc2-n2c3ccccc3c3cc(-n4c5ccccc5c5cc6sc7ccccc7c6cc54)ccc32)cc1 ; c1ccc(-c2ccc(-n3c4ccccc4c4ccccc43)cc2-n2c3ccccc3c3cc(-n4c5ccccc5c5c6c(ccc54)sc4ccccc46)ccc32)cc1]

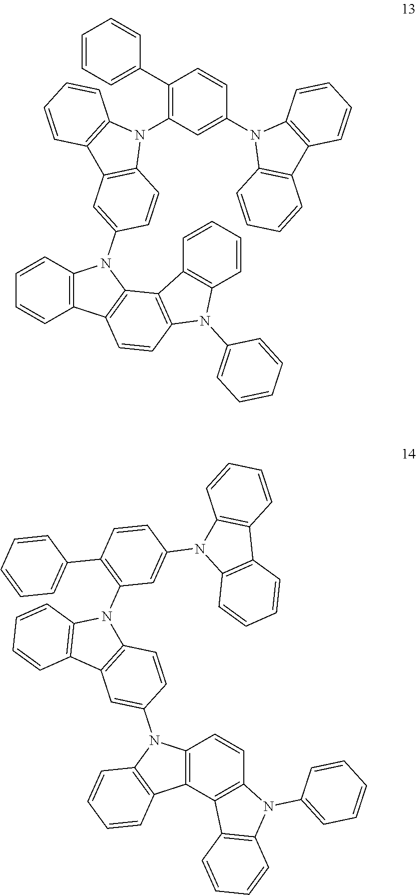 [SMILES: c1ccc(-c2ccc(-n3c4ccccc4c4ccccc43)cc2-n2c3ccccc3c3cc(-n4c5ccccc5c5ccc6c(c7ccccc7n6-c6ccccc6)c54)ccc32)cc1 ; c1ccc(-c2ccc(-n3c4ccccc4c4ccccc43)cc2-n2c3ccccc3c3cc(-n4c5ccccc5c5c6c7ccccc7n(-c7ccccc7)c6ccc54)ccc32)cc1]

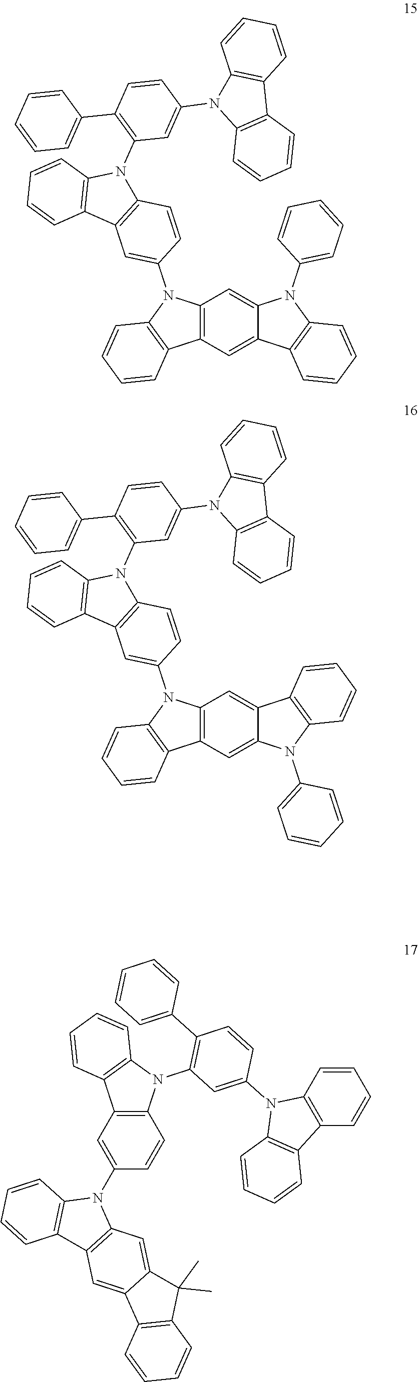 [SMILES: c1ccc(-c2ccc(-n3c4ccccc4c4ccccc43)cc2-n2c3ccccc3c3cc(-n4c5ccccc5c5cc6c7ccccc7n(-c7ccccc7)c6cc54)ccc32)cc1 ; c1ccc(-c2ccc(-n3c4ccccc4c4ccccc43)cc2-n2c3ccccc3c3cc(-n4c5ccccc5c5cc6c(cc54)c4ccccc4n6-c4ccccc4)ccc32)cc1 ; CC1(C)c2ccccc2-c2cc3c4ccccc4n(-c4ccc5c(c4)c4ccccc4n5-c4cc(-n5c6ccccc6c6ccccc65)ccc4-c4ccccc4)c3cc21]

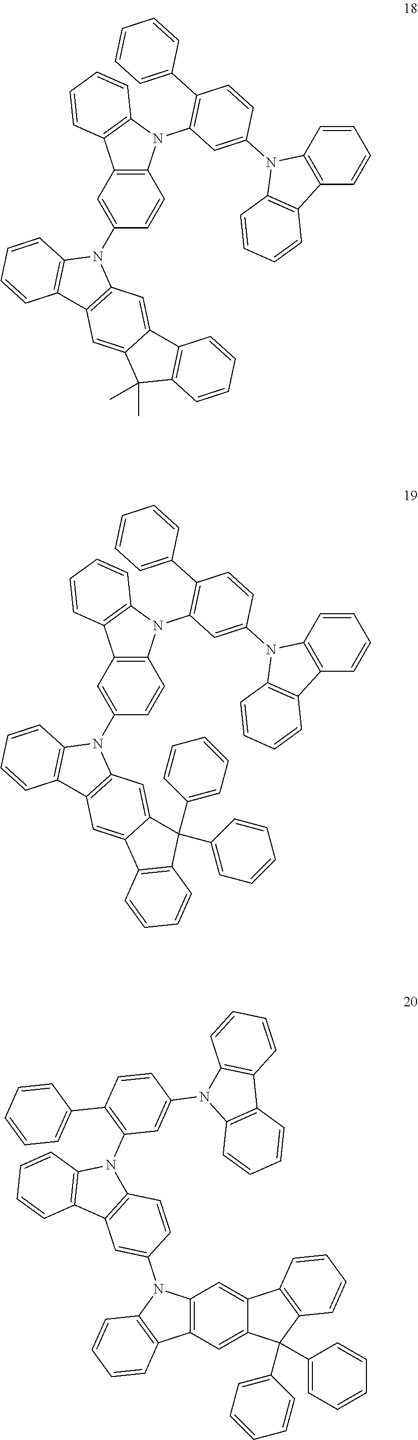 [SMILES: CC1(C)c2ccccc2-c2cc3c(cc21)c1ccccc1n3-c1ccc2c(c1)c1ccccc1n2-c1cc(-n2c3ccccc3c3ccccc32)ccc1-c1ccccc1 ; c1ccc(-c2ccc(-n3c4ccccc4c4ccccc43)cc2-n2c3ccccc3c3cc(-n4c5ccccc5c5cc6c(cc54)C(c4ccccc4)(c4ccccc4)c4ccccc4-6)ccc32)cc1 ; c1ccc(-c2ccc(-n3c4ccccc4c4ccccc43)cc2-n2c3ccccc3c3cc(-n4c5ccccc5c5cc6c(cc54)-c4ccccc4C6(c4ccccc4)c4ccccc4)ccc32)cc1]

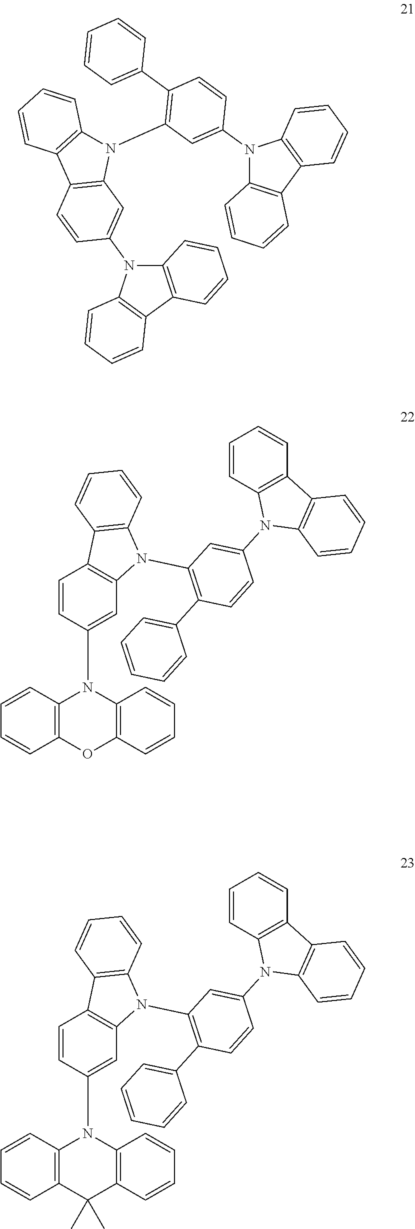 [SMILES: c1ccc(-c2ccc(-n3c4ccccc4c4ccccc43)cc2-n2c3ccccc3c3ccc(-n4c5ccccc5c5ccccc54)cc32)cc1 ; c1ccc(-c2ccc(-n3c4ccccc4c4ccccc43)cc2-n2c3ccccc3c3ccc(N4c5ccccc5Oc5ccccc54)cc32)cc1 ; CC1(C)c2ccccc2N(c2ccc3c4ccccc4n(-c4cc(-n5c6ccccc6c6ccccc65)ccc4-c4ccccc4)c3c2)c2ccccc21]

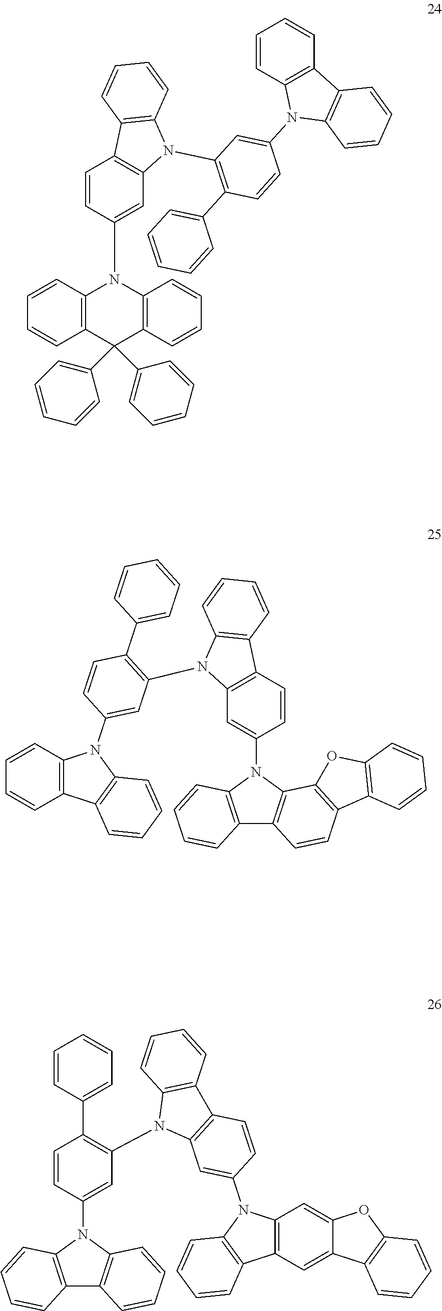 [SMILES: c1ccc(-c2ccc(-n3c4ccccc4c4ccccc43)cc2-n2c3ccccc3c3ccc(N4c5ccccc5C(c5ccccc5)(c5ccccc5)c5ccccc54)cc32)cc1 ; c1ccc(-c2ccc(-n3c4ccccc4c4ccccc43)cc2-n2c3ccccc3c3ccc(-n4c5ccccc5c5ccc6c7ccccc7oc6c54)cc32)cc1 ; c1ccc(-c2ccc(-n3c4ccccc4c4ccccc43)cc2-n2c3ccccc3c3ccc(-n4c5ccccc5c5cc6c(cc54)oc4ccccc46)cc32)cc1]

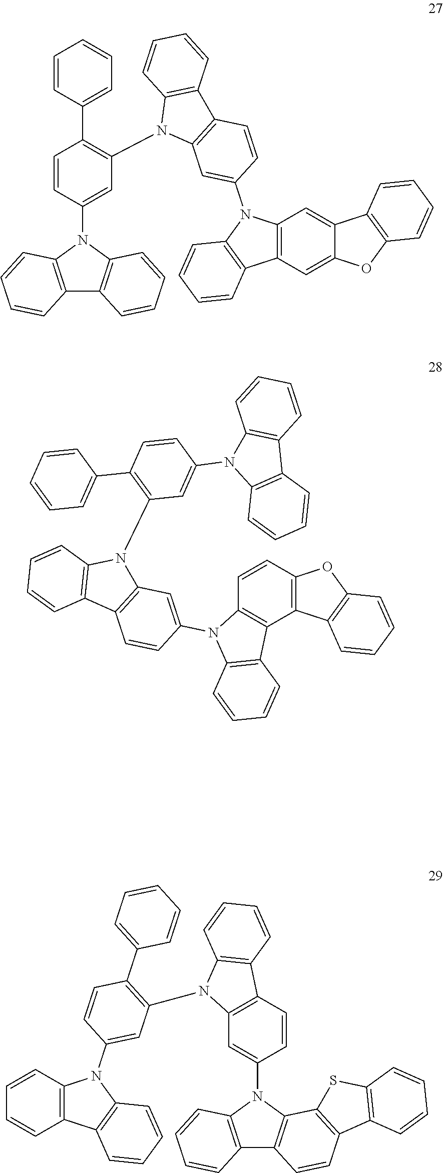 [SMILES: c1ccc(-c2ccc(-n3c4ccccc4c4ccccc43)cc2-n2c3ccccc3c3ccc(-n4c5ccccc5c5cc6oc7ccccc7c6cc54)cc32)cc1 ; c1ccc(-c2ccc(-n3c4ccccc4c4ccccc43)cc2-n2c3ccccc3c3ccc(-n4c5ccccc5c5c6c(ccc54)oc4ccccc46)cc32)cc1 ; c1ccc(-c2ccc(-n3c4ccccc4c4ccccc43)cc2-n2c3ccccc3c3ccc(-n4c5ccccc5c5ccc6c7ccccc7sc6c54)cc32)cc1]

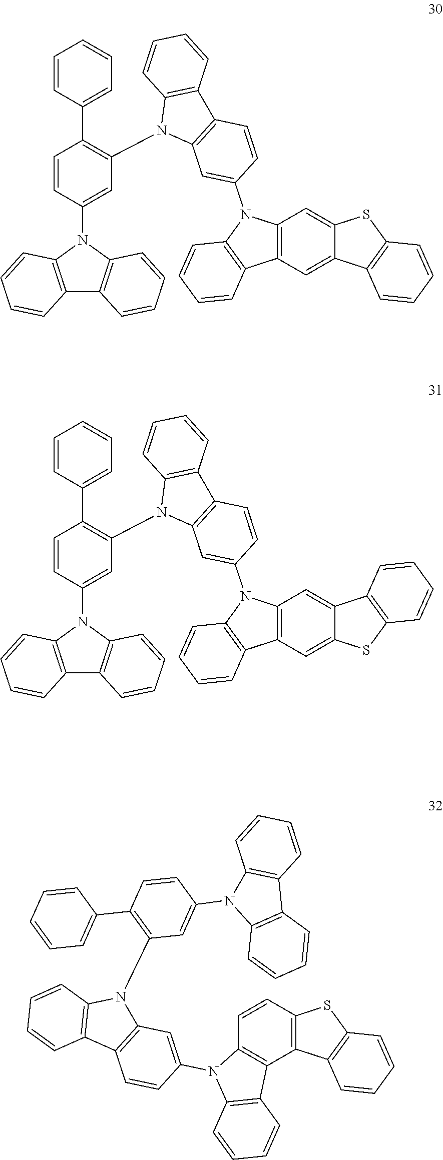 [SMILES: c1ccc(-c2ccc(-n3c4ccccc4c4ccccc43)cc2-n2c3ccccc3c3ccc(-n4c5ccccc5c5cc6c(cc54)sc4ccccc46)cc32)cc1 ; c1ccc(-c2ccc(-n3c4ccccc4c4ccccc43)cc2-n2c3ccccc3c3ccc(-n4c5ccccc5c5cc6sc7ccccc7c6cc54)cc32)cc1 ; c1ccc(-c2ccc(-n3c4ccccc4c4ccccc43)cc2-n2c3ccccc3c3ccc(-n4c5ccccc5c5c6c(ccc54)sc4ccccc46)cc32)cc1]

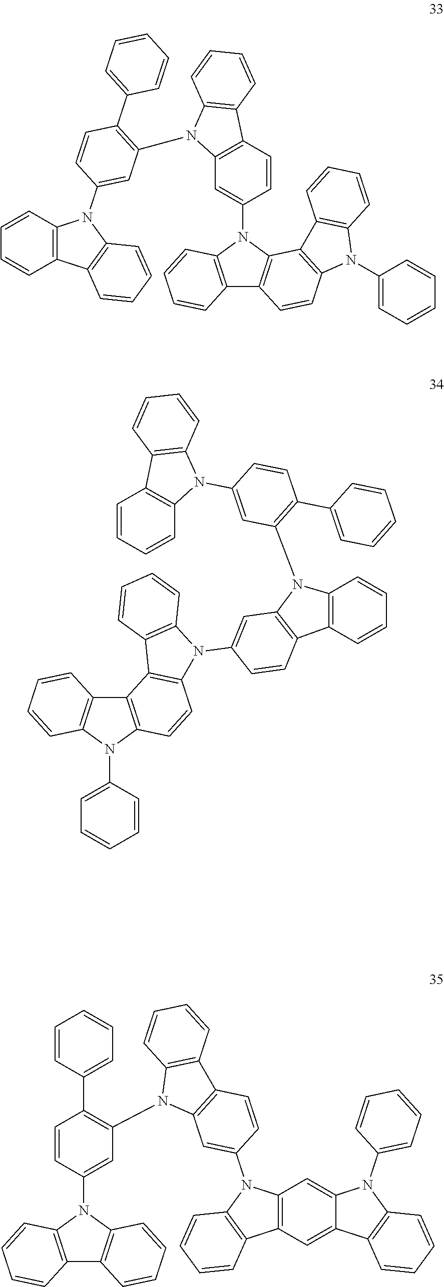 [SMILES: c1ccc(-c2ccc(-n3c4ccccc4c4ccccc43)cc2-n2c3ccccc3c3ccc(-n4c5ccccc5c5ccc6c(c7ccccc7n6-c6ccccc6)c54)cc32)cc1 ; c1ccc(-c2ccc(-n3c4ccccc4c4ccccc43)cc2-n2c3ccccc3c3ccc(-n4c5ccccc5c5c6c7ccccc7n(-c7ccccc7)c6ccc54)cc32)cc1 ; c1ccc(-c2ccc(-n3c4ccccc4c4ccccc43)cc2-n2c3ccccc3c3ccc(-n4c5ccccc5c5cc6c7ccccc7n(-c7ccccc7)c6cc54)cc32)cc1]

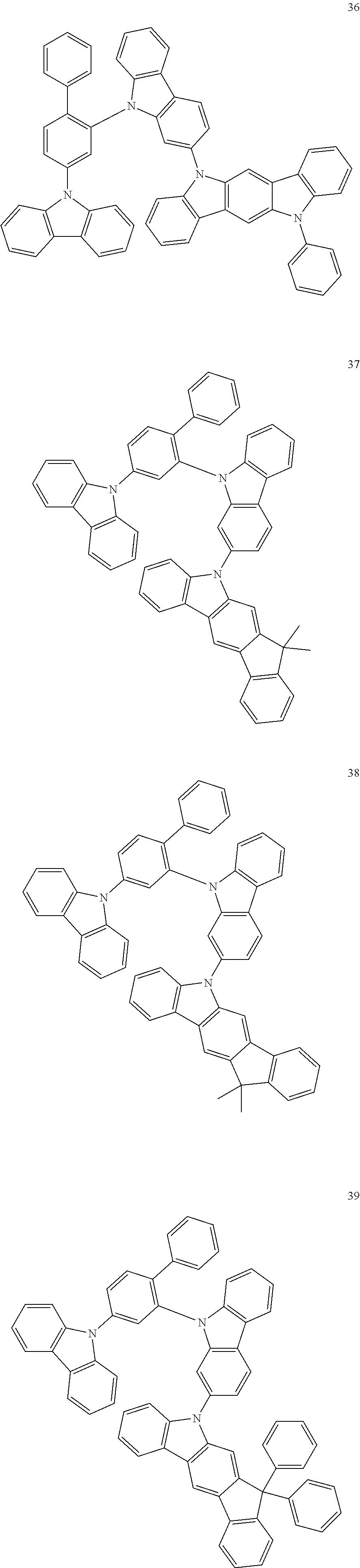 [SMILES: c1ccc(-c2ccc(-n3c4ccccc4c4ccccc43)cc2-n2c3ccccc3c3ccc(-n4c5ccccc5c5cc6c(cc54)c4ccccc4n6-c4ccccc4)cc32)cc1 ; CC1(C)c2ccccc2-c2cc3c4ccccc4n(-c4ccc5c6ccccc6n(-c6cc(-n7c8ccccc8c8ccccc87)ccc6-c6ccccc6)c5c4)c3cc21 ; CC1(C)c2ccccc2-c2cc3c(cc21)c1ccccc1n3-c1ccc2c3ccccc3n(-c3cc(-n4c5ccccc5c5ccccc54)ccc3-c3ccccc3)c2c1 ; c1ccc(-c2ccc(-n3c4ccccc4c4ccccc43)cc2-n2c3ccccc3c3ccc(-n4c5ccccc5c5cc6c(cc54)C(c4ccccc4)(c4ccccc4)c4ccccc4-6)cc32)cc1]

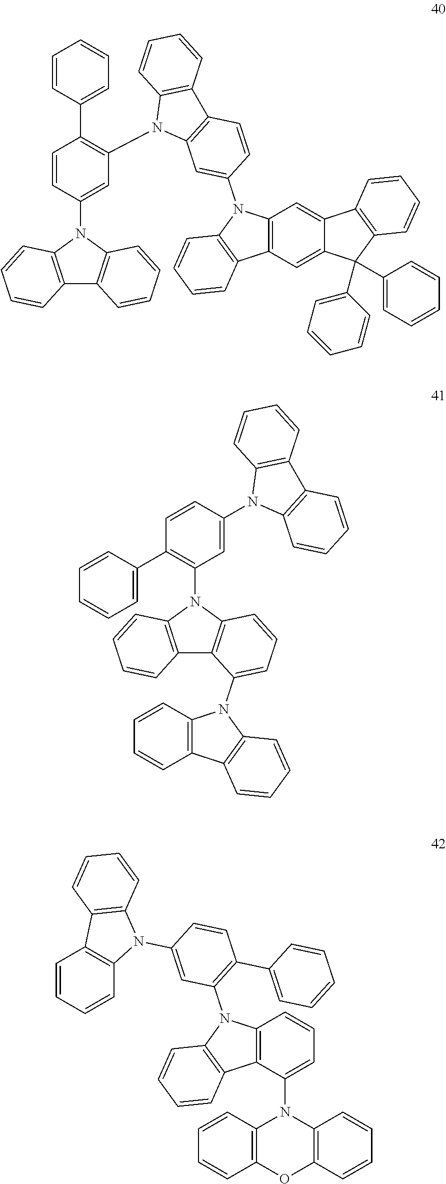 [SMILES: c1ccc(-c2ccc(-n3c4ccccc4c4ccccc43)cc2-n2c3ccccc3c3ccc(-n4c5ccccc5c5cc6c(cc54)-c4ccccc4C6(c4ccccc4)c4ccccc4)cc32)cc1 ; c1ccc(-c2ccc(-n3c4ccccc4c4ccccc43)cc2-n2c3ccccc3c3c(-n4c5ccccc5c5ccccc54)cccc32)cc1 ; c1ccc(-c2ccc(-n3c4ccccc4c4ccccc43)cc2-n2c3ccccc3c3c(N4c5ccccc5Oc5ccccc54)cccc32)cc1]

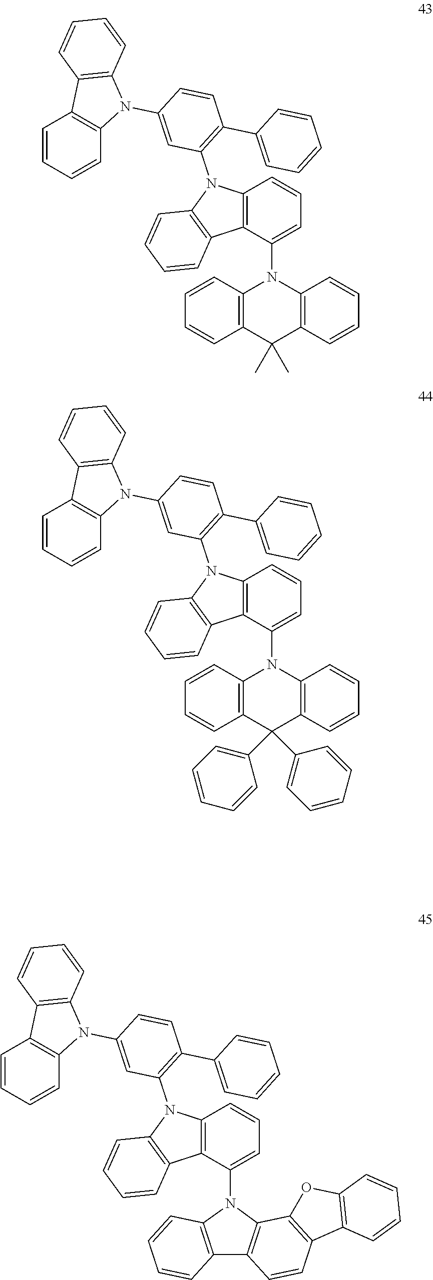 [SMILES: CC1(C)c2ccccc2N(c2cccc3c2c2ccccc2n3-c2cc(-n3c4ccccc4c4ccccc43)ccc2-c2ccccc2)c2ccccc21 ; c1ccc(-c2ccc(-n3c4ccccc4c4ccccc43)cc2-n2c3ccccc3c3c(N4c5ccccc5C(c5ccccc5)(c5ccccc5)c5ccccc54)cccc32)cc1 ; c1ccc(-c2ccc(-n3c4ccccc4c4ccccc43)cc2-n2c3ccccc3c3c(-n4c5ccccc5c5ccc6c7ccccc7oc6c54)cccc32)cc1]

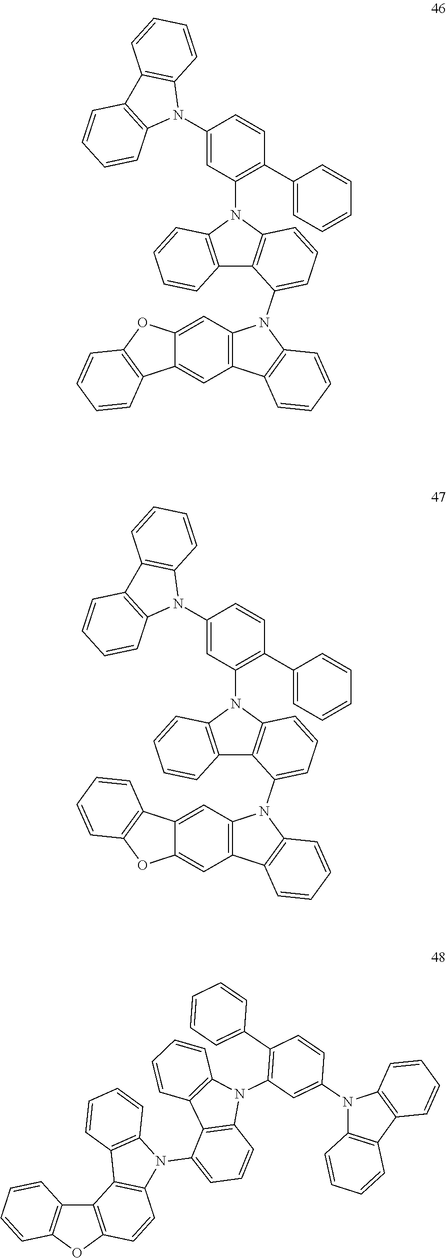 [SMILES: c1ccc(-c2ccc(-n3c4ccccc4c4ccccc43)cc2-n2c3ccccc3c3c(-n4c5ccccc5c5cc6c(cc54)oc4ccccc46)cccc32)cc1 ; c1ccc(-c2ccc(-n3c4ccccc4c4ccccc43)cc2-n2c3ccccc3c3c(-n4c5ccccc5c5cc6oc7ccccc7c6cc54)cccc32)cc1 ; c1ccc(-c2ccc(-n3c4ccccc4c4ccccc43)cc2-n2c3ccccc3c3c(-n4c5ccccc5c5c6c(ccc54)oc4ccccc46)cccc32)cc1]

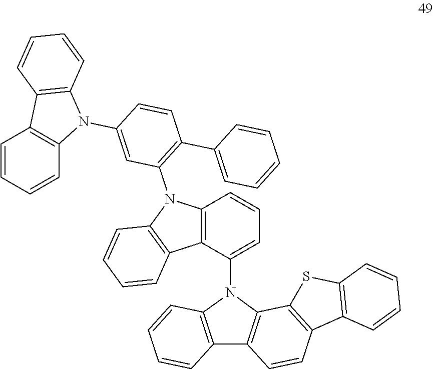 [SMILES: c1ccc(-c2ccc(-n3c4ccccc4c4ccccc43)cc2-n2c3ccccc3c3c(-n4c5ccccc5c5ccc6c7ccccc7sc6c54)cccc32)cc1]

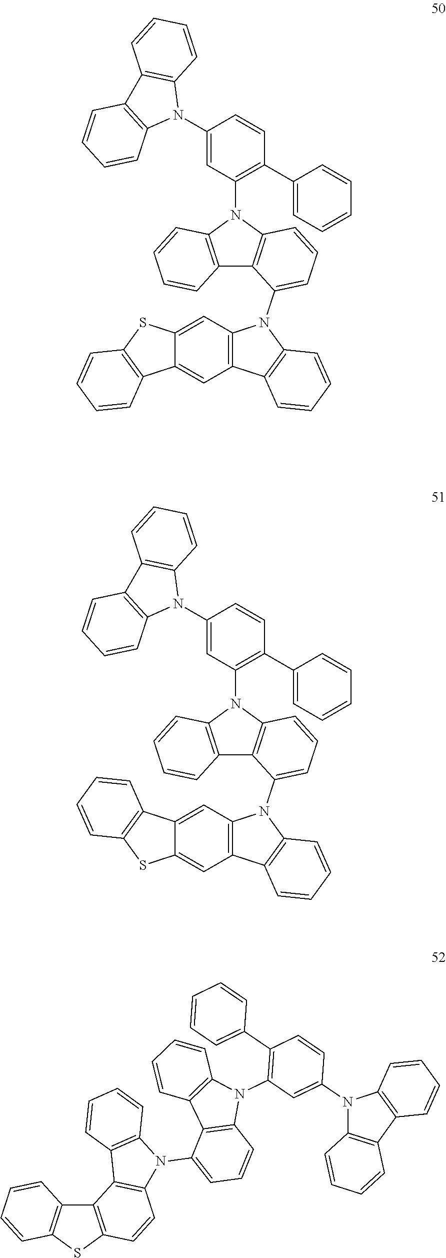 [SMILES: c1ccc(-c2ccc(-n3c4ccccc4c4ccccc43)cc2-n2c3ccccc3c3c(-n4c5ccccc5c5cc6c(cc54)sc4ccccc46)cccc32)cc1 ; c1ccc(-c2ccc(-n3c4ccccc4c4ccccc43)cc2-n2c3ccccc3c3c(-n4c5ccccc5c5cc6sc7ccccc7c6cc54)cccc32)cc1 ; c1ccc(-c2ccc(-n3c4ccccc4c4ccccc43)cc2-n2c3ccccc3c3c(-n4c5ccccc5c5c6c(ccc54)sc4ccccc46)cccc32)cc1]

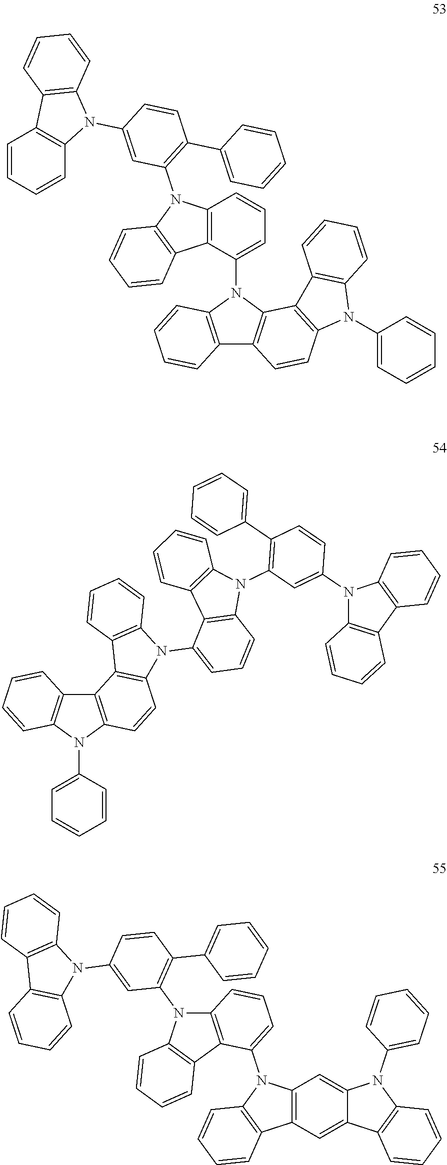 [SMILES: c1ccc(-c2ccc(-n3c4ccccc4c4ccccc43)cc2-n2c3ccccc3c3c(-n4c5ccccc5c5ccc6c(c7ccccc7n6-c6ccccc6)c54)cccc32)cc1 ; c1ccc(-c2ccc(-n3c4ccccc4c4ccccc43)cc2-n2c3ccccc3c3c(-n4c5ccccc5c5c6c7ccccc7n(-c7ccccc7)c6ccc54)cccc32)cc1 ; c1ccc(-c2ccc(-n3c4ccccc4c4ccccc43)cc2-n2c3ccccc3c3c(-n4c5ccccc5c5cc6c7ccccc7n(-c7ccccc7)c6cc54)cccc32)cc1]

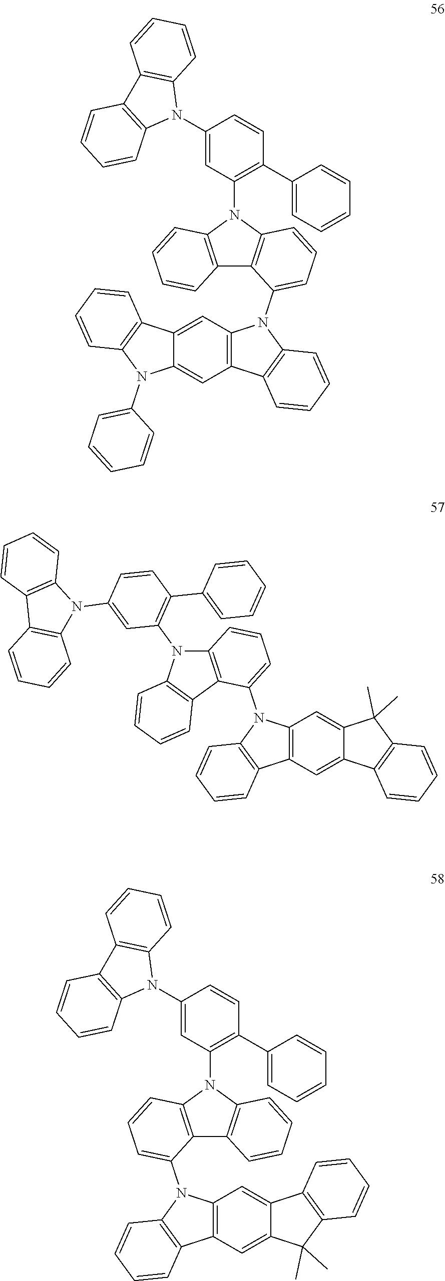 [SMILES: c1ccc(-c2ccc(-n3c4ccccc4c4ccccc43)cc2-n2c3ccccc3c3c(-n4c5ccccc5c5cc6c(cc54)c4ccccc4n6-c4ccccc4)cccc32)cc1 ; CC1(C)c2ccccc2-c2cc3c4ccccc4n(-c4cccc5c4c4ccccc4n5-c4cc(-n5c6ccccc6c6ccccc65)ccc4-c4ccccc4)c3cc21 ; CC1(C)c2ccccc2-c2cc3c(cc21)c1ccccc1n3-c1cccc2c1c1ccccc1n2-c1cc(-n2c3ccccc3c3ccccc32)ccc1-c1ccccc1]

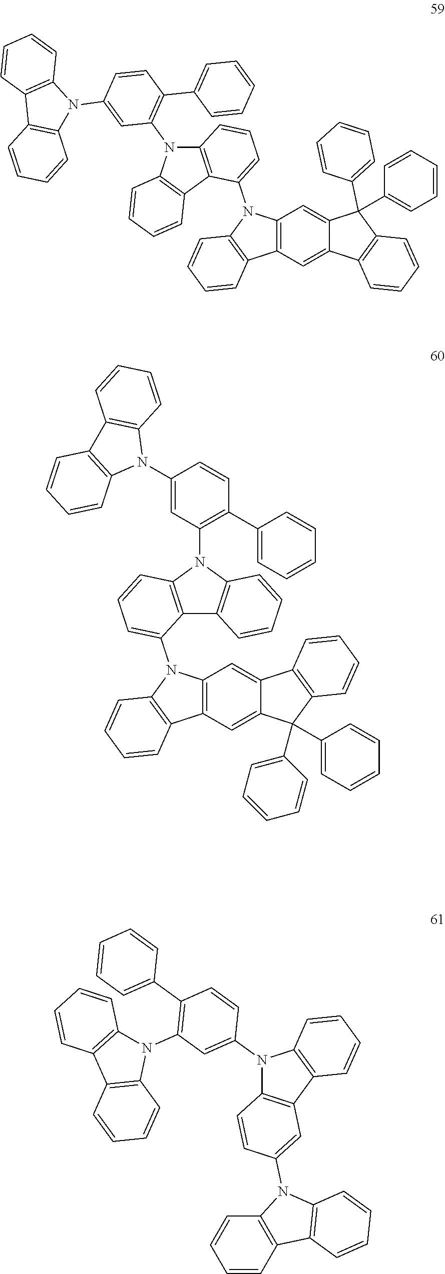 [SMILES: c1ccc(-c2ccc(-n3c4ccccc4c4ccccc43)cc2-n2c3ccccc3c3c(-n4c5ccccc5c5cc6c(cc54)C(c4ccccc4)(c4ccccc4)c4ccccc4-6)cccc32)cc1 ; c1ccc(-c2ccc(-n3c4ccccc4c4ccccc43)cc2-n2c3ccccc3c3c(-n4c5ccccc5c5cc6c(cc54)-c4ccccc4C6(c4ccccc4)c4ccccc4)cccc32)cc1 ; c1ccc(-c2ccc(-n3c4ccccc4c4cc(-n5c6ccccc6c6ccccc65)ccc43)cc2-n2c3ccccc3c3ccccc32)cc1]

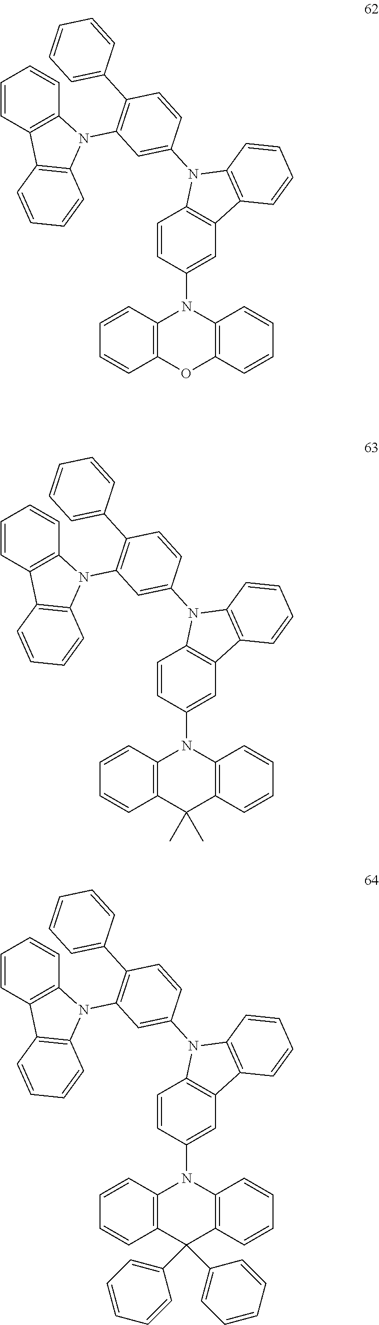 [SMILES: c1ccc(-c2ccc(-n3c4ccccc4c4cc(N5c6ccccc6Oc6ccccc65)ccc43)cc2-n2c3ccccc3c3ccccc32)cc1 ; CC1(C)c2ccccc2N(c2ccc3c(c2)c2ccccc2n3-c2ccc(-c3ccccc3)c(-n3c4ccccc4c4ccccc43)c2)c2ccccc21 ; c1ccc(-c2ccc(-n3c4ccccc4c4cc(N5c6ccccc6C(c6ccccc6)(c6ccccc6)c6ccccc65)ccc43)cc2-n2c3ccccc3c3ccccc32)cc1]

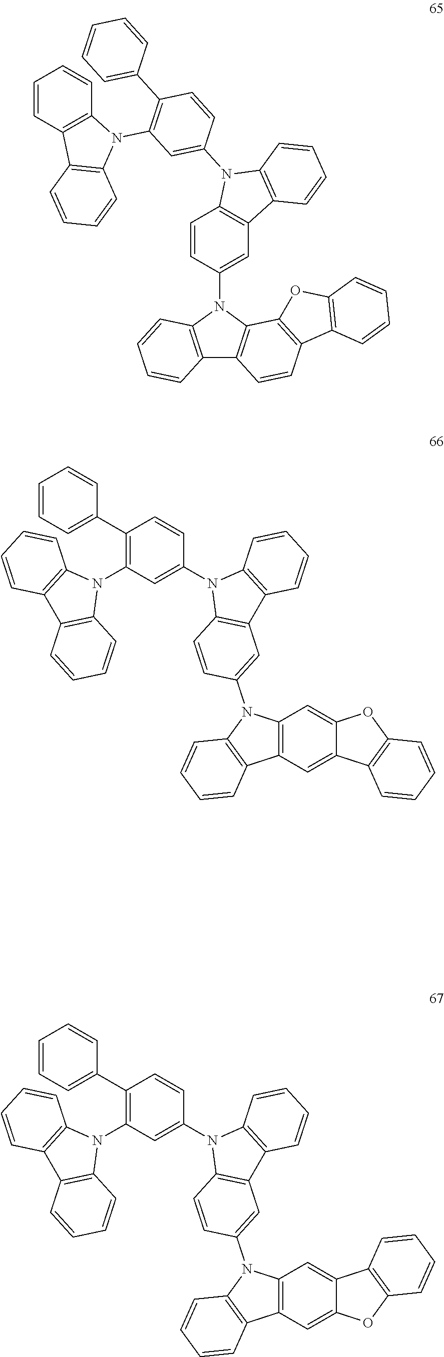 [SMILES: c1ccc(-c2ccc(-n3c4ccccc4c4cc(-n5c6ccccc6c6ccc7c8ccccc8oc7c65)ccc43)cc2-n2c3ccccc3c3ccccc32)cc1 ; c1ccc(-c2ccc(-n3c4ccccc4c4cc(-n5c6ccccc6c6cc7c(cc65)oc5ccccc57)ccc43)cc2-n2c3ccccc3c3ccccc32)cc1 ; c1ccc(-c2ccc(-n3c4ccccc4c4cc(-n5c6ccccc6c6cc7oc8ccccc8c7cc65)ccc43)cc2-n2c3ccccc3c3ccccc32)cc1]

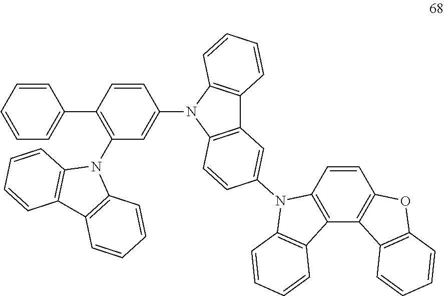 [SMILES: c1ccc(-c2ccc(-n3c4ccccc4c4cc(-n5c6ccccc6c6c7c(ccc65)oc5ccccc57)ccc43)cc2-n2c3ccccc3c3ccccc32)cc1]

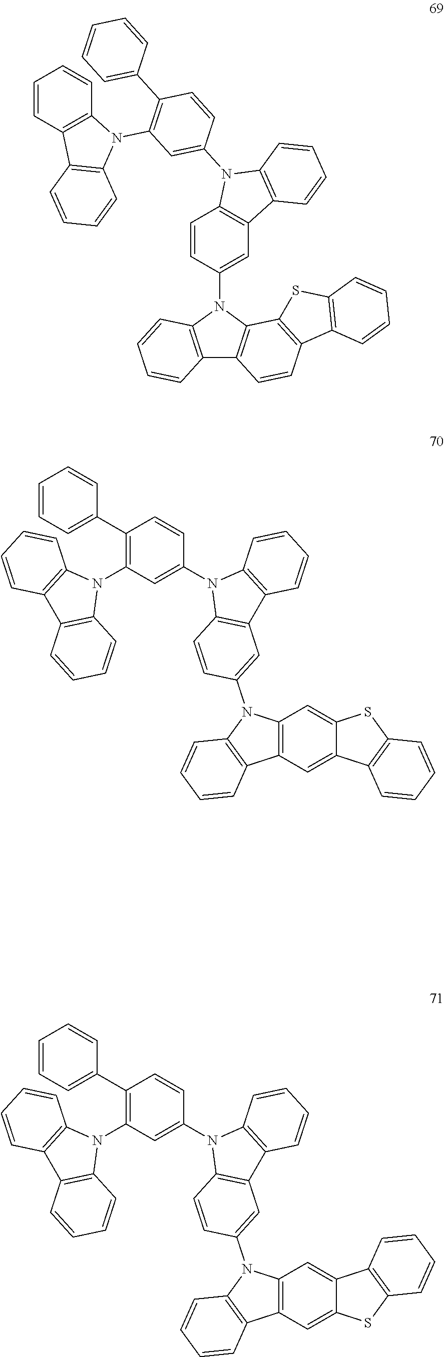 [SMILES: c1ccc(-c2ccc(-n3c4ccccc4c4cc(-n5c6ccccc6c6ccc7c8ccccc8sc7c65)ccc43)cc2-n2c3ccccc3c3ccccc32)cc1 ; c1ccc(-c2ccc(-n3c4ccccc4c4cc(-n5c6ccccc6c6cc7c(cc65)sc5ccccc57)ccc43)cc2-n2c3ccccc3c3ccccc32)cc1 ; c1ccc(-c2ccc(-n3c4ccccc4c4cc(-n5c6ccccc6c6cc7sc8ccccc8c7cc65)ccc43)cc2-n2c3ccccc3c3ccccc32)cc1]

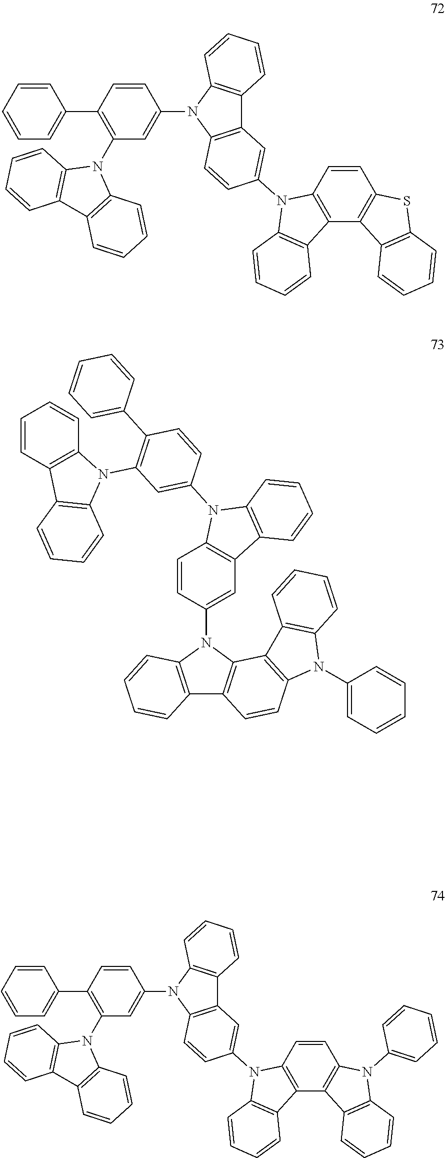 [SMILES: c1ccc(-c2ccc(-n3c4ccccc4c4cc(-n5c6ccccc6c6c7c(ccc65)sc5ccccc57)ccc43)cc2-n2c3ccccc3c3ccccc32)cc1 ; c1ccc(-c2ccc(-n3c4ccccc4c4cc(-n5c6ccccc6c6ccc7c(c8ccccc8n7-c7ccccc7)c65)ccc43)cc2-n2c3ccccc3c3ccccc32)cc1 ; c1ccc(-c2ccc(-n3c4ccccc4c4cc(-n5c6ccccc6c6c7c8ccccc8n(-c8ccccc8)c7ccc65)ccc43)cc2-n2c3ccccc3c3ccccc32)cc1]

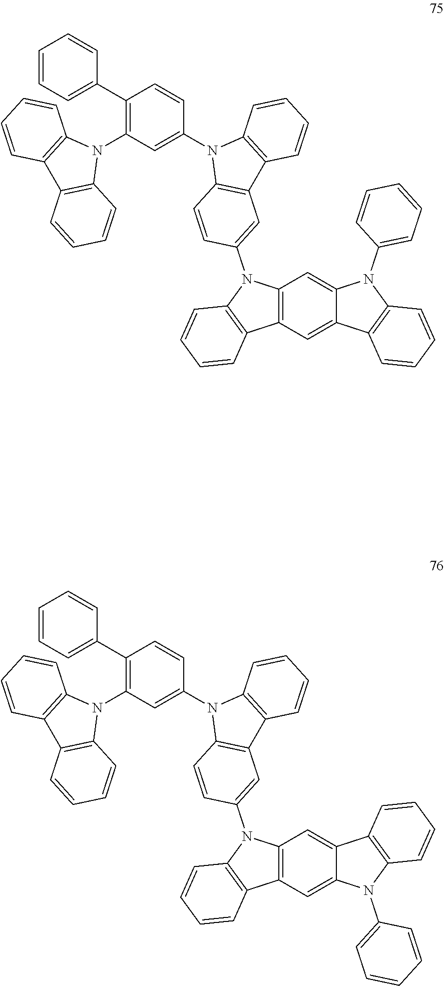 [SMILES: c1ccc(-c2ccc(-n3c4ccccc4c4cc(-n5c6ccccc6c6cc7c8ccccc8n(-c8ccccc8)c7cc65)ccc43)cc2-n2c3ccccc3c3ccccc32)cc1 ; c1ccc(-c2ccc(-n3c4ccccc4c4cc(-n5c6ccccc6c6cc7c(cc65)c5ccccc5n7-c5ccccc5)ccc43)cc2-n2c3ccccc3c3ccccc32)cc1]

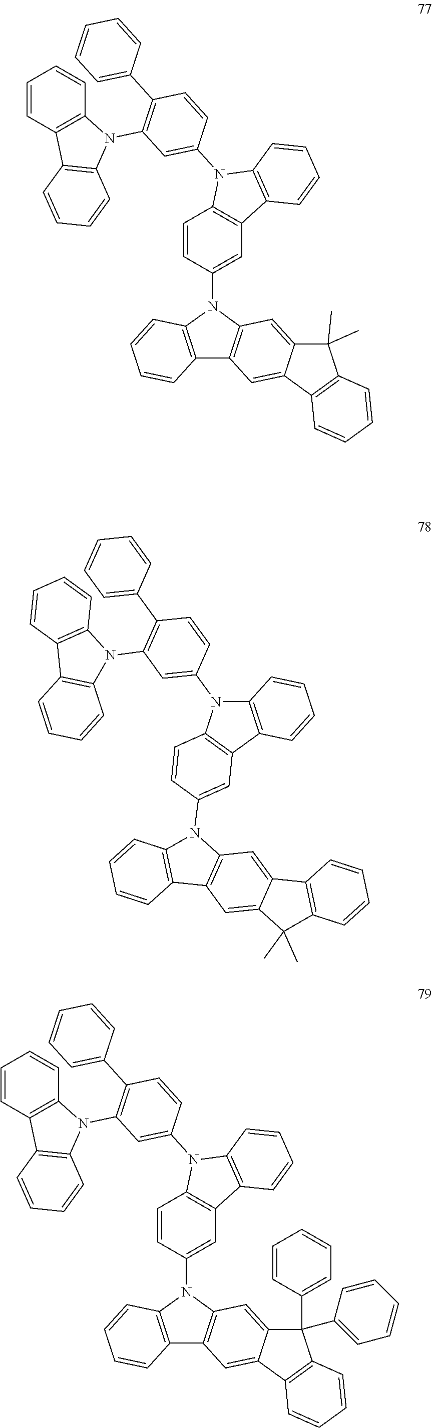 [SMILES: CC1(C)c2ccccc2-c2cc3c4ccccc4n(-c4ccc5c(c4)c4ccccc4n5-c4ccc(-c5ccccc5)c(-n5c6ccccc6c6ccccc65)c4)c3cc21 ; CC1(C)c2ccccc2-c2cc3c(cc21)c1ccccc1n3-c1ccc2c(c1)c1ccccc1n2-c1ccc(-c2ccccc2)c(-n2c3ccccc3c3ccccc32)c1 ; c1ccc(-c2ccc(-n3c4ccccc4c4cc(-n5c6ccccc6c6cc7c(cc65)C(c5ccccc5)(c5ccccc5)c5ccccc5-7)ccc43)cc2-n2c3ccccc3c3ccccc32)cc1]

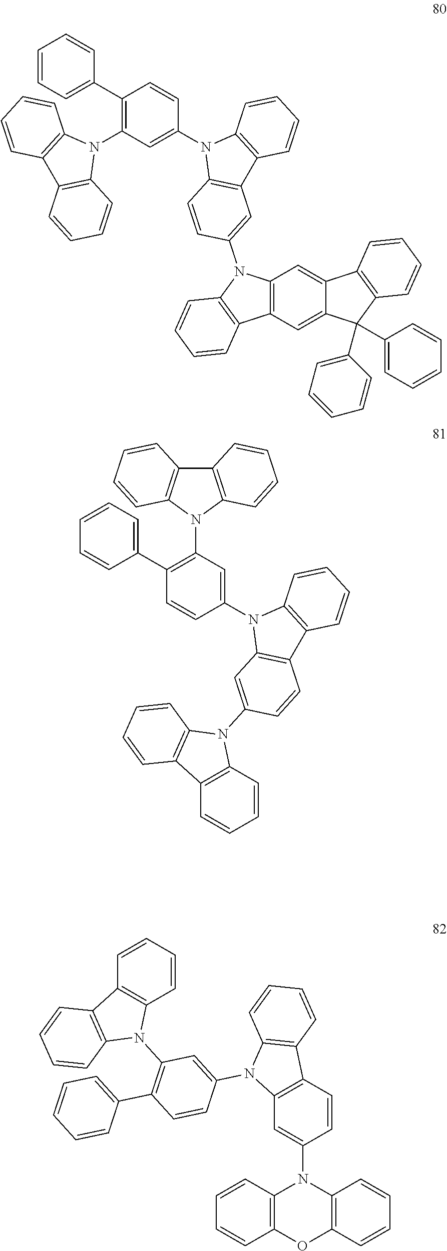 [SMILES: c1ccc(-c2ccc(-n3c4ccccc4c4cc(-n5c6ccccc6c6cc7c(cc65)-c5ccccc5C7(c5ccccc5)c5ccccc5)ccc43)cc2-n2c3ccccc3c3ccccc32)cc1 ; c1ccc(-c2ccc(-n3c4ccccc4c4ccc(-n5c6ccccc6c6ccccc65)cc43)cc2-n2c3ccccc3c3ccccc32)cc1 ; c1ccc(-c2ccc(-n3c4ccccc4c4ccc(N5c6ccccc6Oc6ccccc65)cc43)cc2-n2c3ccccc3c3ccccc32)cc1]

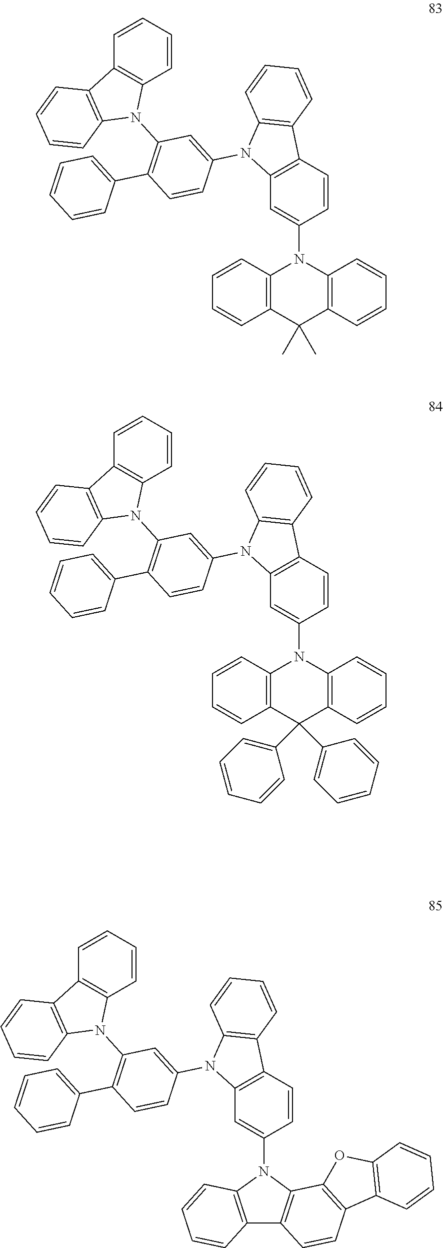 [SMILES: CC1(C)c2ccccc2N(c2ccc3c4ccccc4n(-c4ccc(-c5ccccc5)c(-n5c6ccccc6c6ccccc65)c4)c3c2)c2ccccc21 ; c1ccc(-c2ccc(-n3c4ccccc4c4ccc(N5c6ccccc6C(c6ccccc6)(c6ccccc6)c6ccccc65)cc43)cc2-n2c3ccccc3c3ccccc32)cc1 ; c1ccc(-c2ccc(-n3c4ccccc4c4ccc(-n5c6ccccc6c6ccc7c8ccccc8oc7c65)cc43)cc2-n2c3ccccc3c3ccccc32)cc1]

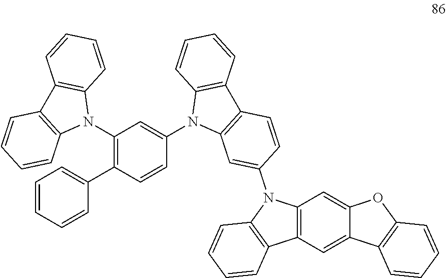 [SMILES: c1ccc(-c2ccc(-n3c4ccccc4c4ccc(-n5c6ccccc6c6cc7c(cc65)oc5ccccc57)cc43)cc2-n2c3ccccc3c3ccccc32)cc1]

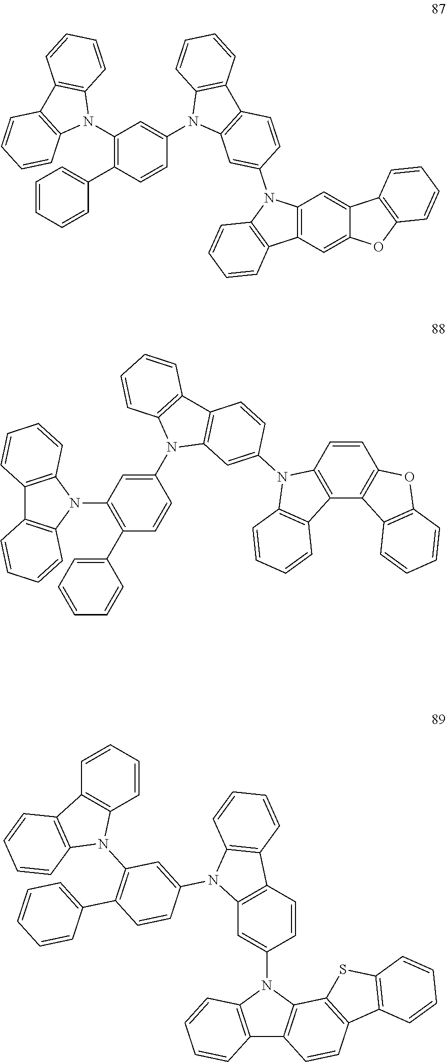 [SMILES: c1ccc(-c2ccc(-n3c4ccccc4c4ccc(-n5c6ccccc6c6cc7oc8ccccc8c7cc65)cc43)cc2-n2c3ccccc3c3ccccc32)cc1 ; c1ccc(-c2ccc(-n3c4ccccc4c4ccc(-n5c6ccccc6c6c7c(ccc65)oc5ccccc57)cc43)cc2-n2c3ccccc3c3ccccc32)cc1 ; c1ccc(-c2ccc(-n3c4ccccc4c4ccc(-n5c6ccccc6c6ccc7c8ccccc8sc7c65)cc43)cc2-n2c3ccccc3c3ccccc32)cc1]

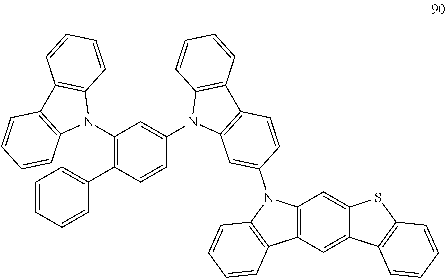 [SMILES: c1ccc(-c2ccc(-n3c4ccccc4c4ccc(-n5c6ccccc6c6cc7c(cc65)sc5ccccc57)cc43)cc2-n2c3ccccc3c3ccccc32)cc1]

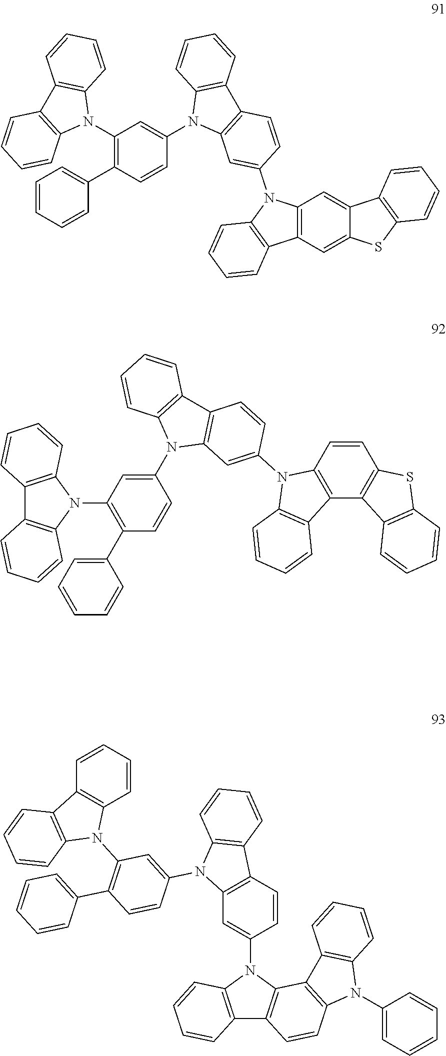 [SMILES: c1ccc(-c2ccc(-n3c4ccccc4c4ccc(-n5c6ccccc6c6cc7sc8ccccc8c7cc65)cc43)cc2-n2c3ccccc3c3ccccc32)cc1 ; c1ccc(-c2ccc(-n3c4ccccc4c4ccc(-n5c6ccccc6c6c7c(ccc65)sc5ccccc57)cc43)cc2-n2c3ccccc3c3ccccc32)cc1 ; c1ccc(-c2ccc(-n3c4ccccc4c4ccc(-n5c6ccccc6c6ccc7c(c8ccccc8n7-c7ccccc7)c65)cc43)cc2-n2c3ccccc3c3ccccc32)cc1]

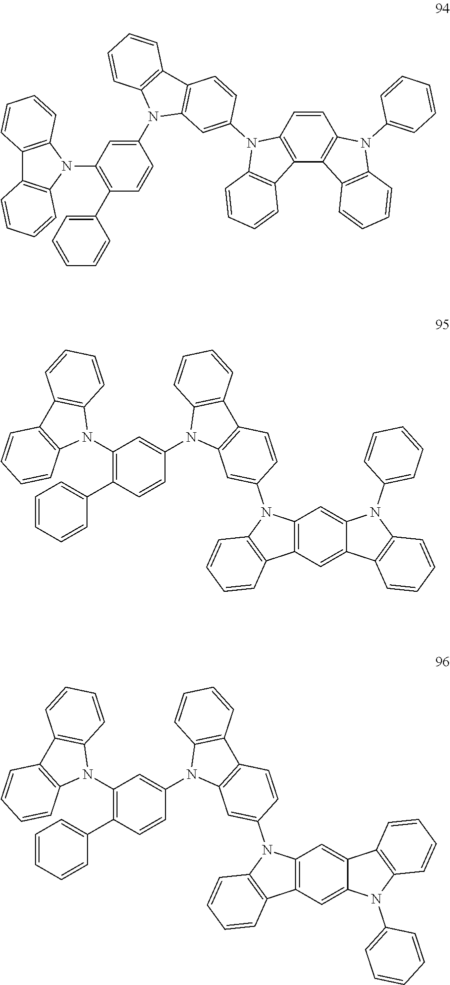 [SMILES: c1ccc(-c2ccc(-n3c4ccccc4c4ccc(-n5c6ccccc6c6c7c8ccccc8n(-c8ccccc8)c7ccc65)cc43)cc2-n2c3ccccc3c3ccccc32)cc1 ; c1ccc(-c2ccc(-n3c4ccccc4c4ccc(-n5c6ccccc6c6cc7c8ccccc8n(-c8ccccc8)c7cc65)cc43)cc2-n2c3ccccc3c3ccccc32)cc1 ; c1ccc(-c2ccc(-n3c4ccccc4c4ccc(-n5c6ccccc6c6cc7c(cc65)c5ccccc5n7-c5ccccc5)cc43)cc2-n2c3ccccc3c3ccccc32)cc1]

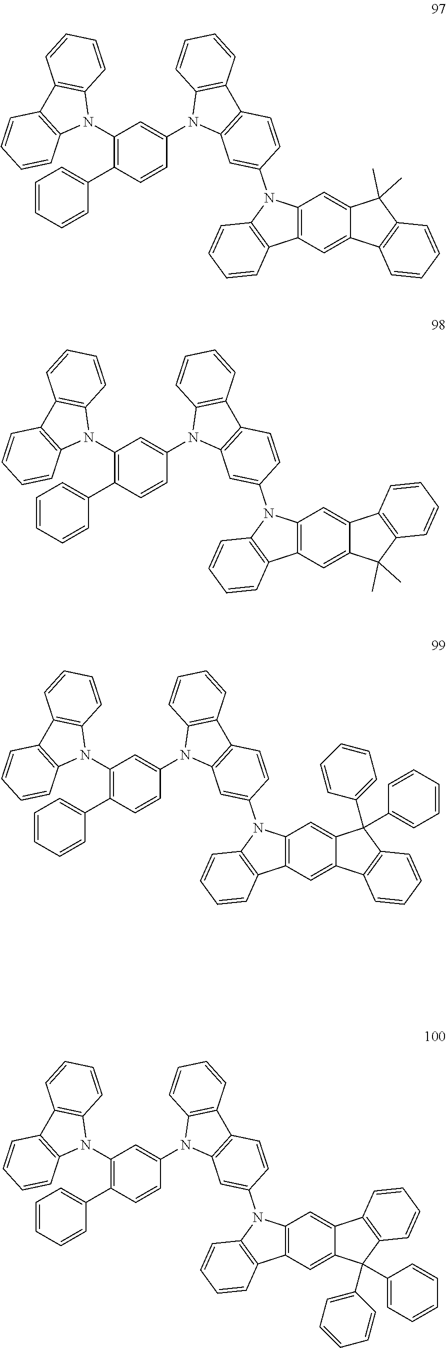 [SMILES: CC1(C)c2ccccc2-c2cc3c4ccccc4n(-c4ccc5c6ccccc6n(-c6ccc(-c7ccccc7)c(-n7c8ccccc8c8ccccc87)c6)c5c4)c3cc21 ; CC1(C)c2ccccc2-c2cc3c(cc21)c1ccccc1n3-c1ccc2c3ccccc3n(-c3ccc(-c4ccccc4)c(-n4c5ccccc5c5ccccc54)c3)c2c1 ; c1ccc(-c2ccc(-n3c4ccccc4c4ccc(-n5c6ccccc6c6cc7c(cc65)C(c5ccccc5)(c5ccccc5)c5ccccc5-7)cc43)cc2-n2c3ccccc3c3ccccc32)cc1 ; c1ccc(-c2ccc(-n3c4ccccc4c4ccc(-n5c6ccccc6c6cc7c(cc65)-c5ccccc5C7(c5ccccc5)c5ccccc5)cc43)cc2-n2c3ccccc3c3ccccc32)cc1]

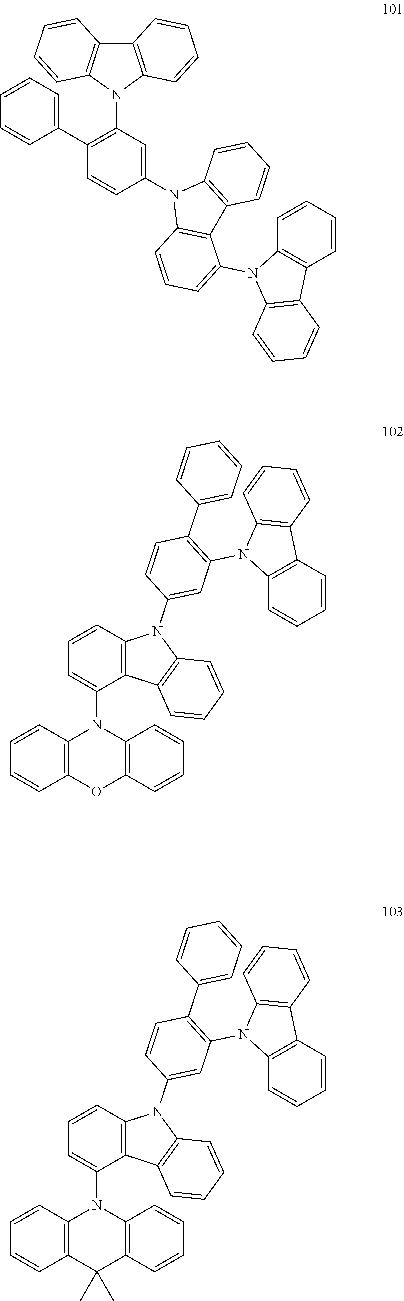 [SMILES: c1ccc(-c2ccc(-n3c4ccccc4c4c(-n5c6ccccc6c6ccccc65)cccc43)cc2-n2c3ccccc3c3ccccc32)cc1 ; c1ccc(-c2ccc(-n3c4ccccc4c4c(N5c6ccccc6Oc6ccccc65)cccc43)cc2-n2c3ccccc3c3ccccc32)cc1 ; CC1(C)c2ccccc2N(c2cccc3c2c2ccccc2n3-c2ccc(-c3ccccc3)c(-n3c4ccccc4c4ccccc43)c2)c2ccccc21]

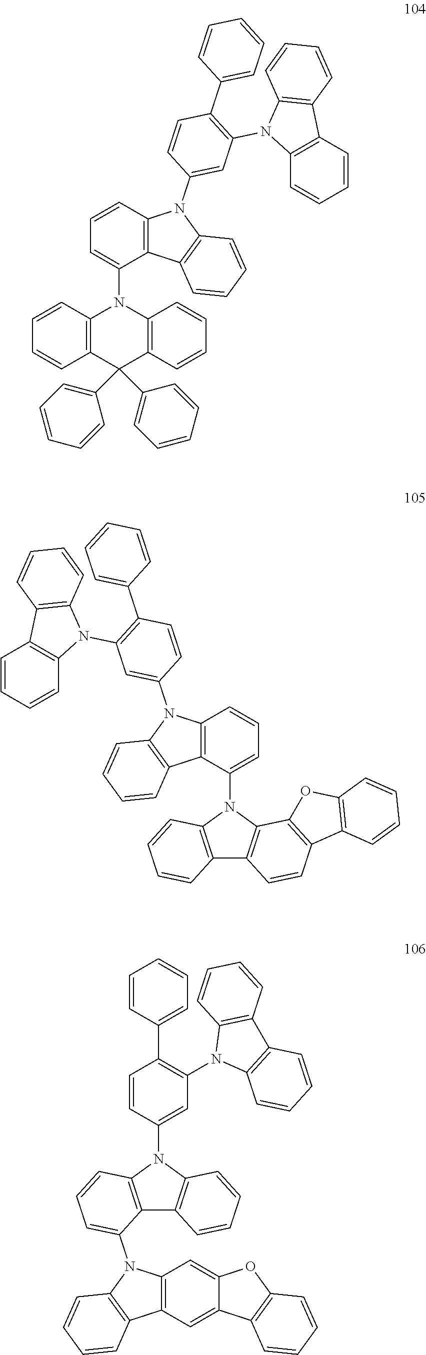 [SMILES: c1ccc(-c2ccc(-n3c4ccccc4c4c(N5c6ccccc6C(c6ccccc6)(c6ccccc6)c6ccccc65)cccc43)cc2-n2c3ccccc3c3ccccc32)cc1 ; c1ccc(-c2ccc(-n3c4ccccc4c4c(-n5c6ccccc6c6ccc7c8ccccc8oc7c65)cccc43)cc2-n2c3ccccc3c3ccccc32)cc1 ; c1ccc(-c2ccc(-n3c4ccccc4c4c(-n5c6ccccc6c6cc7c(cc65)oc5ccccc57)cccc43)cc2-n2c3ccccc3c3ccccc32)cc1]

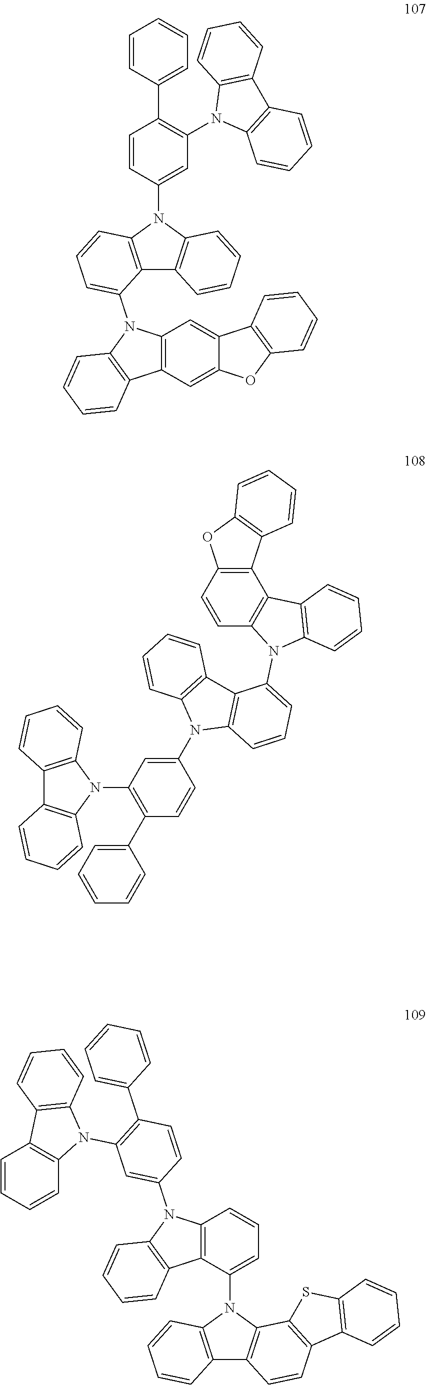 [SMILES: c1ccc(-c2ccc(-n3c4ccccc4c4c(-n5c6ccccc6c6cc7oc8ccccc8c7cc65)cccc43)cc2-n2c3ccccc3c3ccccc32)cc1 ; c1ccc(-c2ccc(-n3c4ccccc4c4c(-n5c6ccccc6c6c7c(ccc65)oc5ccccc57)cccc43)cc2-n2c3ccccc3c3ccccc32)cc1 ; c1ccc(-c2ccc(-n3c4ccccc4c4c(-n5c6ccccc6c6ccc7c8ccccc8sc7c65)cccc43)cc2-n2c3ccccc3c3ccccc32)cc1]

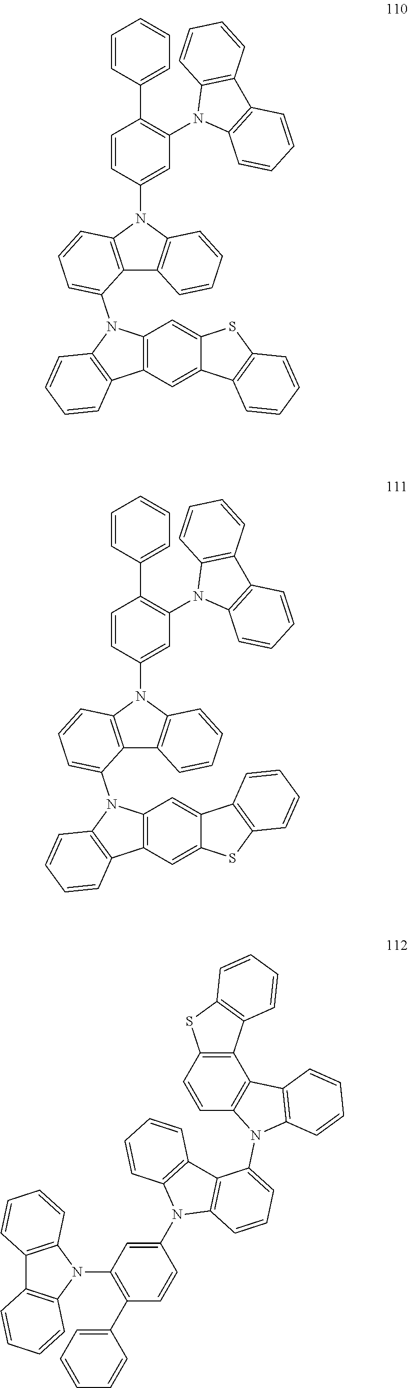 [SMILES: c1ccc(-c2ccc(-n3c4ccccc4c4c(-n5c6ccccc6c6cc7c(cc65)sc5ccccc57)cccc43)cc2-n2c3ccccc3c3ccccc32)cc1 ; c1ccc(-c2ccc(-n3c4ccccc4c4c(-n5c6ccccc6c6cc7sc8ccccc8c7cc65)cccc43)cc2-n2c3ccccc3c3ccccc32)cc1 ; c1ccc(-c2ccc(-n3c4ccccc4c4c(-n5c6ccccc6c6c7c(ccc65)sc5ccccc57)cccc43)cc2-n2c3ccccc3c3ccccc32)cc1]

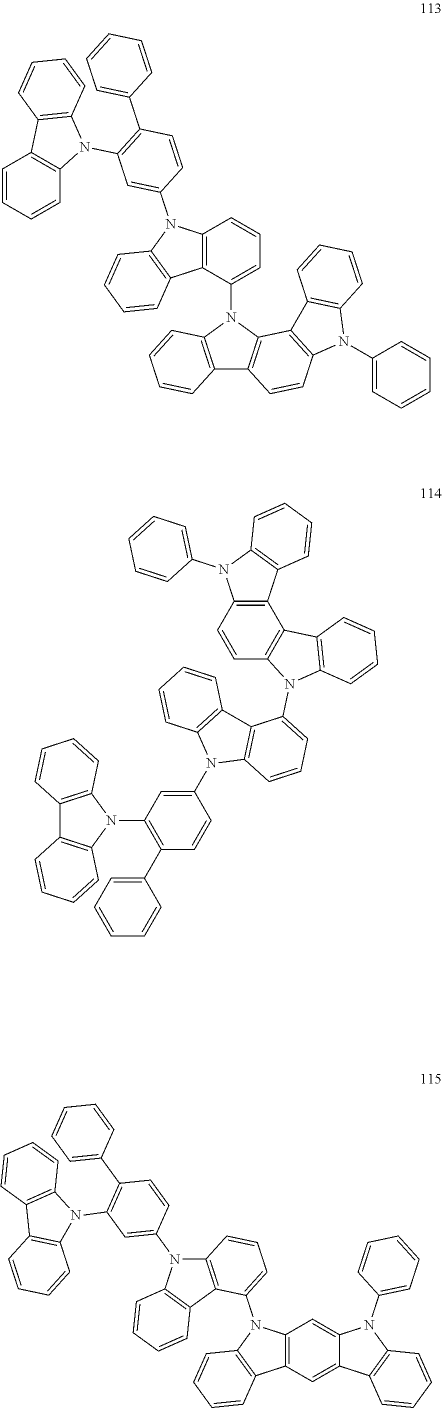 [SMILES: c1ccc(-c2ccc(-n3c4ccccc4c4c(-n5c6ccccc6c6ccc7c(c8ccccc8n7-c7ccccc7)c65)cccc43)cc2-n2c3ccccc3c3ccccc32)cc1 ; c1ccc(-c2ccc(-n3c4ccccc4c4c(-n5c6ccccc6c6c7c8ccccc8n(-c8ccccc8)c7ccc65)cccc43)cc2-n2c3ccccc3c3ccccc32)cc1 ; c1ccc(-c2ccc(-n3c4ccccc4c4c(-n5c6ccccc6c6cc7c8ccccc8n(-c8ccccc8)c7cc65)cccc43)cc2-n2c3ccccc3c3ccccc32)cc1]

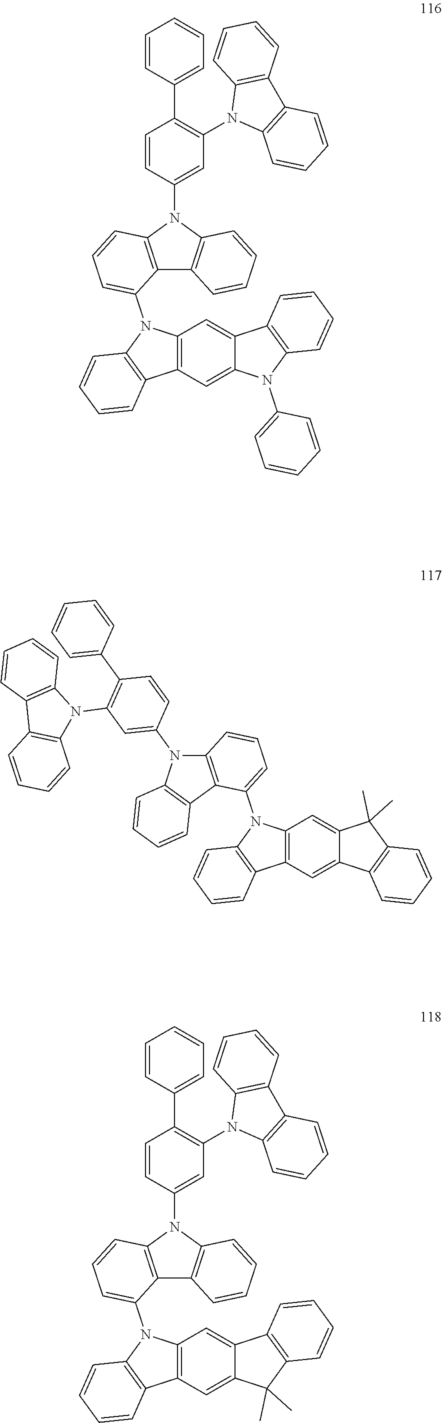 [SMILES: c1ccc(-c2ccc(-n3c4ccccc4c4c(-n5c6ccccc6c6cc7c(cc65)c5ccccc5n7-c5ccccc5)cccc43)cc2-n2c3ccccc3c3ccccc32)cc1 ; CC1(C)c2ccccc2-c2cc3c4ccccc4n(-c4cccc5c4c4ccccc4n5-c4ccc(-c5ccccc5)c(-n5c6ccccc6c6ccccc65)c4)c3cc21 ; CC1(C)c2ccccc2-c2cc3c(cc21)c1ccccc1n3-c1cccc2c1c1ccccc1n2-c1ccc(-c2ccccc2)c(-n2c3ccccc3c3ccccc32)c1]

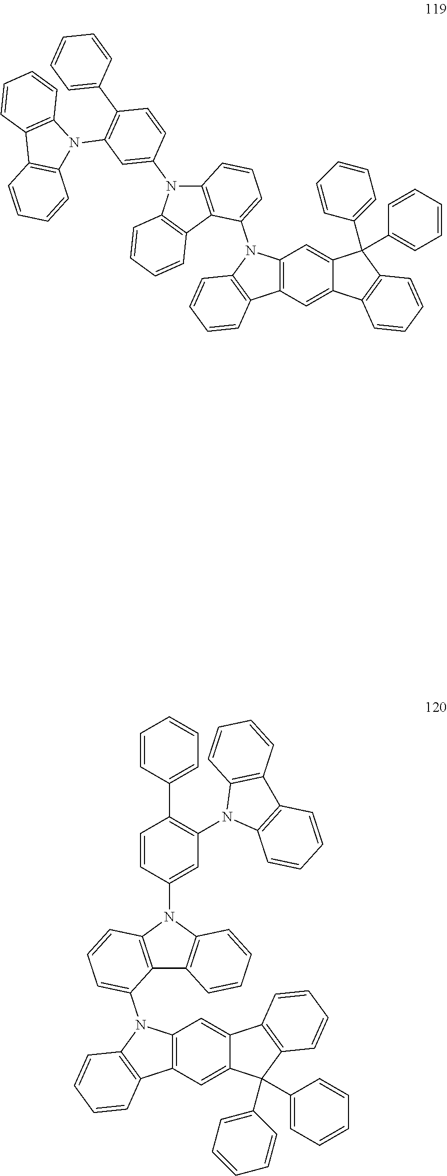 [SMILES: c1ccc(-c2ccc(-n3c4ccccc4c4c(-n5c6ccccc6c6cc7c(cc65)C(c5ccccc5)(c5ccccc5)c5ccccc5-7)cccc43)cc2-n2c3ccccc3c3ccccc32)cc1 ; c1ccc(-c2ccc(-n3c4ccccc4c4c(-n5c6ccccc6c6cc7c(cc65)-c5ccccc5C7(c5ccccc5)c5ccccc5)cccc43)cc2-n2c3ccccc3c3ccccc32)cc1]

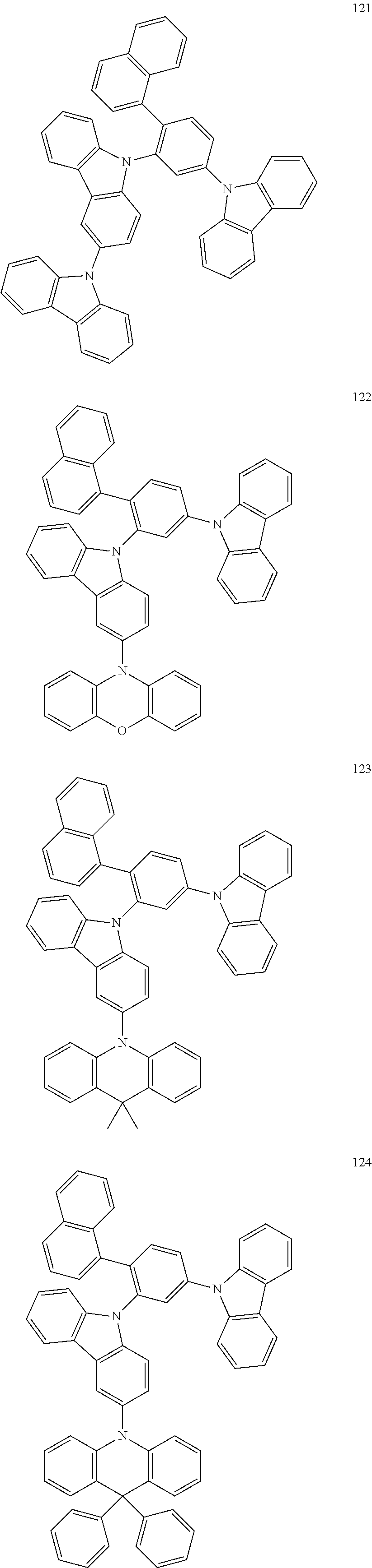 [SMILES: c1ccc2c(-c3ccc(-n4c5ccccc5c5ccccc54)cc3-n3c4ccccc4c4cc(-n5c6ccccc6c6ccccc65)ccc43)cccc2c1 ; c1ccc2c(c1)Oc1ccccc1N2c1ccc2c(c1)c1ccccc1n2-c1cc(-n2c3ccccc3c3ccccc32)ccc1-c1cccc2ccccc12 ; CC1(C)c2ccccc2N(c2ccc3c(c2)c2ccccc2n3-c2cc(-n3c4ccccc4c4ccccc43)ccc2-c2cccc3ccccc23)c2ccccc21 ; c1ccc(C2(c3ccccc3)c3ccccc3N(c3ccc4c(c3)c3ccccc3n4-c3cc(-n4c5ccccc5c5ccccc54)ccc3-c3cccc4ccccc34)c3ccccc32)cc1]

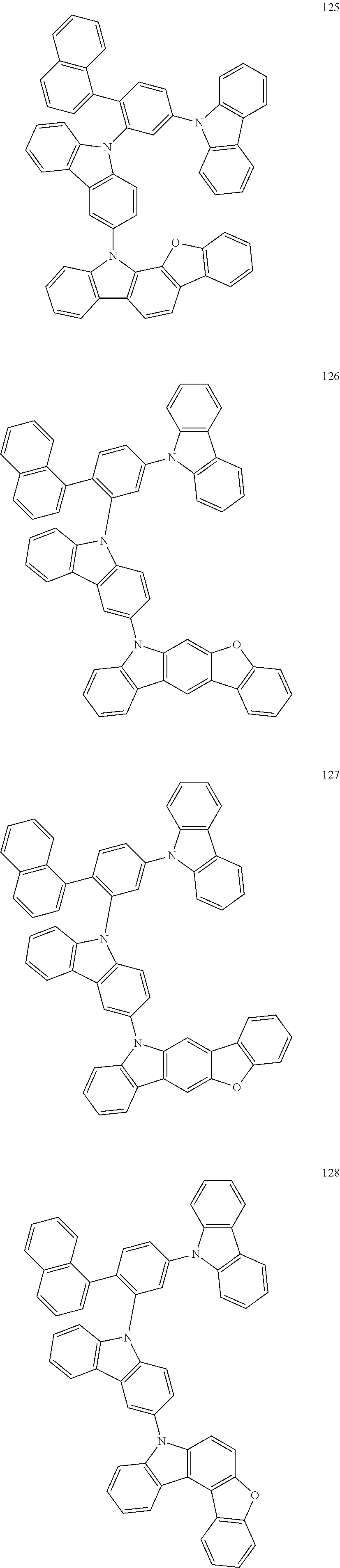 [SMILES: c1ccc2c(-c3ccc(-n4c5ccccc5c5ccccc54)cc3-n3c4ccccc4c4cc(-n5c6ccccc6c6ccc7c8ccccc8oc7c65)ccc43)cccc2c1 ; c1ccc2c(-c3ccc(-n4c5ccccc5c5ccccc54)cc3-n3c4ccccc4c4cc(-n5c6ccccc6c6cc7c(cc65)oc5ccccc57)ccc43)cccc2c1 ; c1ccc2c(-c3ccc(-n4c5ccccc5c5ccccc54)cc3-n3c4ccccc4c4cc(-n5c6ccccc6c6cc7oc8ccccc8c7cc65)ccc43)cccc2c1 ; c1ccc2c(-c3ccc(-n4c5ccccc5c5ccccc54)cc3-n3c4ccccc4c4cc(-n5c6ccccc6c6c7c(ccc65)oc5ccccc57)ccc43)cccc2c1]

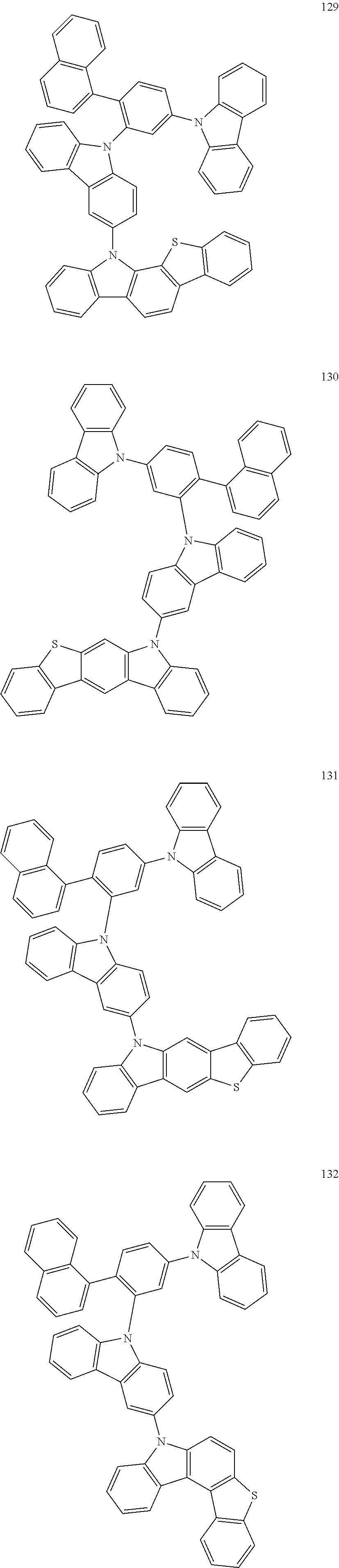 [SMILES: c1ccc2c(-c3ccc(-n4c5ccccc5c5ccccc54)cc3-n3c4ccccc4c4cc(-n5c6ccccc6c6ccc7c8ccccc8sc7c65)ccc43)cccc2c1 ; c1ccc2c(-c3ccc(-n4c5ccccc5c5ccccc54)cc3-n3c4ccccc4c4cc(-n5c6ccccc6c6cc7c(cc65)sc5ccccc57)ccc43)cccc2c1 ; c1ccc2c(-c3ccc(-n4c5ccccc5c5ccccc54)cc3-n3c4ccccc4c4cc(-n5c6ccccc6c6cc7sc8ccccc8c7cc65)ccc43)cccc2c1 ; c1ccc2c(-c3ccc(-n4c5ccccc5c5ccccc54)cc3-n3c4ccccc4c4cc(-n5c6ccccc6c6c7c(ccc65)sc5ccccc57)ccc43)cccc2c1]

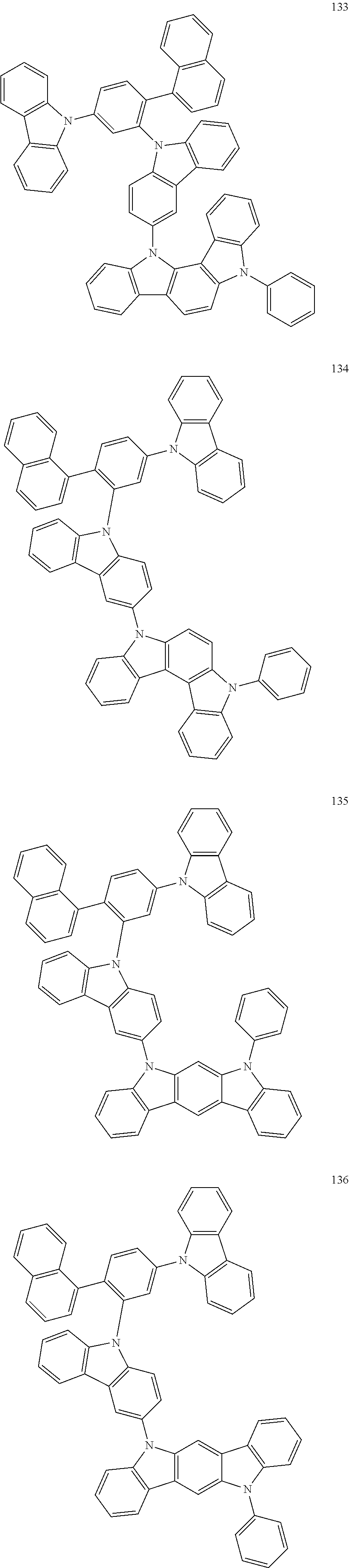 [SMILES: c1ccc(-n2c3ccccc3c3c2ccc2c4ccccc4n(-c4ccc5c(c4)c4ccccc4n5-c4cc(-n5c6ccccc6c6ccccc65)ccc4-c4cccc5ccccc45)c23)cc1 ; c1ccc(-n2c3ccccc3c3c4c5ccccc5n(-c5ccc6c(c5)c5ccccc5n6-c5cc(-n6c7ccccc7c7ccccc76)ccc5-c5cccc6ccccc56)c4ccc32)cc1 ; c1ccc(-n2c3ccccc3c3cc4c5ccccc5n(-c5ccc6c(c5)c5ccccc5n6-c5cc(-n6c7ccccc7c7ccccc76)ccc5-c5cccc6ccccc56)c4cc32)cc1 ; c1ccc(-n2c3ccccc3c3cc4c(cc32)c2ccccc2n4-c2ccc3c(c2)c2ccccc2n3-c2cc(-n3c4ccccc4c4ccccc43)ccc2-c2cccc3ccccc23)cc1]

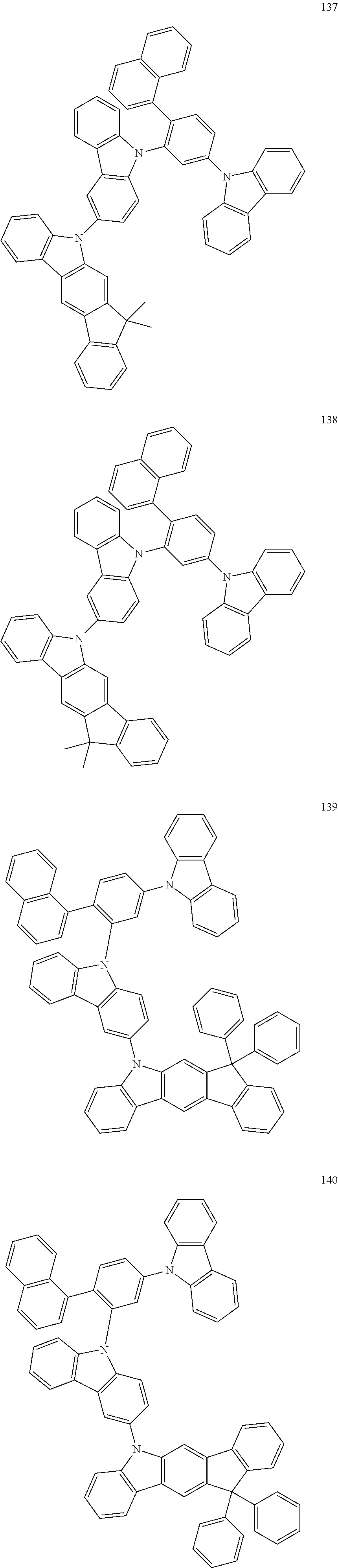 [SMILES: CC1(C)c2ccccc2-c2cc3c4ccccc4n(-c4ccc5c(c4)c4ccccc4n5-c4cc(-n5c6ccccc6c6ccccc65)ccc4-c4cccc5ccccc45)c3cc21 ; CC1(C)c2ccccc2-c2cc3c(cc21)c1ccccc1n3-c1ccc2c(c1)c1ccccc1n2-c1cc(-n2c3ccccc3c3ccccc32)ccc1-c1cccc2ccccc12 ; c1ccc(C2(c3ccccc3)c3ccccc3-c3cc4c5ccccc5n(-c5ccc6c(c5)c5ccccc5n6-c5cc(-n6c7ccccc7c7ccccc76)ccc5-c5cccc6ccccc56)c4cc32)cc1 ; c1ccc(C2(c3ccccc3)c3ccccc3-c3cc4c(cc32)c2ccccc2n4-c2ccc3c(c2)c2ccccc2n3-c2cc(-n3c4ccccc4c4ccccc43)ccc2-c2cccc3ccccc23)cc1]

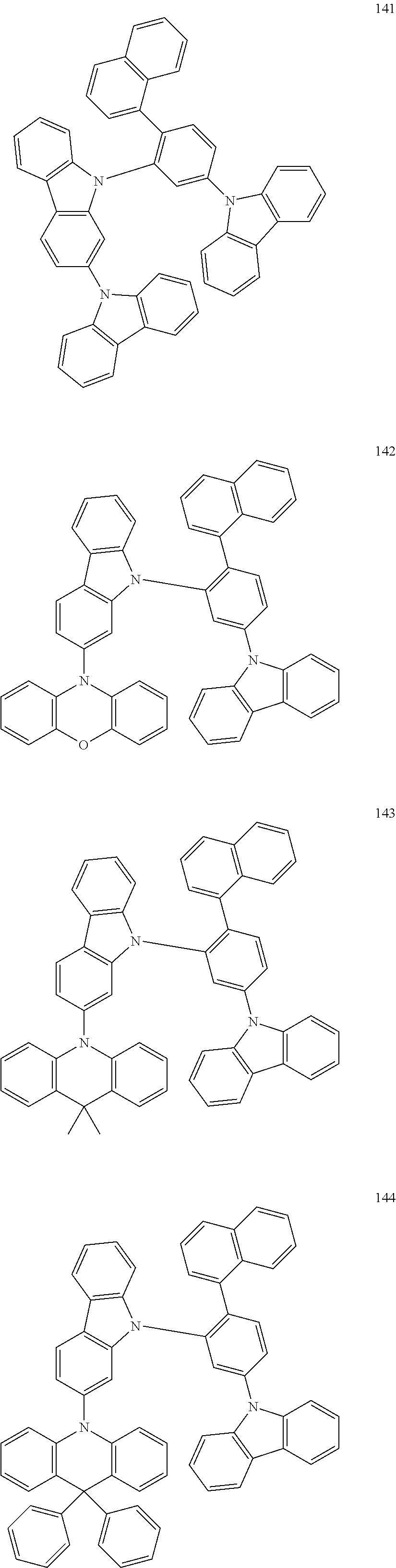 [SMILES: c1ccc2c(-c3ccc(-n4c5ccccc5c5ccccc54)cc3-n3c4ccccc4c4ccc(-n5c6ccccc6c6ccccc65)cc43)cccc2c1 ; c1ccc2c(c1)Oc1ccccc1N2c1ccc2c3ccccc3n(-c3cc(-n4c5ccccc5c5ccccc54)ccc3-c3cccc4ccccc34)c2c1 ; CC1(C)c2ccccc2N(c2ccc3c4ccccc4n(-c4cc(-n5c6ccccc6c6ccccc65)ccc4-c4cccc5ccccc45)c3c2)c2ccccc21 ; c1ccc(C2(c3ccccc3)c3ccccc3N(c3ccc4c5ccccc5n(-c5cc(-n6c7ccccc7c7ccccc76)ccc5-c5cccc6ccccc56)c4c3)c3ccccc32)cc1]

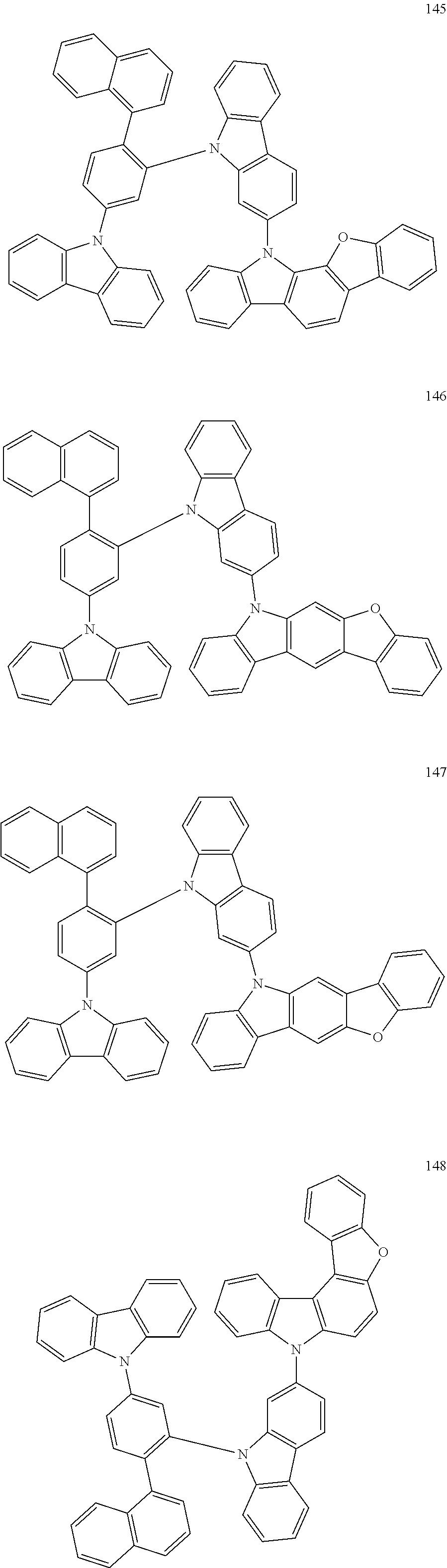 [SMILES: c1ccc2c(-c3ccc(-n4c5ccccc5c5ccccc54)cc3-n3c4ccccc4c4ccc(-n5c6ccccc6c6ccc7c8ccccc8oc7c65)cc43)cccc2c1 ; c1ccc2c(-c3ccc(-n4c5ccccc5c5ccccc54)cc3-n3c4ccccc4c4ccc(-n5c6ccccc6c6cc7c(cc65)oc5ccccc57)cc43)cccc2c1 ; c1ccc2c(-c3ccc(-n4c5ccccc5c5ccccc54)cc3-n3c4ccccc4c4ccc(-n5c6ccccc6c6cc7oc8ccccc8c7cc65)cc43)cccc2c1 ; c1ccc2c(-c3ccc(-n4c5ccccc5c5ccccc54)cc3-n3c4ccccc4c4ccc(-n5c6ccccc6c6c7c(ccc65)oc5ccccc57)cc43)cccc2c1]

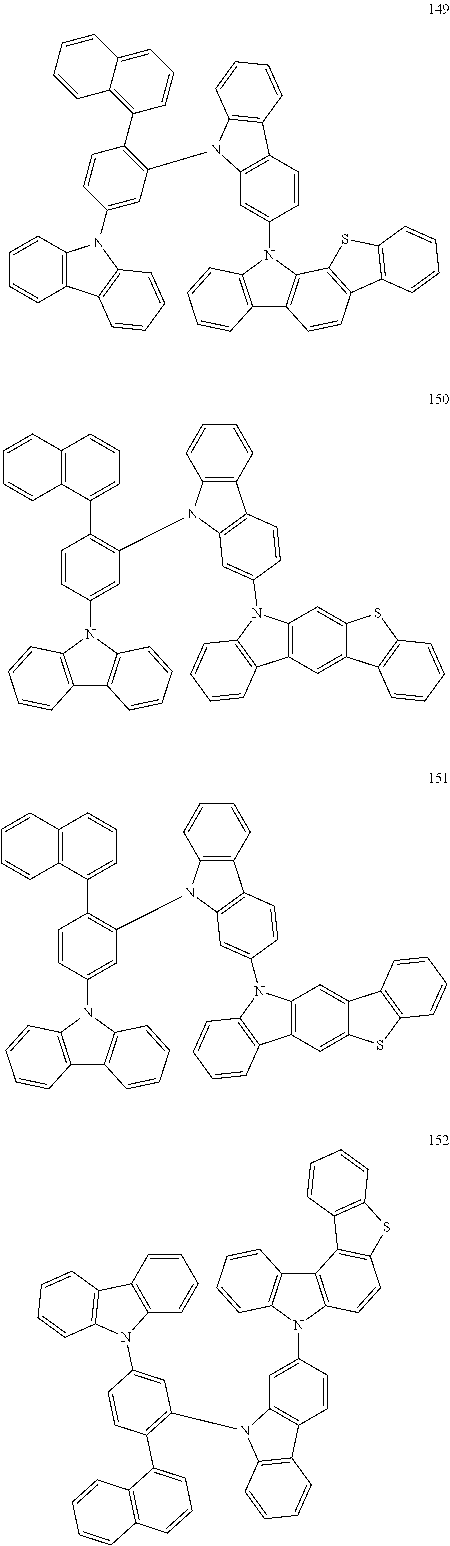 [SMILES: c1ccc2c(-c3ccc(-n4c5ccccc5c5ccccc54)cc3-n3c4ccccc4c4ccc(-n5c6ccccc6c6ccc7c8ccccc8sc7c65)cc43)cccc2c1 ; c1ccc2c(-c3ccc(-n4c5ccccc5c5ccccc54)cc3-n3c4ccccc4c4ccc(-n5c6ccccc6c6cc7c(cc65)sc5ccccc57)cc43)cccc2c1 ; c1ccc2c(-c3ccc(-n4c5ccccc5c5ccccc54)cc3-n3c4ccccc4c4ccc(-n5c6ccccc6c6cc7sc8ccccc8c7cc65)cc43)cccc2c1 ; c1ccc2c(-c3ccc(-n4c5ccccc5c5ccccc54)cc3-n3c4ccccc4c4ccc(-n5c6ccccc6c6c7c(ccc65)sc5ccccc57)cc43)cccc2c1]

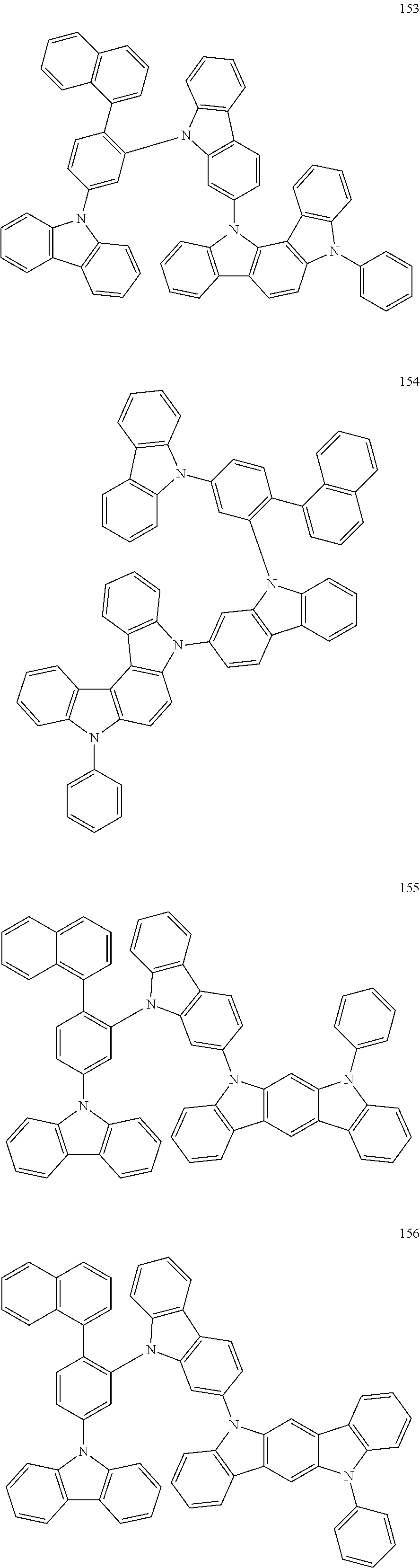

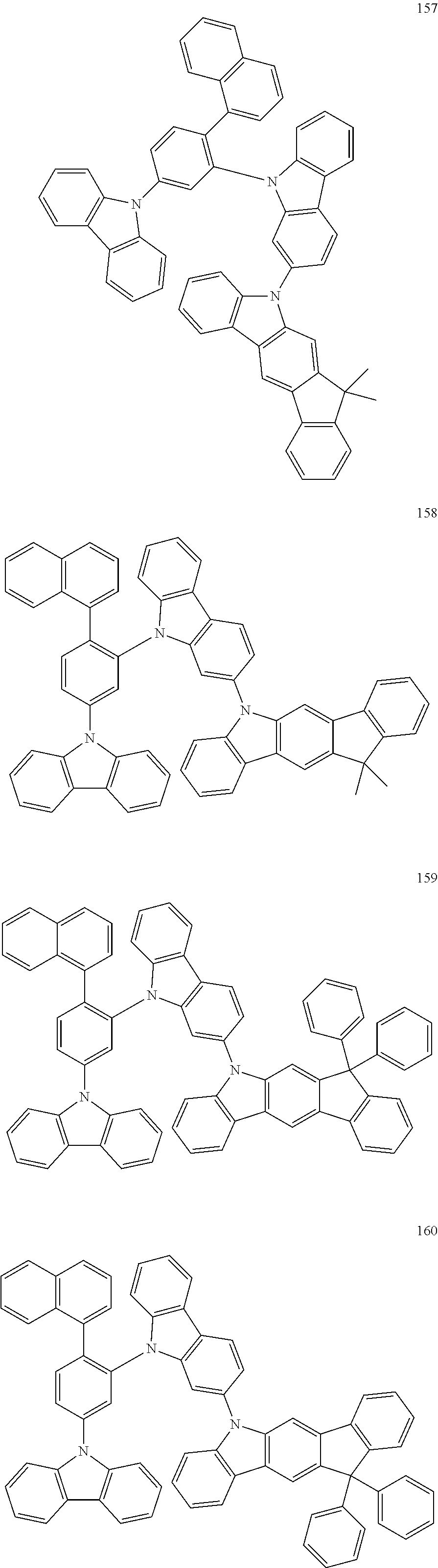 [SMILES: CC1(C)c2ccccc2-c2cc3c4ccccc4n(-c4ccc5c6ccccc6n(-c6cc(-n7c8ccccc8c8ccccc87)ccc6-c6cccc7ccccc67)c5c4)c3cc21 ; CC1(C)c2ccccc2-c2cc3c(cc21)c1ccccc1n3-c1ccc2c3ccccc3n(-c3cc(-n4c5ccccc5c5ccccc54)ccc3-c3cccc4ccccc34)c2c1 ; c1ccc(C2(c3ccccc3)c3ccccc3-c3cc4c5ccccc5n(-c5ccc6c7ccccc7n(-c7cc(-n8c9ccccc9c9ccccc98)ccc7-c7cccc8ccccc78)c6c5)c4cc32)cc1 ; c1ccc(C2(c3ccccc3)c3ccccc3-c3cc4c(cc32)c2ccccc2n4-c2ccc3c4ccccc4n(-c4cc(-n5c6ccccc6c6ccccc65)ccc4-c4cccc5ccccc45)c3c2)cc1]

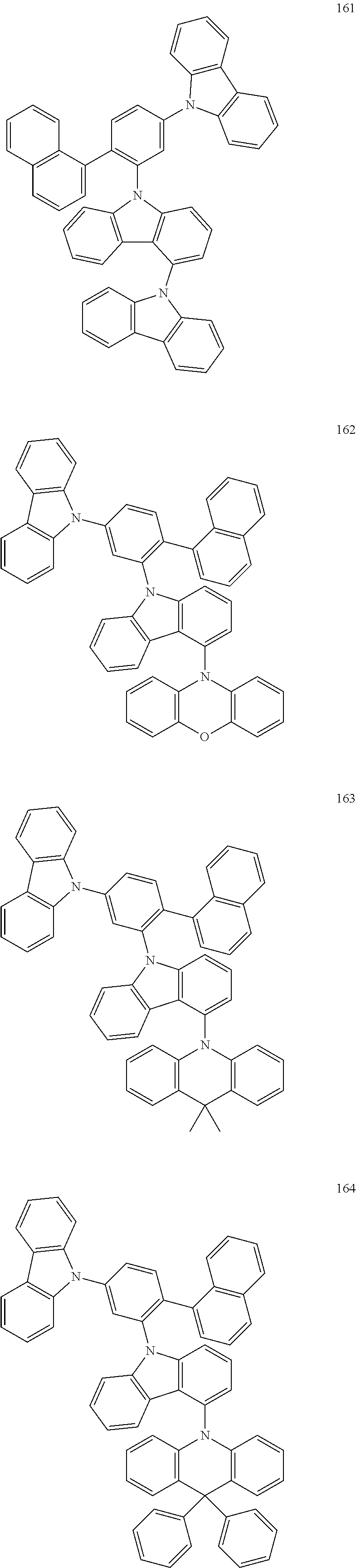 [SMILES: c1ccc2c(-c3ccc(-n4c5ccccc5c5ccccc54)cc3-n3c4ccccc4c4c(-n5c6ccccc6c6ccccc65)cccc43)cccc2c1 ; c1ccc2c(c1)Oc1ccccc1N2c1cccc2c1c1ccccc1n2-c1cc(-n2c3ccccc3c3ccccc32)ccc1-c1cccc2ccccc12 ; CC1(C)c2ccccc2N(c2cccc3c2c2ccccc2n3-c2cc(-n3c4ccccc4c4ccccc43)ccc2-c2cccc3ccccc23)c2ccccc21 ; c1ccc(C2(c3ccccc3)c3ccccc3N(c3cccc4c3c3ccccc3n4-c3cc(-n4c5ccccc5c5ccccc54)ccc3-c3cccc4ccccc34)c3ccccc32)cc1]

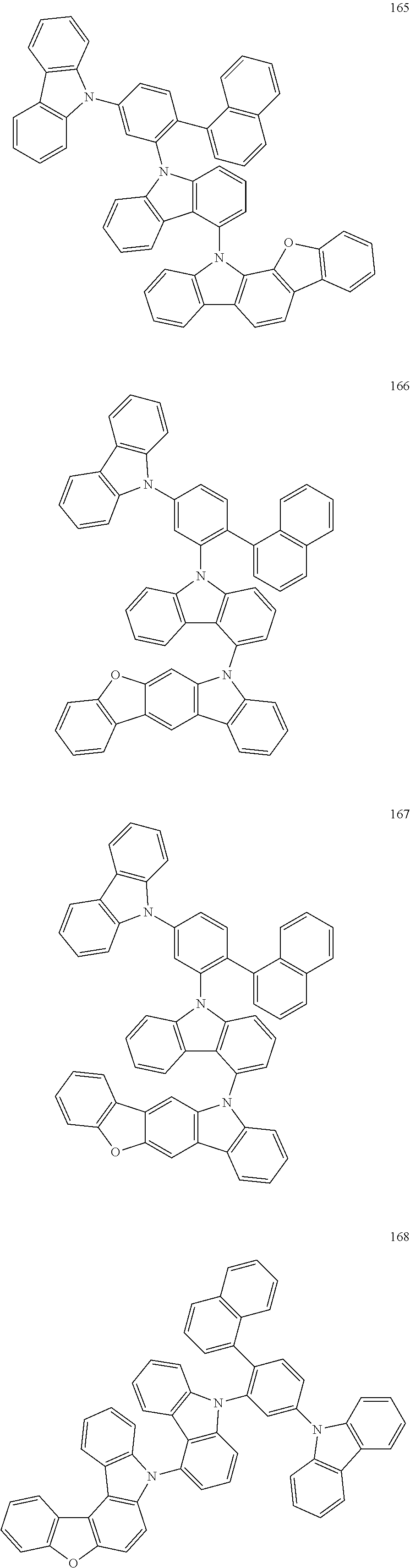 [SMILES: c1ccc2c(-c3ccc(-n4c5ccccc5c5ccccc54)cc3-n3c4ccccc4c4c(-n5c6ccccc6c6ccc7c8ccccc8oc7c65)cccc43)cccc2c1 ; c1ccc2c(-c3ccc(-n4c5ccccc5c5ccccc54)cc3-n3c4ccccc4c4c(-n5c6ccccc6c6cc7c(cc65)oc5ccccc57)cccc43)cccc2c1 ; c1ccc2c(-c3ccc(-n4c5ccccc5c5ccccc54)cc3-n3c4ccccc4c4c(-n5c6ccccc6c6cc7oc8ccccc8c7cc65)cccc43)cccc2c1 ; c1ccc2c(-c3ccc(-n4c5ccccc5c5ccccc54)cc3-n3c4ccccc4c4c(-n5c6ccccc6c6c7c(ccc65)oc5ccccc57)cccc43)cccc2c1]

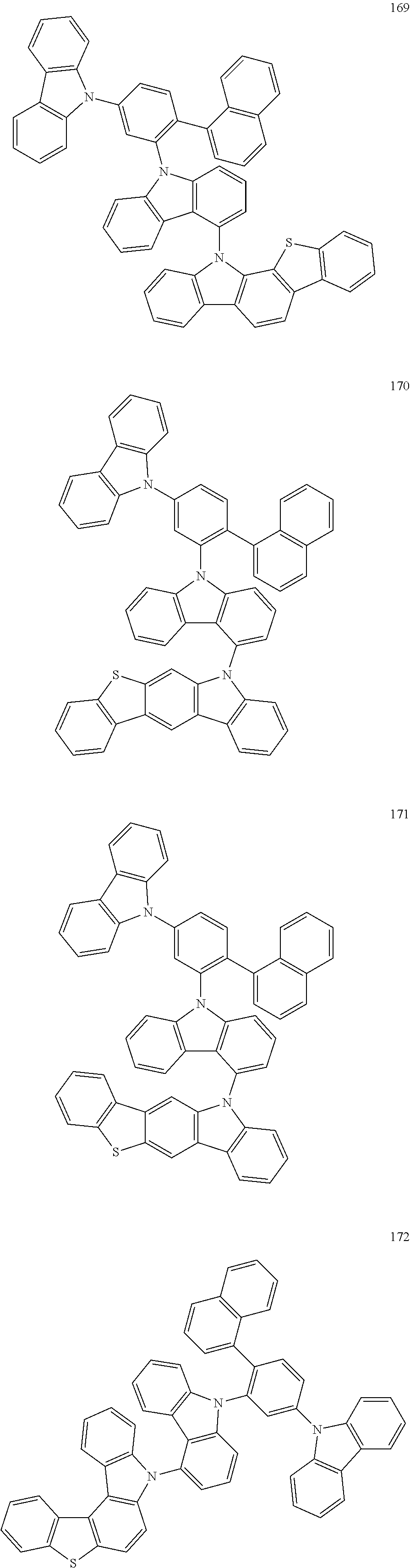 [SMILES: c1ccc2c(-c3ccc(-n4c5ccccc5c5ccccc54)cc3-n3c4ccccc4c4c(-n5c6ccccc6c6ccc7c8ccccc8sc7c65)cccc43)cccc2c1 ; c1ccc2c(-c3ccc(-n4c5ccccc5c5ccccc54)cc3-n3c4ccccc4c4c(-n5c6ccccc6c6cc7c(cc65)sc5ccccc57)cccc43)cccc2c1 ; c1ccc2c(-c3ccc(-n4c5ccccc5c5ccccc54)cc3-n3c4ccccc4c4c(-n5c6ccccc6c6cc7sc8ccccc8c7cc65)cccc43)cccc2c1 ; c1ccc2c(-c3ccc(-n4c5ccccc5c5ccccc54)cc3-n3c4ccccc4c4c(-n5c6ccccc6c6c7c(ccc65)sc5ccccc57)cccc43)cccc2c1]

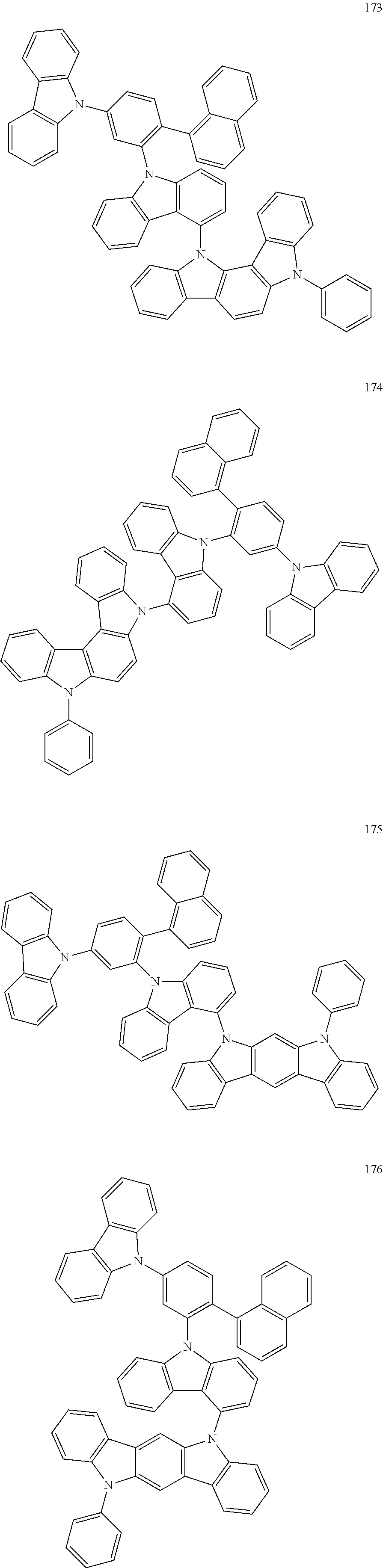 [SMILES: c1ccc(-n2c3ccccc3c3c2ccc2c4ccccc4n(-c4cccc5c4c4ccccc4n5-c4cc(-n5c6ccccc6c6ccccc65)ccc4-c4cccc5ccccc45)c23)cc1 ; c1ccc(-n2c3ccccc3c3c4c5ccccc5n(-c5cccc6c5c5ccccc5n6-c5cc(-n6c7ccccc7c7ccccc76)ccc5-c5cccc6ccccc56)c4ccc32)cc1 ; c1ccc(-n2c3ccccc3c3cc4c5ccccc5n(-c5cccc6c5c5ccccc5n6-c5cc(-n6c7ccccc7c7ccccc76)ccc5-c5cccc6ccccc56)c4cc32)cc1 ; c1ccc(-n2c3ccccc3c3cc4c(cc32)c2ccccc2n4-c2cccc3c2c2ccccc2n3-c2cc(-n3c4ccccc4c4ccccc43)ccc2-c2cccc3ccccc23)cc1]

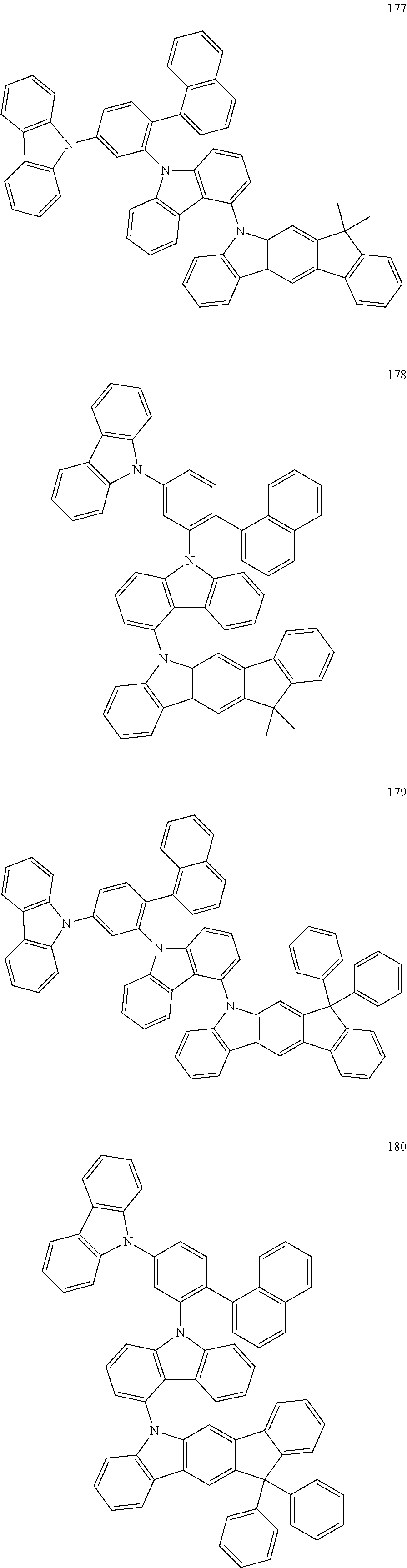 [SMILES: CC1(C)c2ccccc2-c2cc3c4ccccc4n(-c4cccc5c4c4ccccc4n5-c4cc(-n5c6ccccc6c6ccccc65)ccc4-c4cccc5ccccc45)c3cc21 ; CC1(C)c2ccccc2-c2cc3c(cc21)c1ccccc1n3-c1cccc2c1c1ccccc1n2-c1cc(-n2c3ccccc3c3ccccc32)ccc1-c1cccc2ccccc12 ; c1ccc(C2(c3ccccc3)c3ccccc3-c3cc4c5ccccc5n(-c5cccc6c5c5ccccc5n6-c5cc(-n6c7ccccc7c7ccccc76)ccc5-c5cccc6ccccc56)c4cc32)cc1 ; c1ccc(C2(c3ccccc3)c3ccccc3-c3cc4c(cc32)c2ccccc2n4-c2cccc3c2c2ccccc2n3-c2cc(-n3c4ccccc4c4ccccc43)ccc2-c2cccc3ccccc23)cc1]

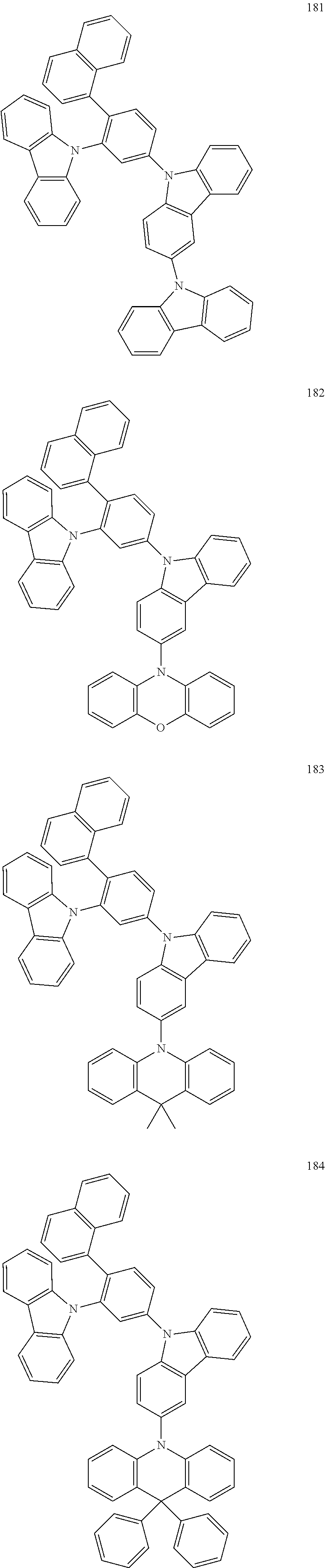 [SMILES: c1ccc2c(-c3ccc(-n4c5ccccc5c5cc(-n6c7ccccc7c7ccccc76)ccc54)cc3-n3c4ccccc4c4ccccc43)cccc2c1 ; c1ccc2c(c1)Oc1ccccc1N2c1ccc2c(c1)c1ccccc1n2-c1ccc(-c2cccc3ccccc23)c(-n2c3ccccc3c3ccccc32)c1 ; CC1(C)c2ccccc2N(c2ccc3c(c2)c2ccccc2n3-c2ccc(-c3cccc4ccccc34)c(-n3c4ccccc4c4ccccc43)c2)c2ccccc21 ; c1ccc(C2(c3ccccc3)c3ccccc3N(c3ccc4c(c3)c3ccccc3n4-c3ccc(-c4cccc5ccccc45)c(-n4c5ccccc5c5ccccc54)c3)c3ccccc32)cc1]

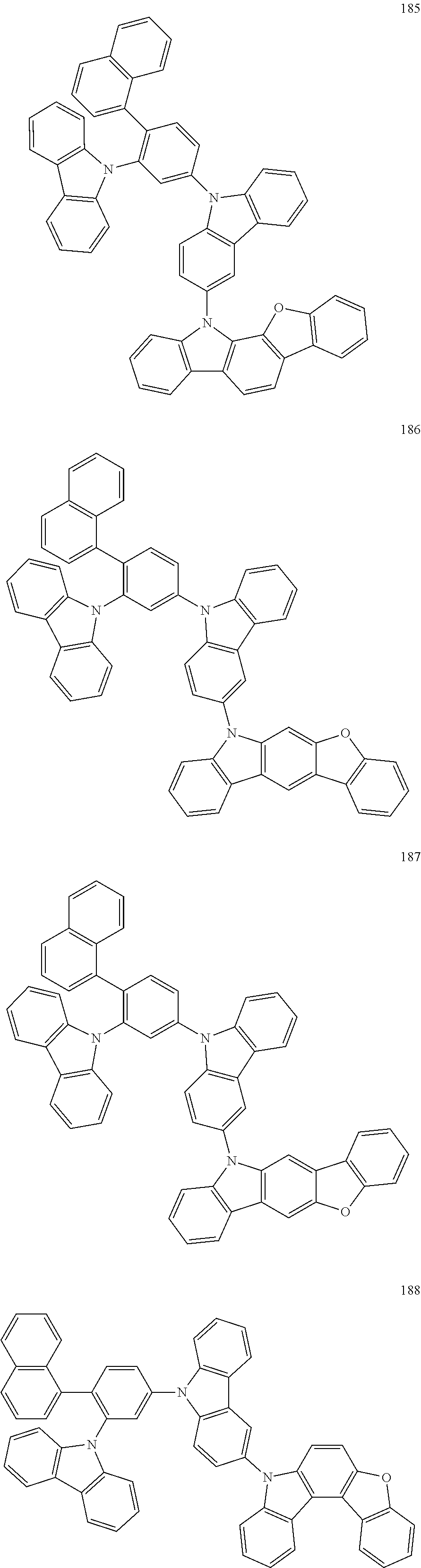 [SMILES: c1ccc2c(-c3ccc(-n4c5ccccc5c5cc(-n6c7ccccc7c7ccc8c9ccccc9oc8c76)ccc54)cc3-n3c4ccccc4c4ccccc43)cccc2c1 ; c1ccc2c(-c3ccc(-n4c5ccccc5c5cc(-n6c7ccccc7c7cc8c(cc76)oc6ccccc68)ccc54)cc3-n3c4ccccc4c4ccccc43)cccc2c1 ; c1ccc2c(-c3ccc(-n4c5ccccc5c5cc(-n6c7ccccc7c7cc8oc9ccccc9c8cc76)ccc54)cc3-n3c4ccccc4c4ccccc43)cccc2c1 ; c1ccc2c(-c3ccc(-n4c5ccccc5c5cc(-n6c7ccccc7c7c8c(ccc76)oc6ccccc68)ccc54)cc3-n3c4ccccc4c4ccccc43)cccc2c1]

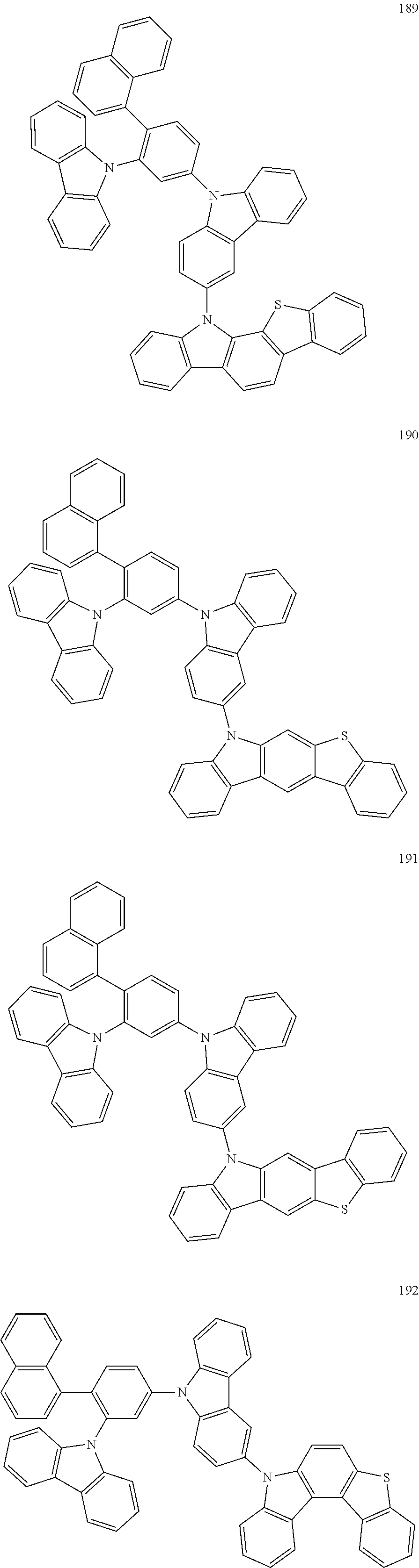 [SMILES: c1ccc2c(-c3ccc(-n4c5ccccc5c5cc(-n6c7ccccc7c7ccc8c9ccccc9sc8c76)ccc54)cc3-n3c4ccccc4c4ccccc43)cccc2c1 ; c1ccc2c(-c3ccc(-n4c5ccccc5c5cc(-n6c7ccccc7c7cc8c(cc76)sc6ccccc68)ccc54)cc3-n3c4ccccc4c4ccccc43)cccc2c1 ; c1ccc2c(-c3ccc(-n4c5ccccc5c5cc(-n6c7ccccc7c7cc8sc9ccccc9c8cc76)ccc54)cc3-n3c4ccccc4c4ccccc43)cccc2c1 ; c1ccc2c(-c3ccc(-n4c5ccccc5c5cc(-n6c7ccccc7c7c8c(ccc76)sc6ccccc68)ccc54)cc3-n3c4ccccc4c4ccccc43)cccc2c1]

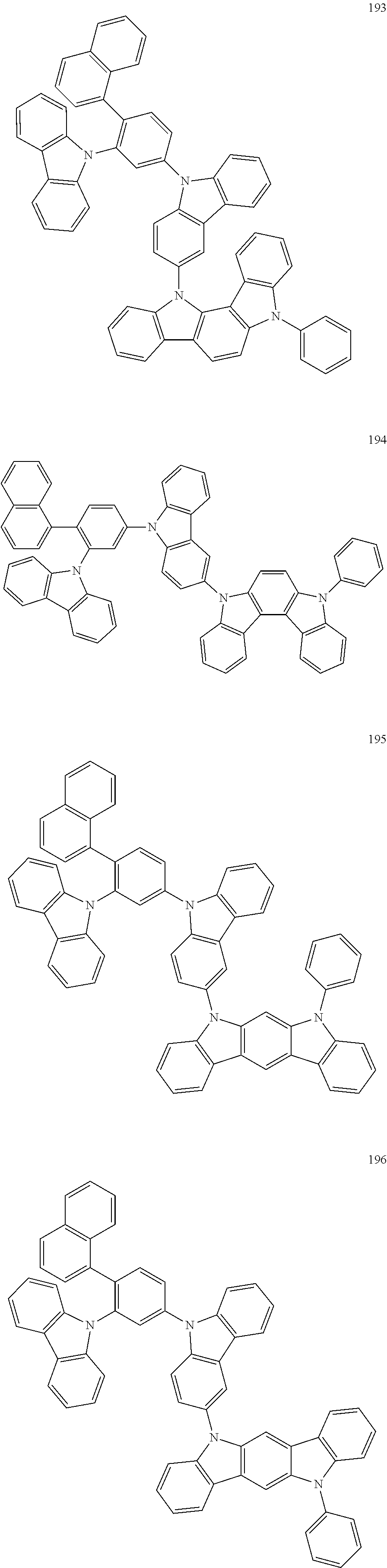 [SMILES: c1ccc(-n2c3ccccc3c3c2ccc2c4ccccc4n(-c4ccc5c(c4)c4ccccc4n5-c4ccc(-c5cccc6ccccc56)c(-n5c6ccccc6c6ccccc65)c4)c23)cc1 ; c1ccc(-n2c3ccccc3c3c4c5ccccc5n(-c5ccc6c(c5)c5ccccc5n6-c5ccc(-c6cccc7ccccc67)c(-n6c7ccccc7c7ccccc76)c5)c4ccc32)cc1 ; c1ccc(-n2c3ccccc3c3cc4c5ccccc5n(-c5ccc6c(c5)c5ccccc5n6-c5ccc(-c6cccc7ccccc67)c(-n6c7ccccc7c7ccccc76)c5)c4cc32)cc1 ; c1ccc(-n2c3ccccc3c3cc4c(cc32)c2ccccc2n4-c2ccc3c(c2)c2ccccc2n3-c2ccc(-c3cccc4ccccc34)c(-n3c4ccccc4c4ccccc43)c2)cc1]

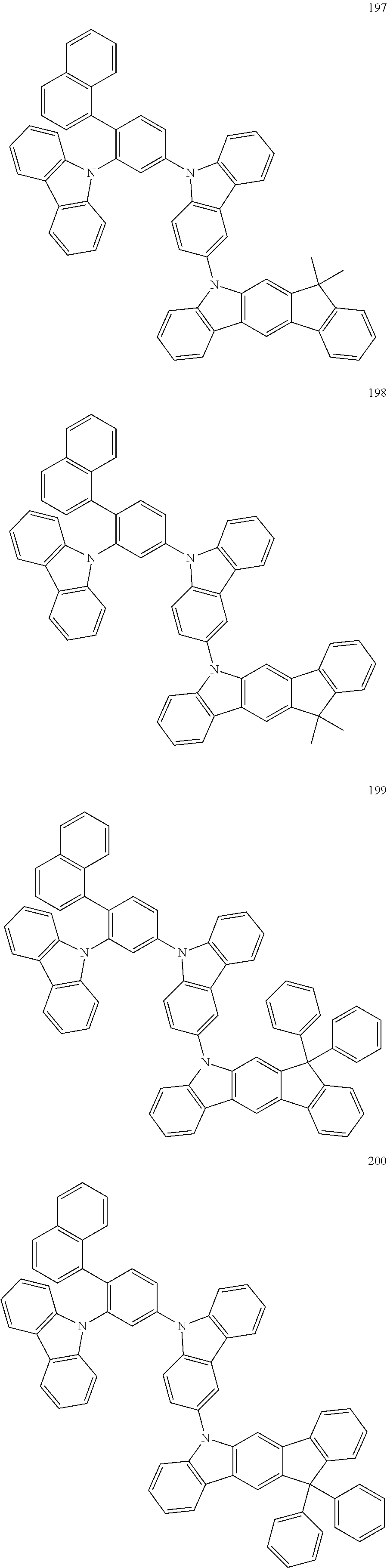 [SMILES: CC1(C)c2ccccc2-c2cc3c4ccccc4n(-c4ccc5c(c4)c4ccccc4n5-c4ccc(-c5cccc6ccccc56)c(-n5c6ccccc6c6ccccc65)c4)c3cc21 ; CC1(C)c2ccccc2-c2cc3c(cc21)c1ccccc1n3-c1ccc2c(c1)c1ccccc1n2-c1ccc(-c2cccc3ccccc23)c(-n2c3ccccc3c3ccccc32)c1 ; c1ccc(C2(c3ccccc3)c3ccccc3-c3cc4c5ccccc5n(-c5ccc6c(c5)c5ccccc5n6-c5ccc(-c6cccc7ccccc67)c(-n6c7ccccc7c7ccccc76)c5)c4cc32)cc1 ; c1ccc(C2(c3ccccc3)c3ccccc3-c3cc4c(cc32)c2ccccc2n4-c2ccc3c(c2)c2ccccc2n3-c2ccc(-c3cccc4ccccc34)c(-n3c4ccccc4c4ccccc43)c2)cc1]

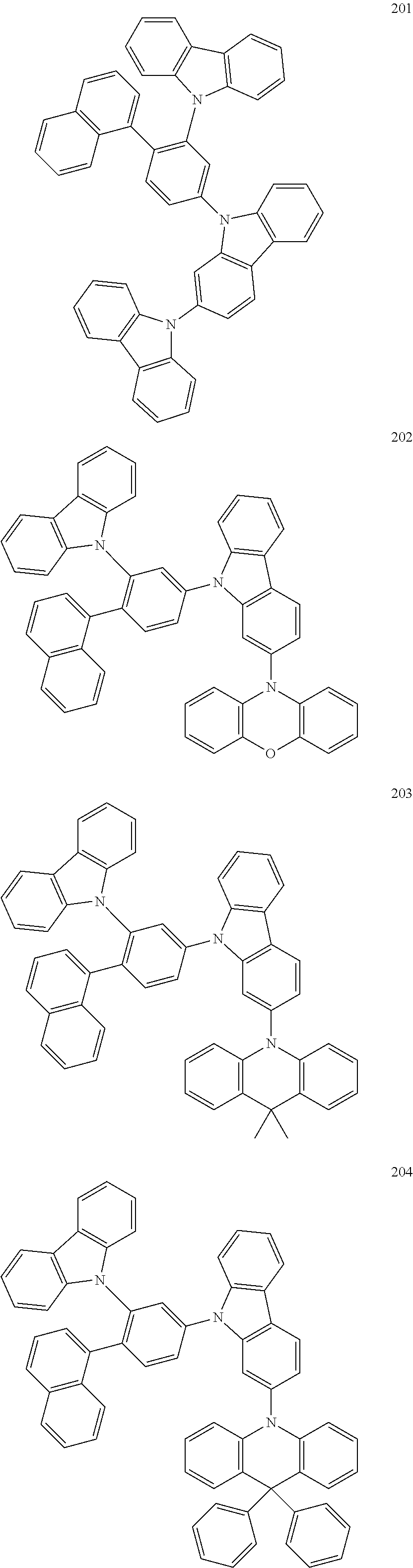

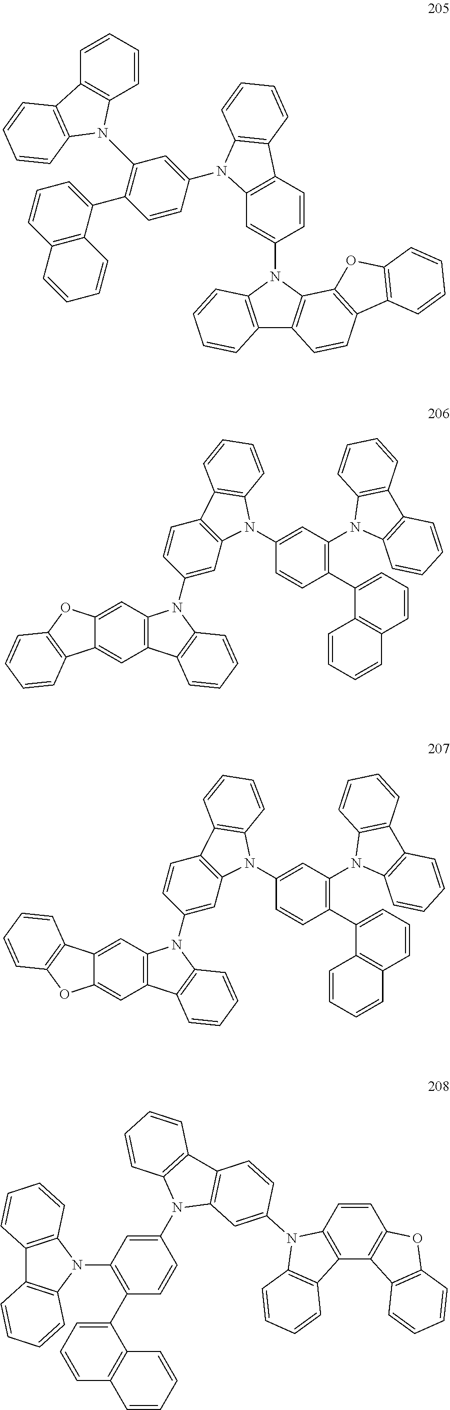 [SMILES: c1ccc2c(-c3ccc(-n4c5ccccc5c5ccc(-n6c7ccccc7c7ccc8c9ccccc9oc8c76)cc54)cc3-n3c4ccccc4c4ccccc43)cccc2c1 ; c1ccc2c(-c3ccc(-n4c5ccccc5c5ccc(-n6c7ccccc7c7cc8c(cc76)oc6ccccc68)cc54)cc3-n3c4ccccc4c4ccccc43)cccc2c1 ; c1ccc2c(-c3ccc(-n4c5ccccc5c5ccc(-n6c7ccccc7c7cc8oc9ccccc9c8cc76)cc54)cc3-n3c4ccccc4c4ccccc43)cccc2c1 ; c1ccc2c(-c3ccc(-n4c5ccccc5c5ccc(-n6c7ccccc7c7c8c(ccc76)oc6ccccc68)cc54)cc3-n3c4ccccc4c4ccccc43)cccc2c1]

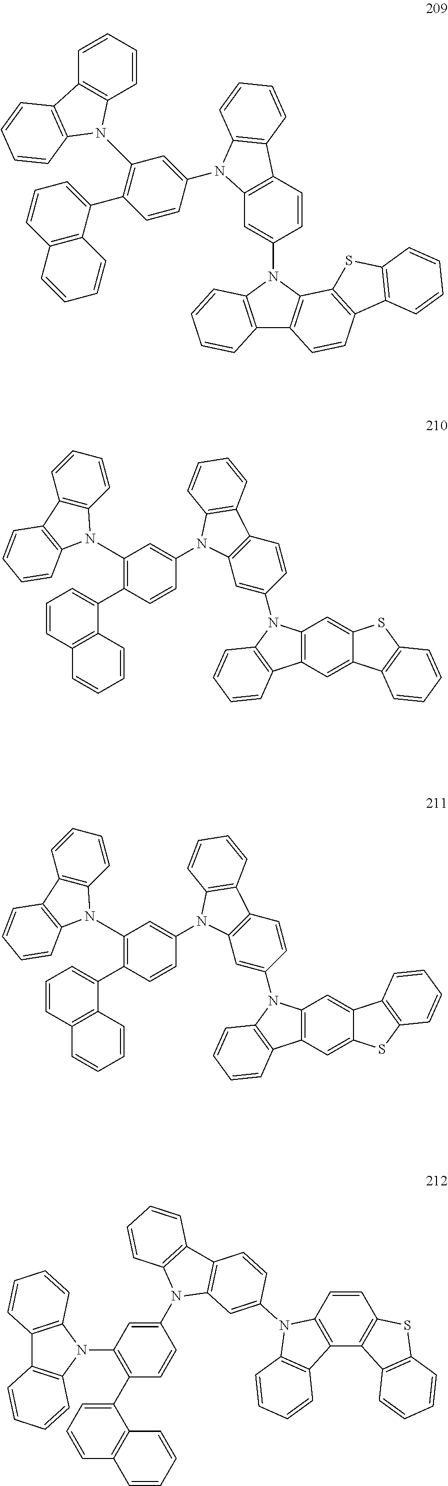 [SMILES: c1ccc2c(-c3ccc(-n4c5ccccc5c5ccc(-n6c7ccccc7c7ccc8c9ccccc9sc8c76)cc54)cc3-n3c4ccccc4c4ccccc43)cccc2c1 ; c1ccc2c(-c3ccc(-n4c5ccccc5c5ccc(-n6c7ccccc7c7cc8c(cc76)sc6ccccc68)cc54)cc3-n3c4ccccc4c4ccccc43)cccc2c1 ; c1ccc2c(-c3ccc(-n4c5ccccc5c5ccc(-n6c7ccccc7c7cc8sc9ccccc9c8cc76)cc54)cc3-n3c4ccccc4c4ccccc43)cccc2c1 ; c1ccc2c(-c3ccc(-n4c5ccccc5c5ccc(-n6c7ccccc7c7c8c(ccc76)sc6ccccc68)cc54)cc3-n3c4ccccc4c4ccccc43)cccc2c1]

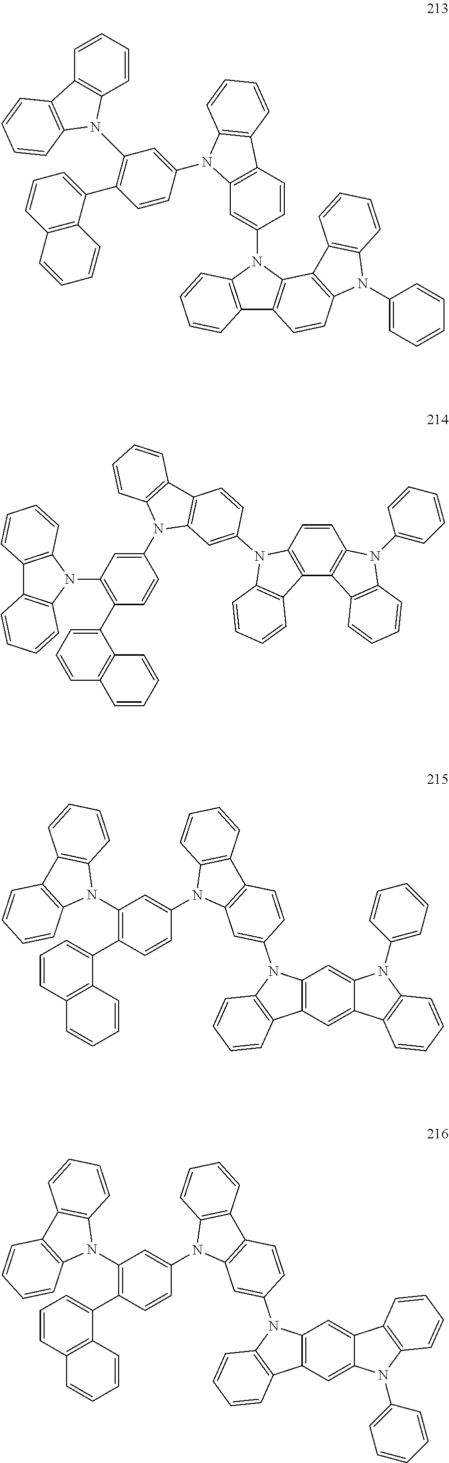 [SMILES: c1ccc(-n2c3ccccc3c3c2ccc2c4ccccc4n(-c4ccc5c6ccccc6n(-c6ccc(-c7cccc8ccccc78)c(-n7c8ccccc8c8ccccc87)c6)c5c4)c23)cc1 ; c1ccc(-n2c3ccccc3c3c4c5ccccc5n(-c5ccc6c7ccccc7n(-c7ccc(-c8cccc9ccccc89)c(-n8c9ccccc9c9ccccc98)c7)c6c5)c4ccc32)cc1 ; c1ccc(-n2c3ccccc3c3cc4c5ccccc5n(-c5ccc6c7ccccc7n(-c7ccc(-c8cccc9ccccc89)c(-n8c9ccccc9c9ccccc98)c7)c6c5)c4cc32)cc1 ; c1ccc(-n2c3ccccc3c3cc4c(cc32)c2ccccc2n4-c2ccc3c4ccccc4n(-c4ccc(-c5cccc6ccccc56)c(-n5c6ccccc6c6ccccc65)c4)c3c2)cc1]

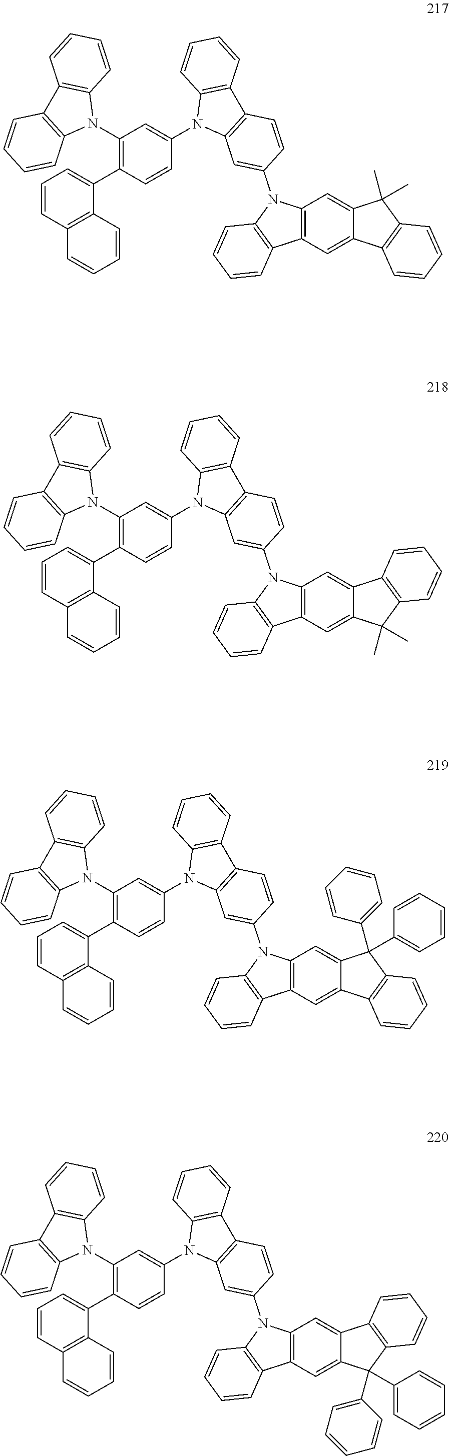 [SMILES: CC1(C)c2ccccc2-c2cc3c4ccccc4n(-c4ccc5c6ccccc6n(-c6ccc(-c7cccc8ccccc78)c(-n7c8ccccc8c8ccccc87)c6)c5c4)c3cc21 ; CC1(C)c2ccccc2-c2cc3c(cc21)c1ccccc1n3-c1ccc2c3ccccc3n(-c3ccc(-c4cccc5ccccc45)c(-n4c5ccccc5c5ccccc54)c3)c2c1 ; c1ccc(C2(c3ccccc3)c3ccccc3-c3cc4c5ccccc5n(-c5ccc6c7ccccc7n(-c7ccc(-c8cccc9ccccc89)c(-n8c9ccccc9c9ccccc98)c7)c6c5)c4cc32)cc1 ; c1ccc(C2(c3ccccc3)c3ccccc3-c3cc4c(cc32)c2ccccc2n4-c2ccc3c4ccccc4n(-c4ccc(-c5cccc6ccccc56)c(-n5c6ccccc6c6ccccc65)c4)c3c2)cc1]

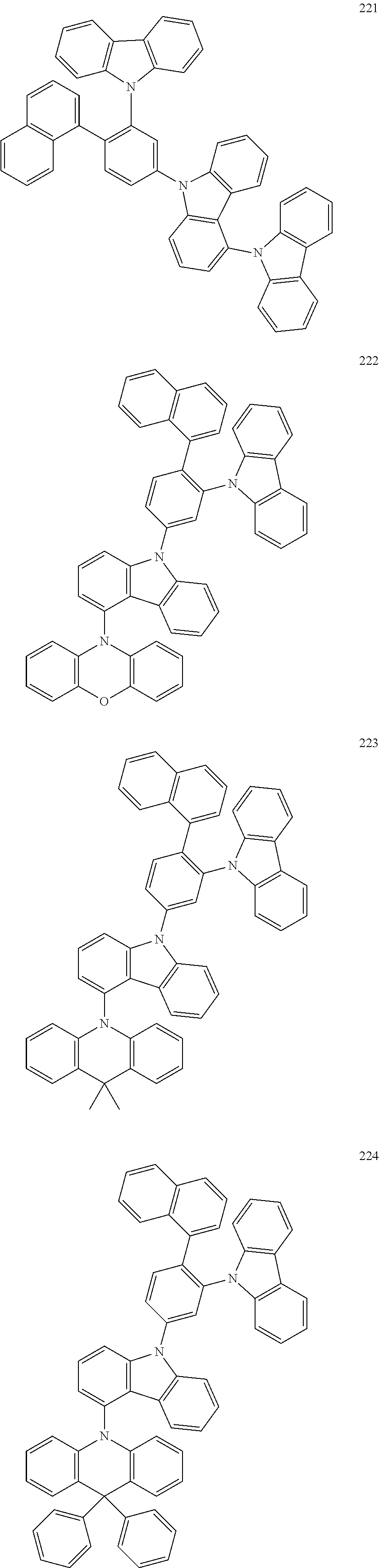 [SMILES: c1ccc2c(-c3ccc(-n4c5ccccc5c5c(-n6c7ccccc7c7ccccc76)cccc54)cc3-n3c4ccccc4c4ccccc43)cccc2c1 ; c1ccc2c(c1)Oc1ccccc1N2c1cccc2c1c1ccccc1n2-c1ccc(-c2cccc3ccccc23)c(-n2c3ccccc3c3ccccc32)c1 ; CC1(C)c2ccccc2N(c2cccc3c2c2ccccc2n3-c2ccc(-c3cccc4ccccc34)c(-n3c4ccccc4c4ccccc43)c2)c2ccccc21 ; c1ccc(C2(c3ccccc3)c3ccccc3N(c3cccc4c3c3ccccc3n4-c3ccc(-c4cccc5ccccc45)c(-n4c5ccccc5c5ccccc54)c3)c3ccccc32)cc1]

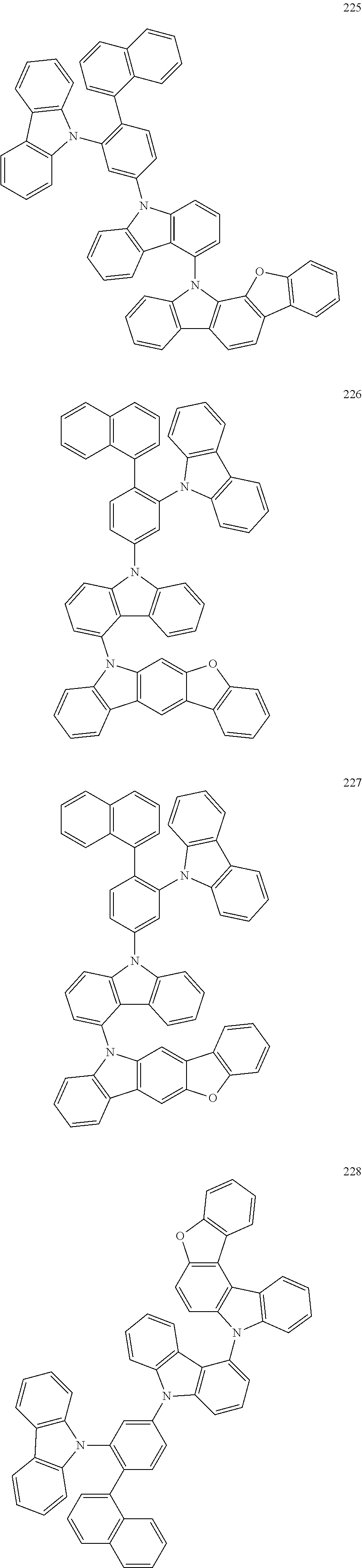 [SMILES: c1ccc2c(-c3ccc(-n4c5ccccc5c5c(-n6c7ccccc7c7ccc8c9ccccc9oc8c76)cccc54)cc3-n3c4ccccc4c4ccccc43)cccc2c1 ; c1ccc2c(-c3ccc(-n4c5ccccc5c5c(-n6c7ccccc7c7cc8c(cc76)oc6ccccc68)cccc54)cc3-n3c4ccccc4c4ccccc43)cccc2c1 ; c1ccc2c(-c3ccc(-n4c5ccccc5c5c(-n6c7ccccc7c7cc8oc9ccccc9c8cc76)cccc54)cc3-n3c4ccccc4c4ccccc43)cccc2c1 ; c1ccc2c(-c3ccc(-n4c5ccccc5c5c(-n6c7ccccc7c7c8c(ccc76)oc6ccccc68)cccc54)cc3-n3c4ccccc4c4ccccc43)cccc2c1]

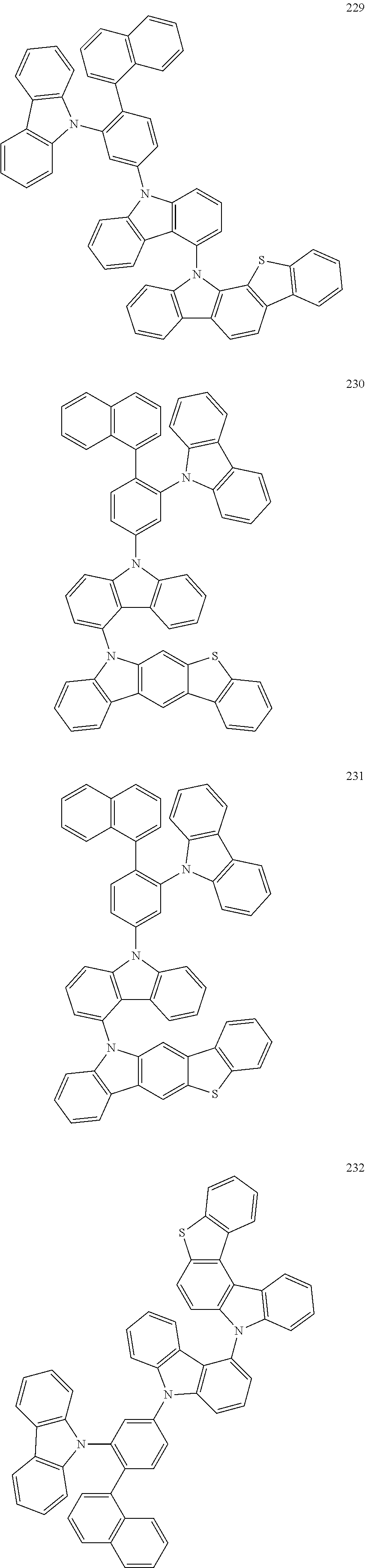 [SMILES: c1ccc2c(-c3ccc(-n4c5ccccc5c5c(-n6c7ccccc7c7ccc8c9ccccc9sc8c76)cccc54)cc3-n3c4ccccc4c4ccccc43)cccc2c1 ; c1ccc2c(-c3ccc(-n4c5ccccc5c5c(-n6c7ccccc7c7cc8c(cc76)sc6ccccc68)cccc54)cc3-n3c4ccccc4c4ccccc43)cccc2c1 ; c1ccc2c(-c3ccc(-n4c5ccccc5c5c(-n6c7ccccc7c7cc8sc9ccccc9c8cc76)cccc54)cc3-n3c4ccccc4c4ccccc43)cccc2c1 ; c1ccc2c(-c3ccc(-n4c5ccccc5c5c(-n6c7ccccc7c7c8c(ccc76)sc6ccccc68)cccc54)cc3-n3c4ccccc4c4ccccc43)cccc2c1]

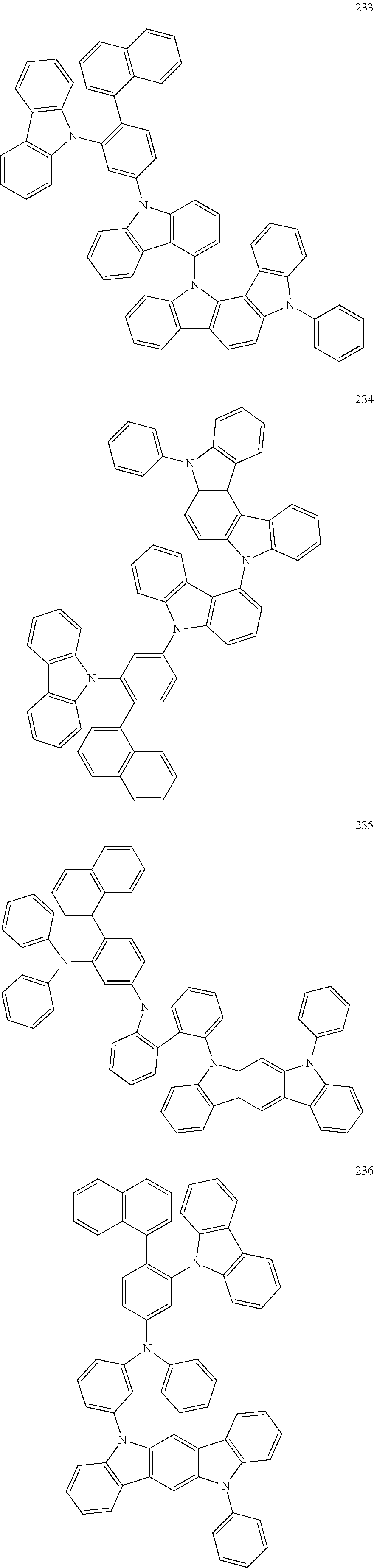 [SMILES: c1ccc(-n2c3ccccc3c3c2ccc2c4ccccc4n(-c4cccc5c4c4ccccc4n5-c4ccc(-c5cccc6ccccc56)c(-n5c6ccccc6c6ccccc65)c4)c23)cc1 ; c1ccc(-n2c3ccccc3c3c4c5ccccc5n(-c5cccc6c5c5ccccc5n6-c5ccc(-c6cccc7ccccc67)c(-n6c7ccccc7c7ccccc76)c5)c4ccc32)cc1 ; c1ccc(-n2c3ccccc3c3cc4c5ccccc5n(-c5cccc6c5c5ccccc5n6-c5ccc(-c6cccc7ccccc67)c(-n6c7ccccc7c7ccccc76)c5)c4cc32)cc1 ; c1ccc(-n2c3ccccc3c3cc4c(cc32)c2ccccc2n4-c2cccc3c2c2ccccc2n3-c2ccc(-c3cccc4ccccc34)c(-n3c4ccccc4c4ccccc43)c2)cc1]

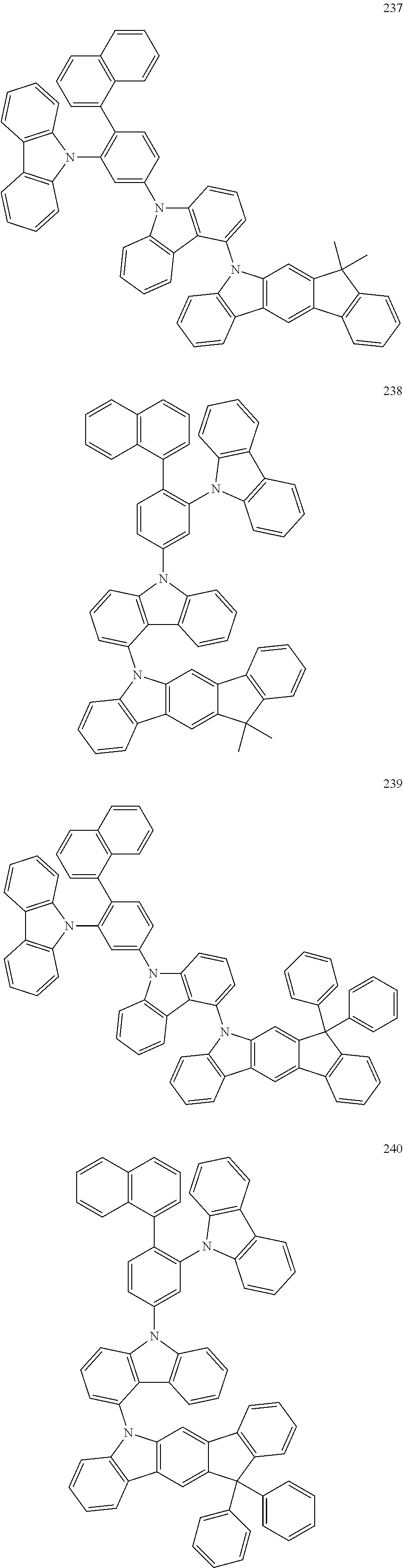 [SMILES: CC1(C)c2ccccc2-c2cc3c4ccccc4n(-c4cccc5c4c4ccccc4n5-c4ccc(-c5cccc6ccccc56)c(-n5c6ccccc6c6ccccc65)c4)c3cc21 ; CC1(C)c2ccccc2-c2cc3c(cc21)c1ccccc1n3-c1cccc2c1c1ccccc1n2-c1ccc(-c2cccc3ccccc23)c(-n2c3ccccc3c3ccccc32)c1 ; c1ccc(C2(c3ccccc3)c3ccccc3-c3cc4c5ccccc5n(-c5cccc6c5c5ccccc5n6-c5ccc(-c6cccc7ccccc67)c(-n6c7ccccc7c7ccccc76)c5)c4cc32)cc1 ; c1ccc(C2(c3ccccc3)c3ccccc3-c3cc4c(cc32)c2ccccc2n4-c2cccc3c2c2ccccc2n3-c2ccc(-c3cccc4ccccc34)c(-n3c4ccccc4c4ccccc43)c2)cc1]

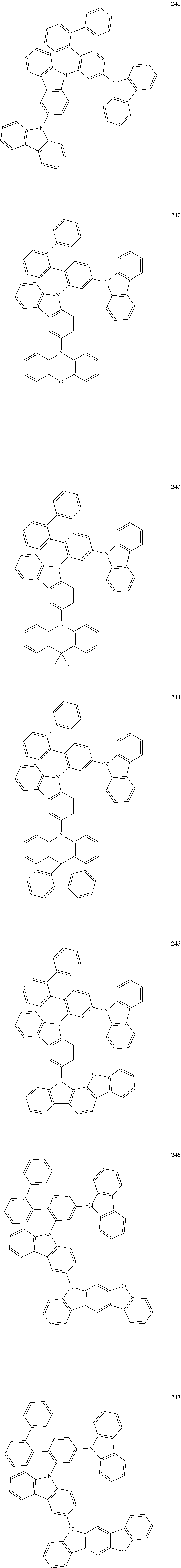

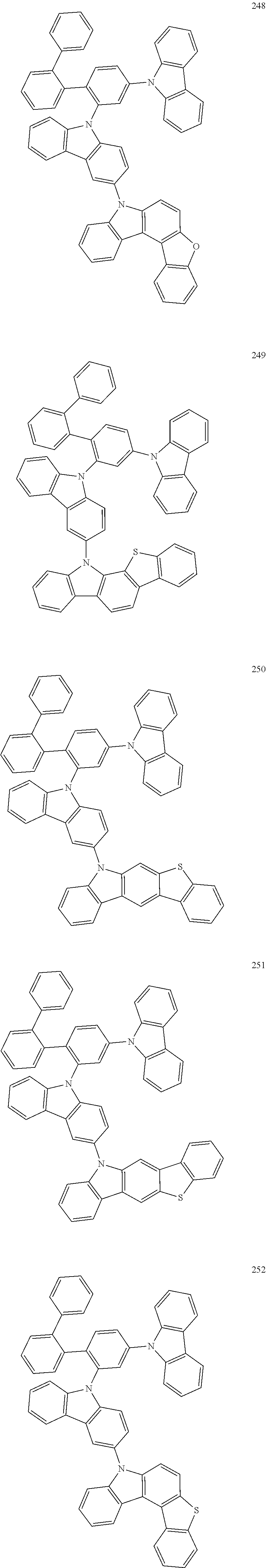

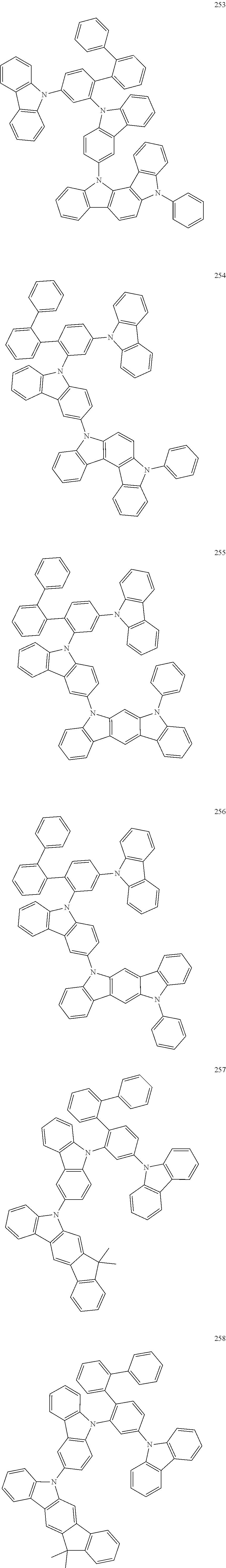

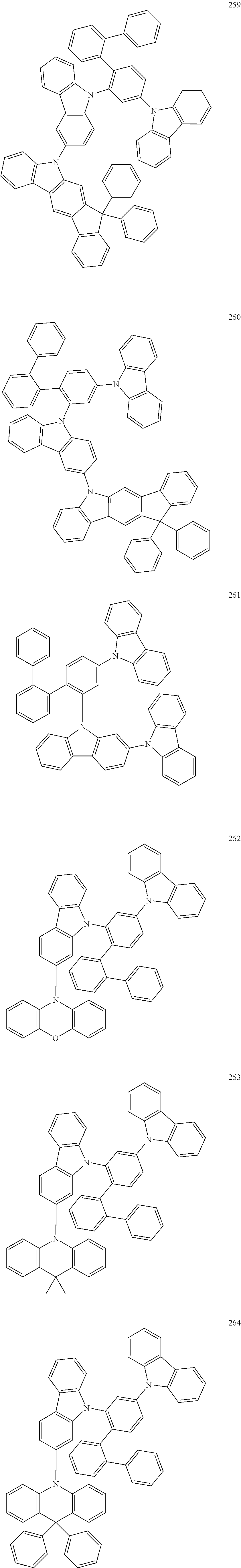

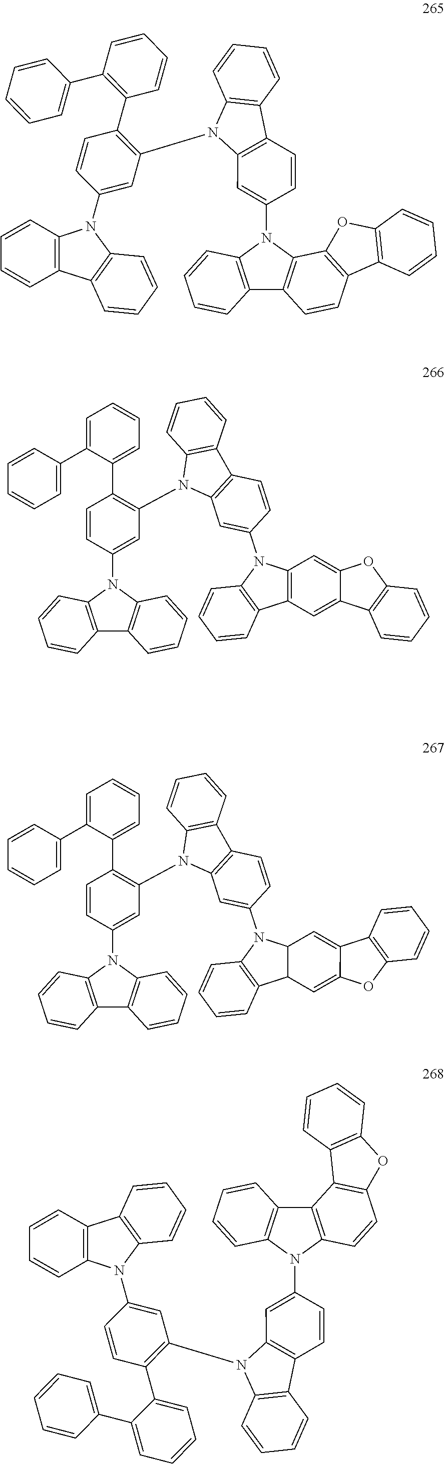 [SMILES: c1ccc(-c2ccccc2-c2ccc(-n3c4ccccc4c4ccccc43)cc2-n2c3ccccc3c3ccc(-n4c5ccccc5c5ccc6c7ccccc7oc6c54)cc32)cc1 ; c1ccc(-c2ccccc2-c2ccc(-n3c4ccccc4c4ccccc43)cc2-n2c3ccccc3c3ccc(-n4c5ccccc5c5cc6c(cc54)oc4ccccc46)cc32)cc1 ; C1=c2oc3ccccc3c2=CC2C1c1ccccc1N2c1ccc2c3ccccc3n(-c3cc(-n4c5ccccc5c5ccccc54)ccc3-c3ccccc3-c3ccccc3)c2c1 ; c1ccc(-c2ccccc2-c2ccc(-n3c4ccccc4c4ccccc43)cc2-n2c3ccccc3c3ccc(-n4c5ccccc5c5c6c(ccc54)oc4ccccc46)cc32)cc1]

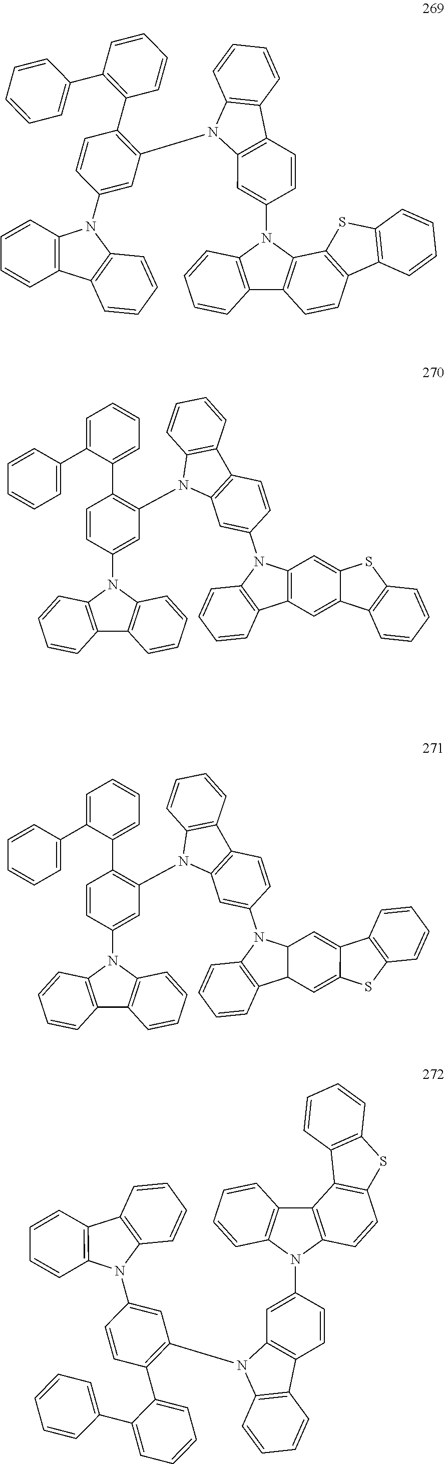 [SMILES: c1ccc(-c2ccccc2-c2ccc(-n3c4ccccc4c4ccccc43)cc2-n2c3ccccc3c3ccc(-n4c5ccccc5c5ccc6c7ccccc7sc6c54)cc32)cc1 ; c1ccc(-c2ccccc2-c2ccc(-n3c4ccccc4c4ccccc43)cc2-n2c3ccccc3c3ccc(-n4c5ccccc5c5cc6c(cc54)sc4ccccc46)cc32)cc1 ; C1=c2sc3ccccc3c2=CC2C1c1ccccc1N2c1ccc2c3ccccc3n(-c3cc(-n4c5ccccc5c5ccccc54)ccc3-c3ccccc3-c3ccccc3)c2c1 ; c1ccc(-c2ccccc2-c2ccc(-n3c4ccccc4c4ccccc43)cc2-n2c3ccccc3c3ccc(-n4c5ccccc5c5c6c(ccc54)sc4ccccc46)cc32)cc1]

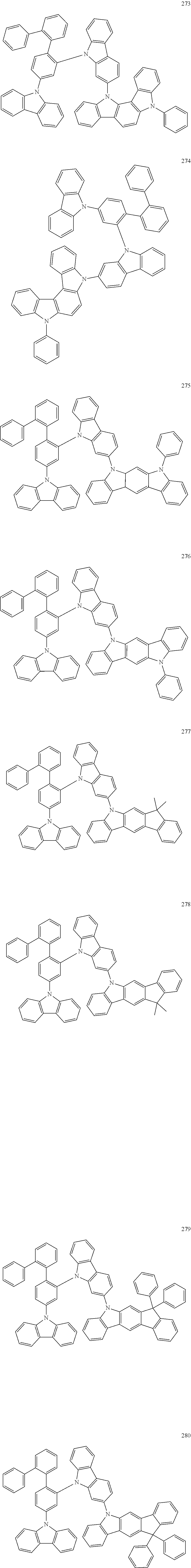

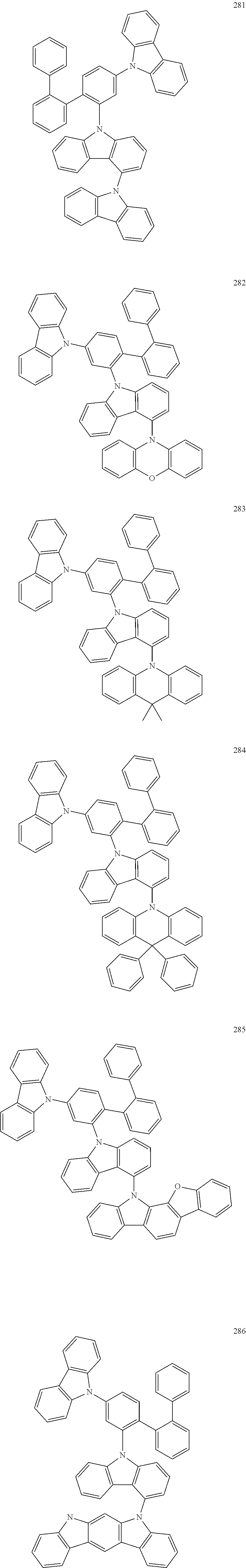

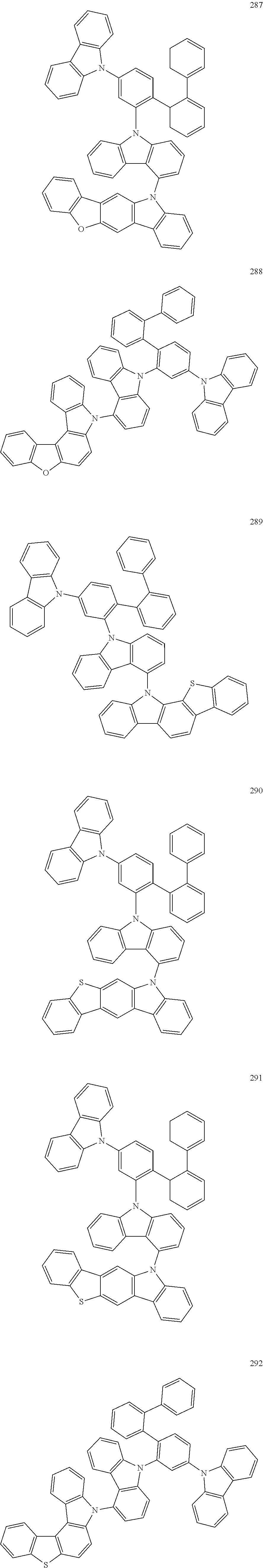

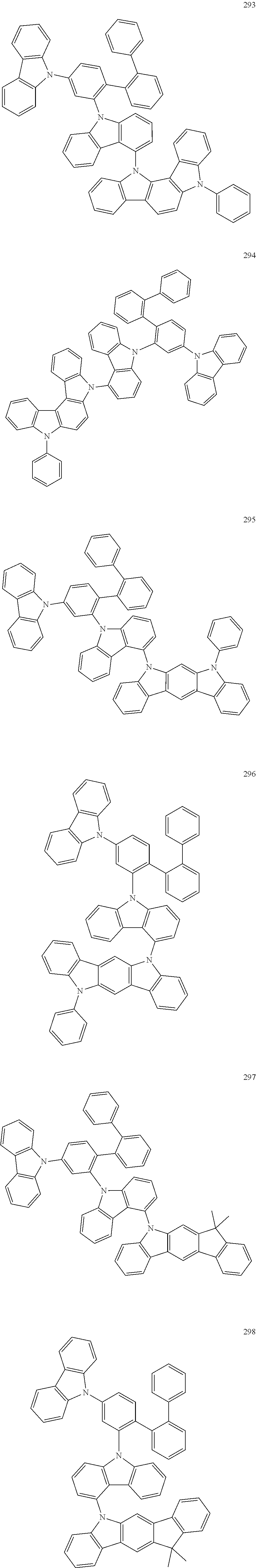

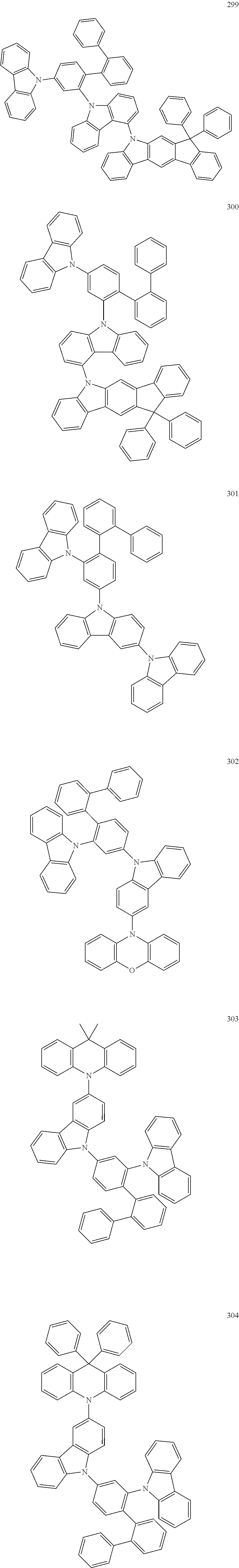

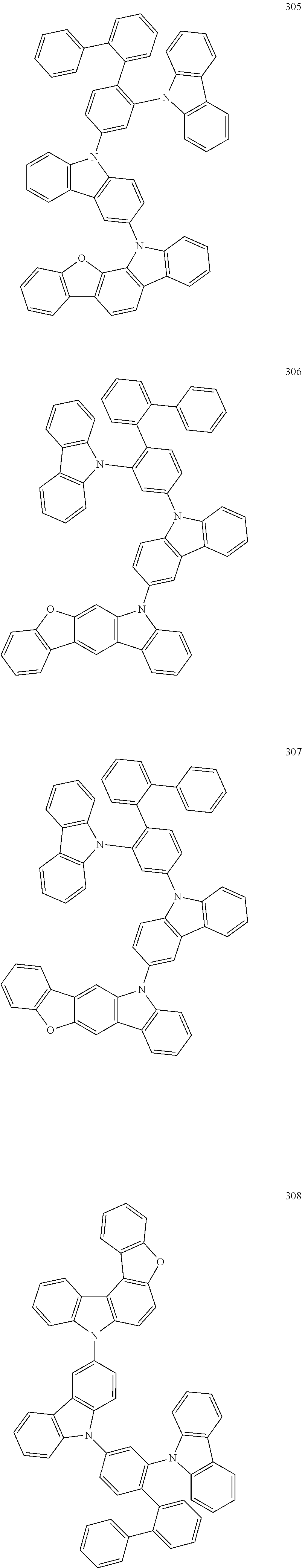 [SMILES: c1ccc(-c2ccccc2-c2ccc(-n3c4ccccc4c4cc(-n5c6ccccc6c6ccc7c8ccccc8oc7c65)ccc43)cc2-n2c3ccccc3c3ccccc32)cc1 ; c1ccc(-c2ccccc2-c2ccc(-n3c4ccccc4c4cc(-n5c6ccccc6c6cc7c(cc65)oc5ccccc57)ccc43)cc2-n2c3ccccc3c3ccccc32)cc1 ; c1ccc(-c2ccccc2-c2ccc(-n3c4ccccc4c4cc(-n5c6ccccc6c6cc7oc8ccccc8c7cc65)ccc43)cc2-n2c3ccccc3c3ccccc32)cc1 ; c1ccc(-c2ccccc2-c2ccc(-n3c4ccccc4c4cc(-n5c6ccccc6c6c7c(ccc65)oc5ccccc57)ccc43)cc2-n2c3ccccc3c3ccccc32)cc1]

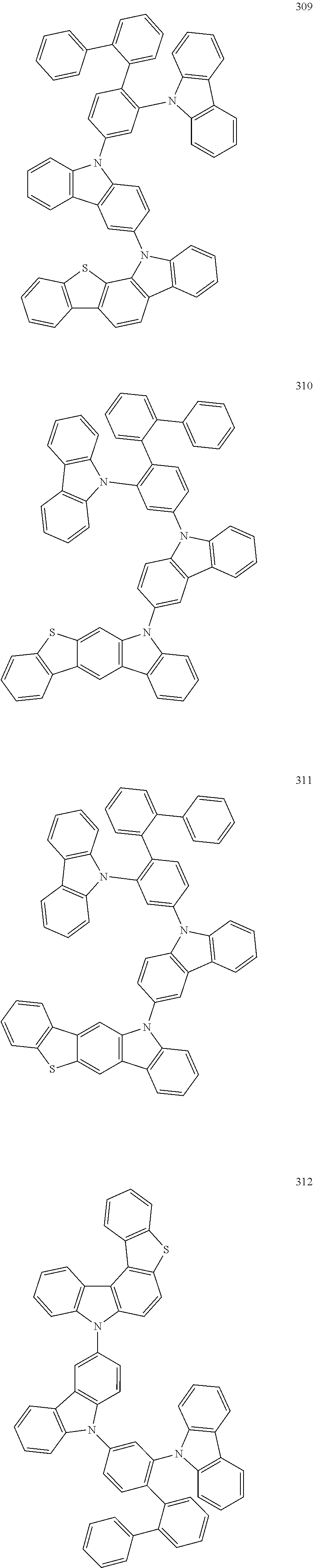 [SMILES: c1ccc(-c2ccccc2-c2ccc(-n3c4ccccc4c4cc(-n5c6ccccc6c6ccc7c8ccccc8sc7c65)ccc43)cc2-n2c3ccccc3c3ccccc32)cc1 ; c1ccc(-c2ccccc2-c2ccc(-n3c4ccccc4c4cc(-n5c6ccccc6c6cc7c(cc65)sc5ccccc57)ccc43)cc2-n2c3ccccc3c3ccccc32)cc1 ; c1ccc(-c2ccccc2-c2ccc(-n3c4ccccc4c4cc(-n5c6ccccc6c6cc7sc8ccccc8c7cc65)ccc43)cc2-n2c3ccccc3c3ccccc32)cc1 ; c1ccc(-c2ccccc2-c2ccc(-n3c4ccccc4c4cc(-n5c6ccccc6c6c7c(ccc65)sc5ccccc57)ccc43)cc2-n2c3ccccc3c3ccccc32)cc1]

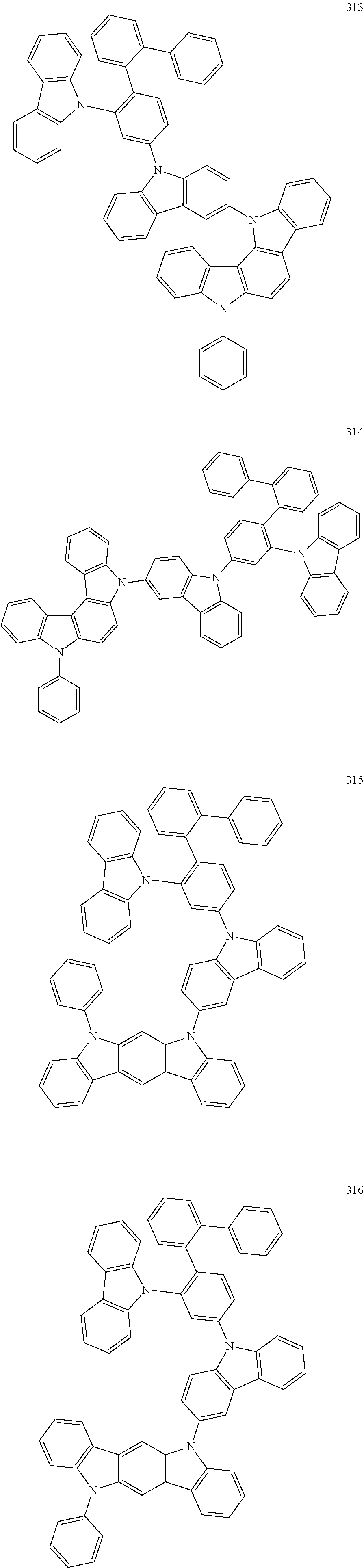 [SMILES: c1ccc(-c2ccccc2-c2ccc(-n3c4ccccc4c4cc(-n5c6ccccc6c6ccc7c(c8ccccc8n7-c7ccccc7)c65)ccc43)cc2-n2c3ccccc3c3ccccc32)cc1 ; c1ccc(-c2ccccc2-c2ccc(-n3c4ccccc4c4cc(-n5c6ccccc6c6c7c8ccccc8n(-c8ccccc8)c7ccc65)ccc43)cc2-n2c3ccccc3c3ccccc32)cc1 ; c1ccc(-c2ccccc2-c2ccc(-n3c4ccccc4c4cc(-n5c6ccccc6c6cc7c8ccccc8n(-c8ccccc8)c7cc65)ccc43)cc2-n2c3ccccc3c3ccccc32)cc1 ; c1ccc(-c2ccccc2-c2ccc(-n3c4ccccc4c4cc(-n5c6ccccc6c6cc7c(cc65)c5ccccc5n7-c5ccccc5)ccc43)cc2-n2c3ccccc3c3ccccc32)cc1]

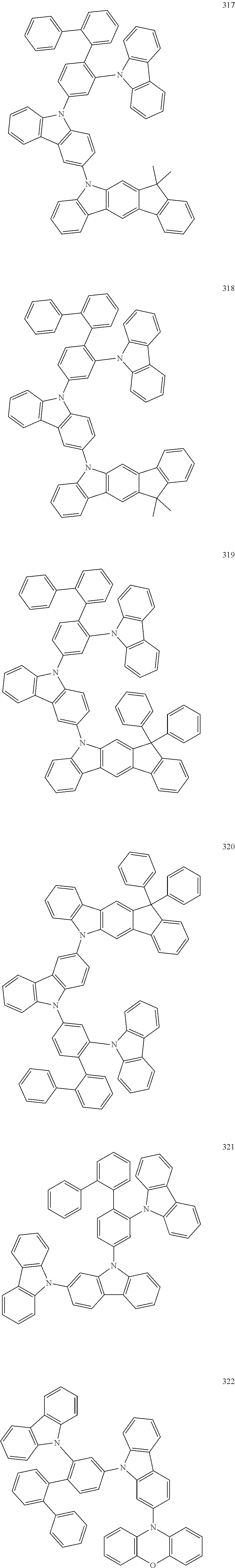

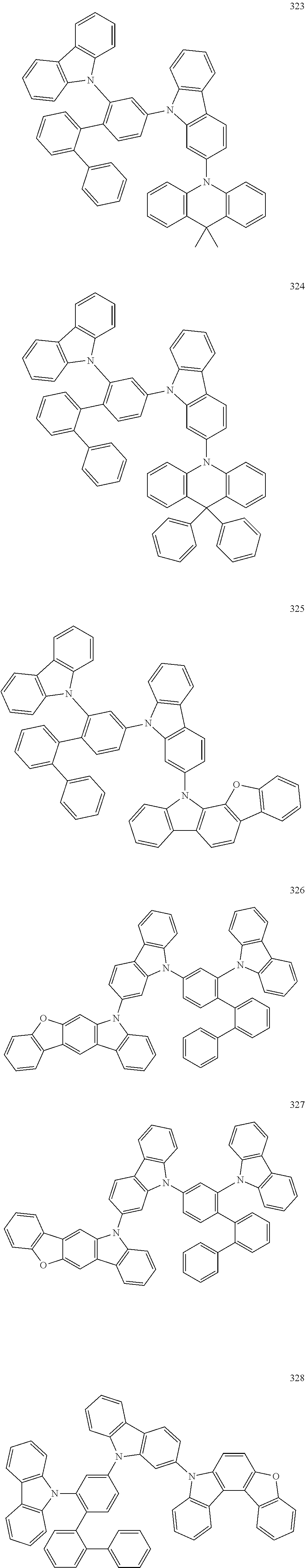

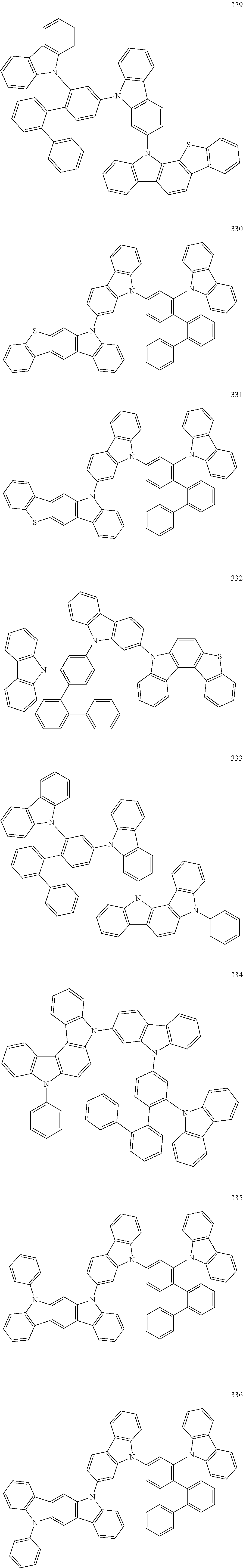

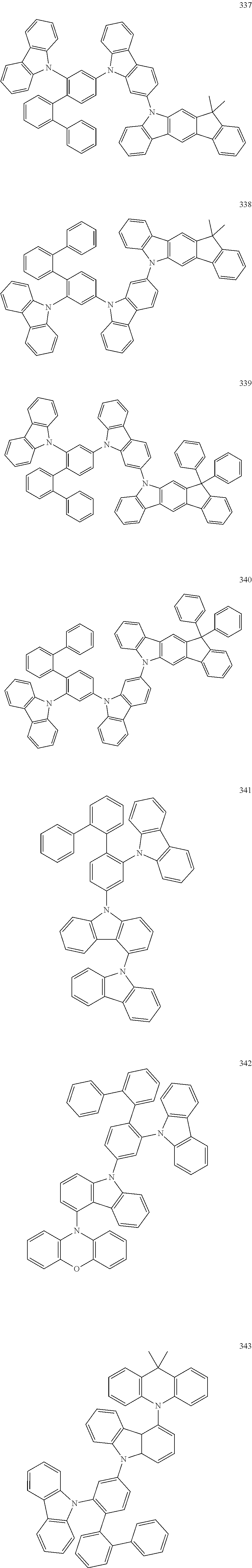

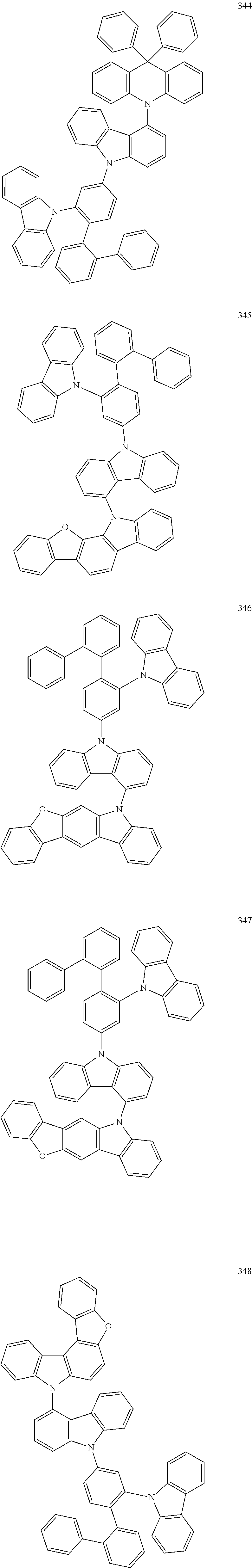

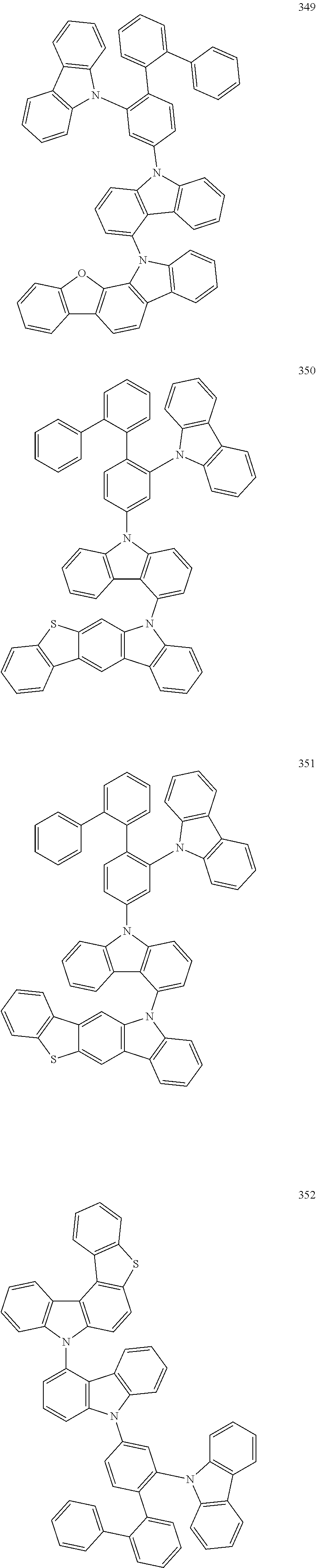 [SMILES: c1ccc(-c2ccccc2-c2ccc(-n3c4ccccc4c4c(-n5c6ccccc6c6ccc7c8ccccc8oc7c65)cccc43)cc2-n2c3ccccc3c3ccccc32)cc1 ; c1ccc(-c2ccccc2-c2ccc(-n3c4ccccc4c4c(-n5c6ccccc6c6cc7c(cc65)sc5ccccc57)cccc43)cc2-n2c3ccccc3c3ccccc32)cc1 ; c1ccc(-c2ccccc2-c2ccc(-n3c4ccccc4c4c(-n5c6ccccc6c6cc7sc8ccccc8c7cc65)cccc43)cc2-n2c3ccccc3c3ccccc32)cc1 ; c1ccc(-c2ccccc2-c2ccc(-n3c4ccccc4c4c(-n5c6ccccc6c6c7c(ccc65)sc5ccccc57)cccc43)cc2-n2c3ccccc3c3ccccc32)cc1]

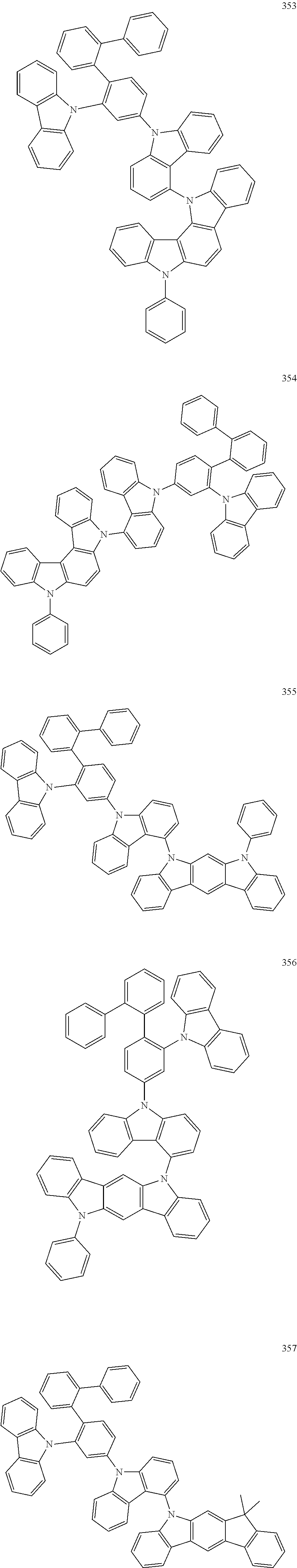

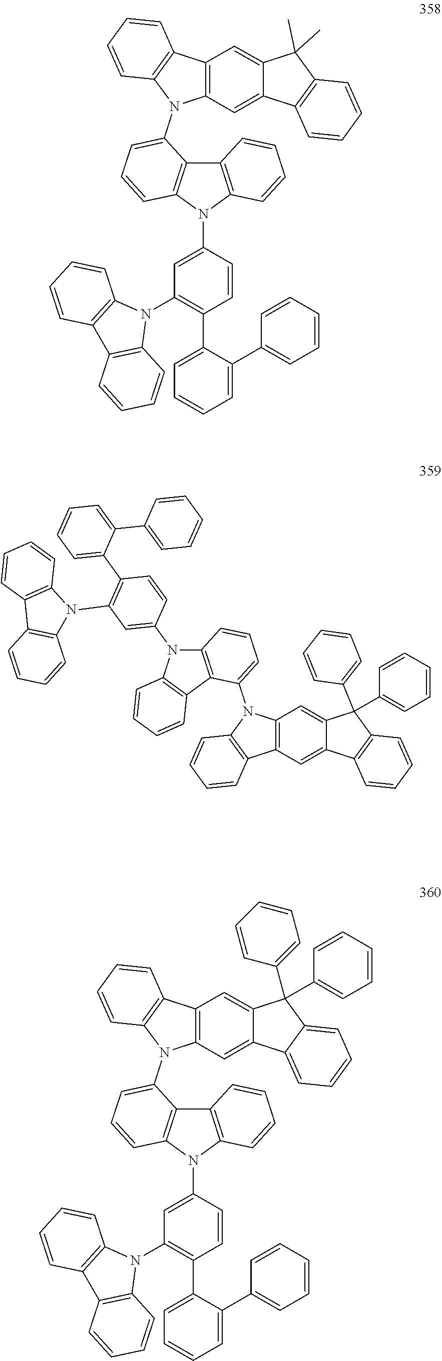 [SMILES: CC1(C)c2ccccc2-c2cc3c(cc21)c1ccccc1n3-c1cccc2c1c1ccccc1n2-c1ccc(-c2ccccc2-c2ccccc2)c(-n2c3ccccc3c3ccccc32)c1 ; c1ccc(-c2ccccc2-c2ccc(-n3c4ccccc4c4c(-n5c6ccccc6c6cc7c(cc65)C(c5ccccc5)(c5ccccc5)c5ccccc5-7)cccc43)cc2-n2c3ccccc3c3ccccc32)cc1 ; c1ccc(-c2ccccc2-c2ccc(-n3c4ccccc4c4c(-n5c6ccccc6c6cc7c(cc65)-c5ccccc5C7(c5ccccc5)c5ccccc5)cccc43)cc2-n2c3ccccc3c3ccccc32)cc1]

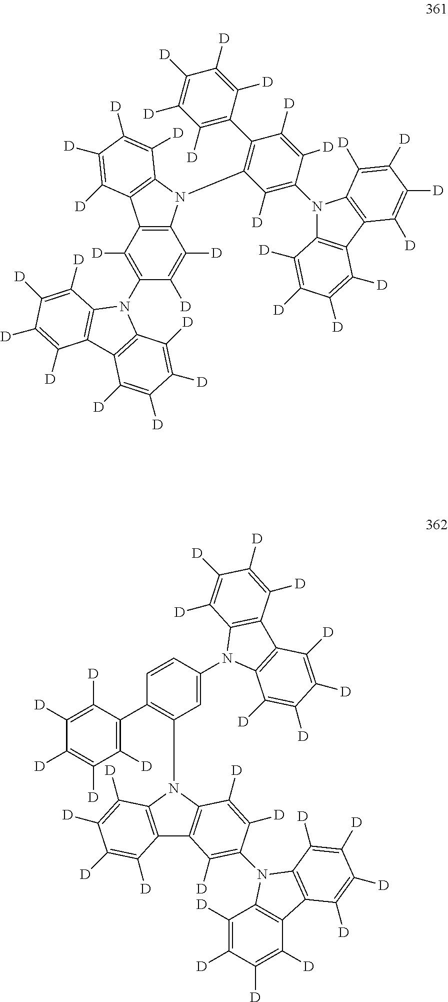

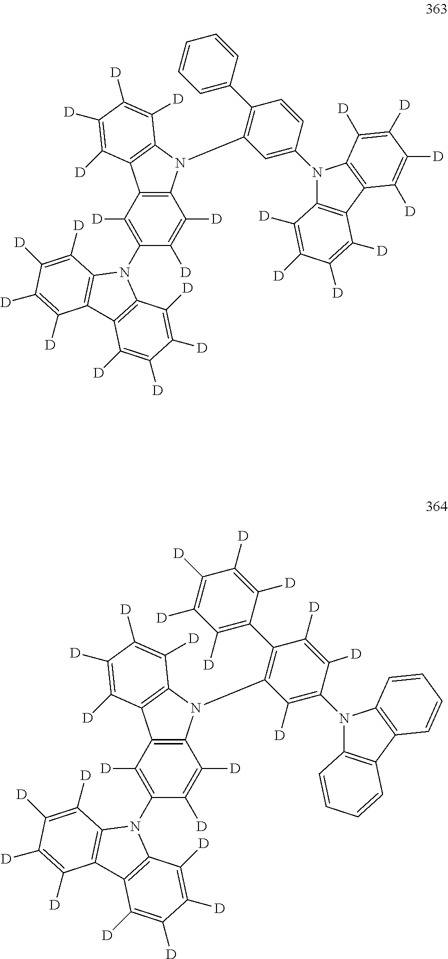 [SMILES: [2H]c1c([2H])c([2H])c2c(c1[2H])c1c([2H])c([2H])c([2H])c([2H])c1n2-c1ccc(-c2ccccc2)c(-n2c3c([2H])c([2H])c([2H])c([2H])c3c3c([2H])c(-n4c5c([2H])c([2H])c([2H])c([2H])c5c5c([2H])c([2H])c([2H])c([2H])c54)c([2H])c([2H])c32)c1 ; [2H]c1c([2H])c([2H])c(-c2c([2H])c([2H])c(-n3c4ccccc4c4ccccc43)c([2H])c2-n2c3c([2H])c([2H])c([2H])c([2H])c3c3c([2H])c(-n4c5c([2H])c([2H])c([2H])c([2H])c5c5c([2H])c([2H])c([2H])c([2H])c54)c([2H])c([2H])c32)c([2H])c1[2H]]

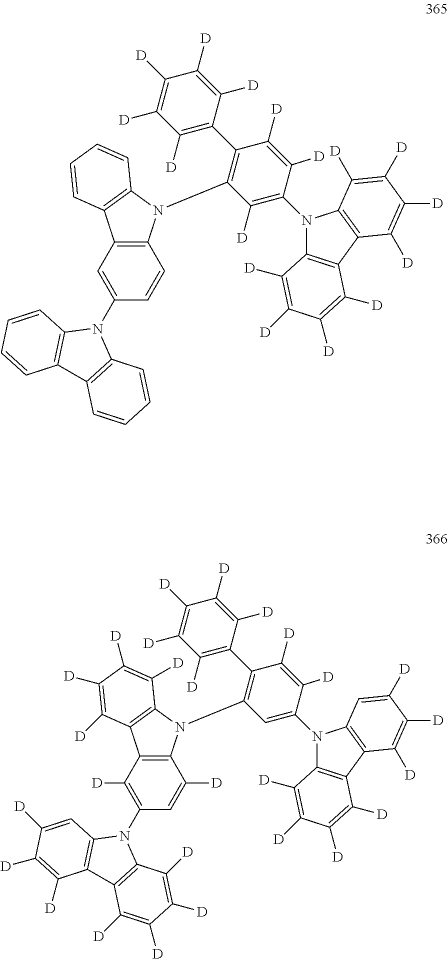 [SMILES: [2H]c1c([2H])c([2H])c(-c2c([2H])c([2H])c(-n3c4c([2H])c([2H])c([2H])c([2H])c4c4c([2H])c([2H])c([2H])c([2H])c43)c([2H])c2-n2c3ccccc3c3cc(-n4c5ccccc5c5ccccc54)ccc32)c([2H])c1[2H] ; [2H]c1cc2c(c([2H])c1[2H])c1c([2H])c([2H])c([2H])c([2H])c1n2-c1cc(-n2c3c([2H])cc(-n4c5cc([2H])c([2H])c([2H])c5c5c([2H])c([2H])c([2H])c([2H])c54)c([2H])c3c3c([2H])c([2H])c([2H])c([2H])c32)c(-c2c([2H])c([2H])c([2H])c([2H])c2[2H])c([2H])c1[2H]]

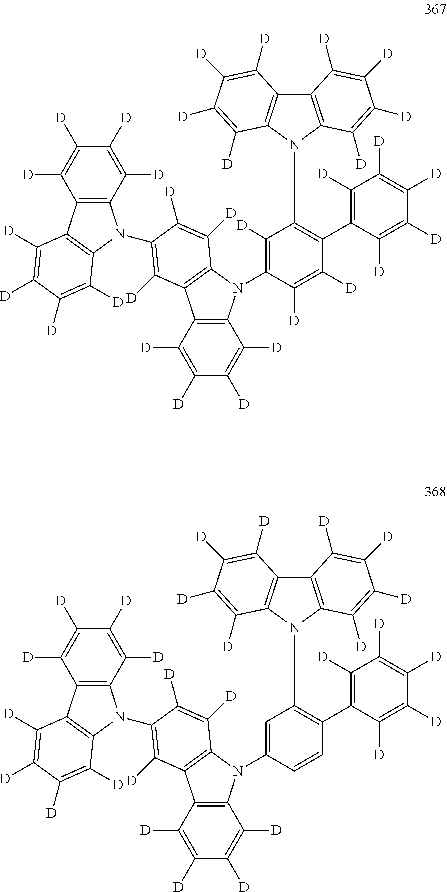

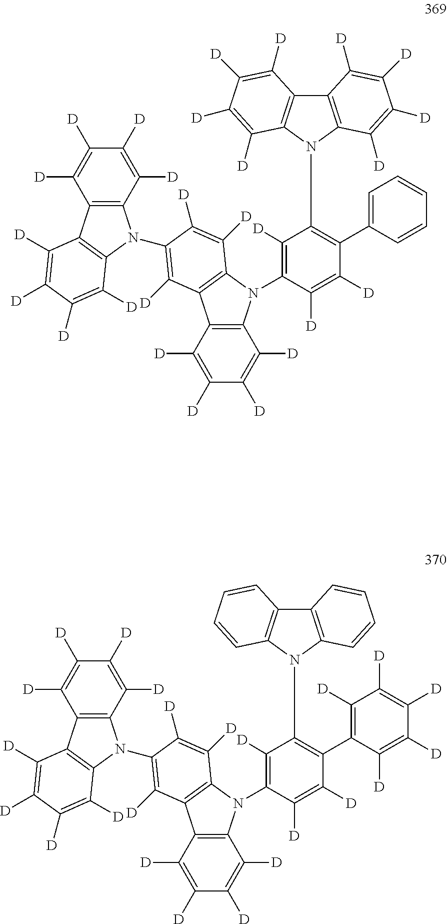 [SMILES: [2H]c1c([2H])c(-n2c3c([2H])c([2H])c([2H])c([2H])c3c3c([2H])c(-n4c5c([2H])c([2H])c([2H])c([2H])c5c5c([2H])c([2H])c([2H])c([2H])c54)c([2H])c([2H])c32)c([2H])c(-n2c3c([2H])c([2H])c([2H])c([2H])c3c3c([2H])c([2H])c([2H])c([2H])c32)c1-c1ccccc1 ; [2H]c1c([2H])c([2H])c(-c2c([2H])c([2H])c(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c([2H])c2-n2c3ccccc3c3ccccc32)c([2H])c1[2H]]

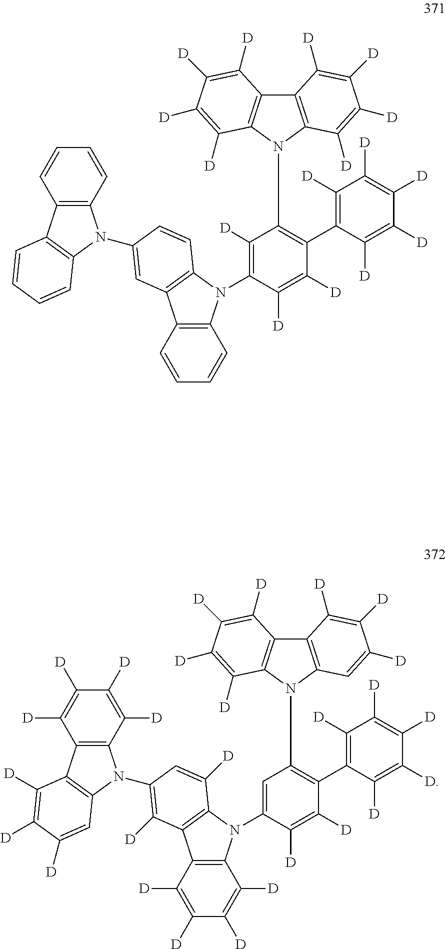

In addition, by introducing various substituents to the structure of chemical formula 1, compounds having unique properties of the introduced substituents may be synthesized. For example, by introducing substituents normally used for a hole injection layer material, a hole transport layer material, a hole transport auxiliary layer material, an electron blocking layer material, a light emitting layer material, an electron transport layer material, a hole blocking layer material and an electron injection layer material used for manufacturing an organic light-emitting device to the core structure, materials satisfying conditions required for each organic material layer may be synthesized.

In addition, by introducing various substituents to the structure of chemical formula 1, the energy band gap may be finely controlled, and meanwhile, properties at interfaces between organic materials may be enhanced, and material applications may become diverse.

One embodiment of the present invention provides an organic light-emitting device including the heterocyclic compound represented by chemical formula 1. The “organic light-emitting device” may be expressed in terms such as an “organic light emitting diode”, an “OLED”, an “OLED device” and an “organic electroluminescent device”.

In addition, one embodiment of the present invention provides an organic light-emitting device including: a first electrode; a second electrode provided opposite to the first electrode; and one or more organic material layers provided between the first electrode and the second electrode, wherein one or more layers of the organic material layers include the heterocyclic compound represented by chemical formula 1.

In one embodiment of the present invention, the first electrode may be a positive electrode, and the second electrode may be a negative electrode.

In one embodiment of the present invention, the first electrode may be a negative electrode, and the second electrode may be a positive electrode.

In one embodiment of the present invention, the organic material layer may include one or more selected from the group consisting of an electron injection layer, an electron transport layer, a hole blocking layer, a light emitting layer, an electron blocking layer, a hole transport layer and a hole injection layer, and the one or more layers selected from the group consisting of an electron injection layer, an electron transport layer, a hole blocking layer, a light emitting layer, an electron blocking layer, a hole transport layer and a hole injection layer may include the heterocyclic compound represented by chemical formula 1.

In one embodiment of the present invention, the organic material layer may include a light emitting layer, and the light emitting layer may include the heterocyclic compound represented by chemical formula 1.

In one embodiment of the present invention, the organic material layer includes a light emitting layer, the light emitting layer includes a host material, and the host material may include the heterocyclic compound represented by chemical formula 1.

In one embodiment of the present invention, the organic light-emitting device may be a red organic light-emitting device, and the heterocyclic compound represented by chemical formula 1 may be used as a material of the red organic light-emitting device.

In one embodiment of the present invention, the organic light-emitting device may be a green organic light-emitting device, and the heterocyclic compound represented by chemical formula 1 may be used as a material of the green organic light-emitting device.

In one embodiment of the present invention, the organic light-emitting device may be a blue organic light-emitting device, and the heterocyclic compound represented by chemical formula 1 may be used as a material of the blue organic light-emitting device.

In one embodiment of the present invention, the organic light-emitting device may be a red organic light-emitting device, and the heterocyclic compound represented by chemical formula 1 may be used as a material of a light emitting layer of the red organic light-emitting device.

In one embodiment of the present invention, the organic light-emitting device may be a green organic light-emitting device, and the heterocyclic compound represented by chemical formula 1 may be used as a material of a light emitting layer of the green organic light-emitting device.

In one embodiment of the present invention, the organic light-emitting device may be a blue organic light-emitting device, and the heterocyclic compound represented by chemical formula 1 may be used as a material of a light emitting layer of the blue organic light-emitting device.

Specific descriptions on the heterocyclic compound represented by chemical formula 1 are the same as the descriptions provided above.

In the organic light-emitting device according to one embodiment of the present invention, the organic material layer includes an electron injection layer or an electron transport layer, and the electron injection layer or the electron transport layer may include the heterocyclic compound.

In the organic light-emitting device according to one embodiment of the present invention, the organic material layer includes an electron blocking layer or a hole blocking layer, and the electron blocking layer or the hole blocking layer may include the heterocyclic compound.

In the organic light-emitting device according to one embodiment of the present invention, the organic material layer includes a hole injection layer or a hole transport layer, and the hole transport layer or the hole injection layer may include the heterocyclic compound.

The organic light-emitting device of the present invention may be manufactured using common organic light-emitting device manufacturing methods and materials except that one or more organic material layers are formed using the heterocyclic compound described above.

The heterocyclic compound may form the organic material layer using a solution coating method as well as a vacuum deposition method when the organic light-emitting device is manufactured. Herein, the solution coating method means spin coating, dip coating, inkjet printing, screen printing, a spray method, roll coating and the like, but is not limited thereto.

The organic material layer of the organic light-emitting device of the present invention may be formed in a single layer structure, but may also be formed in a multilayer structure in which two or more organic material layers are laminated. For example, the organic light-emitting device of the present invention may have a structure including a hole injection layer, a hole transport layer, a hole transport auxiliary layer, a light emitting layer, an electron injection layer, an electron transport layer, an electron blocking layer, a hole blocking layer and the like as the organic material layer. However, the structure of the organic light-emitting device is not limited thereto, and may include a smaller number of organic material layers.

In the organic light-emitting device of the present invention, the organic material layer includes a hole transport layer or an electron blocking layer, and the hole transport layer or the electron blocking layer may include the heterocyclic compound represented by chemical formula 1. When the heterocyclic compound is used in the hole transport layer or the electron blocking layer, electron migration may be effectively controlled due to the structural features of the compound having a LUMO level and a triplet energy level, and as a result, driving efficiency and lifetime of the organic light-emitting device may become superior.

In one embodiment of the present invention, the organic light-emitting device may include one or more organic material layers, the organic material layer may include a light emitting layer, and the light emitting layer may include the heterocyclic compound represented by chemical formula 1.

In one embodiment of the present invention, the organic light-emitting device may further include one, or two or more layers selected from the group consisting of a light emitting layer, a hole injection layer, a hole blocking layer, an electron injection layer, an electron transport layer and a hole blocking layer.

In one embodiment of the present invention, the organic material layer includes the heterocyclic compound represented by chemical formula 1, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present invention is not limited by these examples.

M may be iridium, platinum, osmium or the like.

L is an anionic bidentate ligand coordinated to M by sp² carbon and heteroatom, and X may function to trap electrons or holes. Nonlimiting examples of L, L′ and L″ may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples.

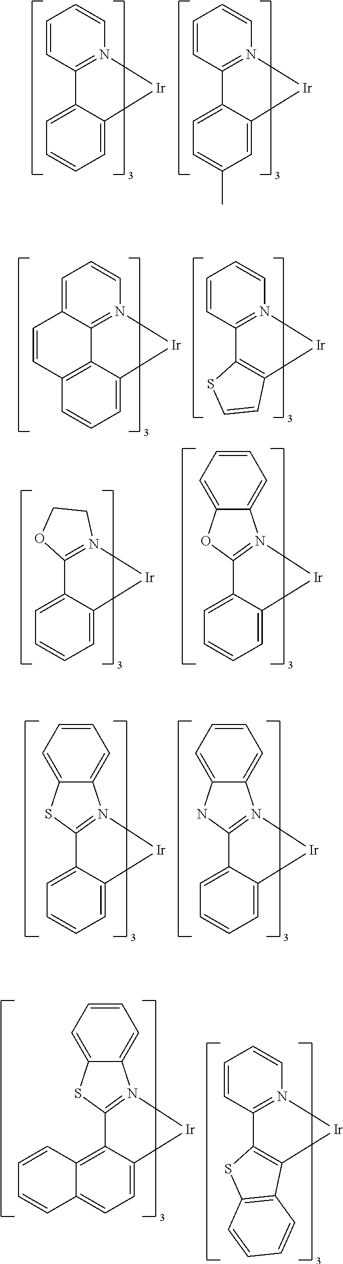

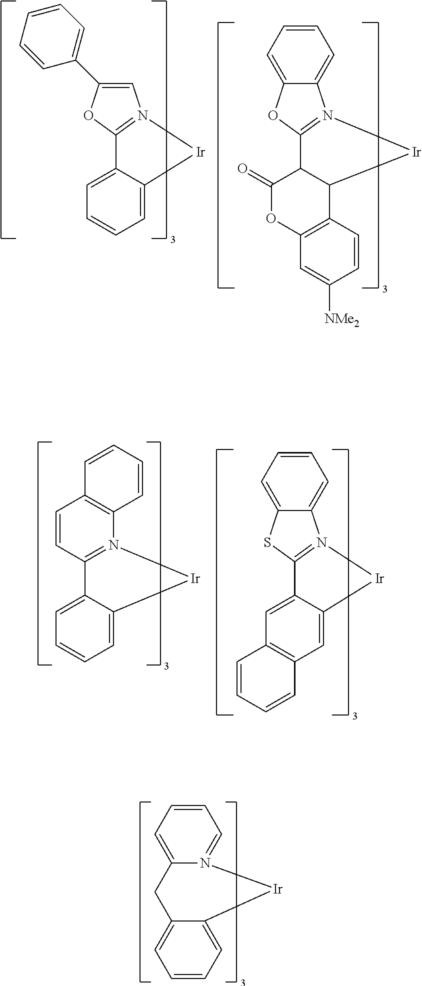

In one embodiment of the present invention, the organic material layer includes the heterocyclic compound represented by chemical formula 1, and a platinum-based dopant may be used therewith.

In one embodiment of the present invention, as the platinum-based dopant, Pt(ppzOczpy-4m) may be used as a blue phosphorescent dopant.

In one embodiment of the present invention, the content of the dopant may be from 1% to 15%, preferably from 2% to 10% and more preferably from 3% to 7% based on the total weight of the light emitting layer.

The organic light-emitting device according to one embodiment of the present invention may further include one, or two or more layers selected from the group consisting of a light emitting layer, a hole injection layer, a hole blocking layer, an electron injection layer, an electron transport layer and a hole blocking layer.

The organic material layer of the organic light-emitting device of the present invention may be formed in a single layer structure, but may also be formed in a multilayer structure in which two or more organic material layers are laminated. For example, the organic light-emitting device of the present invention may have a structure including a hole injection layer, a hole transport layer, a light emitting layer, an electron transport layer, an electron injection layer and the like as the organic material layer. However, the structure of the organic light-emitting device is not limited thereto, and may include a smaller number of organic material layers.

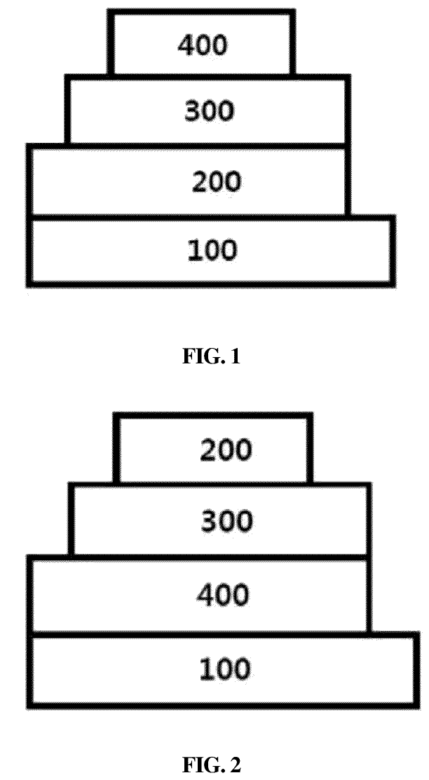

FIGS. 1 to 3 illustrate a lamination order of electrodes and organic material layers of the organic light-emitting device according to one embodiment of the present invention. However, the scope of the present application is not intended to be limited by these drawings, and structures of organic light-emitting devices known in the art may also be applied to the present application.

FIG. 1 illustrates an organic light-emitting device in which a positive electrode 200, an organic material layer 300 and a negative electrode 400 are sequentially laminated on a substrate 100. However, the structure is not limited only to such a structure, and as illustrated in FIG. 2, an organic light-emitting device in which a negative electrode, an organic material layer and a positive electrode are sequentially laminated on a substrate may also be obtained.

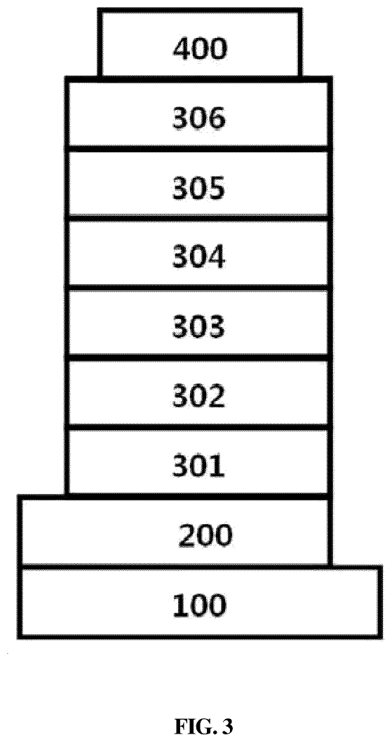

FIG. 3 illustrates a case of the organic material layer being a multilayer. An organic light-emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

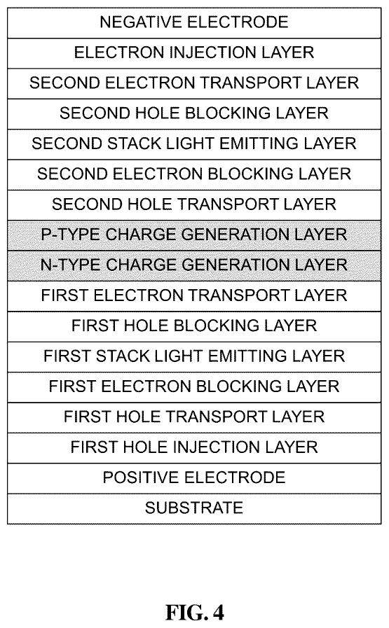

As the organic light-emitting device according to one embodiment of the present application, an organic light-emitting device having a 2-stack tandem structure is schematically illustrated in FIG. 4.

Herein, the first electron blocking layer, the first hole blocking layer, the second hole blocking layer and the like described in FIG. 4 may not be included depending on the case.

One embodiment of the present invention provides a method for manufacturing an organic light-emitting device, the method including: preparing a substrate; forming a first electrode on the substrate; forming one or more organic material layers on the first electrode; and forming a second electrode on the organic material layers, wherein the forming of organic material layers includes forming the one or more organic material layers using a composition for an organic material layer according to one embodiment of the present invention.

The organic material layer including the heterocyclic compound represented by chemical formula 1 may further include other materials as necessary.

In the organic light-emitting device according to one embodiment of the present invention, materials other than the heterocyclic compound represented by chemical formula 1 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involved in light emission together may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light-emitting device according to one embodiment of the present invention may be a top-emission type, a bottom-emission type or a dual-emission type depending on the materials used.

The heterocyclic compound according to one embodiment of the present invention may also be used in an organic electronic device including an organic solar cell, an organic photo conductor, an organic transistor and the like under a principle similar to that used in the organic light-emitting device.

Hereinafter, preferred examples are provided to help to understand the present invention, however, the following examples are only provided to more readily understand the present invention, and the present invention is not limited thereto.

### Preparation Example

### Preparation Example 1: Preparation of Compound 1

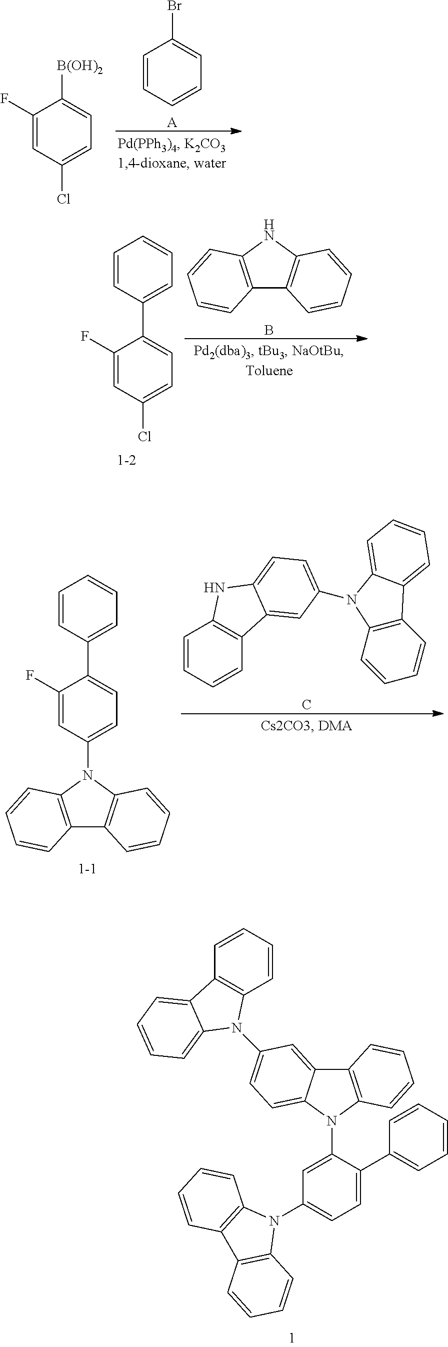

### 1) Preparation of Compound 1-2

Compound (4-chloro-2-fluorophenyl) boronic acid (10 g, 57.35 mM), bromobenzene (9.9 g, 63.8 mM), tetrakis(triphenylphosphine) palladium (Pd(PPh₃)₄) (3.31 g, 2.87 mM) and potassium carbonate (K₂CO₃) (23.78 g, 172.05 mM) were introduced to 1,4-dioxane (300 ml) and distilled water (60 ml) and dissolved therein, and then the mixture was refluxed for 6 hours. After the reaction was completed, the reaction solution was extracted using distilled water and dichloromethane (DCM) at room temperature. After that, moisture of the organic layer was removed using magnesium sulfate (MgSO₄), and then the solvent was removed using a rotary evaporator to obtain target Compound 1-2 (10 g, yield 84.38%).

### 2) Preparation of Compound 1-1

Compound 1-2 (10 g, 48.39 mM), carbazole (8.9 g, 53.23 mM), tris(dibenzylideneacetone) dipalladium (Pd₂(dba)₃) (2.22 g, 2.42 mM), tri-tert-butylphosphine (P (t-Bu) 3) (2.23 ml, 4.84 mM) and sodium tert-butoxide (t-BuONa) (13.95 g, 145.18 mM) were introduced to toluene (250 ml) and dissolved therein, and then the mixture was refluxed for 17 hours. After the reaction was completed, the reaction solution was extracted using distilled water and dichloromethane (DCM). After that, moisture of the organic layer was removed using magnesium sulfate (MgSO₄), and then the solvent was removed using a rotary evaporator. The reaction material was purified by column chromatography (DCM:Hex=1:5) and recrystallized with methanol to obtain target Compound 1-1 (10.3 g, yield 73.5%).

### 3) Preparation of Compound 1

Compound 1-1 (10.25 g, 30.38 mM), 3,9′-bicarbazole (11.11 g, 33.42 mM) and cesium carbonate (Cs₂CO₃) (29.7 g, 91.14 mM) were introduced to dimethylacetamide (DMA) (150 ml) and dissolved therein, and then the mixture was refluxed for 24 hours. After the reaction was completed, the solid was removed through filtration at room temperature, and the concentrate was diluted in methylene chloride (MC), filtered through silica gel, and purified by column chromatography (DCM:Hex=1:3). The purified solution was dissolved in toluene, and recrystallized by adding methanol thereto to obtain target Compound 1 (16 g, yield 81%).

Target compounds were synthesized in the same manner as in Preparation Example 1, except that Intermediate A, Intermediate B and Intermediate C of the following Table 1 were used instead of bromobenzene, carbazole and 3,9′-bicarbazole, respectively.

TABLE 1

| Compound | | | | |
|---|---|---|---|---|
| No. | Intermediate A | Intermediate B | Intermediate C | Yield |
| 2 | 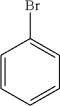 | 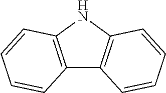 [SMILES: c1ccc2c(c1)[nH]c1ccccc12] | 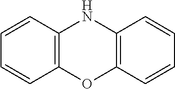 [SMILES: c1ccc2c(c1)Nc1ccccc1O2] | 75% |
| 3 | 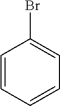 | 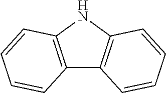 [SMILES: c1ccc2c(c1)[nH]c1ccccc12] | 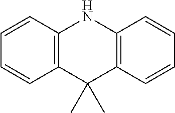 [SMILES: CC1(C)c2ccccc2Nc2ccccc21] | 72% |
| 13 | 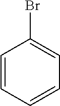 | 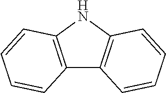 [SMILES: c1ccc2c(c1)[nH]c1ccccc12] | 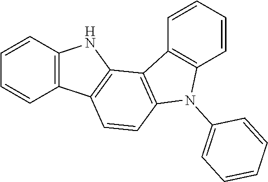 [SMILES: c1ccc(-n2c3ccccc3c3c4[nH]c5ccccc5c4ccc32)cc1] | 79% |
| 17 | 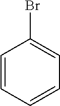 | 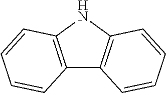 [SMILES: c1ccc2c(c1)[nH]c1ccccc12] | 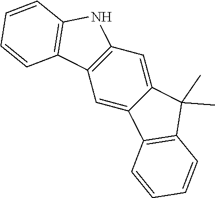 [SMILES: CC1(C)c2ccccc2-c2cc3c(cc21)[nH]c1ccccc13] | 79% |
| 21 | 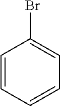 | 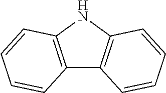 [SMILES: c1ccc2c(c1)[nH]c1ccccc12] | 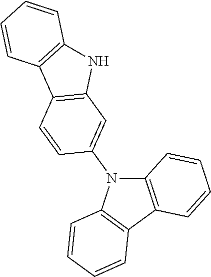 [SMILES: c1ccc2c(c1)[nH]c1cc(-n3c4ccccc4c4ccccc43)ccc12] | 80% |
| 26 | 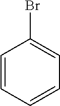 | 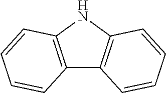 [SMILES: c1ccc2c(c1)[nH]c1ccccc12] | 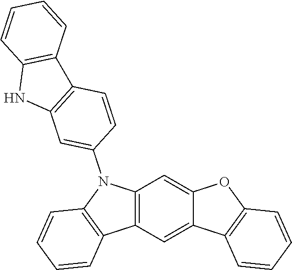 [SMILES: c1ccc2c(c1)[nH]c1cc(-n3c4ccccc4c4cc5c(cc43)oc3ccccc35)ccc12] | 75% |
| 41 | 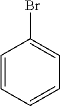 | 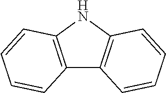 [SMILES: c1ccc2c(c1)[nH]c1ccccc12] | 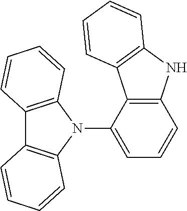 [SMILES: c1ccc2c(c1)[nH]c1cccc(-n3c4ccccc4c4ccccc43)c12] | 83% |
| 51 | 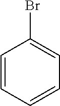 | 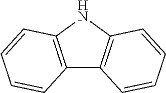 [SMILES: c1ccc2c(c1)[nH]c1ccccc12] | 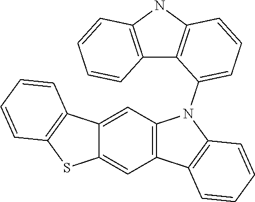 [SMILES: c1ccc2c(c1)[nH]c1cccc(-n3c4ccccc4c4cc5sc6ccccc6c5cc43)c12] | 81% |
| 61 | 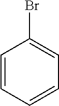 | 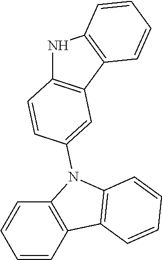 [SMILES: c1ccc2c(c1)[nH]c1ccc(-n3c4ccccc4c4ccccc43)cc12] | 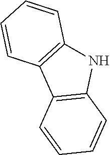 [SMILES: c1ccc2c(c1)[nH]c1ccccc12] | 85% |
| 65 | 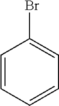 | 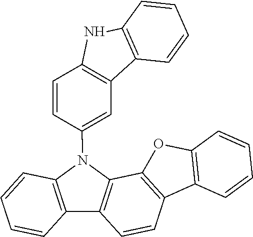 [SMILES: c1ccc2c(c1)[nH]c1ccc(-n3c4ccccc4c4ccc5c6ccccc6oc5c43)cc12] | 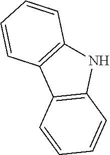 [SMILES: c1ccc2c(c1)[nH]c1ccccc12] | 67% |
| 69 | 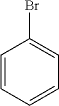 | 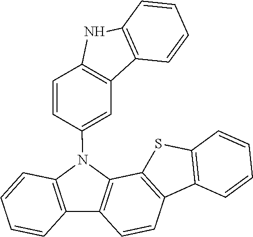 [SMILES: c1ccc2c(c1)[nH]c1ccc(-n3c4ccccc4c4ccc5c6ccccc6sc5c43)cc12] | 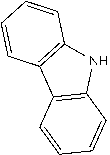 [SMILES: c1ccc2c(c1)[nH]c1ccccc12] | 68% |
| 73 | 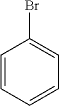 | 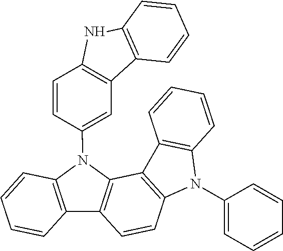 [SMILES: c1ccc(-n2c3ccccc3c3c2ccc2c4ccccc4n(-c4ccc5[nH]c6ccccc6c5c4)c23)cc1] | 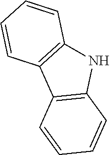 [SMILES: c1ccc2c(c1)[nH]c1ccccc12] | 61% |
| 77 | 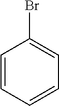 | 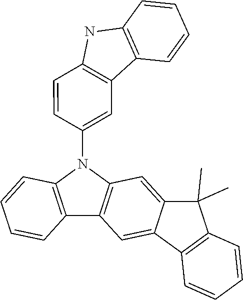 [SMILES: CC1(C)c2ccccc2-c2cc3c4ccccc4n(-c4ccc5[nH]c6ccccc6c5c4)c3cc21] | 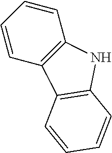 [SMILES: c1ccc2c(c1)[nH]c1ccccc12] | 65% |
| 81 | 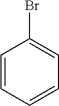 | 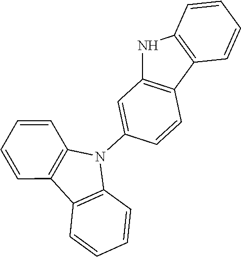 [SMILES: c1ccc2c(c1)[nH]c1cc(-n3c4ccccc4c4ccccc43)ccc12] | 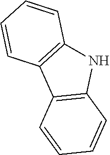 [SMILES: c1ccc2c(c1)[nH]c1ccccc12] | 80% |
| 96 | 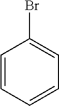 | 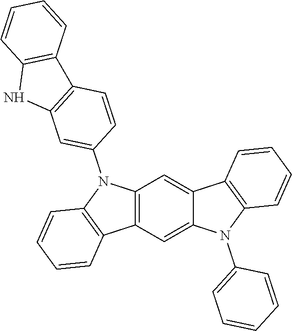 [SMILES: c1ccc(-n2c3ccccc3c3cc4c(cc32)c2ccccc2n4-c2ccc3c(c2)[nH]c2ccccc23)cc1] | 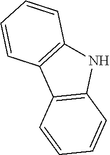 [SMILES: c1ccc2c(c1)[nH]c1ccccc12] | 75% |
| 101 | 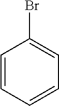 | 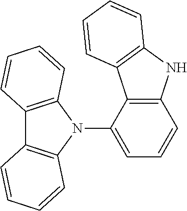 [SMILES: c1ccc2c(c1)[nH]c1cccc(-n3c4ccccc4c4ccccc43)c12] | 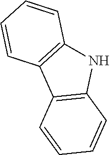 [SMILES: c1ccc2c(c1)[nH]c1ccccc12] | 78% |
| 117 | 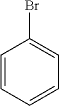 | 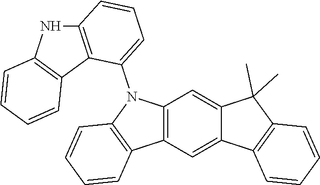 [SMILES: CC1(C)c2ccccc2-c2cc3c4ccccc4n(-c4cccc5[nH]c6ccccc6c45)c3cc21] | 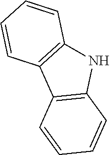 [SMILES: c1ccc2c(c1)[nH]c1ccccc12] | 67% |
| 121 | 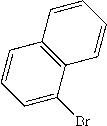 [SMILES: Brc1cccc2ccccc12] | 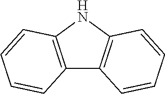 [SMILES: c1ccc2c(c1)[nH]c1ccccc12] | 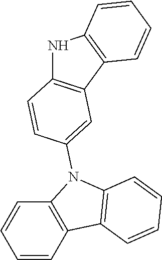 [SMILES: c1ccc2c(c1)[nH]c1ccc(-n3c4ccccc4c4ccccc43)cc12] | 77% |
| 126 | 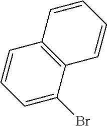 [SMILES: Brc1cccc2ccccc12] | 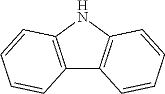 [SMILES: c1ccc2c(c1)[nH]c1ccccc12] | 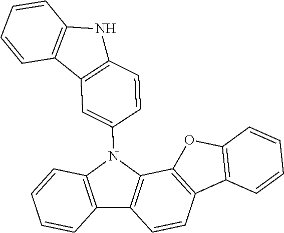 [SMILES: c1ccc2c(c1)[nH]c1ccc(-n3c4ccccc4c4ccc5c6ccccc6oc5c43)cc12] | 64% |
| 141 | 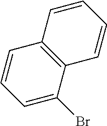 [SMILES: Brc1cccc2ccccc12] | 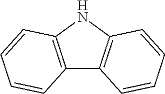 [SMILES: c1ccc2c(c1)[nH]c1ccccc12] | 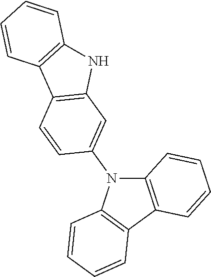 [SMILES: c1ccc2c(c1)[nH]c1cc(-n3c4ccccc4c4ccccc43)ccc12] | 79% |
| 151 | 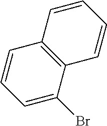 [SMILES: Brc1cccc2ccccc12] | 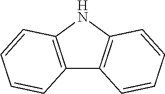 [SMILES: c1ccc2c(c1)[nH]c1ccccc12] | 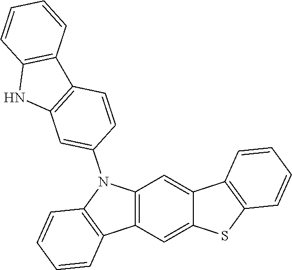 [SMILES: c1ccc2c(c1)[nH]c1cc(-n3c4ccccc4c4cc5sc6ccccc6c5cc43)ccc12] | 66% |
| 161 | 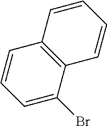 [SMILES: Brc1cccc2ccccc12] | 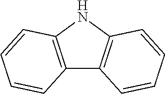 [SMILES: c1ccc2c(c1)[nH]c1ccccc12] | 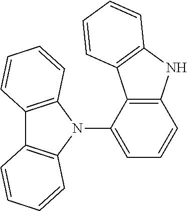 [SMILES: c1ccc2c(c1)[nH]c1cccc(-n3c4ccccc4c4ccccc43)c12] | 76% |
| 176 | 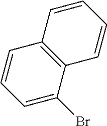 [SMILES: Brc1cccc2ccccc12] | 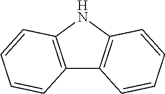 [SMILES: c1ccc2c(c1)[nH]c1ccccc12] | 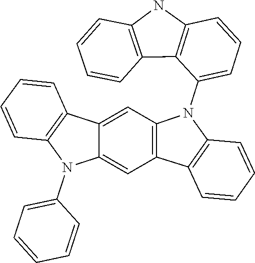 [SMILES: c1ccc(-n2c3ccccc3c3cc4c(cc32)c2ccccc2n4-c2cccc3[nH]c4ccccc4c23)cc1] | 72% |
| 181 | 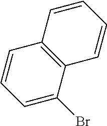 [SMILES: Brc1cccc2ccccc12] | 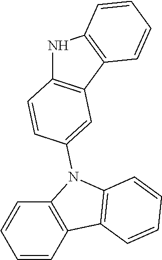 [SMILES: c1ccc2c(c1)[nH]c1ccc(-n3c4ccccc4c4ccccc43)cc12] | 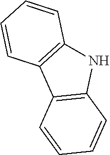 [SMILES: c1ccc2c(c1)[nH]c1ccccc12] | 77% |
| 197 | 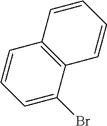 [SMILES: Brc1cccc2ccccc12] | 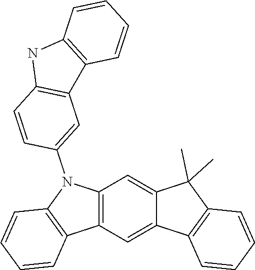 [SMILES: CC1(C)c2ccccc2-c2cc3c4ccccc4n(-c4ccc5[nH]c6ccccc6c5c4)c3cc21] | 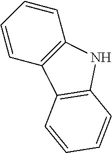 [SMILES: c1ccc2c(c1)[nH]c1ccccc12] | 70% |
| 201 | 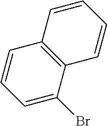 [SMILES: Brc1cccc2ccccc12] | 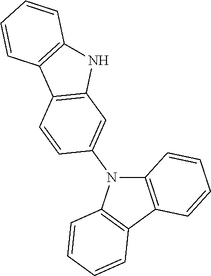 [SMILES: c1ccc2c(c1)[nH]c1cc(-n3c4ccccc4c4ccccc43)ccc12] | 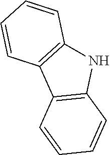 [SMILES: c1ccc2c(c1)[nH]c1ccccc12] | 79% |
| 206 | 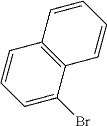 [SMILES: Brc1cccc2ccccc12] | 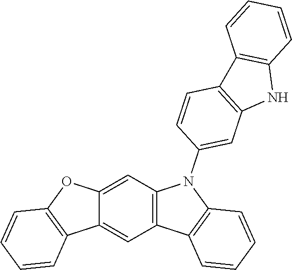 [SMILES: c1ccc2c(c1)[nH]c1cc(-n3c4ccccc4c4cc5c(cc43)oc3ccccc35)ccc12] | 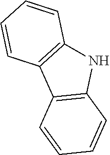 [SMILES: c1ccc2c(c1)[nH]c1ccccc12] | 72% |
| 221 | 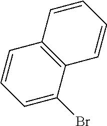 [SMILES: Brc1cccc2ccccc12] | 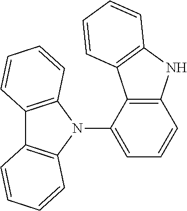 [SMILES: c1ccc2c(c1)[nH]c1cccc(-n3c4ccccc4c4ccccc43)c12] | 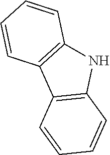 [SMILES: c1ccc2c(c1)[nH]c1ccccc12] | 74% |
| 231 | 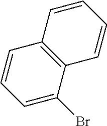 [SMILES: Brc1cccc2ccccc12] | 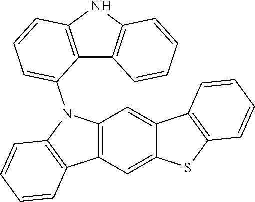 [SMILES: c1ccc2c(c1)[nH]c1cccc(-n3c4ccccc4c4cc5sc6ccccc6c5cc43)c12] | 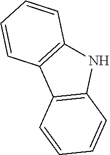 [SMILES: c1ccc2c(c1)[nH]c1ccccc12] | 63% |
| 241 | 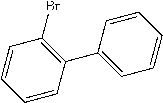 [SMILES: Brc1ccccc1-c1ccccc1] | 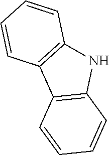 [SMILES: c1ccc2c(c1)[nH]c1ccccc12] | 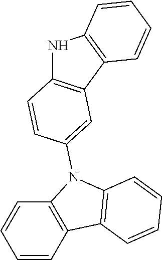 [SMILES: c1ccc2c(c1)[nH]c1ccc(-n3c4ccccc4c4ccccc43)cc12] | 66% |
| 256 | 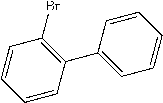 [SMILES: Brc1ccccc1-c1ccccc1] | 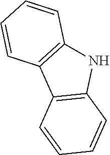 [SMILES: c1ccc2c(c1)[nH]c1ccccc12] | 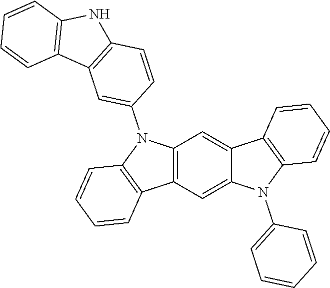 [SMILES: c1ccc(-n2c3ccccc3c3cc4c(cc32)c2ccccc2n4-c2ccc3[nH]c4ccccc4c3c2)cc1] | 77% |
| 261 | 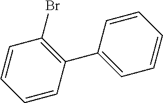 [SMILES: Brc1ccccc1-c1ccccc1] | 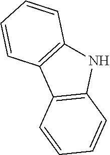 [SMILES: c1ccc2c(c1)[nH]c1ccccc12] | 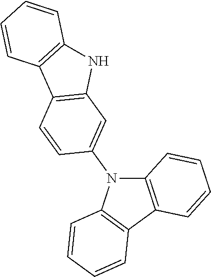 [SMILES: c1ccc2c(c1)[nH]c1cc(-n3c4ccccc4c4ccccc43)ccc12] | 67% |
| 277 | 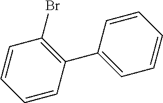 [SMILES: Brc1ccccc1-c1ccccc1] | 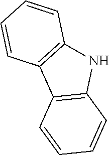 [SMILES: c1ccc2c(c1)[nH]c1ccccc12] | 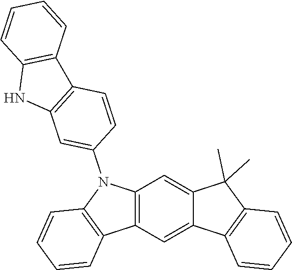 [SMILES: CC1(C)c2ccccc2-c2cc3c4ccccc4n(-c4ccc5c(c4)[nH]c4ccccc45)c3cc21] | 78% |
| 281 | 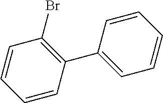 [SMILES: Brc1ccccc1-c1ccccc1] | 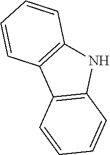 [SMILES: c1ccc2c(c1)[nH]c1ccccc12] | 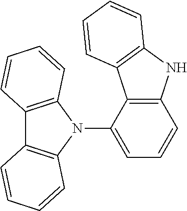 [SMILES: c1ccc2c(c1)[nH]c1cccc(-n3c4ccccc4c4ccccc43)c12] | 77% |
| 286 | 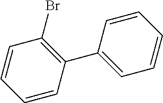 [SMILES: Brc1ccccc1-c1ccccc1] | 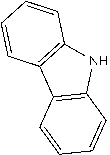 [SMILES: c1ccc2c(c1)[nH]c1ccccc12] | 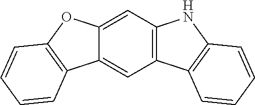 [SMILES: c1ccc2c(c1)[nH]c1cc3oc4ccccc4c3cc12] | 60% |
| 301 | 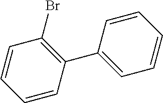 [SMILES: Brc1ccccc1-c1ccccc1] | 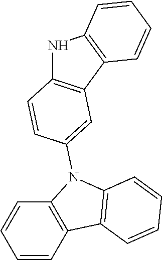 [SMILES: c1ccc2c(c1)[nH]c1ccc(-n3c4ccccc4c4ccccc43)cc12] | 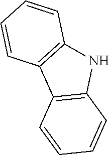 [SMILES: c1ccc2c(c1)[nH]c1ccccc12] | 74% |
| 311 | 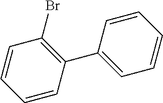 [SMILES: Brc1ccccc1-c1ccccc1] | 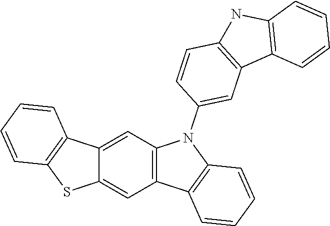 [SMILES: c1ccc2c(c1)[nH]c1ccc(-n3c4ccccc4c4cc5sc6ccccc6c5cc43)cc12] | 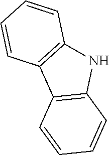 [SMILES: c1ccc2c(c1)[nH]c1ccccc12] | 69% |
| 321 | 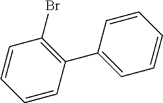 [SMILES: Brc1ccccc1-c1ccccc1] | 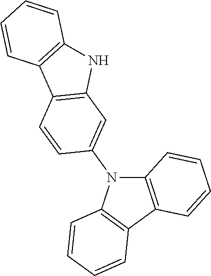 [SMILES: c1ccc2c(c1)[nH]c1cc(-n3c4ccccc4c4ccccc43)ccc12] | 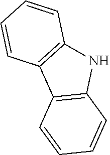 [SMILES: c1ccc2c(c1)[nH]c1ccccc12] | 74% |
| 336 | 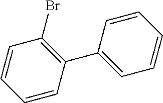 [SMILES: Brc1ccccc1-c1ccccc1] | 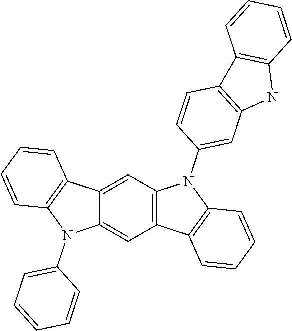 [SMILES: c1ccc(-n2c3ccccc3c3cc4c(cc32)c2ccccc2n4-c2ccc3c(c2)[nH]c2ccccc23)cc1] | 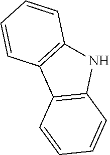 [SMILES: c1ccc2c(c1)[nH]c1ccccc12] | 63% |
| 341 | 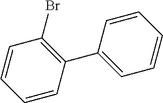 [SMILES: Brc1ccccc1-c1ccccc1] | 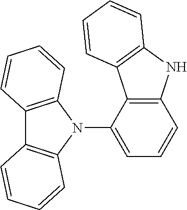 [SMILES: c1ccc2c(c1)[nH]c1cccc(-n3c4ccccc4c4ccccc43)c12] | 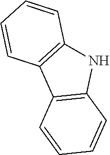 [SMILES: c1ccc2c(c1)[nH]c1ccccc12] | 72% |
| 357 | 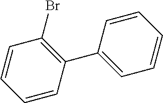 [SMILES: Brc1ccccc1-c1ccccc1] | 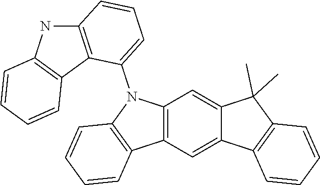 [SMILES: CC1(C)c2ccccc2-c2cc3c4ccccc4n(-c4cccc5[nH]c6ccccc6c45)c3cc21] | 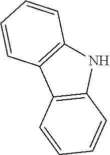 [SMILES: c1ccc2c(c1)[nH]c1ccccc12] | 61% |

### Preparation Example 2: Preparation of Compound 361

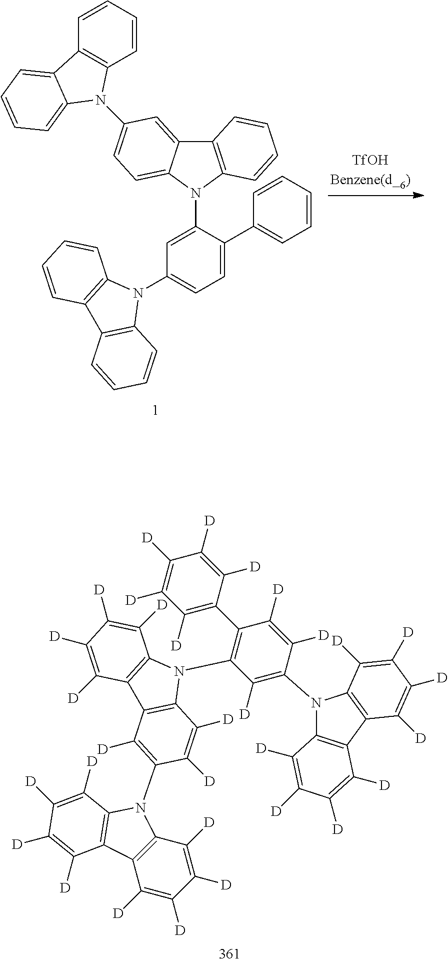 [SMILES: c1ccc(-c2ccc(-n3c4ccccc4c4ccccc43)cc2-n2c3ccccc3c3cc(-n4c5ccccc5c5ccccc54)ccc32)cc1 ; [2H]c1c([2H])c([2H])c(-c2c([2H])c([2H])c(-n3c4c([2H])c([2H])c([2H])c([2H])c4c4c([2H])c([2H])c([2H])c([2H])c43)c([2H])c2-n2c3c([2H])c([2H])c([2H])c([2H])c3c3c([2H])c(-n4c5c([2H])c([2H])c([2H])c([2H])c5c5c([2H])c([2H])c([2H])c([2H])c54)c([2H])c([2H])c32)c([2H])c1[2H]]

Compound 1 (7 g, 10.77 mM) and triflic acid (TfOH) (2.9 mL, 32.32 mM) were introduced to benzene-d₆ (105 ml) and dissolved therein, and then the mixture was stirred for 1 hour at 60° C. After the reaction was completed, distilled water was added thereto at a low temperature to separate the organic layer, and then the organic layer was concentrated. The reaction solution was purified by column chromatography (DCM:Hex=1:3), and recrystallized with ethyl acetate (EA)/methanol to obtain target Compound 361 (6.8 g, yield 92%).

### Preparation Example 3: Preparation of Compound 367

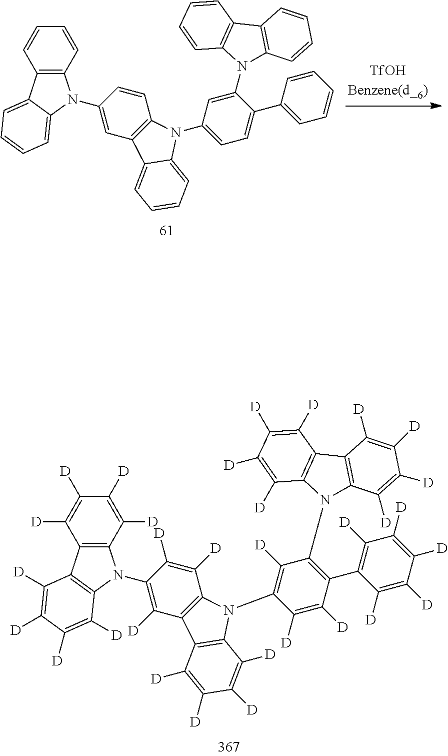 [SMILES: c1ccc(-c2ccc(-n3c4ccccc4c4cc(-n5c6ccccc6c6ccccc65)ccc43)cc2-n2c3ccccc3c3ccccc32)cc1 ; [2H]c1c([2H])c([2H])c(-c2c([2H])c([2H])c(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c([2H])c2-n2c3c([2H])c([2H])c([2H])c([2H])c3c3c([2H])c([2H])c([2H])c([2H])c32)c([2H])c1[2H]]

Compound 367 was prepared in the same manner as in Preparation Example 2, except that Compound 61 was used instead of Compound 1.

The rest of compounds other than the compounds described in Preparation Examples 1 to 3 and Table 1 were also prepared in the same manner as in the methods described in the Preparation Examples described above, and the synthesis results are shown in the following Tables 2 and 3. The following Table 2 shows measurement values of 1H NMR (CDCl₃, 200 Mz) of the compounds, and the following Table 3 shows measurement values of field desorption mass spectrometry (FD-MS).

TABLE 2

| Compound | |
|---|---|
| No. | ¹H NMR(CDCl₃, 200 Mz) |
| 1 | δ = 8.55 (3 H, d), 8.19 (2 H, d), 8.05 (1 H, d), 7.94-7.88 (4 H, m), 7.72-7.67 (3 H, m), |
| | 7.58-7.35 (9 H, m), 7.20-7.16 (9 H, m) |
| 2 | δ = 8.55 (2 H, d), 8.19 (1 H, d), 8.05 (1 H, d), 7.94-7.88 (3 H, m), 7.69 (1 H, s), 7.58-7.33 (8 H, |
| | m), 7.20-7.14 (9 H, m), 7.01-6.96 (6 H, m) |
| 3 | δ = 8.55 (2 H, d), 8.19 (1 H, d), 8.05 (1 H, d), 7.94-7.88 (3 H, m), 7.69 (1 H, s), 7.58-7.41 (4 H, |
| | m), 7.35-7.33 (4 H, m), 7.2-7.14 (13 H, m), 6.95 (2 H, m), 1.69 (6 H, s) |
| 13 | δ = 8.55 (4 H, d), 8.19-8.12 (2 H, m), 8.05 (1 H, d), 7.94-7.88 (5 H, m), 7.72-7.35 (16 H, m), |
| | 7.20-7.16 (10 H, m) |
| 17 | δ = 8.55 (3 H, d), 8.24-8.19 (2 H, m), 8.05 (1 H, d), 7.94-7.88 (5 H, m), 7.74-7.67 (4 H, m), |
| | 7.58-7.35 (10 H, m), 7.20-7.16 (8 H, m), 1.67 (6 H, s) |
| 21 | δ = 8.55 (3 H, d), 8.26-8.19 (3 H, m), 8.05 (1 H, d), 7.94-7.88 (4 H, m), 7.69 (1 H, s), 7.58- |
| | 7.35 (10 H, m), 7.20-7.16 (9 H, m) |
| 26 | δ = 8.55 (3 H, d), 8.26-8.19 (2 H, m), 8.05-7.88 (6 H, m), 7.69 (1 H, s), 7.58-7.35 (13 H, m), |
| | 7.20-7.16 (8 H, m) |
| 41 | δ = 8.55 (3 H, d), 8.19 (2 H, m), 8.05 (1 H, d), 7.94-7.88 (4 H, m), 7.69 (1 H, s), 7.58- |
| | 7.35 (11 H, m), 7.20-7.16 (9 H, m) |
| 51 | δ = 8.55 (3 H, d), 8.45 (1 H, d), 8.19 (1 H, d), 8.05 (1 H, d), 7.94-7.86 (6 H, m), 7.78 (1 H, d), |
| | 7.69 (1 H, s), 7.58-7.35 (11 H, m), 7.20-7.16 (8 H, m) |
| 61 | δ = 8.55 (3 H, d), 8.19 (2 H, m), 8.05 (1 H, d), 7.94-7.88 (4 H, m), 7.72-7.67 (3 H, m), |
| | 7.58-7.35 (9 H, m), 7.20-7.16 (9 H, m) |
| 65 | δ = 8.55 (3 H, d), 8.19 (1 H, d), 8.05-7.84 (7 H, m), 7.69-7.67 (3 H, m), 7.58-7.35 (11 H, m), |
| | 7.20-7.16 (8 H, m) |
| 69 | δ = 8.55 (3 H, d), 8.45 (1 H, d), 8.19 (1 H, d), 8.05 (2 H, d), 7.94-7.88 (5 H, m), 7.72-7.67 (3 H, |
| | m), 7.60-7.35 (10 H, m), 7.20-7.16 (8 H, m) |
| 73 | δ = 8.55 (4 H, d), 8.19-8.12 (2 H, m), 8.05 (1 H, d), 7.94-7.88 (5 H, m), 7.72-7.35 (16 H, m), |
| | 7.20-7.16 (10 H, m) |
| 77 | δ = 8.55 (3 H, d), 8.24-8.19 (2 H, m), 8.05 (1 H, d), 7.94-7.88 (5 H, m), 7.72-7.67 (4 H, m), |
| | 7.58-7.35 (10 H, m), 7.20-7.16 (8 H, m), 1.69 (6 H, s) |
| 81 | δ = 8.55 (3 H, d), 8.26-8.19 (3 H, m), 8.05 (1 H, d), 7.94-7.88 (4 H, m), 7.69 (1 H, s), |
| | 7.58-7.35 (10 H, m), 7.20-7.16 (9 H, m) |
| 96 | δ = 8.55 (3 H, d), 8.26-8.19 (3 H, m), 8.05 (1 H, d), 7.94-7.88 (4 H, m), 7.69-7.35 (18 H, m), |
| | 7.20-7.16 (9 H, m) |
| 101 | δ = 8.55 (3 H, d), 8.19 (2 H, m), 8.05 (1 H, d), 7.94-7.88 (4 H, m), 7.69 (1 H, s), |
| | 7.58-7.35 (11 H, m), 7.20-7.16 (9 H, m) |
| 117 | δ = 8.55 (3 H, d), 8.24-8.19 (2 H, m), 8.05 (1 H, d), 7.94-7.88 (5 H, m), 7.74-7.69 (2 H, m), |
| | 7.58-7.35 (12 H, m), 7.20-7.16 (8 H, m), 1.69 (6 H, s) |
| 121 | δ = 8.95 (1 H, d), 8.55-8.50 (4 H, m), 8.20-8.19 (3 H, m), 8.09-8.05 (2 H, m), 7.94-7.88 (4 H, |
| | m), 7.72-7.67 (4 H, m), 7.58-7.35 (10 H, m), 7.20-7.16 (5 H, m) |
| 126 | δ = 8.95 (1 H, d), 8.55-8.50 (4 H, m), 8.20-8.19 (2 H, m), 8.05-7.88 (7 H, m), 7.77-7.67 (4 H, |
| | m), 7.58-7.35 (13 H, m), 7.20-7.16 (4 H, m) |
| 141 | δ = 8.95 (1 H, d), 8.55-8.50 (4 H, m), 8.20-8.19 (4 H, m), 8.09-8.05 (2 H, m), 7.94-7.88 (4 H, |
| | m), 7.77 (1 H, m), 7.69 (1 H, s), 7.58-7.50 (6 H, m), 7.39-7.30 (5 H, m), 7.20-7.16 (5 H, m) |
| 151 | δ = 8.95 (1 H, d), 8.55-8.45 (5 H, m), 8.26-8.19 (3 H, m), 8.09-8.05 (2 H, m), 7.94-7.86 (6 H, |
| | m), 7.77-7.78 (2 H, m), 7.69 (1 H, s), 7.58-7.35 (11 H, m), 7.20-7.16 (4 H, m) |
| 161 | δ = 8.95 (1 H, d), 8.55-8.50 (4 H, m), 8.20-8.19 (3 H, m), 8.09-8.05 (2 H, m), 7.94-7.88 (4 H, |
| | m), 7.77 (1 H, m), 7.69 (1 H, s), 7.58-7.50 (7 H, m), 7.39-7.30 (5 H, m), 7.20-7.16 (5 H, m) |
| 176 | δ = 8.95 (1 H, d), 8.55-8.50 (4 H, m), 8.20-8.19 (3 H, m), 8.09-8.05 (2 H, m), 7.94-7.88 (4 H, |
| | m), 7.77 (1 H, m), 7.69-7.50 (14 H, m), 7.40-7.30 (6 H, m), 7.20-7.16 (5 H, m) |
| 181 | δ= 8.95 (1 H, d), 8.55-8.50 (4 H, m), 8.20-8.19 (3 H, m), 8.09-8.05 (2 H, m), 7.94-7.88 (4 H, |
| | m), 7.77-7.67 (4 H, m), 7.58-7.35 (10 H, m), 7.20-7.16 (5 H, m) |
| 197 | δ= 8.95 (1 H, d), 8.55 (4 H, d), 8.24-8.19 (3 H, m), 8.09-8.05 (2 H, m), 7.94-7.88 (5 H, m), |
| | 7.74-7.69 (5 H, m), 7.58-7.49 (5 H, m), 7.39-7.35 (6 H, m), 7.20-7.16 (4 H, m), 1.69 (6 H, s) |
| 201 | δ = 8.95 (1 H, d), 8.55-8.50 (4 H, m), 8.26-8.19 (4 H, m), 8.09-8.05 (2 H, m), 7.94-7.88 (4 H, |
| | m), 7.77 (1 H, m), 7.69 (1 H, s), 7.58-7.50 (6 H, m), 7.39-7.30 (5 H, m), 7.20-7.16 (5 H, m) |
| 206 | δ = 8.95 (1 H, d), 8.55-8.50 (4 H, m), 8.26-8.19 (3 H, m), 8.09-7.94 (7 H, m), 7.77 (1 H, m), |
| | 7.69 (1 H, s), 7.58-7.35 (14 H, m), 7.20-7.16 (4 H, m) |
| 221 | δ = 8.95 (1 H, d), 8.55-8.50 (4 H, m), 8.20-8.19 (3 H, m), 8.09-8.05 (2 H, m), 7.94-7.88 (4 H, |
| | m), 7.77 (1 H, m), 7.69 (1 H, s), 7.58-7.50 (7 H, m), 7.39-7.30 (5 H, m), 7.20-7.16 (5 H, m) |
| 231 | δ = 8.95 (1 H, d), 8.55-8.45 (5 H, m), 8.26-8.19 (2 H, m), 8.09-8.05 (2 H, m), 7.94-7.86 (6 H, |
| | m), 7.77-7.78 (2 H, m), 7.69 (1 H, s), 7.58-7.35 (12 H, m), 7.20-7.16 (4 H, m) |
| 241 | δ = 8.55 (3 H, d), 8.19 (2 H, d), 8.05 (1 H, d), 7.96-7.88 (6 H, m), 7.79-7.35 (18 H, m), |
| | 7.20-7.16 (5 H, m) |
| 256 | δ = 8.55 (3 H, d), 8.19 (2 H, d), 8.05 (1 H, d), 7.96-7.88 (6 H, m), 7.79-7.35 (25 H, m), |
| | 7.20-7.16 (5 H, m) |
| 261 | δ = 8.55 (3 H, d), 8.26 (1 H, d), 8.19 (2 H, d), 8.05 (1 H, d), 7.96-7.88 (6 H, m), 7.79 (2 H, d), |
| | 7.69 (1 H, s), 7.60-7.16 (14 H, m), 7.20-7.16 (5 H, m) |
| 277 | δ = 8.55 (3 H, d), 8.26-8.19 (3 H, m), 8.05 (1 H, d), 7.96-7.88 (7 H, m), 7.79-7.69 (4 H, m), |
| | 7.60-7.35 (15 H, m), 7.20-7.16 (4 H, m) |
| 281 | δ = 8.55 (3 H, d), 8.19 (2 H, d), 8.05 (1 H, d), 7.96-7.88 (6 H, m), 7.79 (2 H, d), 7.69 ( 1H, s), |
| | 7.60-7.30 (15 H, m), 7.20-7.16 (5 H, m) |
| 286 | δ = 8.55 (3 H, d), 8.19 (1 H, d), 8.05-7.88 (8 H, m), 7.79 (2 H, d), 7.69 (1 H, s), |
| | 7.60-7.30 (18 H, m), 7.20-7.16 (4 H, m) |
| 301 | δ = 8.55 (3 H, d), 8.19 (2 H, d), 8.05 (1 H, d), 7.96-7.88 (6 H, m), 7.79-7.35 (18 H, m), |
| | 7.20-7.16 (5 H, m) |
| 311 | δ = 8.55 (3 H, d), 8.45 (1 H, s), 8.19 (1 H, d), 8.05 (1 H, d), 7.96-7.35 (27 H, m), 7.20- |
| | 7.16 (4 H, m) |
| 321 | δ = 8.55 (3 H, d), 8.26 (1 H, d), 8.19 (2 H, d), 8.05 (1 H, d), 7.96-7.88 (6 H, m), 7.79 (2 H, d), |
| | 7.69 (1 H, s), 7.60-7.30 (14 H, m), 7.20-7.16 (5 H, m) |
| 336 | δ = 8.55 (3 H, d), 8.26 (1 H, d), 8.19 (2 H, d), 8.05 (1 H, d), 7.96-7.88 (6 H, m), 7.79 (2 H, d), |
| | 7.69 (1 H, s), 7.60-7.30 (21 H, m), 7.20-7.16 (5 H, m) |
| 341 | δ = 8.55 (3 H, d), 8.19 (2 H, d), 8.05 (1 H, d), 7.96-7.88 (6 H, m), 7.79 (2 H, d), 7.69 (1 H, s), |
| | 7.60-7.30 (15 H, m), 7.20-7.16 (5 H, m) |
| 357 | δ = 8.55 (3 H, d), 8.24-8.19 (2 H, m), 8.05 (1 H, d), 7.94-7.88 (7 H, m), 7.79-7.69 (4 H, m), |
| | 7.60-7.35 (16 H, m), 7.20-7.16 (4 H, m), 1.69 (6 H, s) |
| 361 | - |
| 367 | - |

TABLE 3

| Compound | | Compound | |
|---|---|---|---|
| No. | FD-MS | No. | FD-MS |
| 1 | m/z = 649.25(C₄₈H₃₁N₃ = 649.80) | 2 | m/z = 665.25(C₄₈H₃₁N₃O = 665.80) |
| 3 | m/z = 691.30(C₅₁H₃₇N₃ = 691.88) | 13 | m/z = 814.31(C₆₀H₃₈N₄ = 814.99) |
| 17 | m/z = 765.31(C₅₇H₃₉N₃ = 765.96) | 21 | m/z = 649.25(C₄₈H₃₁N₃ = 649.80) |
| 26 | m/z = 739.26(C₅₄H₃₃N₃O = 739.88) | 41 | m/z = 649.25(C₄₈H₃₁N₃ = 649.80) |
| 51 | m/z = 755.24(C₅₄H₃₃N₃S = 755.94) | 61 | m/z = 649.25(C₄₈H₃₁N₃ = 649.80) |
| 65 | m/z = 739.26(C₅₄H₃₃N₃O = 739.88) | 69 | m/z = 755.24(C₅₄H₃₃N₃S = 755.94) |
| 73 | m/z = 814.31(C₆₀H₃₈N₄ = 814.99) | 77 | m/z = 765.31(C₅₇H₃₉N₃ = 765.96) |
| 81 | m/z = 649.25(C₄₈H₃₁N₃ = 649.80) | 96 | m/z = 814.31(C₆₀H₃₈N₄ = 814.99) |
| 101 | m/z = 649.25(C₄₈H₃₁N₃ = 649.80) | 117 | m/z = 765.31(C₅₇H₃₉N₃ = 765.96) |
| 121 | m/z = 699.27(C₅₂H₃₃N₃ = 699.86) | 126 | m/z = 789.28(C₅₈H₃₅N₃O = 789.94) |
| 141 | m/z = 699.27(C₅₂H₃₃N₃ = 699.86) | 151 | m/z = 805.26(C₅₈H₃₅N₃S = 806.00) |
| 161 | m/z = 699.27(C₅₂H₃₃N₃ = 699.86) | 176 | m/z = 864.33(C₆₄H₄₀N₄ = 865.05) |
| 181 | m/z = 699.27(C₅₂H₃₃N₃ = 699.86) | 197 | m/z = 815.33(C₆₁H₄₁N₃ = 816.02) |
| 201 | m/z = 699.27(C₅₂H₃₃N₃ = 699.86) | 206 | m/z = 789.28(C₅₈H₃₅N₃O = 789.94) |
| 221 | m/z = 699.27(C₅₂H₃₃N₃ = 699.86) | 231 | m/z = 805.26(C₅₈H₃₅N₃S = 806.00) |
| 241 | m/z = 725.28(C₅₄H₃₅N₃ = 725.89) | 256 | m/z = 890.34(C₆₆H₄₂N₄ = 891.09) |
| 261 | m/z = 725.28(C₅₄H₃₅N₃ = 725.89) | 277 | m/z = 841.35(C₆₃H₄₃N₃ = 842.06) |
| 281 | m/z = 725.28(C₅₄H₃₅N₃ = 725.89) | 286 | m/z = 815.29(C₆₀H₃₇N₃O = 815.98) |
| 301 | m/z = 725.28(C₅₄H₃₅N₃ = 725.89) | 311 | m/z = 831.27(C₆₀H₃₇N₃S = 832.04) |
| 321 | m/z = 725.28(C₅₄H₃₅N₃ = 725.89) | 336 | m/z = 890.34(C₆₆H₄₂N₄ = 891.09) |
| 341 | m/z = 725.28(C₅₄H₃₅N₃ = 725.89) | 357 | m/z = 841.35(C₆₃H₄₃N₃ = 842.06) |
| 361 | m/z = 680.45(C₄₈D₃₁N₃ = 680.99) | 367 | m/z = 680.45(C₄₈D₃₁N₃ = 680.99) |

### Experimental Example

### Experimental Example 1

### (1) Manufacture of Organic Light-Emitting Device

A glass substrate on which indium tin oxide (ITO) having a thickness of 1,500 Å was coated as a thin film was ultrasonic cleaned with distilled water. When the cleaning with distilled water was finished, the substrate was ultrasonic cleaned with solvents such as acetone, methanol and isopropyl alcohol, then dried, and then subjected to UVO (ultraviolet ozone) treatment for 5 minutes using UV (ultraviolet) in a UV cleaner. After that, the substrate was transferred to a plasma cleaner (PT), then subjected to plasma treatment under vacuum for ITO work function increase and residual film removal, and transferred to a thermal deposition apparatus for organic deposition.

On the transport ITO electrode (positive electrode), 4,4′,4″-tris[2-naphthyl(phenyl)amino]triphenylamine (2-TNATA) and N,N′-bis(a-naphthyl)-N,N′-diphenyl-4,4′-diamine (NPB) were deposited as a hole injection layer and a hole transport layer that are common layers.

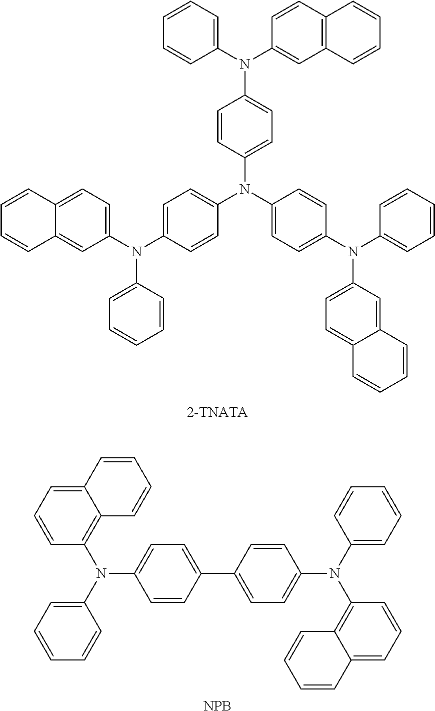 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transport layer as above, a light emitting layer was thermal vacuum deposited thereon as follows. As the light emitting layer, a compound described in the following Table 4 was deposited to a thickness of 400 Å as a host, and, using Pt(ppzOczpy-4m) as a blue phosphorescent dopant, the Pt Compound was doped and deposited on the host in an amount of 7 wt % of the deposited thickness of the light emitting layer.

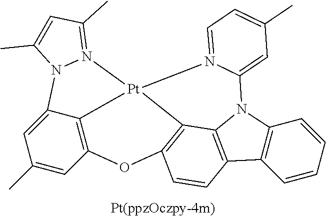

After that, bathocuproine (BCP) was deposited to a thickness of 60 Å as a hole blocking layer, and tris(8-hydroxyquinolinato)aluminum (Alq₃) was deposited to a thickness of 200 Å thereon as an electron transport layer.

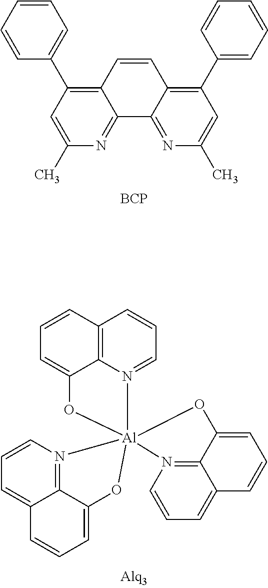

Lastly, lithium fluoride (LiF) was deposited to a thickness of 10 Å on the electron transport layer to form an electron injection layer, and then an aluminum (Al) negative electrode was deposited to a thickness of 1,200 Å on the electron injection layer to form a negative electrode, and as a result, an organic light-emitting device was manufactured.

Meanwhile, all the organic compounds required to manufacture the organic light-emitting device were vacuum sublimation purified under 108 torr to 106 torr for each material to be used in the manufacture of the organic light-emitting device.

### (2) Driving Voltage and Light Emission Efficiency of Organic Light-Emitting Device

For each of the organic light-emitting devices manufactured as above, electroluminescent (EL) properties were measured using M7000 manufactured by McScience Inc., and with the measurement results, a lifetime T₉₀ that is a time taken for luminance to become 90% with respect to initial luminance was measured when standard luminance was 700 cd/m² through a lifetime measurement system (M6000) manufactured by McScience Inc.

Results of measuring driving voltage, light emission efficiency, color coordinate (CIE) and lifetime of the blue organic light-emitting device manufactured according to the present invention are shown in the following Table 4.

TABLE 4

| | Light | | | | |
|---|---|---|---|---|---|
| | Emitting | Driving | Light Emission | Color | |
| | Layer | Voltage | Efficiency | Coordinate | Lifetime |
| | Compound | (V) | (cd/A) | (x, y) | (T₉₀) |
| Example 1 | 1 | 3.81 | 19.6 | (0.135, 0.140) | 40 |
| Example 2 | 2 | 3.87 | 16.7 | (0.133, 0.139) | 35 |
| Example 3 | 3 | 3.90 | 17.5 | (0.136, 0.141) | 34 |
| Example 4 | 13 | 3.91 | 15.2 | (0.133, 0.146) | 32 |
| Example 5 | 17 | 3.95 | 17.5 | (0.133, 0.138) | 36 |
| Example 6 | 21 | 3.87 | 16.4 | (0.130, 0.141) | 34 |
| Example 7 | 26 | 3.83 | 18.8 | (0.131, 0.141) | 34 |
| Example 8 | 41 | 3.95 | 16.8 | (0.133, 0.141) | 32 |
| Example 9 | 51 | 3.85 | 18.8 | (0.132, 0.143) | 35 |
| Example 10 | 61 | 3.75 | 17.2 | (0.135, 0.139) | 38 |
| Example 11 | 65 | 3.85 | 18.8 | (0.132, 0.140) | 33 |
| Example 12 | 69 | 3.75 | 17.2 | (0.135, 0.139) | 38 |
| Example 13 | 73 | 3.83 | 16.8 | (0.133, 0.141) | 36 |
| Example 14 | 77 | 3.85 | 17.7 | (0.132, 0.139) | 36 |
| Example 15 | 81 | 3.80 | 17.4 | (0.134, 0.141) | 36 |
| Example 16 | 96 | 3.82 | 17.3 | (0.136, 0.140) | 32 |
| Example 17 | 101 | 3.82 | 17.2 | (0.132, 0.139) | 32 |
| Example 18 | 117 | 3.86 | 18.4 | (0.133, 0.138) | 37 |
| Example 19 | 121 | 3.87 | 18.1 | (0.138, 0.140) | 37 |
| Example 20 | 126 | 3.81 | 18.5 | (0.134, 0.140) | 37 |
| Example 21 | 141 | 3.82 | 17.4 | (0.134, 0.139) | 35 |
| Example 22 | 151 | 3.80 | 17.2 | (0.134, 0.140) | 36 |
| Example 23 | 161 | 3.83 | 17.4 | (0.132, 0.141) | 36 |
| Example 24 | 176 | 3.81 | 16.8 | (0.132, 0.143) | 36 |
| Example 25 | 181 | 3.83 | 17.4 | (0.135, 0.140) | 36 |
| Example 26 | 197 | 3.82 | 16.6 | (0.137, 0.141) | 32 |
| Example 27 | 201 | 3.82 | 17.0 | (0.138, 0.141) | 32 |
| Example 28 | 206 | 3.80 | 16.9 | (0.133, 0.142) | 31 |
| Example 29 | 221 | 3.87 | 16.5 | (0.133, 0.142) | 31 |
| Example 30 | 231 | 3.89 | 16.8 | (0.135, 0.141) | 31 |
| Example 31 | 241 | 3.79 | 17.2 | (0.136, 0.142) | 32 |
| Example 32 | 256 | 3.83 | 17.9 | (0.134, 0.143) | 35 |
| Example 33 | 261 | 3.82 | 17.5 | (0.134, 0.141) | 35 |
| Example 34 | 277 | 3.89 | 19.2 | (0.131, 0.138) | 32 |
| Example 35 | 281 | 3.81 | 19.2 | (0.131, 0.139) | 32 |
| Example 36 | 286 | 3.86 | 18.0 | (0.130, 0.141) | 35 |
| Example 37 | 301 | 3.82 | 18.6 | (0.130, 0.144) | 35 |
| Example 38 | 311 | 3.88 | 18.2 | (0.132, 0.140) | 34 |
| Example 39 | 321 | 3.86 | 18.6 | (0.130, 0.141) | 35 |
| Example 40 | 336 | 3.82 | 17.9 | (0.132, 0.140) | 34 |
| Example 41 | 341 | 3.83 | 17.3 | (0.136, 0.140) | 36 |
| Example 42 | 357 | 3.86 | 17.1 | (0.137, 0.151) | 31 |
| Example 43 | 361 | 3.81 | 17.2 | (0.135, 0.140) | 48 |
| Example 44 | 367 | 3.75 | 17.1 | (0.135, 0.139) | 46 |
| Comparative | ref. 1 | 3.97 | 15.4 | (0.201, 0.150) | 22 |
| Example 1 | | | | | |
| Comparative | ref. 2 | 4.05 | 10.0 | (0.135, 0.140) | 14 |
| Example 2 | | | | | |
| Comparative | ref. 3 | 4.25 | 11.5 | (0.145, 0.170) | 2 |
| Example 3 | | | | | |
| Comparative | ref. 4 | 3.98 | 12.5 | (0.190, 0.174) | 8 |
| Example 4 | | | | | |
| Comparative | ref. 5 | 4.05 | 10.0 | (0.135, 0.140) | 6 |
| Example 5 | | | | | |
| Comparative | ref. 6 | 4.25 | 12.1 | (0.145, 0.170) | 2 |
| Example 6 | | | | | |
| Comparative | ref. 7 | 3.94 | 10.3 | (0.240, 0.184) | 5 |
| Example 7 | | | | | |

The compounds of Comparative Examples 1 to 7 are compounds of the following ref. 1 to ref. 7:

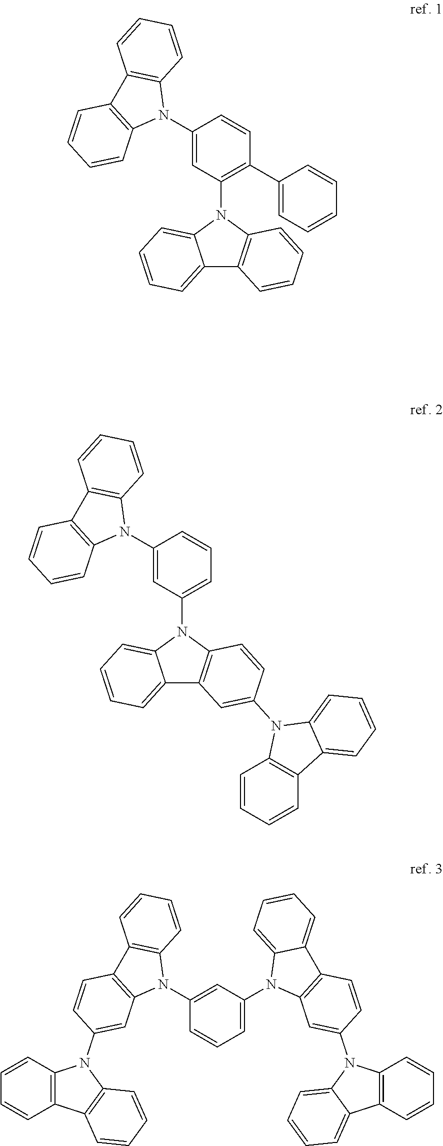 [SMILES: c1ccc(-c2ccc(-n3c4ccccc4c4ccccc43)cc2-n2c3ccccc3c3ccccc32)cc1 ; c1cc(-n2c3ccccc3c3ccccc32)cc(-n2c3ccccc3c3cc(-n4c5ccccc5c5ccccc54)ccc32)c1 ; c1cc(-n2c3ccccc3c3ccc(-n4c5ccccc5c5ccccc54)cc32)cc(-n2c3ccccc3c3ccc(-n4c5ccccc5c5ccccc54)cc32)c1]

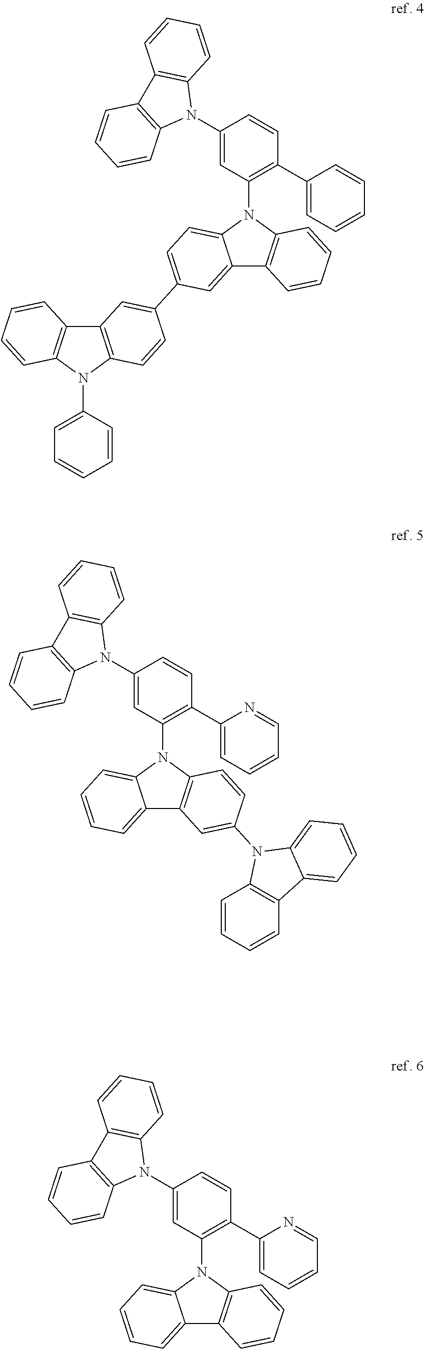 [SMILES: c1ccc(-c2ccc(-n3c4ccccc4c4ccccc43)cc2-n2c3ccccc3c3cc(-c4ccc5c(c4)c4ccccc4n5-c4ccccc4)ccc32)cc1 ; c1ccc(-c2ccc(-n3c4ccccc4c4ccccc43)cc2-n2c3ccccc3c3cc(-n4c5ccccc5c5ccccc54)ccc32)nc1 ; c1ccc(-c2ccc(-n3c4ccccc4c4ccccc43)cc2-n2c3ccccc3c3ccccc32)nc1]

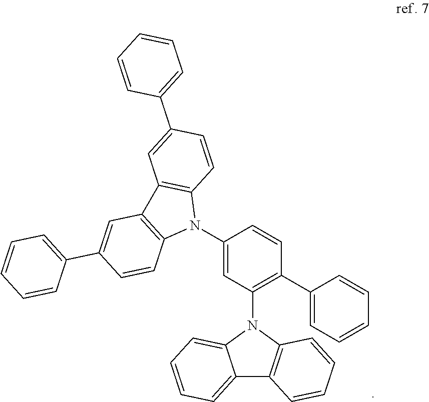 [SMILES: c1ccc(-c2ccc3c(c2)c2cc(-c4ccccc4)ccc2n3-c2ccc(-c3ccccc3)c(-n3c4ccccc4c4ccccc43)c2)cc1]

From the results of Table 4, it could be identified that the blue organic light-emitting devices of Examples 1 to 44 using the heterocyclic compounds according to the present invention as a host material exhibited lower driving voltage, and significantly improved light emission efficiency and lifetime compared to the organic light-emitting devices using the compounds described in Comparative Examples 1 to 7.

It could be identified that the organic light-emitting device using the compound of Comparative Example 1 exhibited a reduced lifetime since the balance between holes and electrons was broken in the light emitting layer.

It could be identified that the organic light-emitting devices using the compounds of Comparative Example 2 and Comparative Example 3 exhibited a reduced lifetime since the balance between holes and electrons were broken due to changes in the steric structure during the vacuum deposition.

The compound of Comparative Example 4 has C—C bond linkage at the No. 3 position of the carbazole, resulting in a decrease in T₁. In addition, it could be identified that the organic light-emitting device using the compound of Comparative Example 4 exhibited a reduced lifetime since the migration rate of holes increased due to the expansion of HOMO energy, and the balance was broken due to the fast migration rate of each electron and hole.

It could be identified that the organic light-emitting devices using the compounds of Comparative Example 5 and Comparative Example 6 exhibited a reduced lifetime since the migration rate of electrons increased due to the expansion of LUMO energy, and the balance with holes was broken.

The compound of Comparative Example 7 exhibited a reduced T1 due to the expansion of HOMO energy caused by the substitution of a phenyl group at the No. 3 position of the carbazole. Accordingly, it could be identified that the organic light-emitting device using the compound of Comparative Example 7 exhibited a reduced lifetime since the desired wavelength was not achieved, and the balance was broken as well due to fast migration rate of electrons and holes.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

1. A heterocyclic compound represented by the following chemical formula 1: 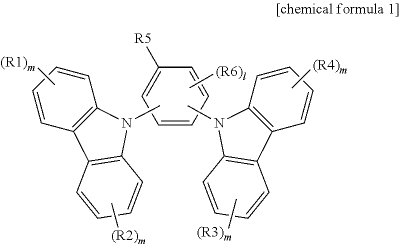
wherein, in chemical formula 1,
R1 to R4 are the same or different, and are each independently selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; a substituted or unsubstituted C2-C60 heterocycloalkyl group; and HAr;
the HAr is a substituted or unsubstituted C2-C60 heteroaryl group containing at least one nitrogen;
any one of R1 to R4 is HAr;
R5 is a substituted or unsubstituted C6-C60 aryl group;
R6 is selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; and a substituted or unsubstituted C2-C60 heterocycloalkyl group;
1 is an integer of 1-3; and
m is an integer of 1-4.

2. The heterocyclic compound of claim 1, wherein the compound of chemical formula 1 is represented by the following chemical formula 1-a: 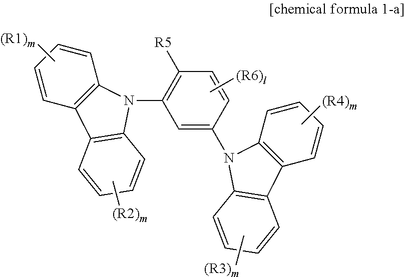
in chemical formula 1-a,
R1 to R6, 1 and m have the same definitions as in chemical formula 1.

3. The heterocyclic compound of claim 1, wherein R5 is any one selected from among the following structural formulae: 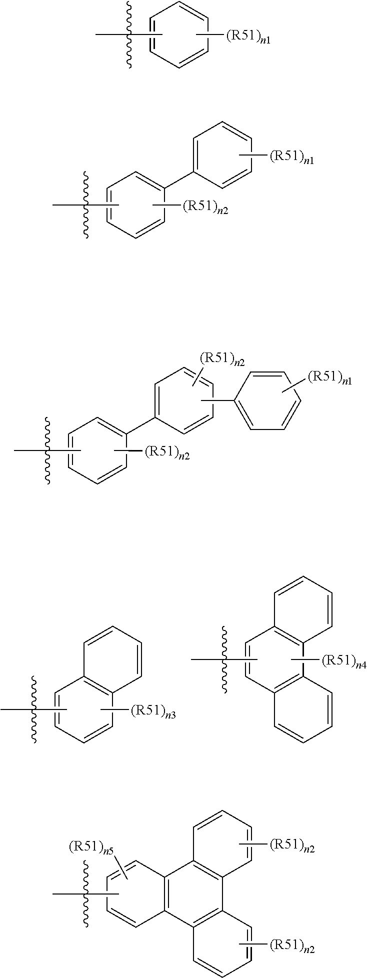
herein,
R51 is selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; and a substituted or unsubstituted C2-C60 heterocycloalkyl group, or two or more groups adjacent to each other bond to each other to form a substituted or unsubstituted C6-C60 aromatic hydrocarbon ring or a substituted or unsubstituted C2-C60 heteroring;
n1 is an integer of 1-5;
n2 is an integer of 1-4;
n3 is an integer of 1-7;
n4 is an integer of 1-9; and
n5 is an integer of 1-3.

4. The heterocyclic compound of claim 1, wherein the HAr is represented by the following chemical formula 1-2: 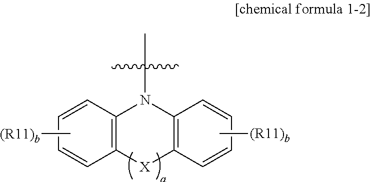
in chemical formula 1-2,
R11s are each independently selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; and a substituted or unsubstituted C2-C60 heterocycloalkyl group, or two or more groups adjacent to each other bond to each other to form a substituted or unsubstituted C6-C60 aromatic hydrocarbon ring or a substituted or unsubstituted C2-C60 heteroring;
X is O or CRaRb;
Ra and Rb are each independently selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; a substituted or unsubstituted C2-C60 heterocycloalkyl group; and a substituted or unsubstituted C6-C60 aryl group;
a is an integer of 0 or 1; and
b is an integer of 1-4.

5. The heterocyclic compound of claim 4, wherein the group represented by chemical formula 1-2 is represented by any one of the following chemical formula 1-2-a to chemical formula 1-2-e: 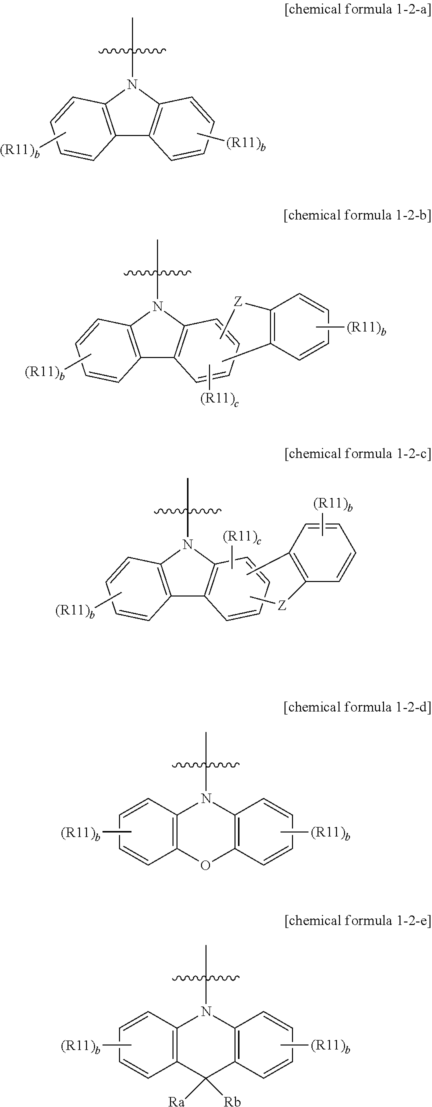
in chemical formulae 1-2-a to 1-2-e,
Z is NRc, O, S or CRdRe;
Rc, Rd and Re are each independently selected from the group consisting of: hydrogen; deuterium; halogen; a cyano group; a substituted or unsubstituted C1-C60 alkyl group; a substituted or unsubstituted C2-C60 alkenyl group; a substituted or unsubstituted C2-C60 alkynyl group; a substituted or unsubstituted C1-C60 alkoxy group; a substituted or unsubstituted C3-C60 cycloalkyl group; a substituted or unsubstituted C2-C60 heterocycloalkyl group; and a substituted or unsubstituted C6-C60 aryl group;
c is an integer of 1 or 2; and
Ra, Rb, R11 and b have the same definitions as in chemical formula 1-2.

6. The heterocyclic compound of claim 5, wherein the groups represented by chemical formulae 1-2-b and 1-2-c are respectively represented by any one of the following chemical formulae 1-2-b-1 to 1-2-b-3 and the following chemical formulae 1-2-c-1 to 1-2-c-3: 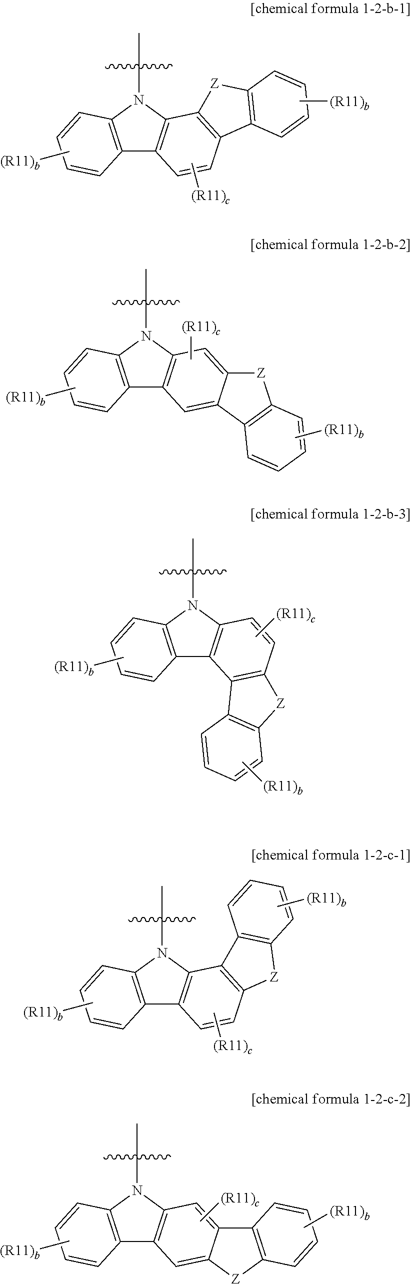 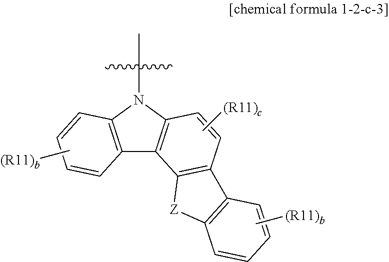
in chemical formulae 1-2-b-1 to 1-2-b-3 and chemical formulae 1-2-c-1 to 1-2-c-3,
R11, Z, b and c have the same definitions as in chemical formulae 1-2-b and 1-2-c.

7. The heterocyclic compound of claim 1, wherein the heterocyclic compound represented by chemical formula 1 does not include deuterium as a substituent, or has a deuterium content of 1% to 100% based on a total number of hydrogen atoms and deuterium atoms.

8. The heterocyclic compound of claim 1, wherein the heterocyclic compound represented by chemical formula 1 is any one selected from the group consisting of the following compounds:

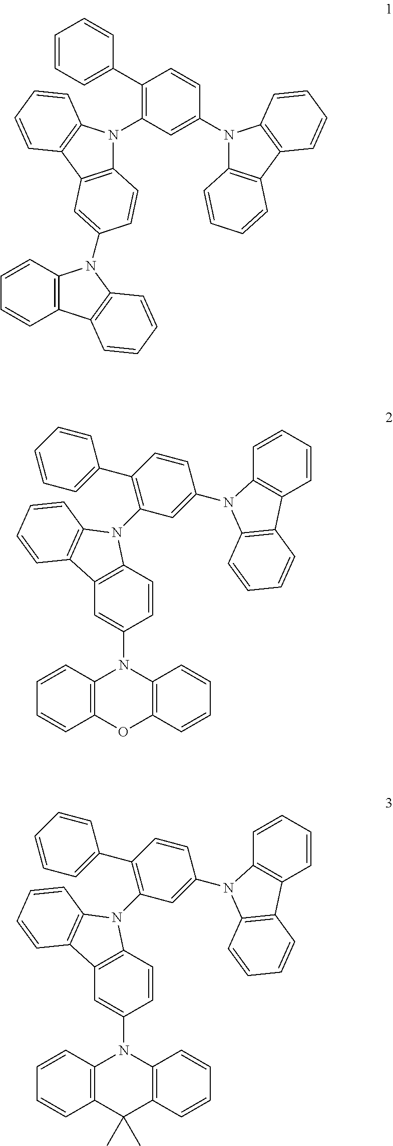 [SMILES: c1ccc(-c2ccc(-n3c4ccccc4c4ccccc43)cc2-n2c3ccccc3c3cc(-n4c5ccccc5c5ccccc54)ccc32)cc1 ; c1ccc(-c2ccc(-n3c4ccccc4c4ccccc43)cc2-n2c3ccccc3c3cc(N4c5ccccc5Oc5ccccc54)ccc32)cc1 ; CC1(C)c2ccccc2N(c2ccc3c(c2)c2ccccc2n3-c2cc(-n3c4ccccc4c4ccccc43)ccc2-c2ccccc2)c2ccccc21]

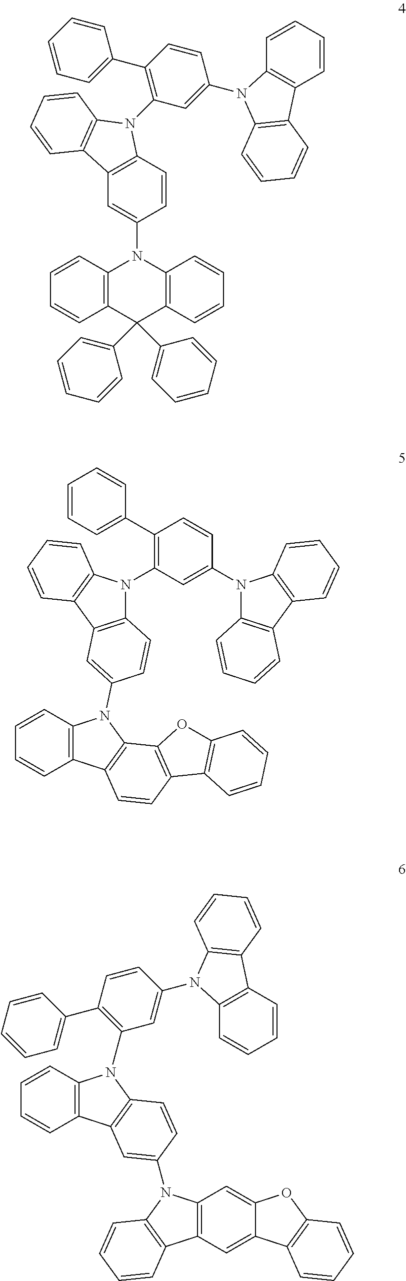 [SMILES: c1ccc(-c2ccc(-n3c4ccccc4c4ccccc43)cc2-n2c3ccccc3c3cc(N4c5ccccc5C(c5ccccc5)(c5ccccc5)c5ccccc54)ccc32)cc1 ; c1ccc(-c2ccc(-n3c4ccccc4c4ccccc43)cc2-n2c3ccccc3c3cc(-n4c5ccccc5c5ccc6c7ccccc7oc6c54)ccc32)cc1 ; c1ccc(-c2ccc(-n3c4ccccc4c4ccccc43)cc2-n2c3ccccc3c3cc(-n4c5ccccc5c5cc6c(cc54)oc4ccccc46)ccc32)cc1]

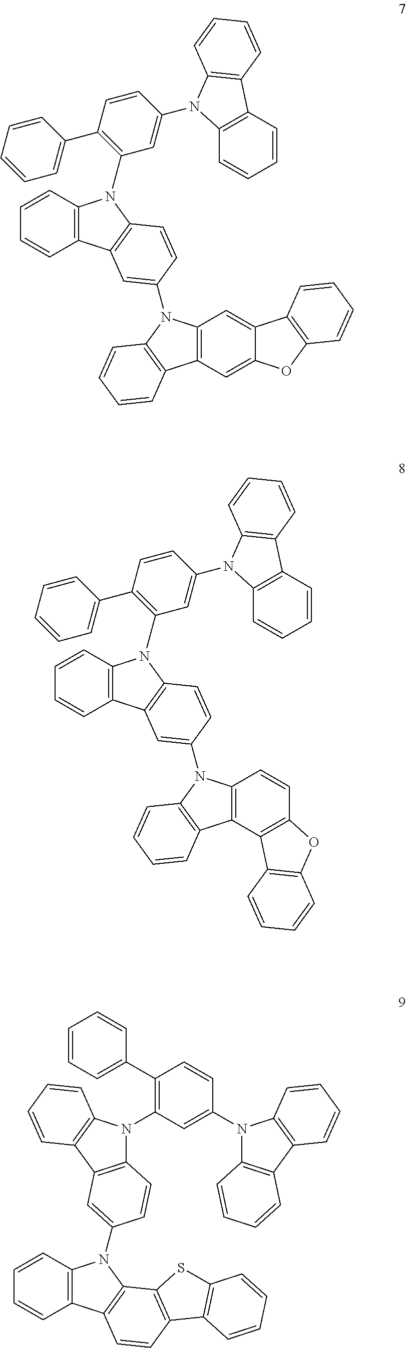 [SMILES: c1ccc(-c2ccc(-n3c4ccccc4c4ccccc43)cc2-n2c3ccccc3c3cc(-n4c5ccccc5c5cc6oc7ccccc7c6cc54)ccc32)cc1 ; c1ccc(-c2ccc(-n3c4ccccc4c4ccccc43)cc2-n2c3ccccc3c3cc(-n4c5ccccc5c5c6c(ccc54)oc4ccccc46)ccc32)cc1 ; c1ccc(-c2ccc(-n3c4ccccc4c4ccccc43)cc2-n2c3ccccc3c3cc(-n4c5ccccc5c5ccc6c7ccccc7sc6c54)ccc32)cc1]

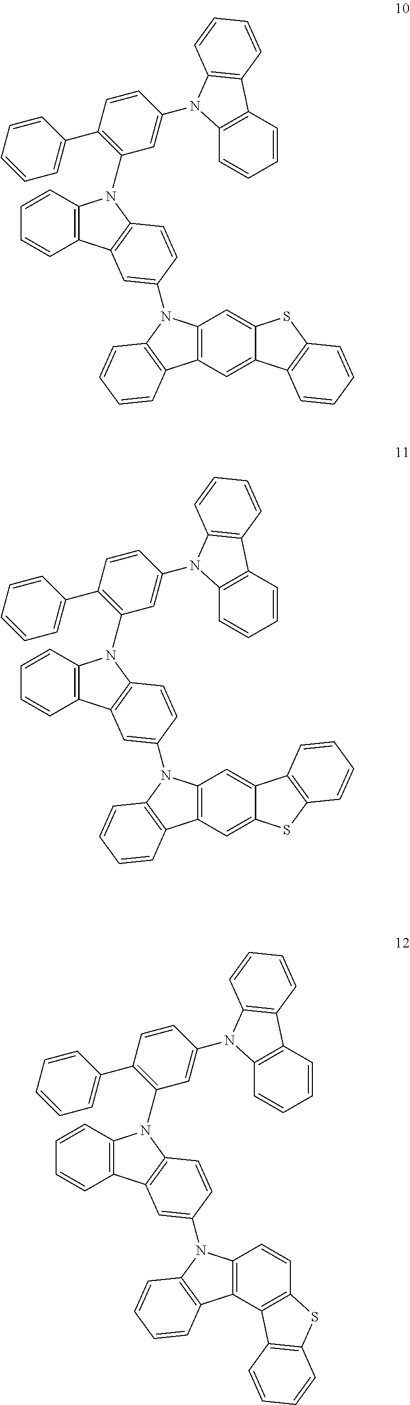 [SMILES: c1ccc(-c2ccc(-n3c4ccccc4c4ccccc43)cc2-n2c3ccccc3c3cc(-n4c5ccccc5c5cc6c(cc54)sc4ccccc46)ccc32)cc1 ; c1ccc(-c2ccc(-n3c4ccccc4c4ccccc43)cc2-n2c3ccccc3c3cc(-n4c5ccccc5c5cc6sc7ccccc7c6cc54)ccc32)cc1 ; c1ccc(-c2ccc(-n3c4ccccc4c4ccccc43)cc2-n2c3ccccc3c3cc(-n4c5ccccc5c5c6c(ccc54)sc4ccccc46)ccc32)cc1]

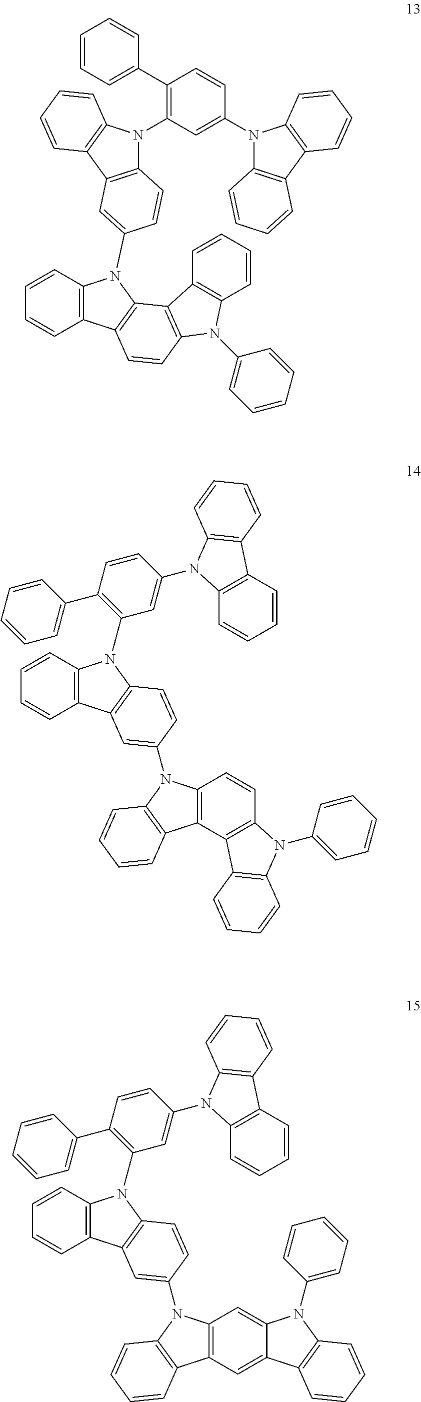 [SMILES: c1ccc(-c2ccc(-n3c4ccccc4c4ccccc43)cc2-n2c3ccccc3c3cc(-n4c5ccccc5c5ccc6c(c7ccccc7n6-c6ccccc6)c54)ccc32)cc1 ; c1ccc(-c2ccc(-n3c4ccccc4c4ccccc43)cc2-n2c3ccccc3c3cc(-n4c5ccccc5c5c6c7ccccc7n(-c7ccccc7)c6ccc54)ccc32)cc1 ; c1ccc(-c2ccc(-n3c4ccccc4c4ccccc43)cc2-n2c3ccccc3c3cc(-n4c5ccccc5c5cc6c7ccccc7n(-c7ccccc7)c6cc54)ccc32)cc1]

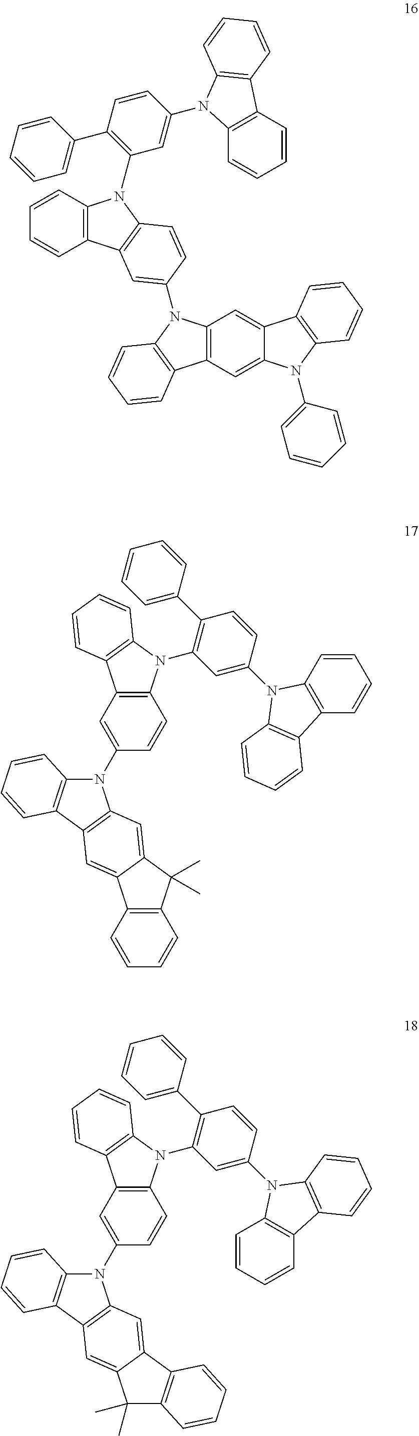 [SMILES: c1ccc(-c2ccc(-n3c4ccccc4c4ccccc43)cc2-n2c3ccccc3c3cc(-n4c5ccccc5c5cc6c(cc54)c4ccccc4n6-c4ccccc4)ccc32)cc1 ; CC1(C)c2ccccc2-c2cc3c4ccccc4n(-c4ccc5c(c4)c4ccccc4n5-c4cc(-n5c6ccccc6c6ccccc65)ccc4-c4ccccc4)c3cc21 ; CC1(C)c2ccccc2-c2cc3c(cc21)c1ccccc1n3-c1ccc2c(c1)c1ccccc1n2-c1cc(-n2c3ccccc3c3ccccc32)ccc1-c1ccccc1]

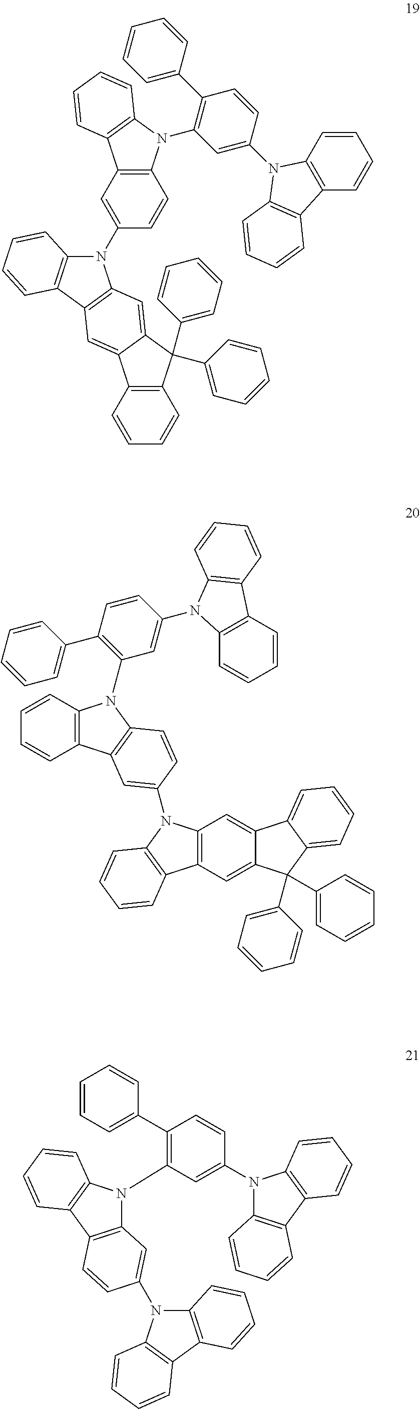 [SMILES: c1ccc(-c2ccc(-n3c4ccccc4c4ccccc43)cc2-n2c3ccccc3c3cc(-n4c5ccccc5c5cc6c(cc54)C(c4ccccc4)(c4ccccc4)c4ccccc4-6)ccc32)cc1 ; c1ccc(-c2ccc(-n3c4ccccc4c4ccccc43)cc2-n2c3ccccc3c3cc(-n4c5ccccc5c5cc6c(cc54)-c4ccccc4C6(c4ccccc4)c4ccccc4)ccc32)cc1 ; c1ccc(-c2ccc(-n3c4ccccc4c4ccccc43)cc2-n2c3ccccc3c3ccc(-n4c5ccccc5c5ccccc54)cc32)cc1]

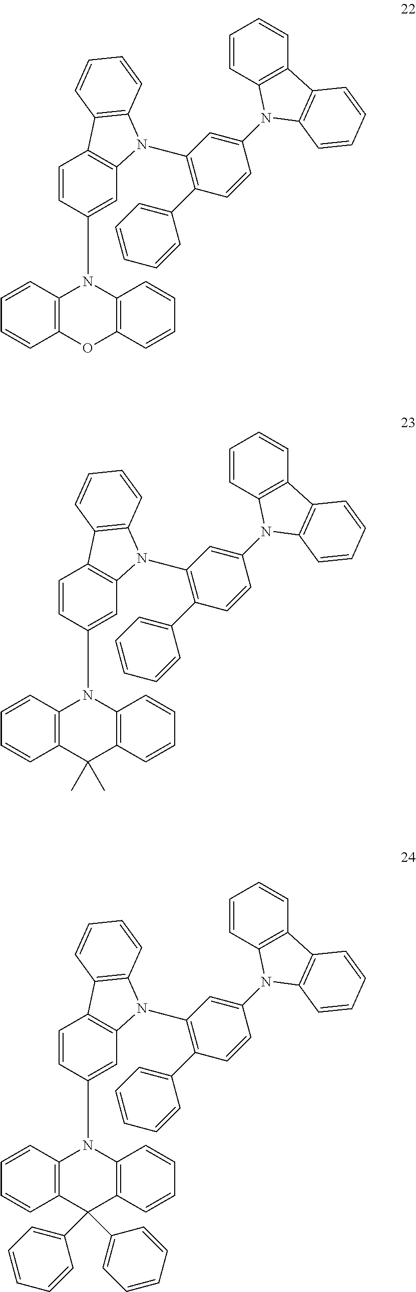 [SMILES: c1ccc(-c2ccc(-n3c4ccccc4c4ccccc43)cc2-n2c3ccccc3c3ccc(N4c5ccccc5Oc5ccccc54)cc32)cc1 ; CC1(C)c2ccccc2N(c2ccc3c4ccccc4n(-c4cc(-n5c6ccccc6c6ccccc65)ccc4-c4ccccc4)c3c2)c2ccccc21 ; c1ccc(-c2ccc(-n3c4ccccc4c4ccccc43)cc2-n2c3ccccc3c3ccc(N4c5ccccc5C(c5ccccc5)(c5ccccc5)c5ccccc54)cc32)cc1]

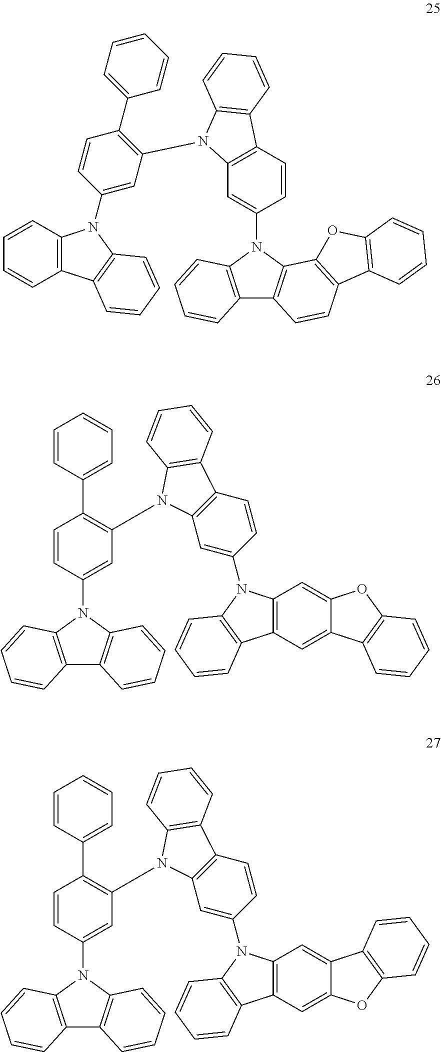 [SMILES: c1ccc(-c2ccc(-n3c4ccccc4c4ccccc43)cc2-n2c3ccccc3c3ccc(-n4c5ccccc5c5ccc6c7ccccc7oc6c54)cc32)cc1 ; c1ccc(-c2ccc(-n3c4ccccc4c4ccccc43)cc2-n2c3ccccc3c3ccc(-n4c5ccccc5c5cc6c(cc54)oc4ccccc46)cc32)cc1 ; c1ccc(-c2ccc(-n3c4ccccc4c4ccccc43)cc2-n2c3ccccc3c3ccc(-n4c5ccccc5c5cc6oc7ccccc7c6cc54)cc32)cc1]

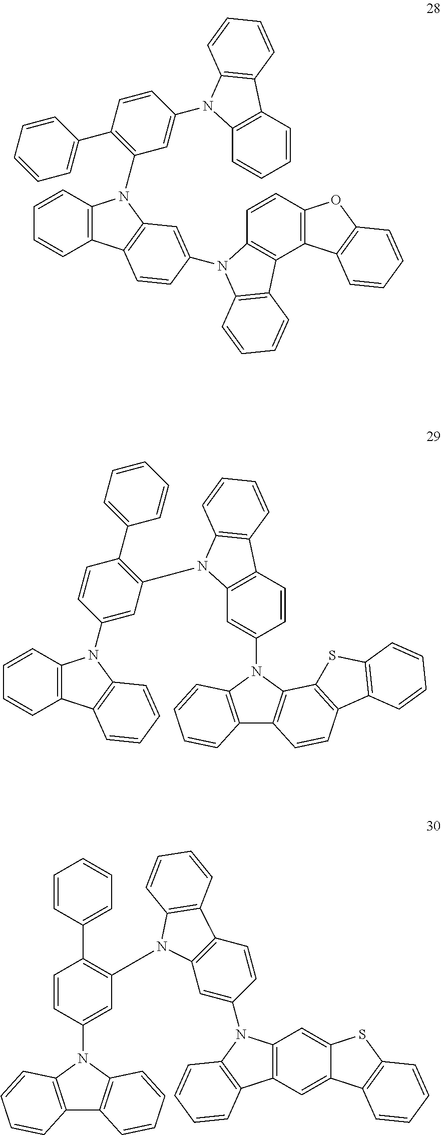 [SMILES: c1ccc(-c2ccc(-n3c4ccccc4c4ccccc43)cc2-n2c3ccccc3c3ccc(-n4c5ccccc5c5c6c(ccc54)oc4ccccc46)cc32)cc1 ; c1ccc(-c2ccc(-n3c4ccccc4c4ccccc43)cc2-n2c3ccccc3c3ccc(-n4c5ccccc5c5ccc6c7ccccc7sc6c54)cc32)cc1 ; c1ccc(-c2ccc(-n3c4ccccc4c4ccccc43)cc2-n2c3ccccc3c3ccc(-n4c5ccccc5c5cc6c(cc54)sc4ccccc46)cc32)cc1]

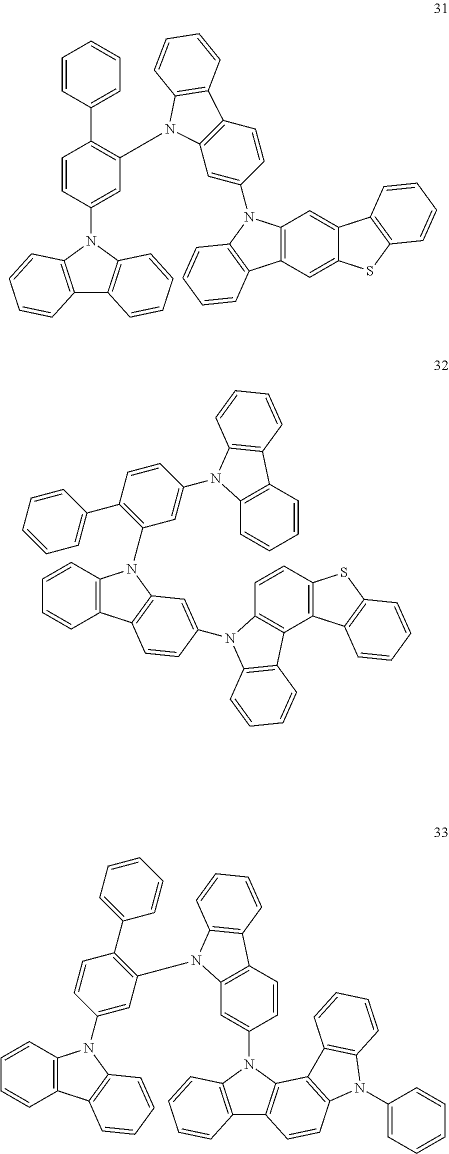 [SMILES: c1ccc(-c2ccc(-n3c4ccccc4c4ccccc43)cc2-n2c3ccccc3c3ccc(-n4c5ccccc5c5cc6sc7ccccc7c6cc54)cc32)cc1 ; c1ccc(-c2ccc(-n3c4ccccc4c4ccccc43)cc2-n2c3ccccc3c3ccc(-n4c5ccccc5c5c6c(ccc54)sc4ccccc46)cc32)cc1 ; c1ccc(-c2ccc(-n3c4ccccc4c4ccccc43)cc2-n2c3ccccc3c3ccc(-n4c5ccccc5c5ccc6c(c7ccccc7n6-c6ccccc6)c54)cc32)cc1]

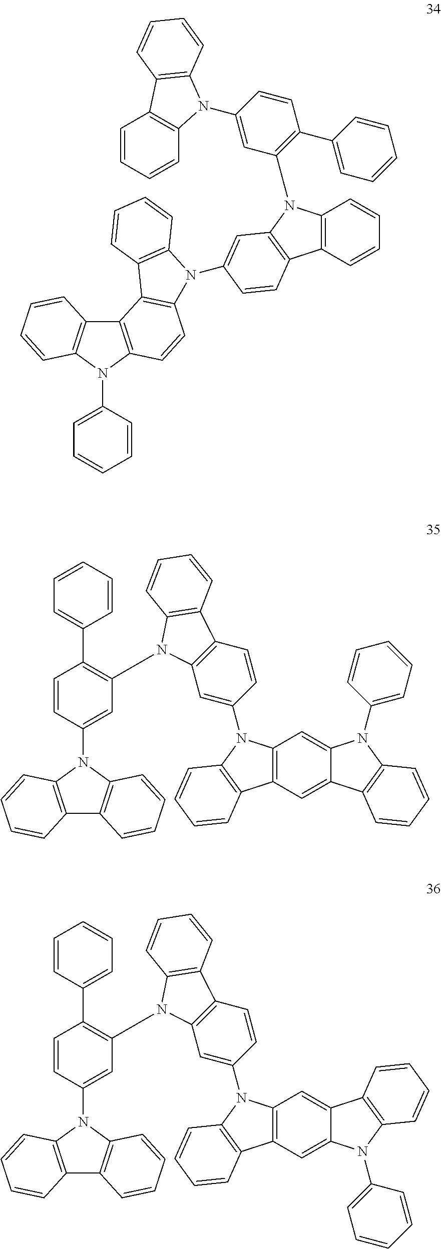 [SMILES: c1ccc(-c2ccc(-n3c4ccccc4c4ccccc43)cc2-n2c3ccccc3c3ccc(-n4c5ccccc5c5c6c7ccccc7n(-c7ccccc7)c6ccc54)cc32)cc1 ; c1ccc(-c2ccc(-n3c4ccccc4c4ccccc43)cc2-n2c3ccccc3c3ccc(-n4c5ccccc5c5cc6c7ccccc7n(-c7ccccc7)c6cc54)cc32)cc1 ; c1ccc(-c2ccc(-n3c4ccccc4c4ccccc43)cc2-n2c3ccccc3c3ccc(-n4c5ccccc5c5cc6c(cc54)c4ccccc4n6-c4ccccc4)cc32)cc1]

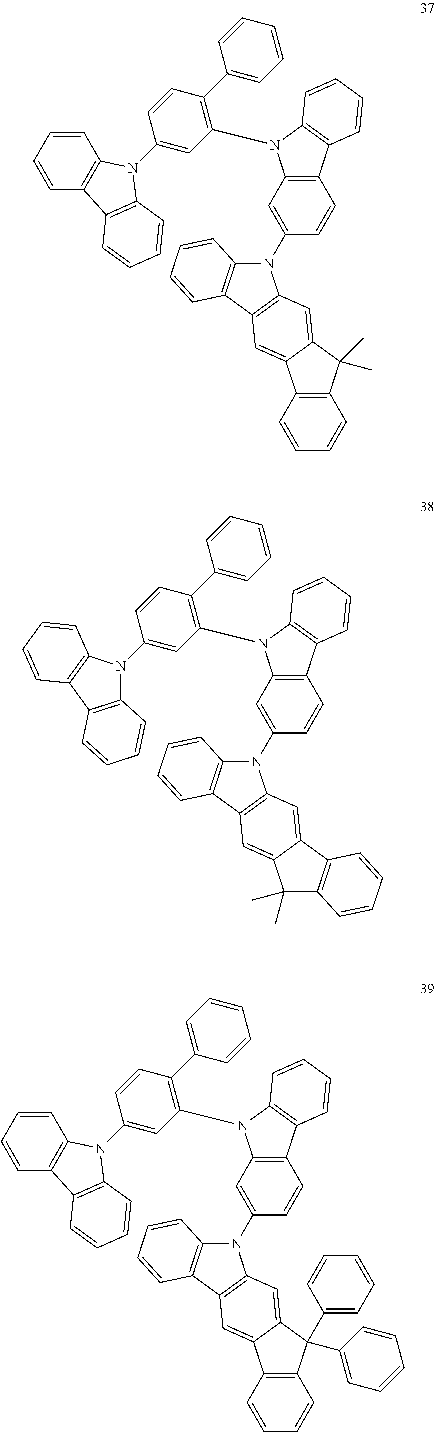 [SMILES: CC1(C)c2ccccc2-c2cc3c4ccccc4n(-c4ccc5c6ccccc6n(-c6cc(-n7c8ccccc8c8ccccc87)ccc6-c6ccccc6)c5c4)c3cc21 ; CC1(C)c2ccccc2-c2cc3c(cc21)c1ccccc1n3-c1ccc2c3ccccc3n(-c3cc(-n4c5ccccc5c5ccccc54)ccc3-c3ccccc3)c2c1 ; c1ccc(-c2ccc(-n3c4ccccc4c4ccccc43)cc2-n2c3ccccc3c3ccc(-n4c5ccccc5c5cc6c(cc54)C(c4ccccc4)(c4ccccc4)c4ccccc4-6)cc32)cc1]

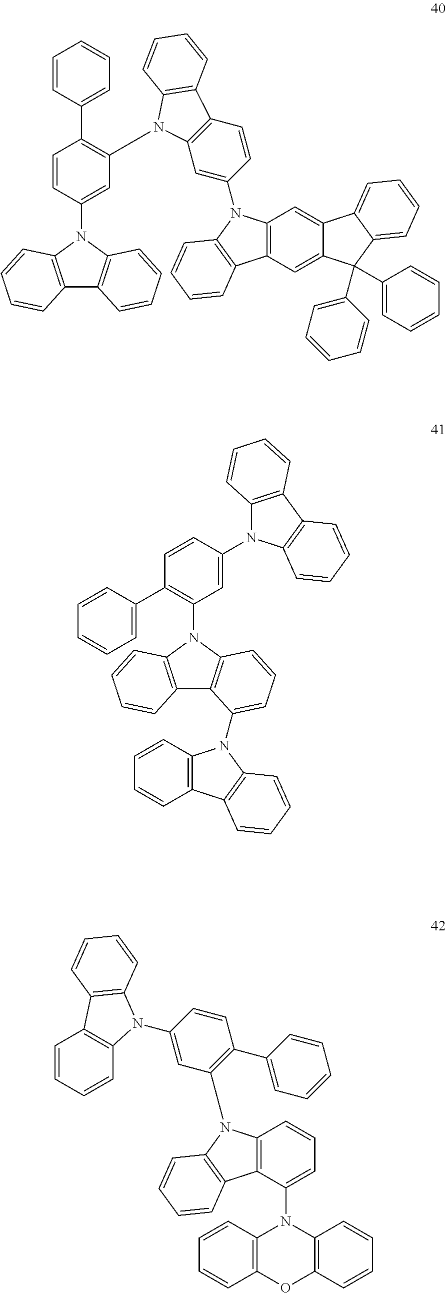 [SMILES: c1ccc(-c2ccc(-n3c4ccccc4c4ccccc43)cc2-n2c3ccccc3c3ccc(-n4c5ccccc5c5cc6c(cc54)-c4ccccc4C6(c4ccccc4)c4ccccc4)cc32)cc1 ; c1ccc(-c2ccc(-n3c4ccccc4c4ccccc43)cc2-n2c3ccccc3c3c(-n4c5ccccc5c5ccccc54)cccc32)cc1 ; c1ccc(-c2ccc(-n3c4ccccc4c4ccccc43)cc2-n2c3ccccc3c3c(N4c5ccccc5Oc5ccccc54)cccc32)cc1]

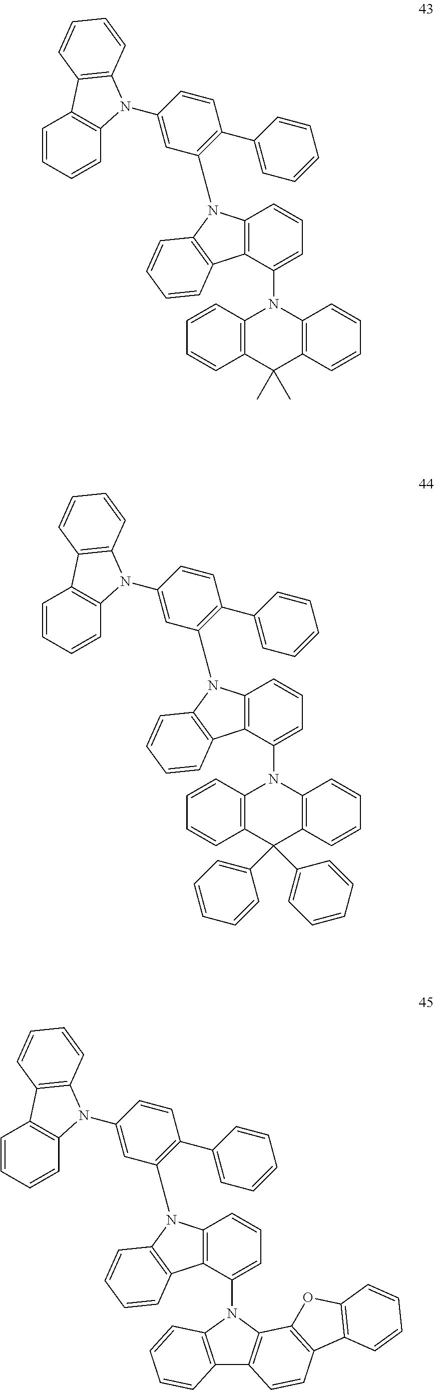 [SMILES: CC1(C)c2ccccc2N(c2cccc3c2c2ccccc2n3-c2cc(-n3c4ccccc4c4ccccc43)ccc2-c2ccccc2)c2ccccc21 ; c1ccc(-c2ccc(-n3c4ccccc4c4ccccc43)cc2-n2c3ccccc3c3c(N4c5ccccc5C(c5ccccc5)(c5ccccc5)c5ccccc54)cccc32)cc1 ; c1ccc(-c2ccc(-n3c4ccccc4c4ccccc43)cc2-n2c3ccccc3c3c(-n4c5ccccc5c5ccc6c7ccccc7oc6c54)cccc32)cc1]

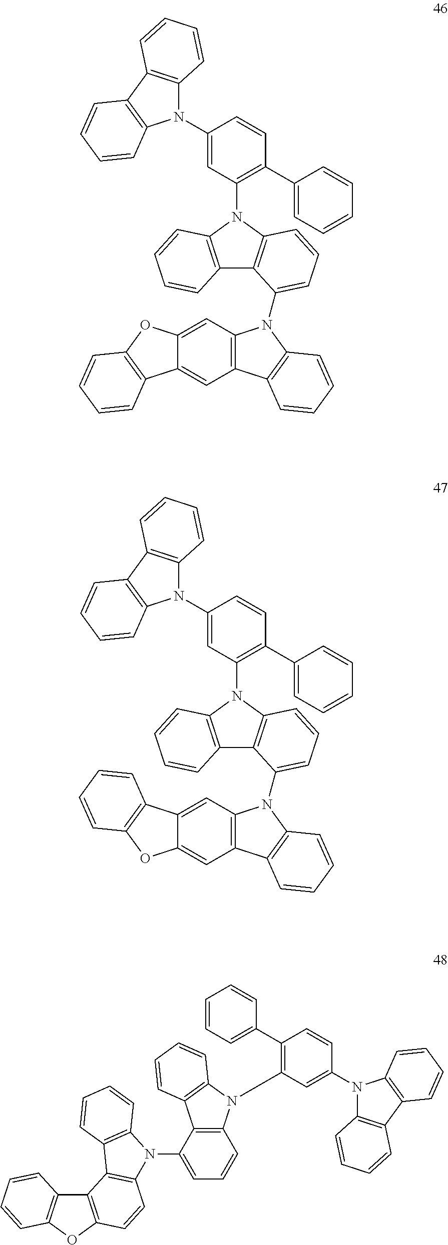 [SMILES: c1ccc(-c2ccc(-n3c4ccccc4c4ccccc43)cc2-n2c3ccccc3c3c(-n4c5ccccc5c5cc6c(cc54)oc4ccccc46)cccc32)cc1 ; c1ccc(-c2ccc(-n3c4ccccc4c4ccccc43)cc2-n2c3ccccc3c3c(-n4c5ccccc5c5cc6oc7ccccc7c6cc54)cccc32)cc1 ; c1ccc(-c2ccc(-n3c4ccccc4c4ccccc43)cc2-n2c3ccccc3c3c(-n4c5ccccc5c5c6c(ccc54)oc4ccccc46)cccc32)cc1]

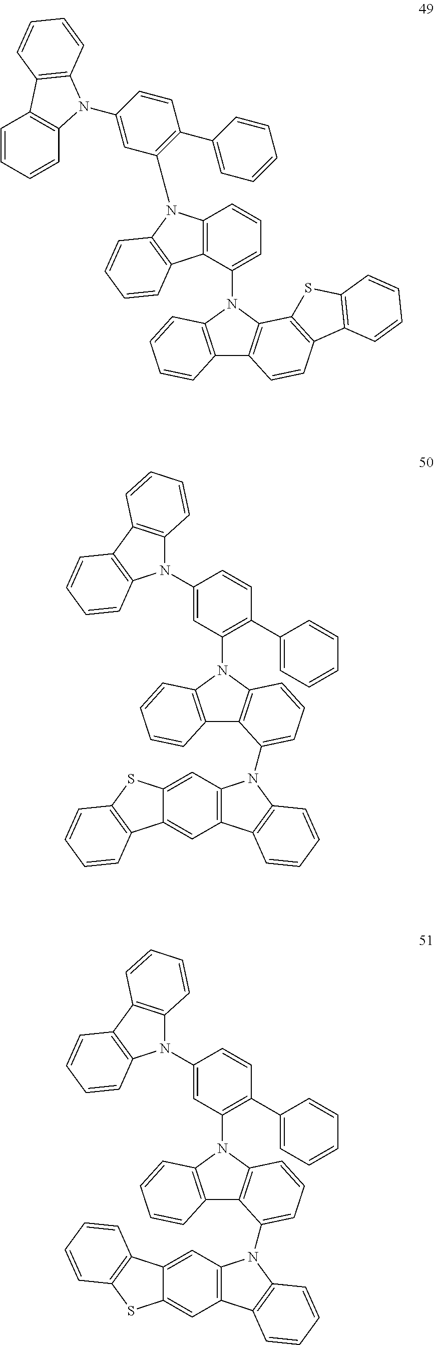 [SMILES: c1ccc(-c2ccc(-n3c4ccccc4c4ccccc43)cc2-n2c3ccccc3c3c(-n4c5ccccc5c5ccc6c7ccccc7sc6c54)cccc32)cc1 ; c1ccc(-c2ccc(-n3c4ccccc4c4ccccc43)cc2-n2c3ccccc3c3c(-n4c5ccccc5c5cc6c(cc54)sc4ccccc46)cccc32)cc1 ; c1ccc(-c2ccc(-n3c4ccccc4c4ccccc43)cc2-n2c3ccccc3c3c(-n4c5ccccc5c5cc6sc7ccccc7c6cc54)cccc32)cc1]

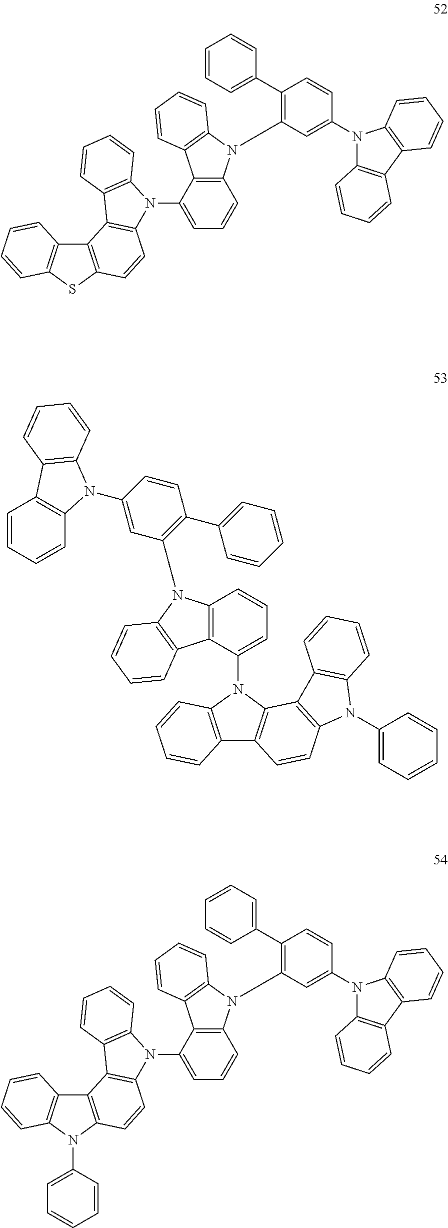 [SMILES: c1ccc(-c2ccc(-n3c4ccccc4c4ccccc43)cc2-n2c3ccccc3c3c(-n4c5ccccc5c5c6c(ccc54)sc4ccccc46)cccc32)cc1 ; c1ccc(-c2ccc(-n3c4ccccc4c4ccccc43)cc2-n2c3ccccc3c3c(-n4c5ccccc5c5ccc6c(c7ccccc7n6-c6ccccc6)c54)cccc32)cc1 ; c1ccc(-c2ccc(-n3c4ccccc4c4ccccc43)cc2-n2c3ccccc3c3c(-n4c5ccccc5c5c6c7ccccc7n(-c7ccccc7)c6ccc54)cccc32)cc1]

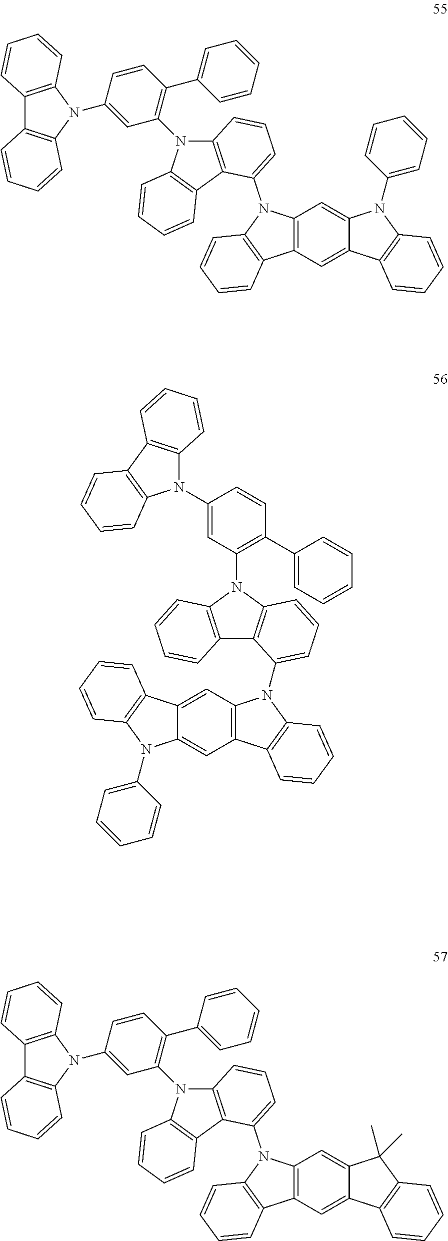 [SMILES: c1ccc(-c2ccc(-n3c4ccccc4c4ccccc43)cc2-n2c3ccccc3c3c(-n4c5ccccc5c5cc6c7ccccc7n(-c7ccccc7)c6cc54)cccc32)cc1 ; c1ccc(-c2ccc(-n3c4ccccc4c4ccccc43)cc2-n2c3ccccc3c3c(-n4c5ccccc5c5cc6c(cc54)c4ccccc4n6-c4ccccc4)cccc32)cc1 ; CC1(C)c2ccccc2-c2cc3c4ccccc4n(-c4cccc5c4c4ccccc4n5-c4cc(-n5c6ccccc6c6ccccc65)ccc4-c4ccccc4)c3cc21]

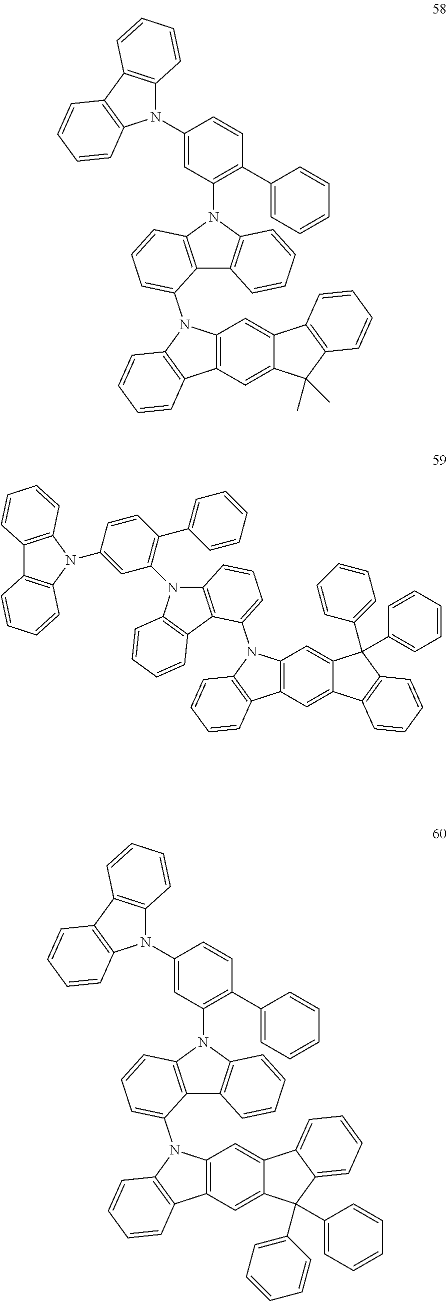 [SMILES: CC1(C)c2ccccc2-c2cc3c(cc21)c1ccccc1n3-c1cccc2c1c1ccccc1n2-c1cc(-n2c3ccccc3c3ccccc32)ccc1-c1ccccc1 ; c1ccc(-c2ccc(-n3c4ccccc4c4ccccc43)cc2-n2c3ccccc3c3c(-n4c5ccccc5c5cc6c(cc54)C(c4ccccc4)(c4ccccc4)c4ccccc4-6)cccc32)cc1 ; c1ccc(-c2ccc(-n3c4ccccc4c4ccccc43)cc2-n2c3ccccc3c3c(-n4c5ccccc5c5cc6c(cc54)-c4ccccc4C6(c4ccccc4)c4ccccc4)cccc32)cc1]

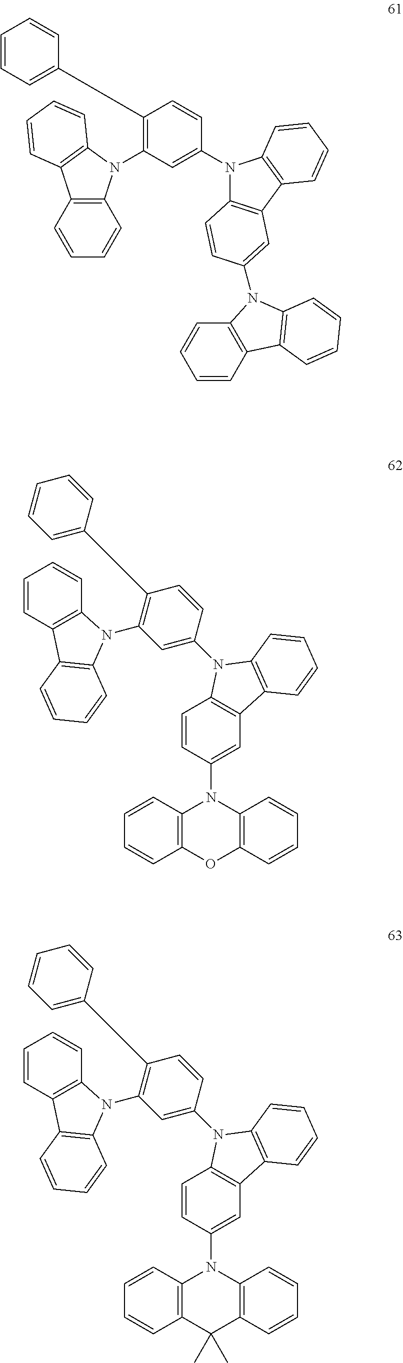 [SMILES: c1ccc(-c2ccc(-n3c4ccccc4c4cc(-n5c6ccccc6c6ccccc65)ccc43)cc2-n2c3ccccc3c3ccccc32)cc1 ; c1ccc(-c2ccc(-n3c4ccccc4c4cc(N5c6ccccc6Oc6ccccc65)ccc43)cc2-n2c3ccccc3c3ccccc32)cc1 ; CC1(C)c2ccccc2N(c2ccc3c(c2)c2ccccc2n3-c2ccc(-c3ccccc3)c(-n3c4ccccc4c4ccccc43)c2)c2ccccc21]

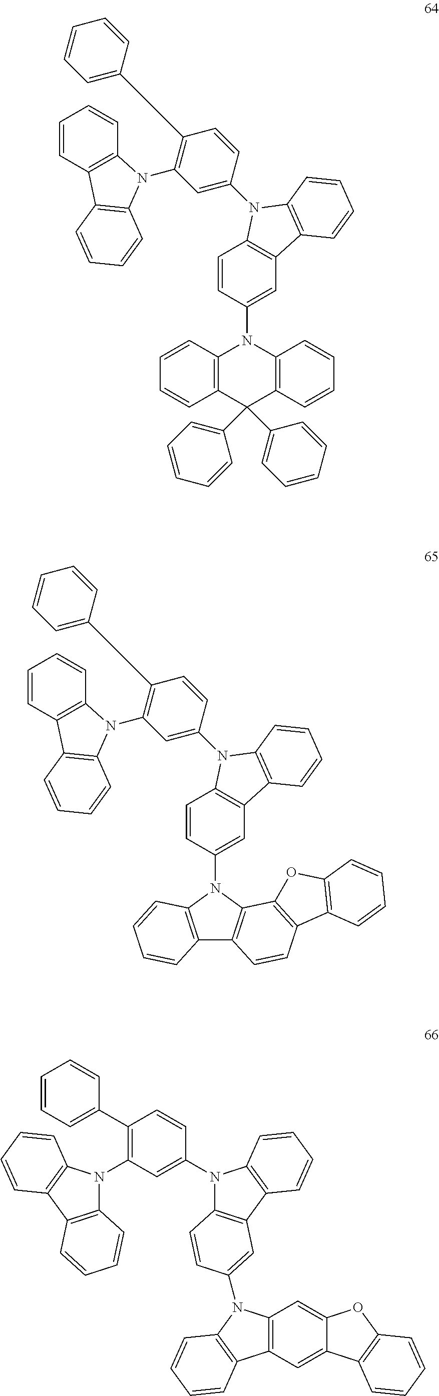 [SMILES: c1ccc(-c2ccc(-n3c4ccccc4c4cc(N5c6ccccc6C(c6ccccc6)(c6ccccc6)c6ccccc65)ccc43)cc2-n2c3ccccc3c3ccccc32)cc1 ; c1ccc(-c2ccc(-n3c4ccccc4c4cc(-n5c6ccccc6c6ccc7c8ccccc8oc7c65)ccc43)cc2-n2c3ccccc3c3ccccc32)cc1 ; c1ccc(-c2ccc(-n3c4ccccc4c4cc(-n5c6ccccc6c6cc7c(cc65)oc5ccccc57)ccc43)cc2-n2c3ccccc3c3ccccc32)cc1]

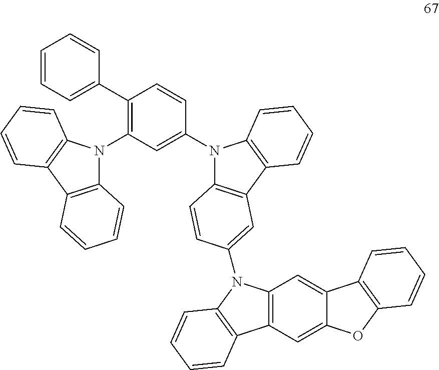 [SMILES: c1ccc(-c2ccc(-n3c4ccccc4c4cc(-n5c6ccccc6c6cc7oc8ccccc8c7cc65)ccc43)cc2-n2c3ccccc3c3ccccc32)cc1]

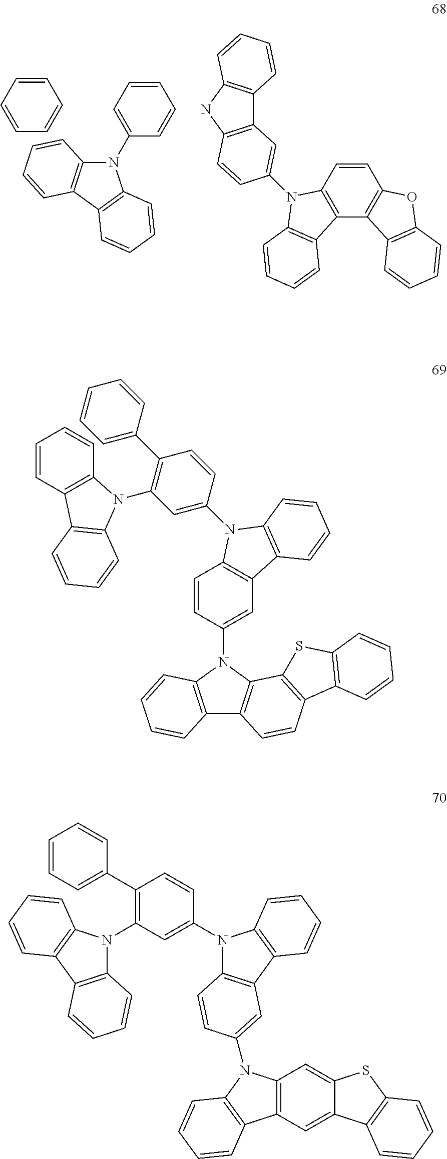

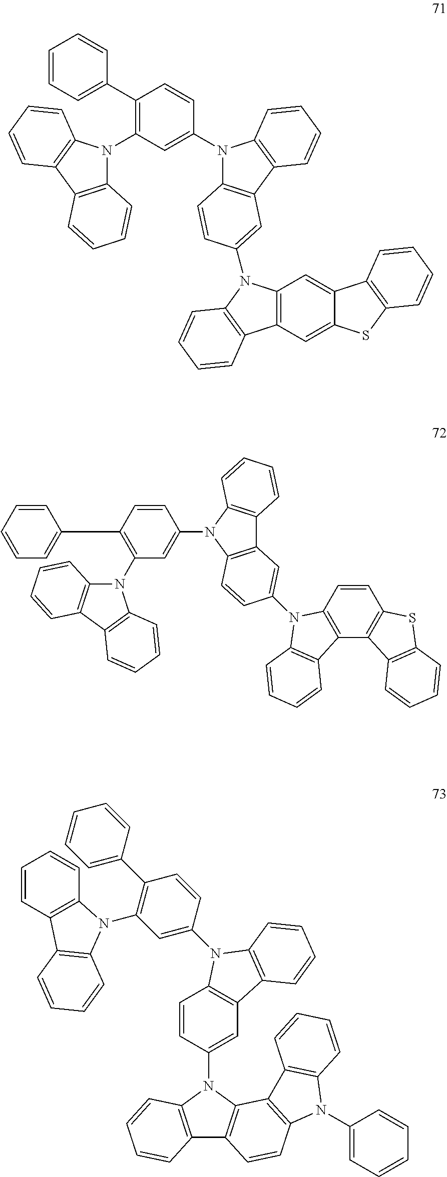 [SMILES: c1ccc(-c2ccc(-n3c4ccccc4c4cc(-n5c6ccccc6c6cc7sc8ccccc8c7cc65)ccc43)cc2-n2c3ccccc3c3ccccc32)cc1 ; c1ccc(-c2ccc(-n3c4ccccc4c4cc(-n5c6ccccc6c6c7c(ccc65)sc5ccccc57)ccc43)cc2-n2c3ccccc3c3ccccc32)cc1 ; c1ccc(-c2ccc(-n3c4ccccc4c4cc(-n5c6ccccc6c6ccc7c(c8ccccc8n7-c7ccccc7)c65)ccc43)cc2-n2c3ccccc3c3ccccc32)cc1]

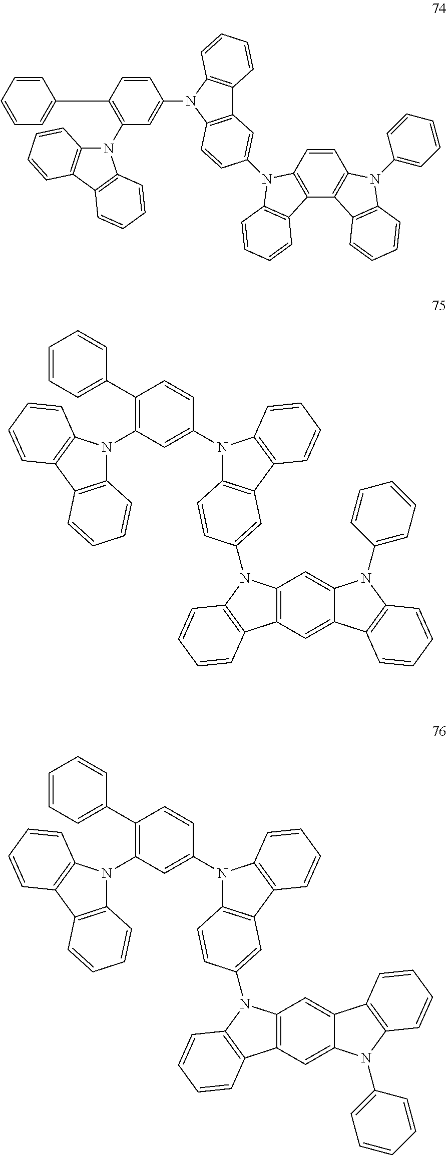 [SMILES: c1ccc(-c2ccc(-n3c4ccccc4c4cc(-n5c6ccccc6c6c7c8ccccc8n(-c8ccccc8)c7ccc65)ccc43)cc2-n2c3ccccc3c3ccccc32)cc1 ; c1ccc(-c2ccc(-n3c4ccccc4c4cc(-n5c6ccccc6c6cc7c8ccccc8n(-c8ccccc8)c7cc65)ccc43)cc2-n2c3ccccc3c3ccccc32)cc1 ; c1ccc(-c2ccc(-n3c4ccccc4c4cc(-n5c6ccccc6c6cc7c(cc65)c5ccccc5n7-c5ccccc5)ccc43)cc2-n2c3ccccc3c3ccccc32)cc1]

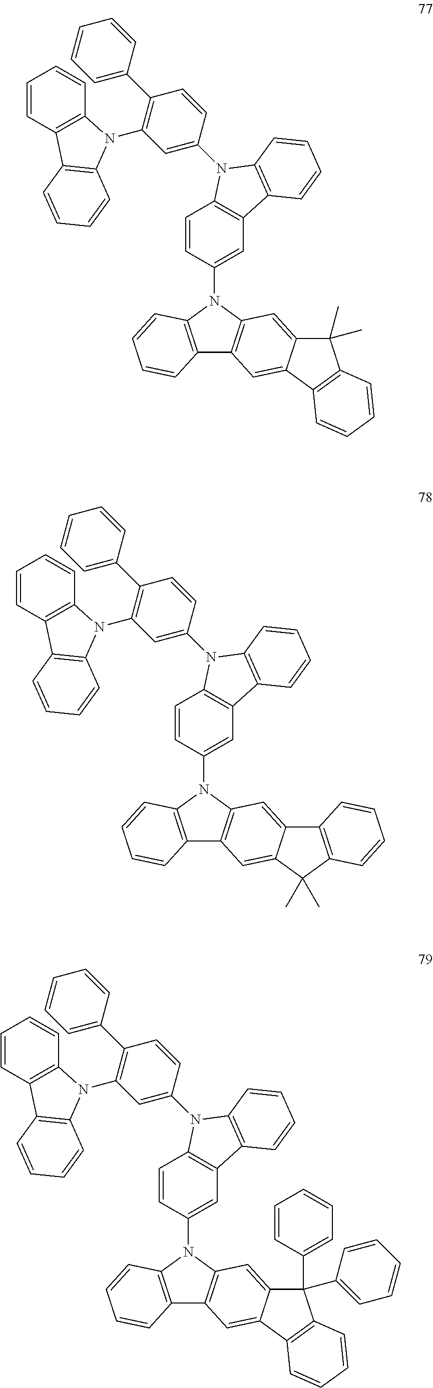 [SMILES: CC1(C)c2ccccc2-c2cc3c4ccccc4n(-c4ccc5c(c4)c4ccccc4n5-c4ccc(-c5ccccc5)c(-n5c6ccccc6c6ccccc65)c4)c3cc21 ; CC1(C)c2ccccc2-c2cc3c(cc21)c1ccccc1n3-c1ccc2c(c1)c1ccccc1n2-c1ccc(-c2ccccc2)c(-n2c3ccccc3c3ccccc32)c1 ; c1ccc(-c2ccc(-n3c4ccccc4c4cc(-n5c6ccccc6c6cc7c(cc65)C(c5ccccc5)(c5ccccc5)c5ccccc5-7)ccc43)cc2-n2c3ccccc3c3ccccc32)cc1]

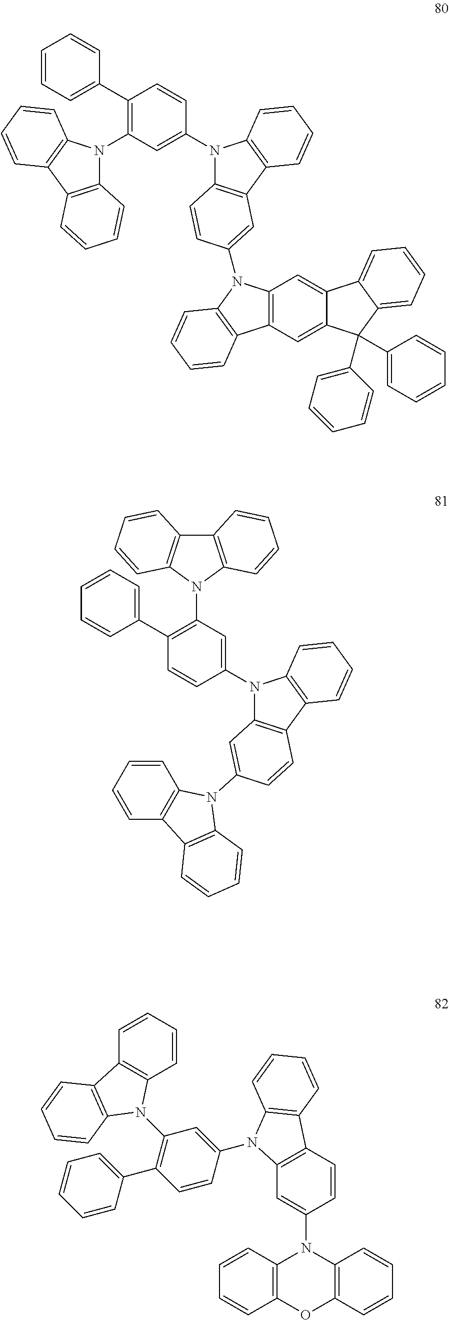 [SMILES: c1ccc(-c2ccc(-n3c4ccccc4c4cc(-n5c6ccccc6c6cc7c(cc65)-c5ccccc5C7(c5ccccc5)c5ccccc5)ccc43)cc2-n2c3ccccc3c3ccccc32)cc1 ; c1ccc(-c2ccc(-n3c4ccccc4c4ccc(-n5c6ccccc6c6ccccc65)cc43)cc2-n2c3ccccc3c3ccccc32)cc1 ; c1ccc(-c2ccc(-n3c4ccccc4c4ccc(N5c6ccccc6Oc6ccccc65)cc43)cc2-n2c3ccccc3c3ccccc32)cc1]

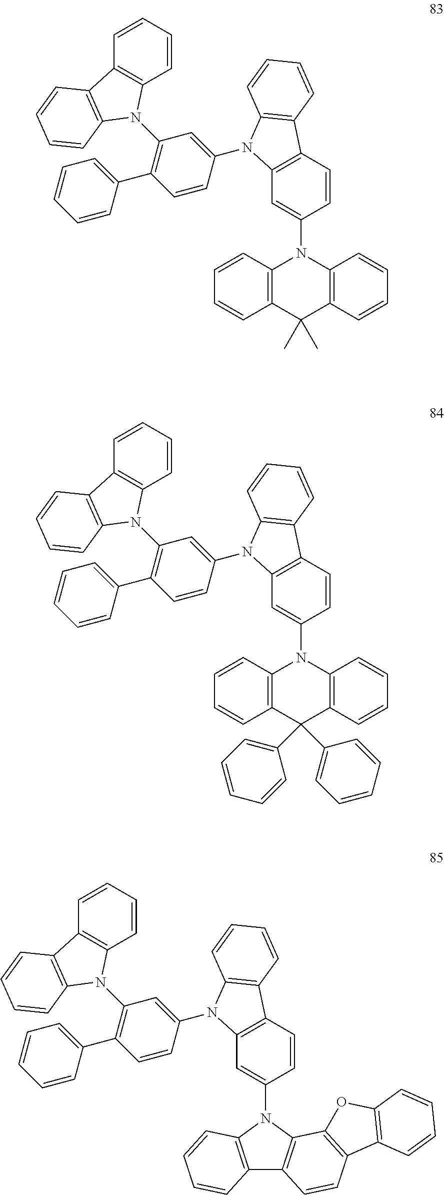 [SMILES: CC1(C)c2ccccc2N(c2ccc3c4ccccc4n(-c4ccc(-c5ccccc5)c(-n5c6ccccc6c6ccccc65)c4)c3c2)c2ccccc21 ; c1ccc(-c2ccc(-n3c4ccccc4c4ccc(N5c6ccccc6C(c6ccccc6)(c6ccccc6)c6ccccc65)cc43)cc2-n2c3ccccc3c3ccccc32)cc1 ; c1ccc(-c2ccc(-n3c4ccccc4c4ccc(-n5c6ccccc6c6ccc7c8ccccc8oc7c65)cc43)cc2-n2c3ccccc3c3ccccc32)cc1]

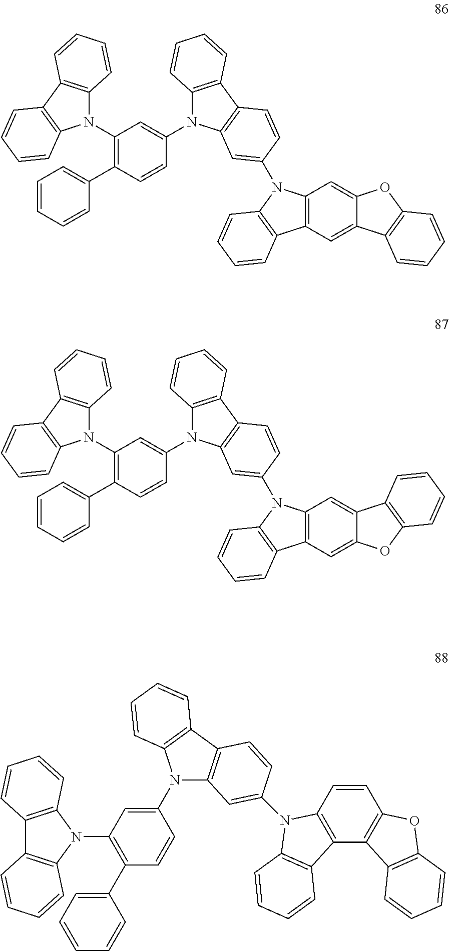 [SMILES: c1ccc(-c2ccc(-n3c4ccccc4c4ccc(-n5c6ccccc6c6cc7c(cc65)oc5ccccc57)cc43)cc2-n2c3ccccc3c3ccccc32)cc1 ; c1ccc(-c2ccc(-n3c4ccccc4c4ccc(-n5c6ccccc6c6cc7oc8ccccc8c7cc65)cc43)cc2-n2c3ccccc3c3ccccc32)cc1 ; c1ccc(-c2ccc(-n3c4ccccc4c4ccc(-n5c6ccccc6c6c7c(ccc65)oc5ccccc57)cc43)cc2-n2c3ccccc3c3ccccc32)cc1]

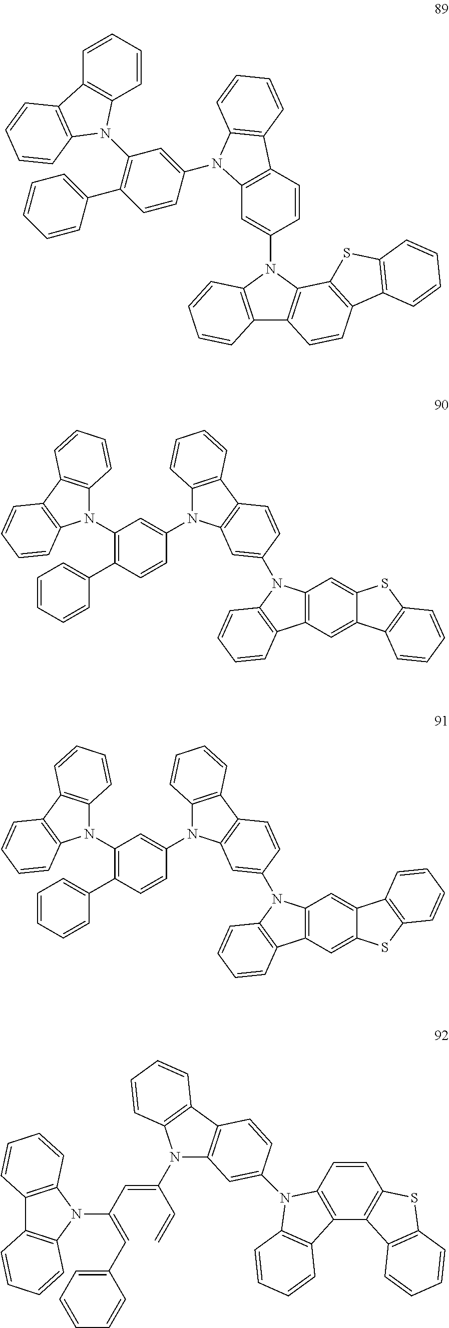 [SMILES: c1ccc(-c2ccc(-n3c4ccccc4c4ccc(-n5c6ccccc6c6ccc7c8ccccc8sc7c65)cc43)cc2-n2c3ccccc3c3ccccc32)cc1 ; c1ccc(-c2ccc(-n3c4ccccc4c4ccc(-n5c6ccccc6c6cc7c(cc65)sc5ccccc57)cc43)cc2-n2c3ccccc3c3ccccc32)cc1 ; c1ccc(-c2ccc(-n3c4ccccc4c4ccc(-n5c6ccccc6c6cc7sc8ccccc8c7cc65)cc43)cc2-n2c3ccccc3c3ccccc32)cc1 ; C=C/C(=C\C(=C\c1ccccc1)n1c2ccccc2c2ccccc21)n1c2ccccc2c2ccc(-n3c4ccccc4c4c5c(ccc43)sc3ccccc35)cc21]

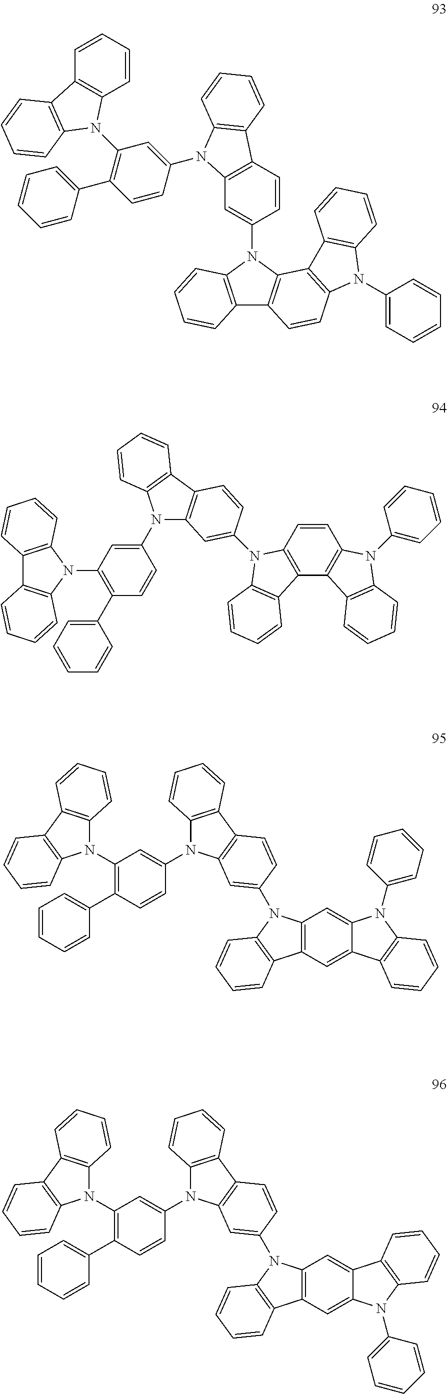 [SMILES: c1ccc(-c2ccc(-n3c4ccccc4c4ccc(-n5c6ccccc6c6ccc7c(c8ccccc8n7-c7ccccc7)c65)cc43)cc2-n2c3ccccc3c3ccccc32)cc1 ; c1ccc(-c2ccc(-n3c4ccccc4c4ccc(-n5c6ccccc6c6c7c8ccccc8n(-c8ccccc8)c7ccc65)cc43)cc2-n2c3ccccc3c3ccccc32)cc1 ; c1ccc(-c2ccc(-n3c4ccccc4c4ccc(-n5c6ccccc6c6cc7c8ccccc8n(-c8ccccc8)c7cc65)cc43)cc2-n2c3ccccc3c3ccccc32)cc1 ; c1ccc(-c2ccc(-n3c4ccccc4c4ccc(-n5c6ccccc6c6cc7c(cc65)c5ccccc5n7-c5ccccc5)cc43)cc2-n2c3ccccc3c3ccccc32)cc1]

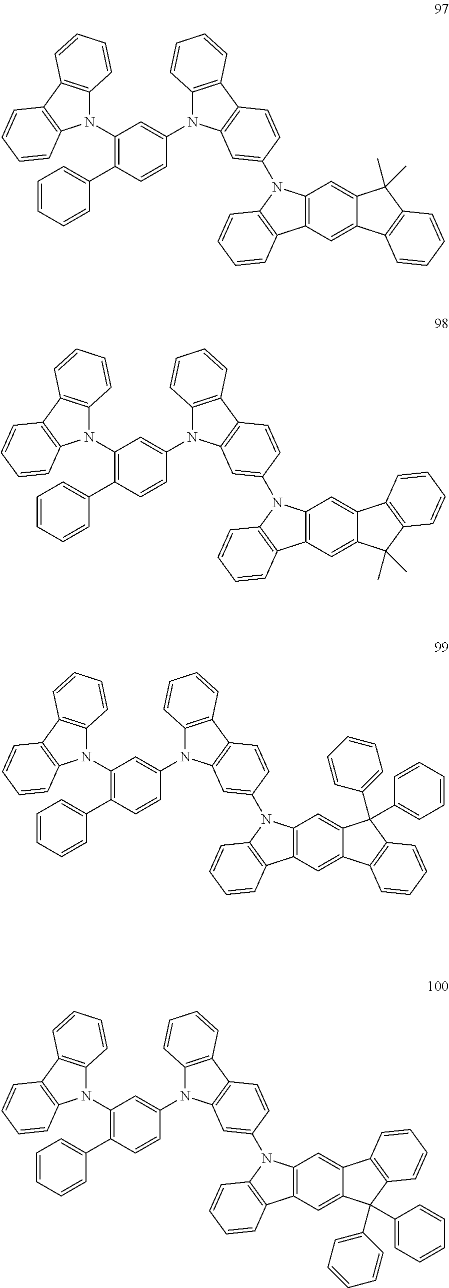 [SMILES: CC1(C)c2ccccc2-c2cc3c4ccccc4n(-c4ccc5c6ccccc6n(-c6ccc(-c7ccccc7)c(-n7c8ccccc8c8ccccc87)c6)c5c4)c3cc21 ; CC1(C)c2ccccc2-c2cc3c(cc21)c1ccccc1n3-c1ccc2c3ccccc3n(-c3ccc(-c4ccccc4)c(-n4c5ccccc5c5ccccc54)c3)c2c1 ; c1ccc(-c2ccc(-n3c4ccccc4c4ccc(-n5c6ccccc6c6cc7c(cc65)C(c5ccccc5)(c5ccccc5)c5ccccc5-7)cc43)cc2-n2c3ccccc3c3ccccc32)cc1 ; c1ccc(-c2ccc(-n3c4ccccc4c4ccc(-n5c6ccccc6c6cc7c(cc65)-c5ccccc5C7(c5ccccc5)c5ccccc5)cc43)cc2-n2c3ccccc3c3ccccc32)cc1]

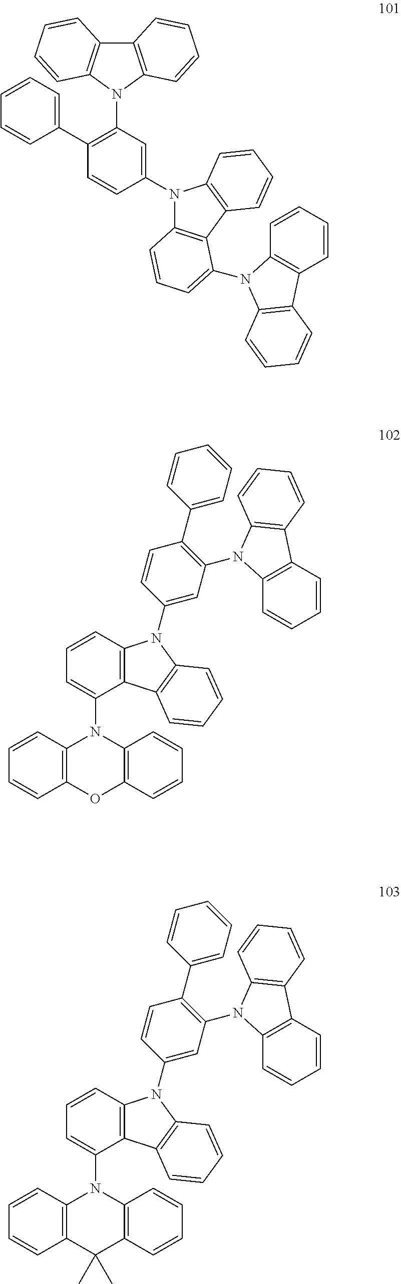 [SMILES: c1ccc(-c2ccc(-n3c4ccccc4c4c(-n5c6ccccc6c6ccccc65)cccc43)cc2-n2c3ccccc3c3ccccc32)cc1 ; c1ccc(-c2ccc(-n3c4ccccc4c4c(N5c6ccccc6Oc6ccccc65)cccc43)cc2-n2c3ccccc3c3ccccc32)cc1 ; CC1(C)c2ccccc2N(c2cccc3c2c2ccccc2n3-c2ccc(-c3ccccc3)c(-n3c4ccccc4c4ccccc43)c2)c2ccccc21]

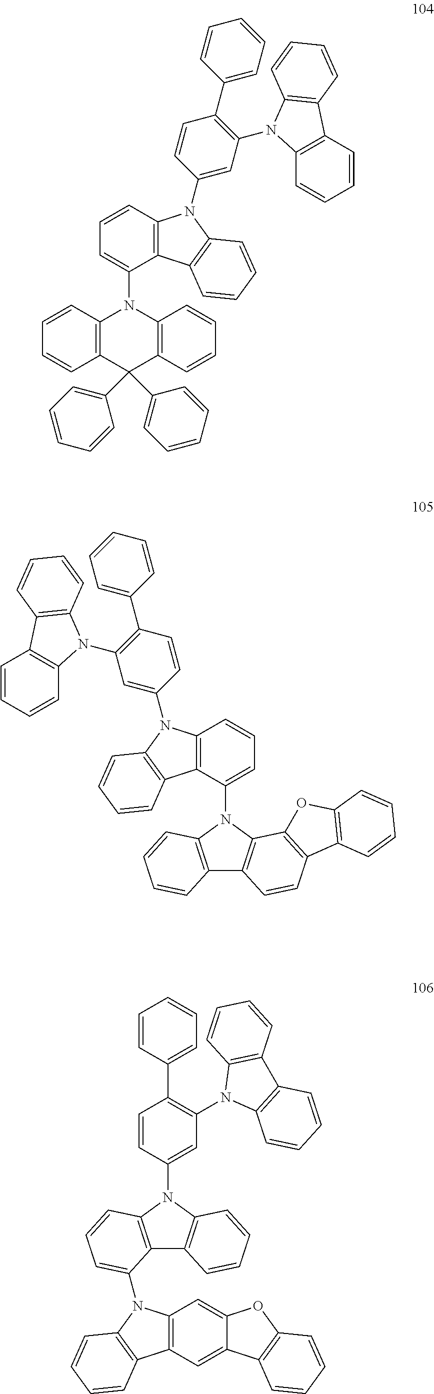 [SMILES: c1ccc(-c2ccc(-n3c4ccccc4c4c(N5c6ccccc6C(c6ccccc6)(c6ccccc6)c6ccccc65)cccc43)cc2-n2c3ccccc3c3ccccc32)cc1 ; c1ccc(-c2ccc(-n3c4ccccc4c4c(-n5c6ccccc6c6ccc7c8ccccc8oc7c65)cccc43)cc2-n2c3ccccc3c3ccccc32)cc1 ; c1ccc(-c2ccc(-n3c4ccccc4c4c(-n5c6ccccc6c6cc7c(cc65)oc5ccccc57)cccc43)cc2-n2c3ccccc3c3ccccc32)cc1]

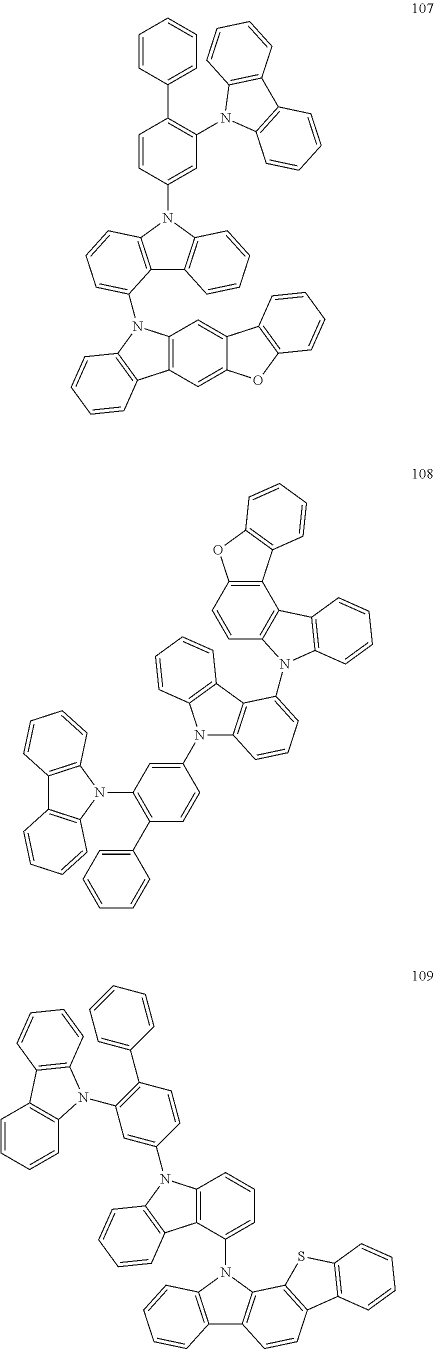 [SMILES: c1ccc(-c2ccc(-n3c4ccccc4c4c(-n5c6ccccc6c6cc7oc8ccccc8c7cc65)cccc43)cc2-n2c3ccccc3c3ccccc32)cc1 ; c1ccc(-c2ccc(-n3c4ccccc4c4c(-n5c6ccccc6c6c7c(ccc65)oc5ccccc57)cccc43)cc2-n2c3ccccc3c3ccccc32)cc1 ; c1ccc(-c2ccc(-n3c4ccccc4c4c(-n5c6ccccc6c6ccc7c8ccccc8sc7c65)cccc43)cc2-n2c3ccccc3c3ccccc32)cc1]

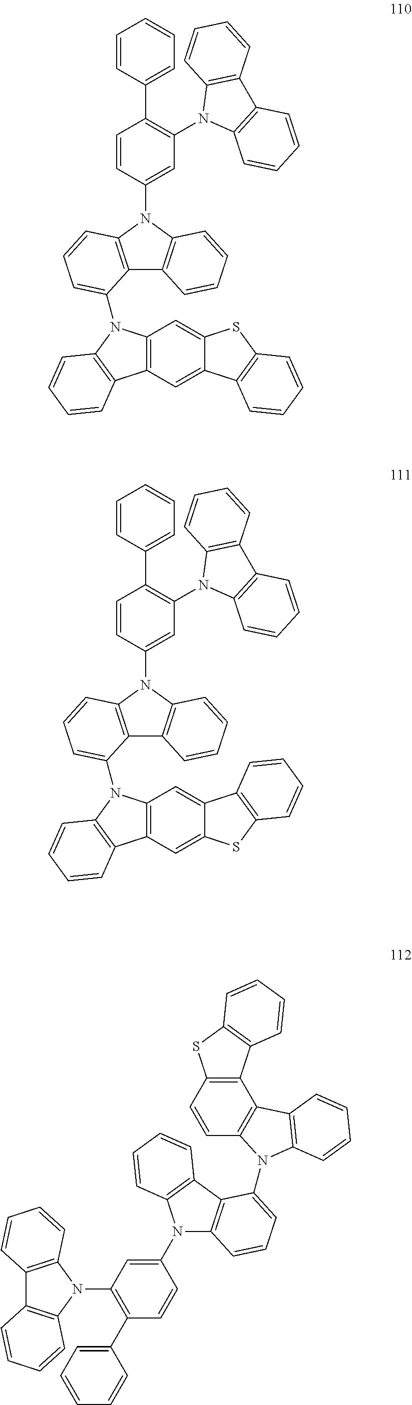 [SMILES: c1ccc(-c2ccc(-n3c4ccccc4c4c(-n5c6ccccc6c6cc7c(cc65)sc5ccccc57)cccc43)cc2-n2c3ccccc3c3ccccc32)cc1 ; c1ccc(-c2ccc(-n3c4ccccc4c4c(-n5c6ccccc6c6cc7sc8ccccc8c7cc65)cccc43)cc2-n2c3ccccc3c3ccccc32)cc1 ; c1ccc(-c2ccc(-n3c4ccccc4c4c(-n5c6ccccc6c6c7c(ccc65)sc5ccccc57)cccc43)cc2-n2c3ccccc3c3ccccc32)cc1]

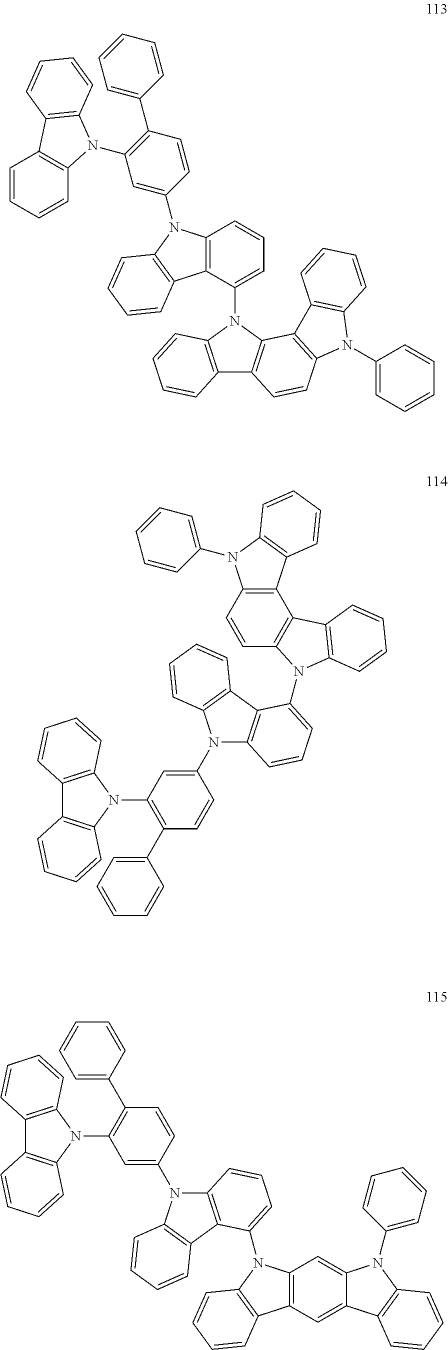 [SMILES: c1ccc(-c2ccc(-n3c4ccccc4c4c(-n5c6ccccc6c6ccc7c(c8ccccc8n7-c7ccccc7)c65)cccc43)cc2-n2c3ccccc3c3ccccc32)cc1 ; c1ccc(-c2ccc(-n3c4ccccc4c4c(-n5c6ccccc6c6c7c8ccccc8n(-c8ccccc8)c7ccc65)cccc43)cc2-n2c3ccccc3c3ccccc32)cc1 ; c1ccc(-c2ccc(-n3c4ccccc4c4c(-n5c6ccccc6c6cc7c8ccccc8n(-c8ccccc8)c7cc65)cccc43)cc2-n2c3ccccc3c3ccccc32)cc1]

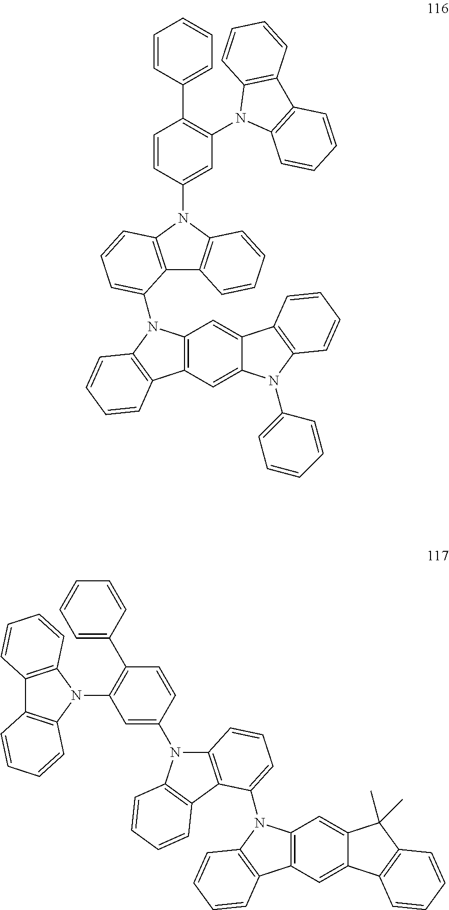 [SMILES: c1ccc(-c2ccc(-n3c4ccccc4c4c(-n5c6ccccc6c6cc7c(cc65)c5ccccc5n7-c5ccccc5)cccc43)cc2-n2c3ccccc3c3ccccc32)cc1 ; CC1(C)c2ccccc2-c2cc3c4ccccc4n(-c4cccc5c4c4ccccc4n5-c4ccc(-c5ccccc5)c(-n5c6ccccc6c6ccccc65)c4)c3cc21]

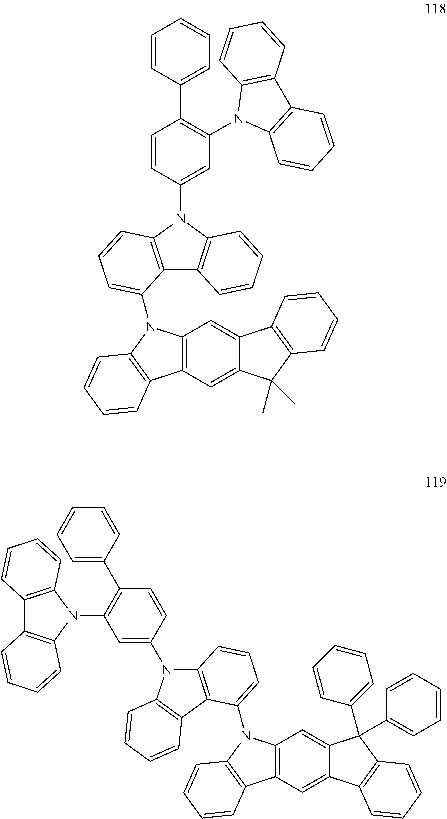 [SMILES: CC1(C)c2ccccc2-c2cc3c(cc21)c1ccccc1n3-c1cccc2c1c1ccccc1n2-c1ccc(-c2ccccc2)c(-n2c3ccccc3c3ccccc32)c1 ; c1ccc(-c2ccc(-n3c4ccccc4c4c(-n5c6ccccc6c6cc7c(cc65)C(c5ccccc5)(c5ccccc5)c5ccccc5-7)cccc43)cc2-n2c3ccccc3c3ccccc32)cc1]

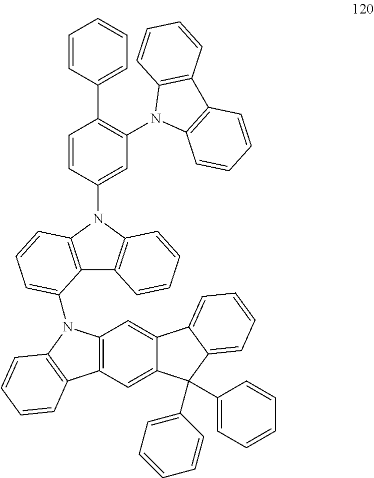 [SMILES: c1ccc(-c2ccc(-n3c4ccccc4c4c(-n5c6ccccc6c6cc7c(cc65)-c5ccccc5C7(c5ccccc5)c5ccccc5)cccc43)cc2-n2c3ccccc3c3ccccc32)cc1]

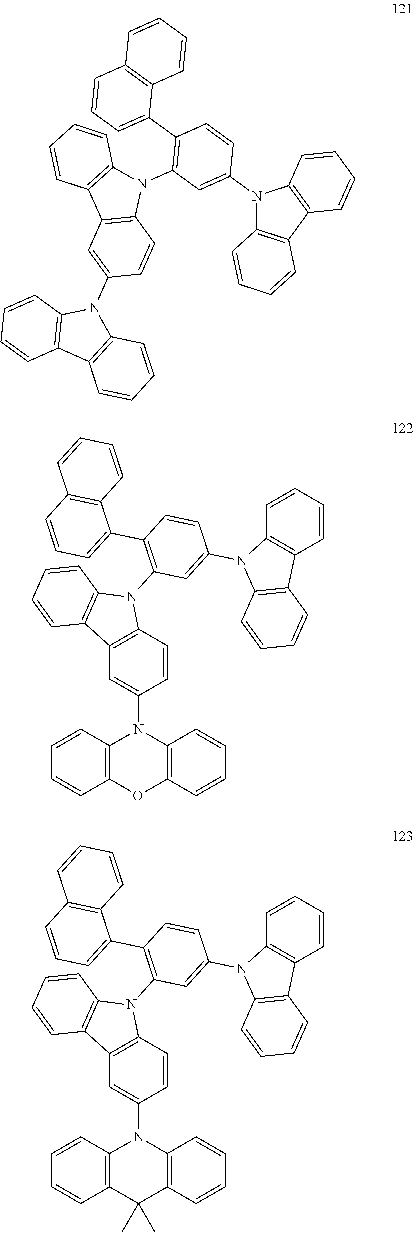 [SMILES: c1ccc2c(-c3ccc(-n4c5ccccc5c5ccccc54)cc3-n3c4ccccc4c4cc(-n5c6ccccc6c6ccccc65)ccc43)cccc2c1 ; c1ccc2c(c1)Oc1ccccc1N2c1ccc2c(c1)c1ccccc1n2-c1cc(-n2c3ccccc3c3ccccc32)ccc1-c1cccc2ccccc12 ; CC1(C)c2ccccc2N(c2ccc3c(c2)c2ccccc2n3-c2cc(-n3c4ccccc4c4ccccc43)ccc2-c2cccc3ccccc23)c2ccccc21]

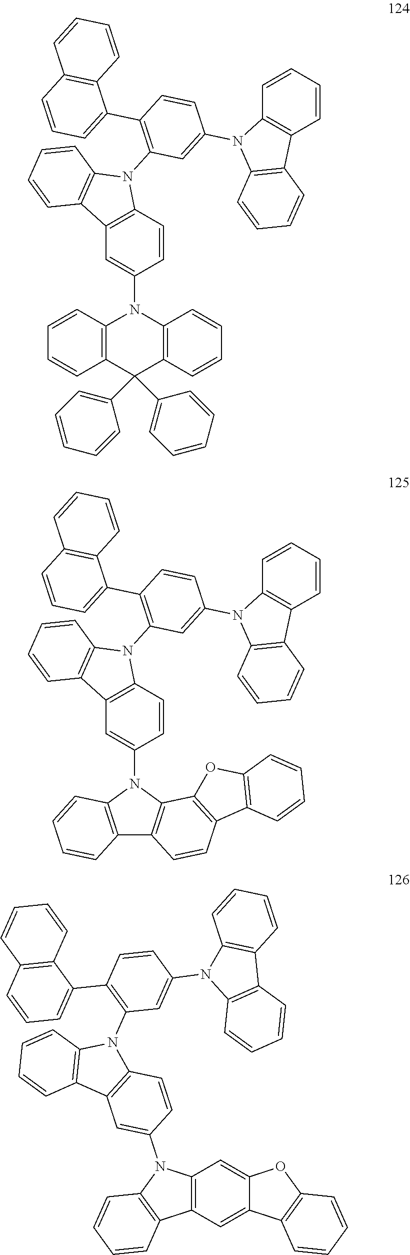

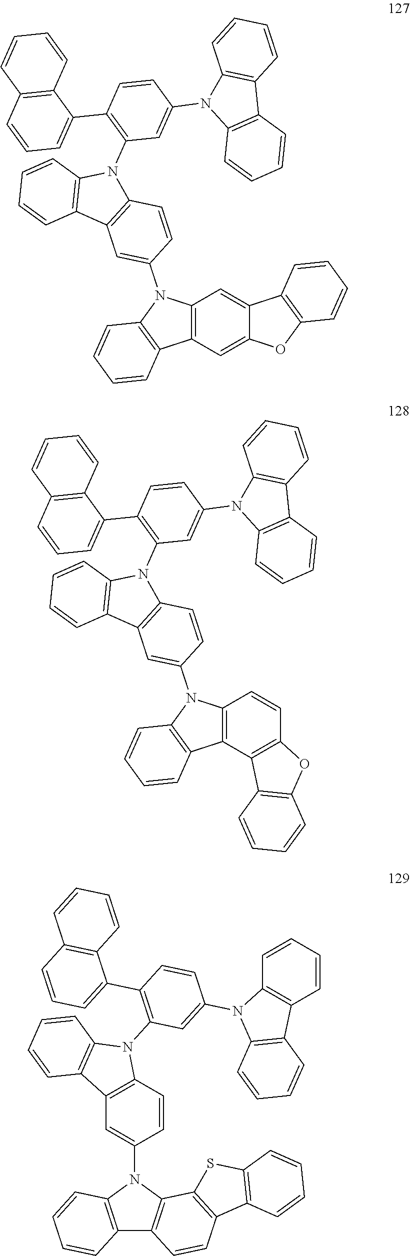 [SMILES: c1ccc2c(-c3ccc(-n4c5ccccc5c5ccccc54)cc3-n3c4ccccc4c4cc(-n5c6ccccc6c6cc7oc8ccccc8c7cc65)ccc43)cccc2c1 ; c1ccc2c(-c3ccc(-n4c5ccccc5c5ccccc54)cc3-n3c4ccccc4c4cc(-n5c6ccccc6c6c7c(ccc65)oc5ccccc57)ccc43)cccc2c1 ; c1ccc2c(-c3ccc(-n4c5ccccc5c5ccccc54)cc3-n3c4ccccc4c4cc(-n5c6ccccc6c6ccc7c8ccccc8sc7c65)ccc43)cccc2c1]

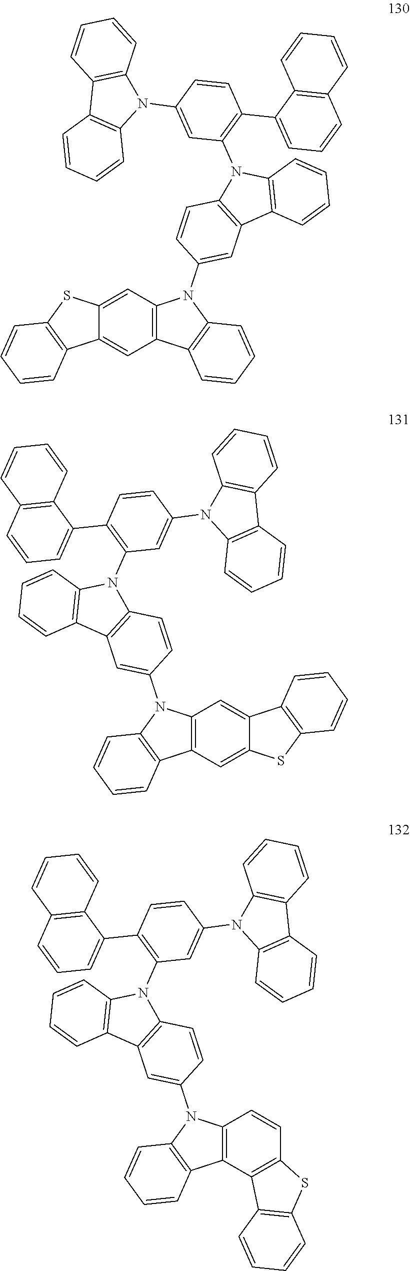

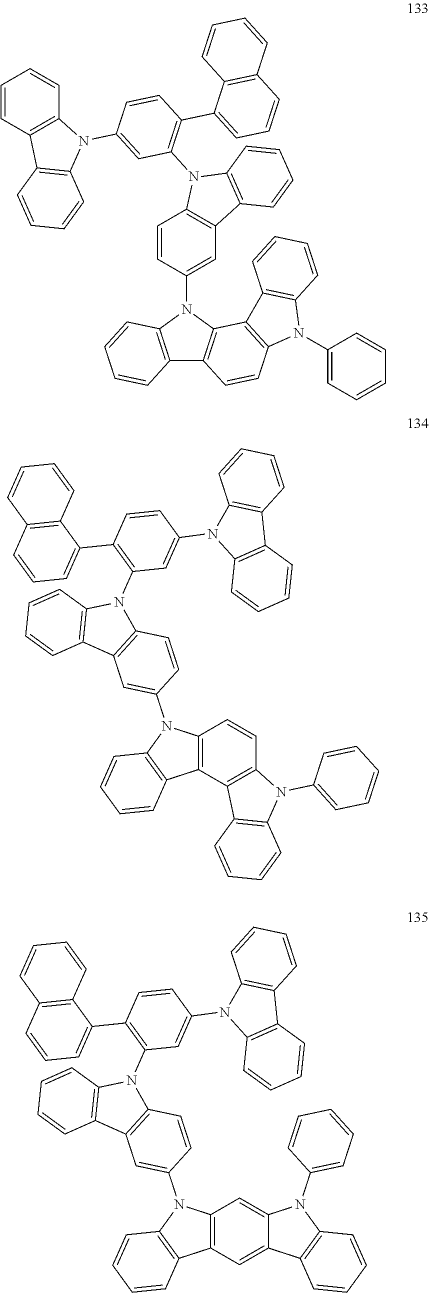 [SMILES: c1ccc(-n2c3ccccc3c3c2ccc2c4ccccc4n(-c4ccc5c(c4)c4ccccc4n5-c4cc(-n5c6ccccc6c6ccccc65)ccc4-c4cccc5ccccc45)c23)cc1 ; c1ccc(-n2c3ccccc3c3c4c5ccccc5n(-c5ccc6c(c5)c5ccccc5n6-c5cc(-n6c7ccccc7c7ccccc76)ccc5-c5cccc6ccccc56)c4ccc32)cc1 ; c1ccc(-n2c3ccccc3c3cc4c5ccccc5n(-c5ccc6c(c5)c5ccccc5n6-c5cc(-n6c7ccccc7c7ccccc76)ccc5-c5cccc6ccccc56)c4cc32)cc1]

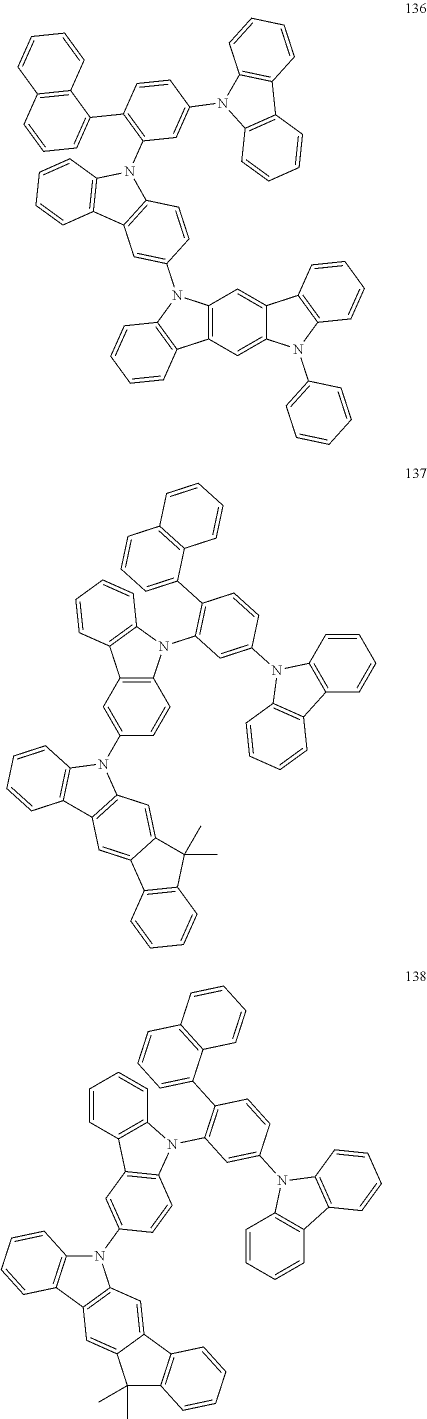 [SMILES: c1ccc(-n2c3ccccc3c3cc4c(cc32)c2ccccc2n4-c2ccc3c(c2)c2ccccc2n3-c2cc(-n3c4ccccc4c4ccccc43)ccc2-c2cccc3ccccc23)cc1 ; CC1(C)c2ccccc2-c2cc3c4ccccc4n(-c4ccc5c(c4)c4ccccc4n5-c4cc(-n5c6ccccc6c6ccccc65)ccc4-c4cccc5ccccc45)c3cc21 ; CC1(C)c2ccccc2-c2cc3c(cc21)c1ccccc1n3-c1ccc2c(c1)c1ccccc1n2-c1cc(-n2c3ccccc3c3ccccc32)ccc1-c1cccc2ccccc12]

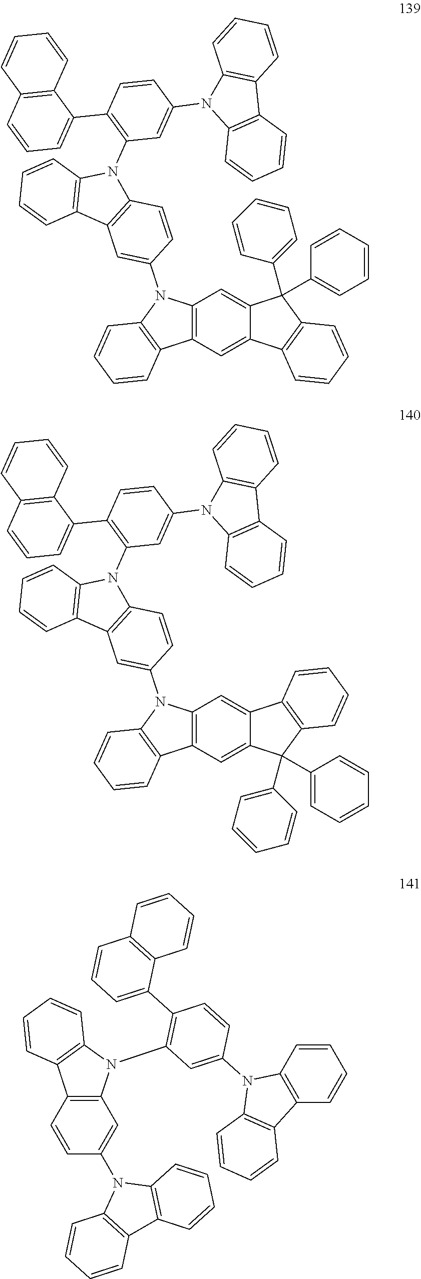 [SMILES: c1ccc(C2(c3ccccc3)c3ccccc3-c3cc4c5ccccc5n(-c5ccc6c(c5)c5ccccc5n6-c5cc(-n6c7ccccc7c7ccccc76)ccc5-c5cccc6ccccc56)c4cc32)cc1 ; c1ccc(C2(c3ccccc3)c3ccccc3-c3cc4c(cc32)c2ccccc2n4-c2ccc3c(c2)c2ccccc2n3-c2cc(-n3c4ccccc4c4ccccc43)ccc2-c2cccc3ccccc23)cc1 ; c1ccc2c(-c3ccc(-n4c5ccccc5c5ccccc54)cc3-n3c4ccccc4c4ccc(-n5c6ccccc6c6ccccc65)cc43)cccc2c1]

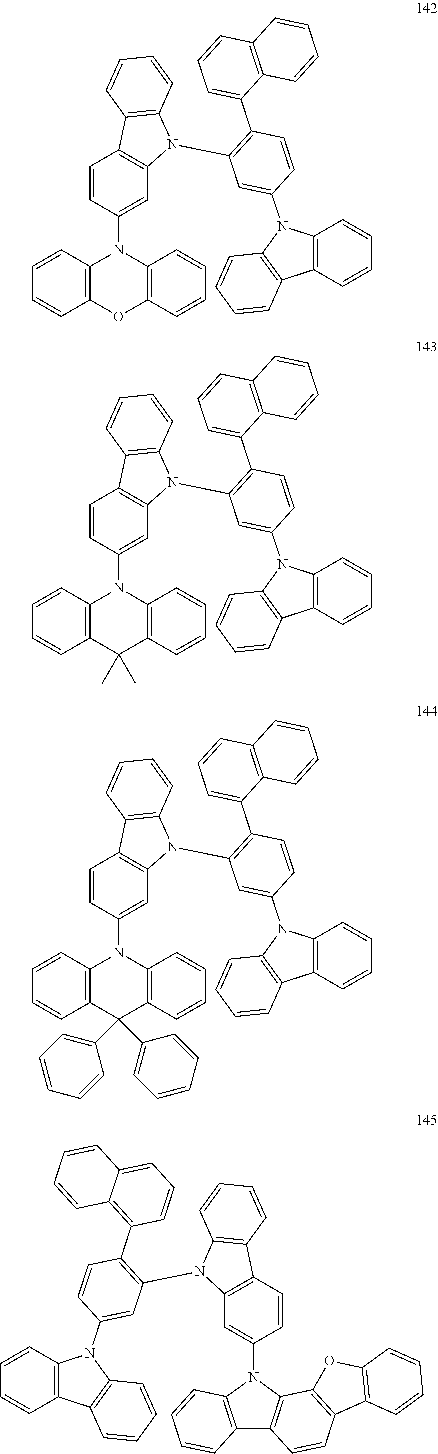

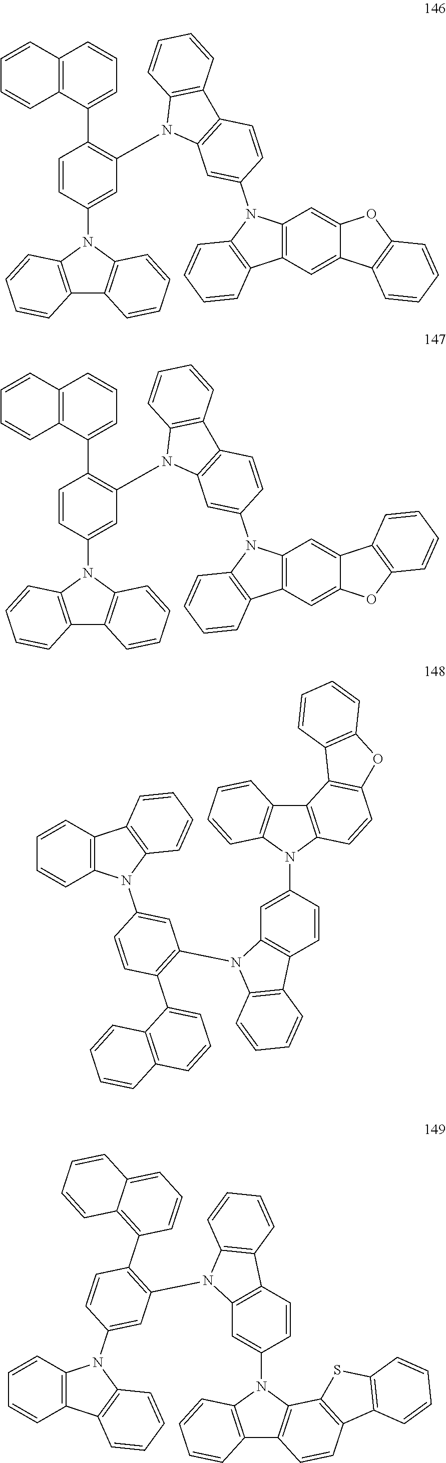 [SMILES: c1ccc2c(-c3ccc(-n4c5ccccc5c5ccccc54)cc3-n3c4ccccc4c4ccc(-n5c6ccccc6c6cc7c(cc65)oc5ccccc57)cc43)cccc2c1 ; c1ccc2c(-c3ccc(-n4c5ccccc5c5ccccc54)cc3-n3c4ccccc4c4ccc(-n5c6ccccc6c6cc7oc8ccccc8c7cc65)cc43)cccc2c1 ; c1ccc2c(-c3ccc(-n4c5ccccc5c5ccccc54)cc3-n3c4ccccc4c4ccc(-n5c6ccccc6c6c7c(ccc65)oc5ccccc57)cc43)cccc2c1 ; c1ccc2c(-c3ccc(-n4c5ccccc5c5ccccc54)cc3-n3c4ccccc4c4ccc(-n5c6ccccc6c6ccc7c8ccccc8sc7c65)cc43)cccc2c1]

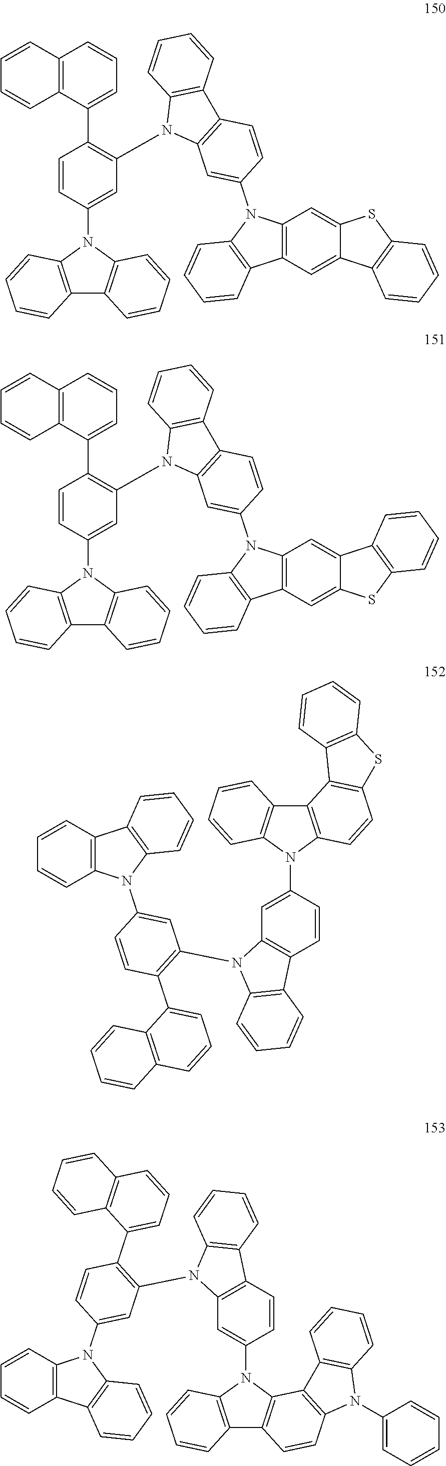 [SMILES: c1ccc2c(-c3ccc(-n4c5ccccc5c5ccccc54)cc3-n3c4ccccc4c4ccc(-n5c6ccccc6c6cc7c(cc65)sc5ccccc57)cc43)cccc2c1 ; c1ccc2c(-c3ccc(-n4c5ccccc5c5ccccc54)cc3-n3c4ccccc4c4ccc(-n5c6ccccc6c6cc7sc8ccccc8c7cc65)cc43)cccc2c1 ; c1ccc2c(-c3ccc(-n4c5ccccc5c5ccccc54)cc3-n3c4ccccc4c4ccc(-n5c6ccccc6c6c7c(ccc65)sc5ccccc57)cc43)cccc2c1 ; c1ccc(-n2c3ccccc3c3c2ccc2c4ccccc4n(-c4ccc5c6ccccc6n(-c6cc(-n7c8ccccc8c8ccccc87)ccc6-c6cccc7ccccc67)c5c4)c23)cc1]

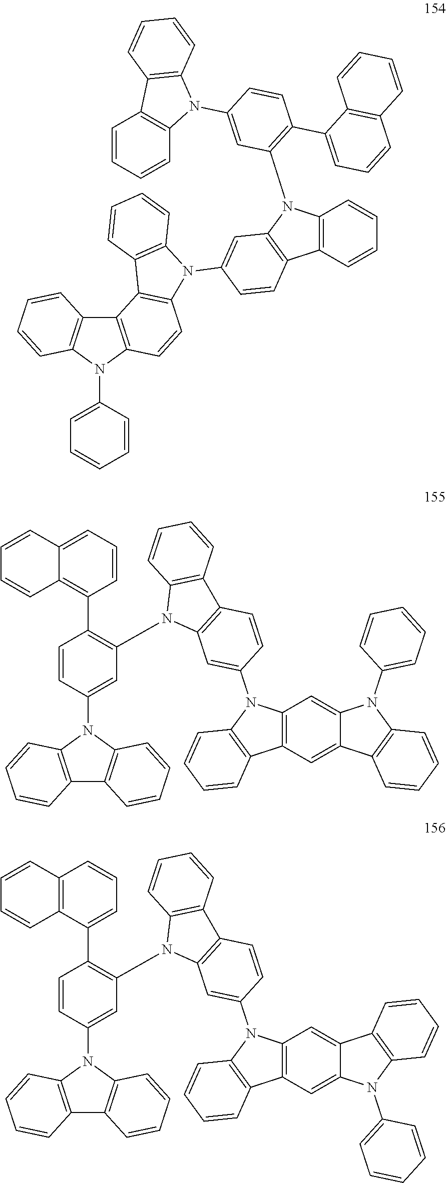 [SMILES: c1ccc(-n2c3ccccc3c3c4c5ccccc5n(-c5ccc6c7ccccc7n(-c7cc(-n8c9ccccc9c9ccccc98)ccc7-c7cccc8ccccc78)c6c5)c4ccc32)cc1 ; c1ccc(-n2c3ccccc3c3cc4c5ccccc5n(-c5ccc6c7ccccc7n(-c7cc(-n8c9ccccc9c9ccccc98)ccc7-c7cccc8ccccc78)c6c5)c4cc32)cc1 ; c1ccc(-n2c3ccccc3c3cc4c(cc32)c2ccccc2n4-c2ccc3c4ccccc4n(-c4cc(-n5c6ccccc6c6ccccc65)ccc4-c4cccc5ccccc45)c3c2)cc1]

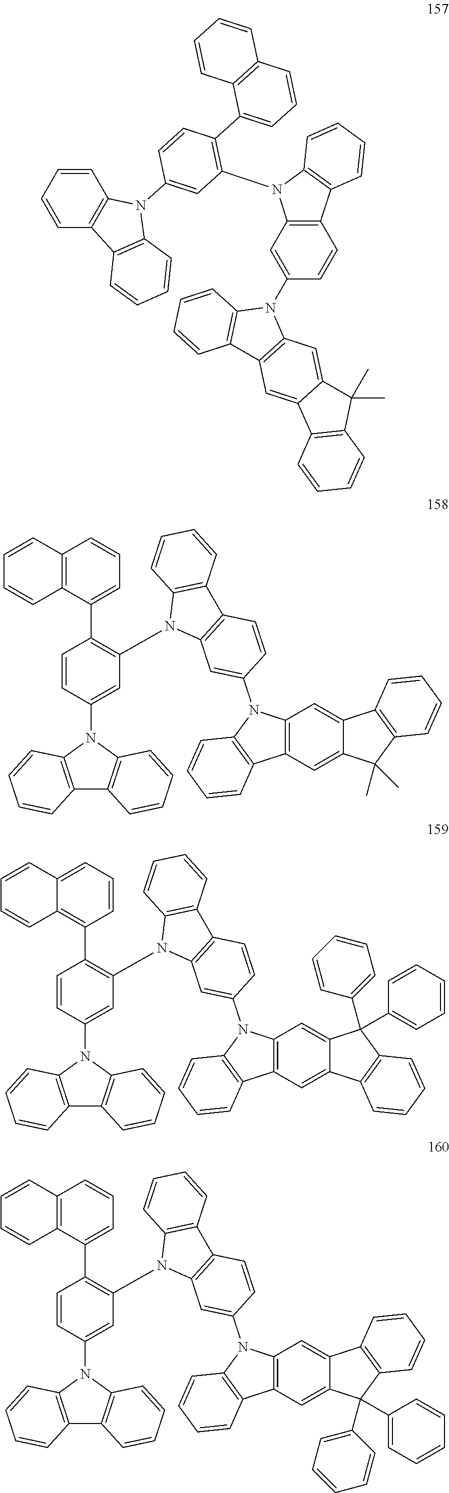 [SMILES: CC1(C)c2ccccc2-c2cc3c4ccccc4n(-c4ccc5c6ccccc6n(-c6cc(-n7c8ccccc8c8ccccc87)ccc6-c6cccc7ccccc67)c5c4)c3cc21 ; CC1(C)c2ccccc2-c2cc3c(cc21)c1ccccc1n3-c1ccc2c3ccccc3n(-c3cc(-n4c5ccccc5c5ccccc54)ccc3-c3cccc4ccccc34)c2c1 ; c1ccc(C2(c3ccccc3)c3ccccc3-c3cc4c5ccccc5n(-c5ccc6c7ccccc7n(-c7cc(-n8c9ccccc9c9ccccc98)ccc7-c7cccc8ccccc78)c6c5)c4cc32)cc1 ; c1ccc(C2(c3ccccc3)c3ccccc3-c3cc4c(cc32)c2ccccc2n4-c2ccc3c4ccccc4n(-c4cc(-n5c6ccccc6c6ccccc65)ccc4-c4cccc5ccccc45)c3c2)cc1]

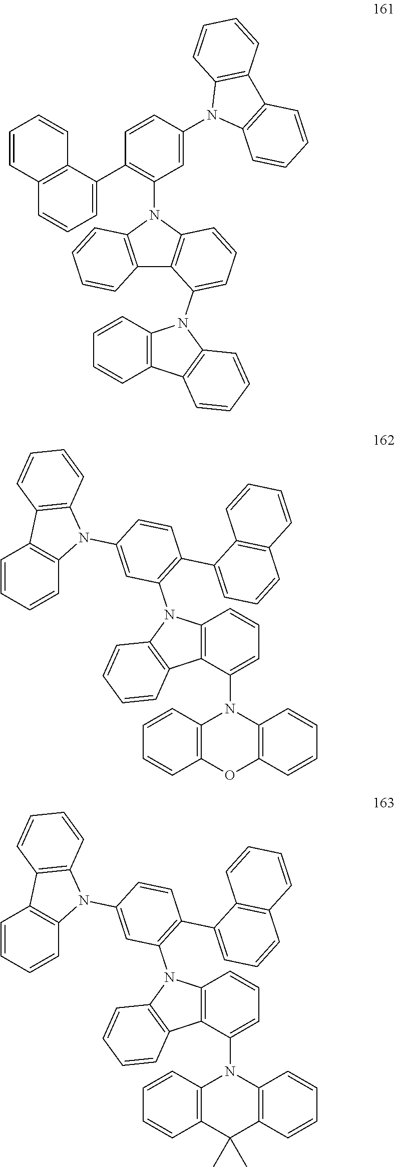 [SMILES: c1ccc2c(-c3ccc(-n4c5ccccc5c5ccccc54)cc3-n3c4ccccc4c4c(-n5c6ccccc6c6ccccc65)cccc43)cccc2c1 ; c1ccc2c(c1)Oc1ccccc1N2c1cccc2c1c1ccccc1n2-c1cc(-n2c3ccccc3c3ccccc32)ccc1-c1cccc2ccccc12 ; CC1(C)c2ccccc2N(c2cccc3c2c2ccccc2n3-c2cc(-n3c4ccccc4c4ccccc43)ccc2-c2cccc3ccccc23)c2ccccc21]

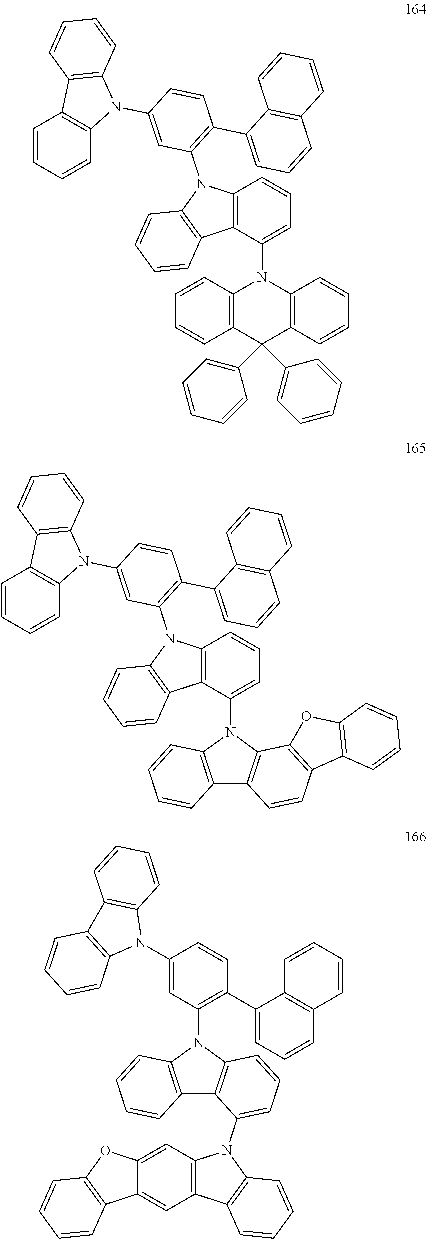 [SMILES: c1ccc(C2(c3ccccc3)c3ccccc3N(c3cccc4c3c3ccccc3n4-c3cc(-n4c5ccccc5c5ccccc54)ccc3-c3cccc4ccccc34)c3ccccc32)cc1 ; c1ccc2c(-c3ccc(-n4c5ccccc5c5ccccc54)cc3-n3c4ccccc4c4c(-n5c6ccccc6c6ccc7c8ccccc8oc7c65)cccc43)cccc2c1 ; c1ccc2c(-c3ccc(-n4c5ccccc5c5ccccc54)cc3-n3c4ccccc4c4c(-n5c6ccccc6c6cc7c(cc65)oc5ccccc57)cccc43)cccc2c1]

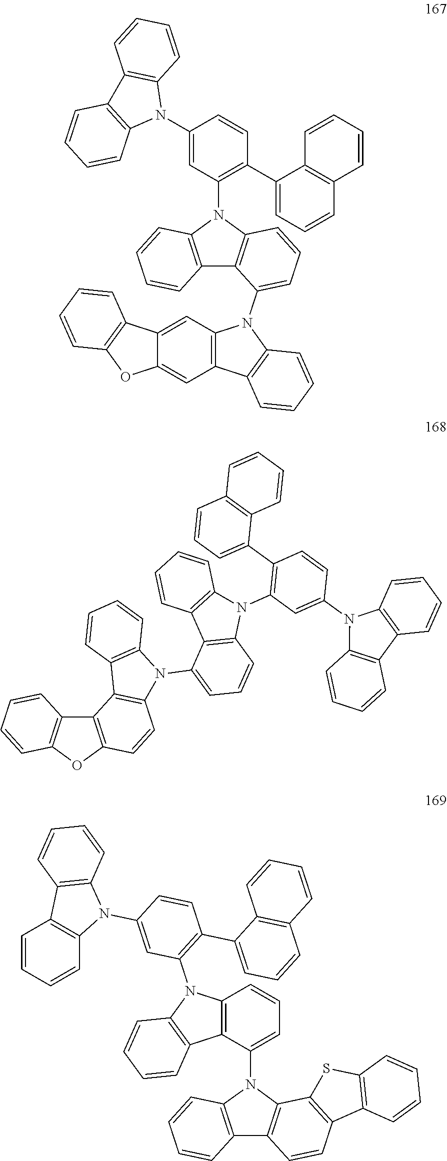

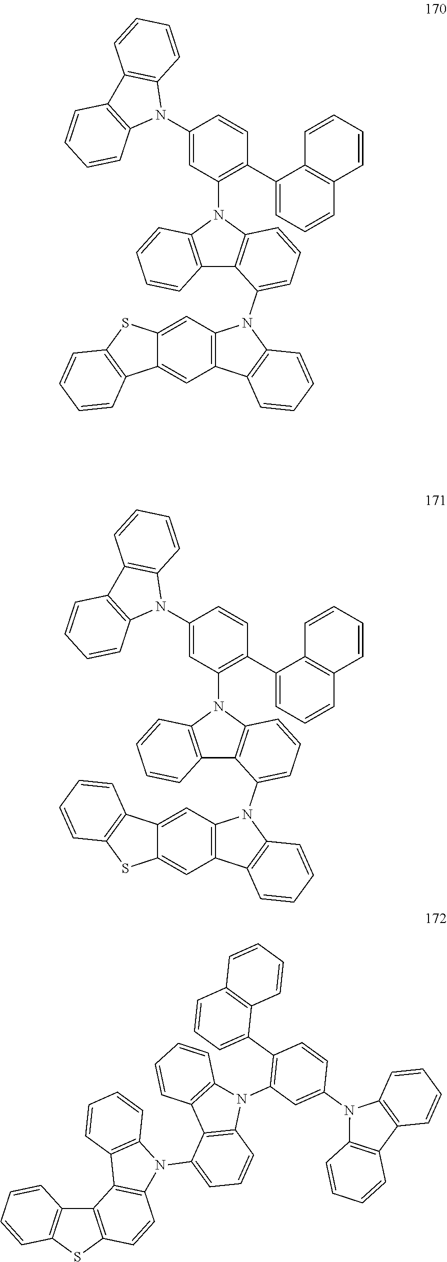

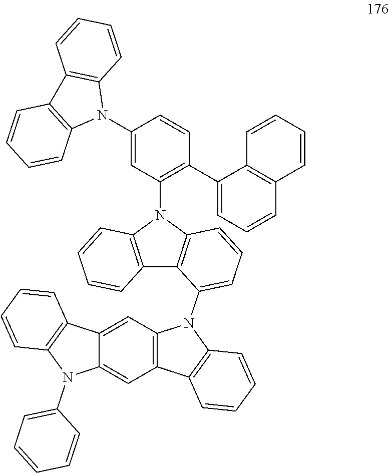 [SMILES: c1ccc(-n2c3ccccc3c3cc4c(cc32)c2ccccc2n4-c2cccc3c2c2ccccc2n3-c2cc(-n3c4ccccc4c4ccccc43)ccc2-c2cccc3ccccc23)cc1]

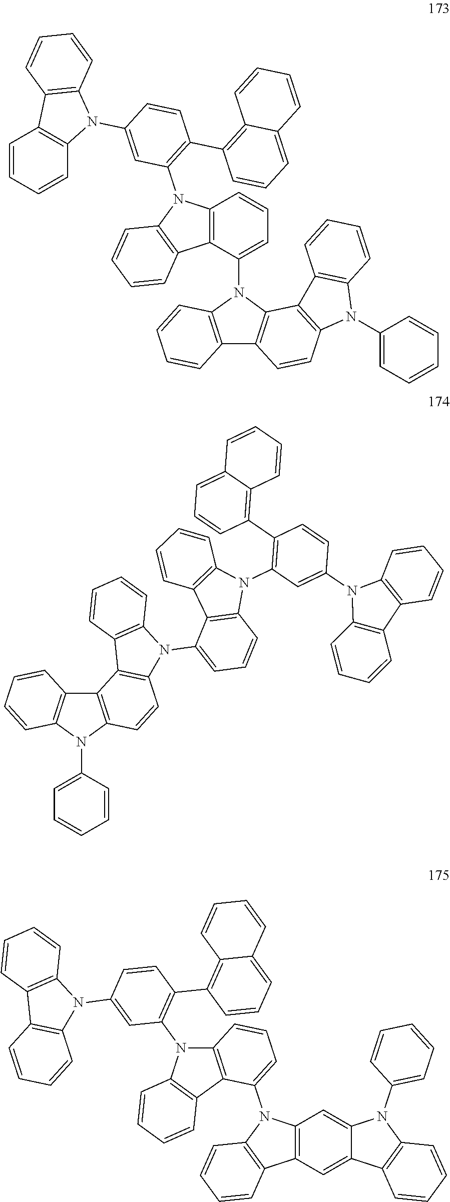 [SMILES: c1ccc(-n2c3ccccc3c3c2ccc2c4ccccc4n(-c4cccc5c4c4ccccc4n5-c4cc(-n5c6ccccc6c6ccccc65)ccc4-c4cccc5ccccc45)c23)cc1 ; c1ccc(-n2c3ccccc3c3c4c5ccccc5n(-c5cccc6c5c5ccccc5n6-c5cc(-n6c7ccccc7c7ccccc76)ccc5-c5cccc6ccccc56)c4ccc32)cc1 ; c1ccc(-n2c3ccccc3c3cc4c5ccccc5n(-c5cccc6c5c5ccccc5n6-c5cc(-n6c7ccccc7c7ccccc76)ccc5-c5cccc6ccccc56)c4cc32)cc1]

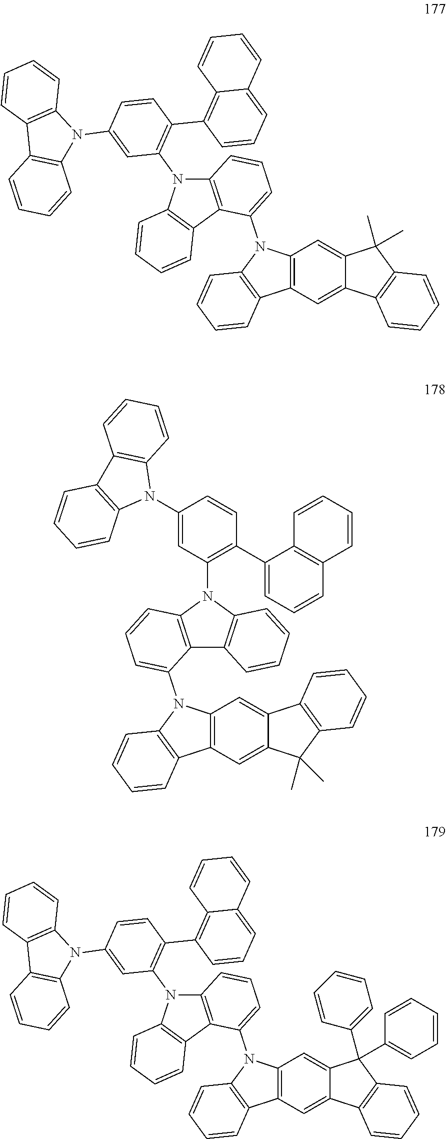 [SMILES: CC1(C)c2ccccc2-c2cc3c4ccccc4n(-c4cccc5c4c4ccccc4n5-c4cc(-n5c6ccccc6c6ccccc65)ccc4-c4cccc5ccccc45)c3cc21 ; CC1(C)c2ccccc2-c2cc3c(cc21)c1ccccc1n3-c1cccc2c1c1ccccc1n2-c1cc(-n2c3ccccc3c3ccccc32)ccc1-c1cccc2ccccc12 ; c1ccc(C2(c3ccccc3)c3ccccc3-c3cc4c5ccccc5n(-c5cccc6c5c5ccccc5n6-c5cc(-n6c7ccccc7c7ccccc76)ccc5-c5cccc6ccccc56)c4cc32)cc1]

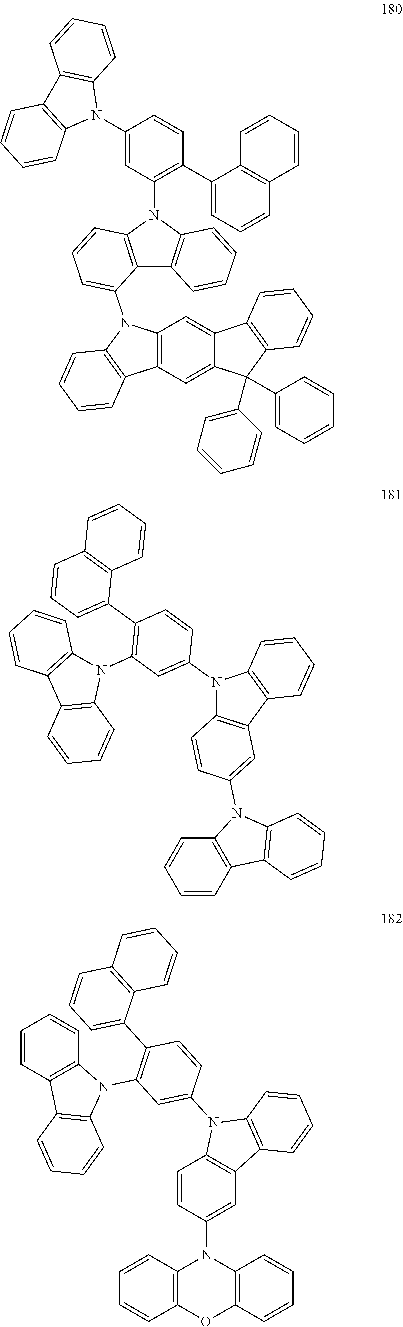

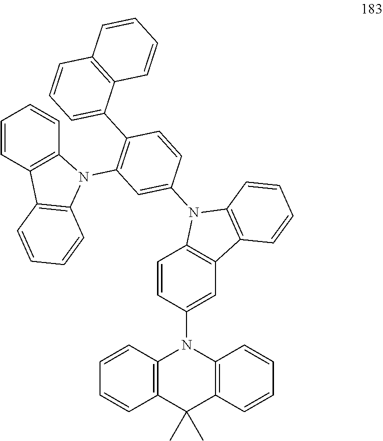 [SMILES: CC1(C)c2ccccc2N(c2ccc3c(c2)c2ccccc2n3-c2ccc(-c3cccc4ccccc34)c(-n3c4ccccc4c4ccccc43)c2)c2ccccc21]

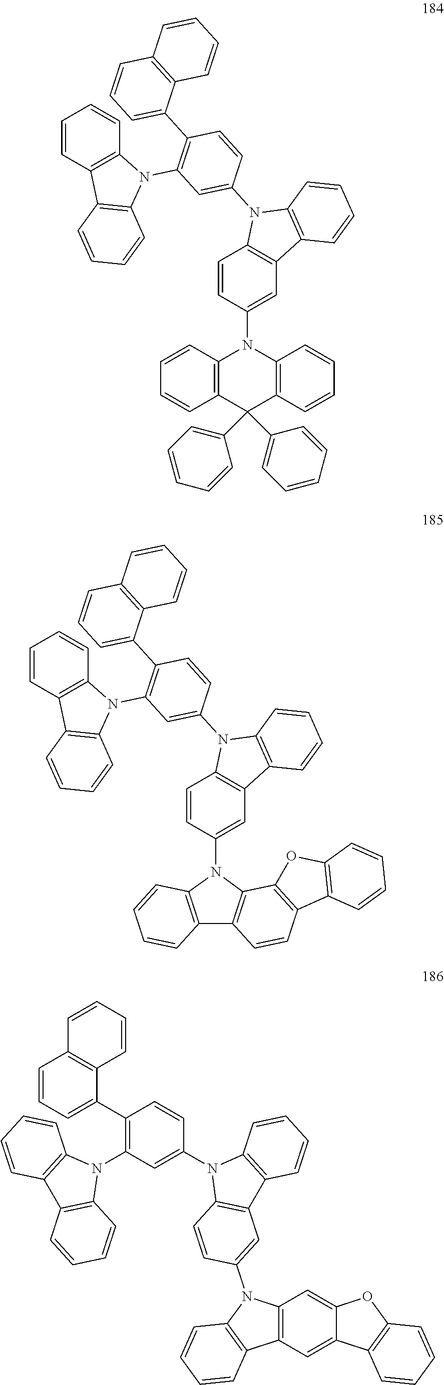 [SMILES: c1ccc(C2(c3ccccc3)c3ccccc3N(c3ccc4c(c3)c3ccccc3n4-c3ccc(-c4cccc5ccccc45)c(-n4c5ccccc5c5ccccc54)c3)c3ccccc32)cc1 ; c1ccc2c(-c3ccc(-n4c5ccccc5c5cc(-n6c7ccccc7c7ccc8c9ccccc9oc8c76)ccc54)cc3-n3c4ccccc4c4ccccc43)cccc2c1 ; c1ccc2c(-c3ccc(-n4c5ccccc5c5cc(-n6c7ccccc7c7cc8c(cc76)oc6ccccc68)ccc54)cc3-n3c4ccccc4c4ccccc43)cccc2c1]

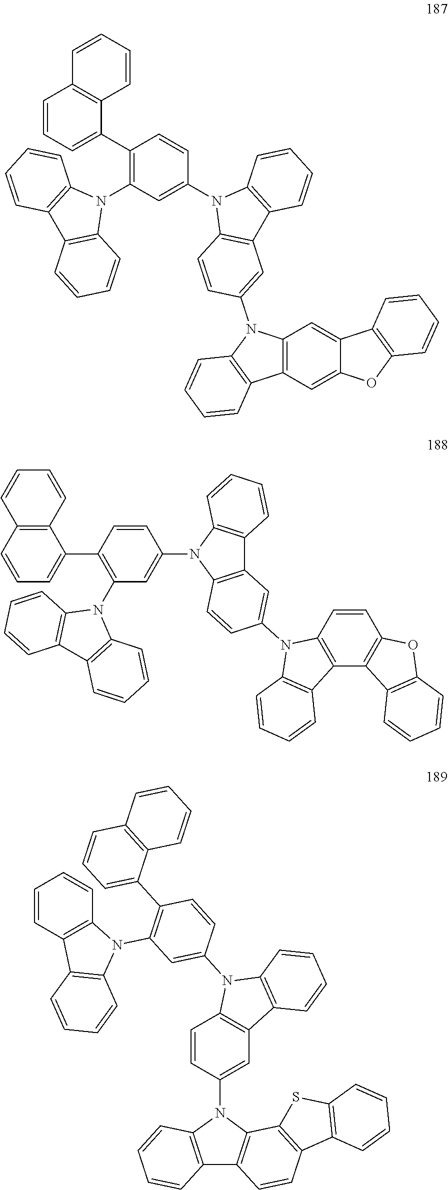 [SMILES: c1ccc2c(-c3ccc(-n4c5ccccc5c5cc(-n6c7ccccc7c7cc8oc9ccccc9c8cc76)ccc54)cc3-n3c4ccccc4c4ccccc43)cccc2c1 ; c1ccc2c(-c3ccc(-n4c5ccccc5c5cc(-n6c7ccccc7c7c8c(ccc76)oc6ccccc68)ccc54)cc3-n3c4ccccc4c4ccccc43)cccc2c1 ; c1ccc2c(-c3ccc(-n4c5ccccc5c5cc(-n6c7ccccc7c7ccc8c9ccccc9sc8c76)ccc54)cc3-n3c4ccccc4c4ccccc43)cccc2c1]

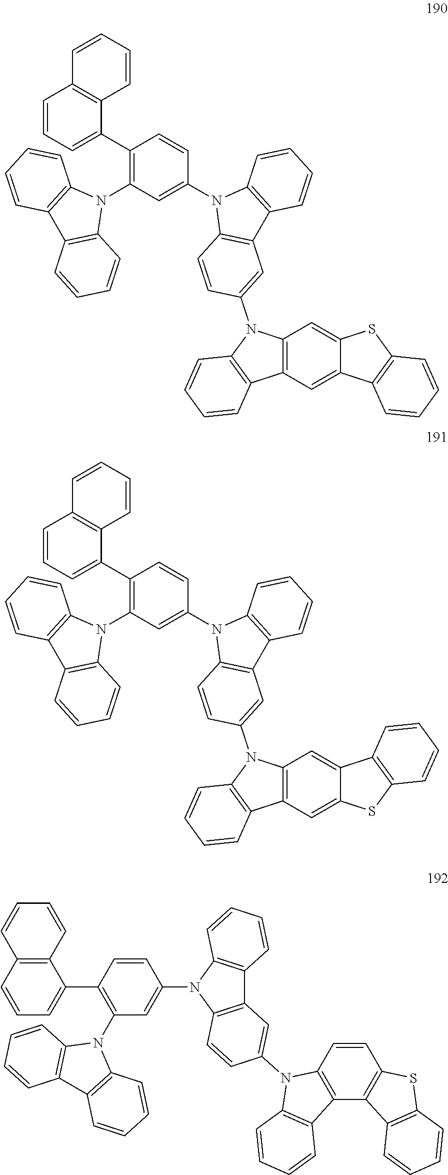 [SMILES: c1ccc2c(-c3ccc(-n4c5ccccc5c5cc(-n6c7ccccc7c7cc8c(cc76)sc6ccccc68)ccc54)cc3-n3c4ccccc4c4ccccc43)cccc2c1 ; c1ccc2c(-c3ccc(-n4c5ccccc5c5cc(-n6c7ccccc7c7cc8sc9ccccc9c8cc76)ccc54)cc3-n3c4ccccc4c4ccccc43)cccc2c1 ; c1ccc2c(-c3ccc(-n4c5ccccc5c5cc(-n6c7ccccc7c7c8c(ccc76)sc6ccccc68)ccc54)cc3-n3c4ccccc4c4ccccc43)cccc2c1]

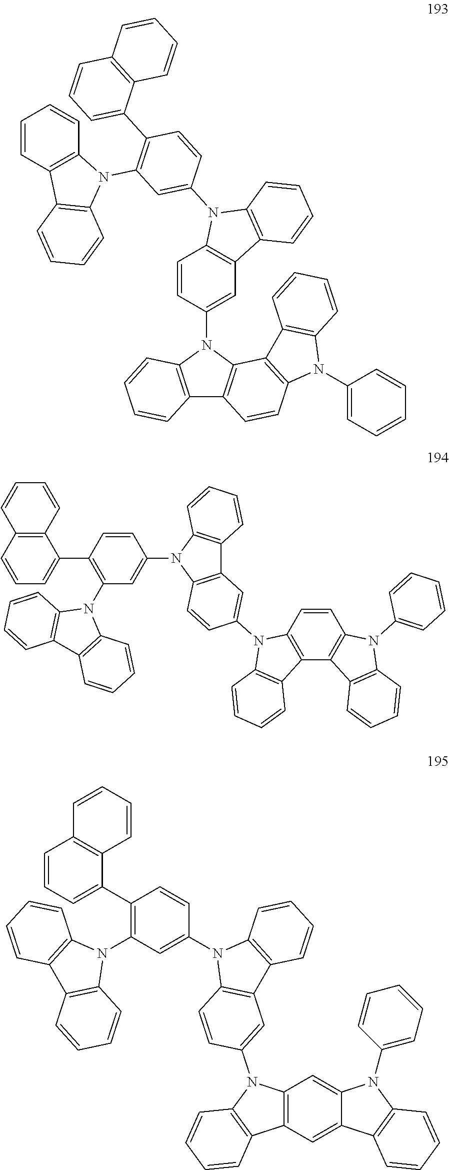 [SMILES: c1ccc(-n2c3ccccc3c3c2ccc2c4ccccc4n(-c4ccc5c(c4)c4ccccc4n5-c4ccc(-c5cccc6ccccc56)c(-n5c6ccccc6c6ccccc65)c4)c23)cc1 ; c1ccc(-n2c3ccccc3c3c4c5ccccc5n(-c5ccc6c(c5)c5ccccc5n6-c5ccc(-c6cccc7ccccc67)c(-n6c7ccccc7c7ccccc76)c5)c4ccc32)cc1 ; c1ccc(-n2c3ccccc3c3cc4c5ccccc5n(-c5ccc6c(c5)c5ccccc5n6-c5ccc(-c6cccc7ccccc67)c(-n6c7ccccc7c7ccccc76)c5)c4cc32)cc1]

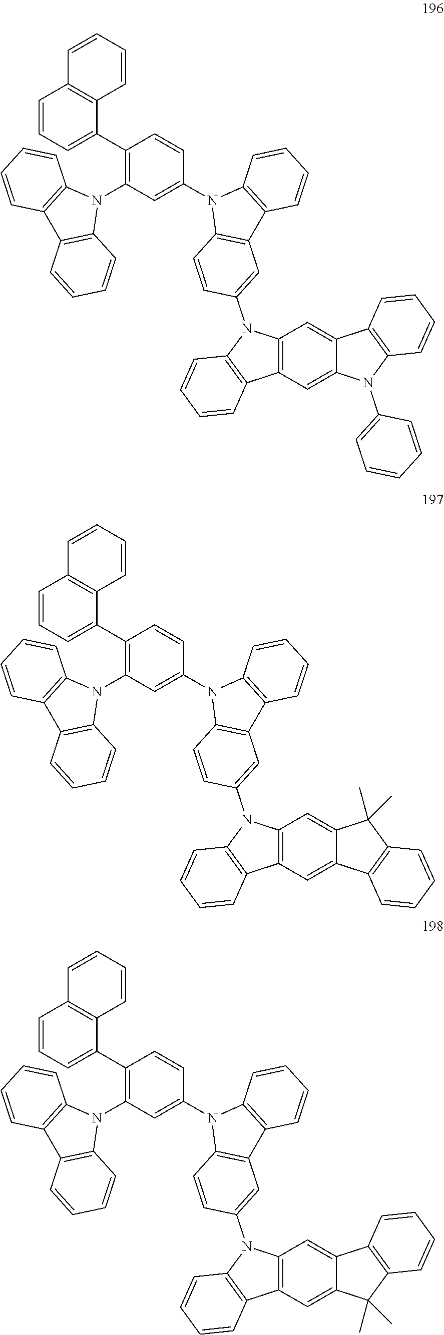 [SMILES: c1ccc(-n2c3ccccc3c3cc4c(cc32)c2ccccc2n4-c2ccc3c(c2)c2ccccc2n3-c2ccc(-c3cccc4ccccc34)c(-n3c4ccccc4c4ccccc43)c2)cc1 ; CC1(C)c2ccccc2-c2cc3c4ccccc4n(-c4ccc5c(c4)c4ccccc4n5-c4ccc(-c5cccc6ccccc56)c(-n5c6ccccc6c6ccccc65)c4)c3cc21 ; CC1(C)c2ccccc2-c2cc3c(cc21)c1ccccc1n3-c1ccc2c(c1)c1ccccc1n2-c1ccc(-c2cccc3ccccc23)c(-n2c3ccccc3c3ccccc32)c1]

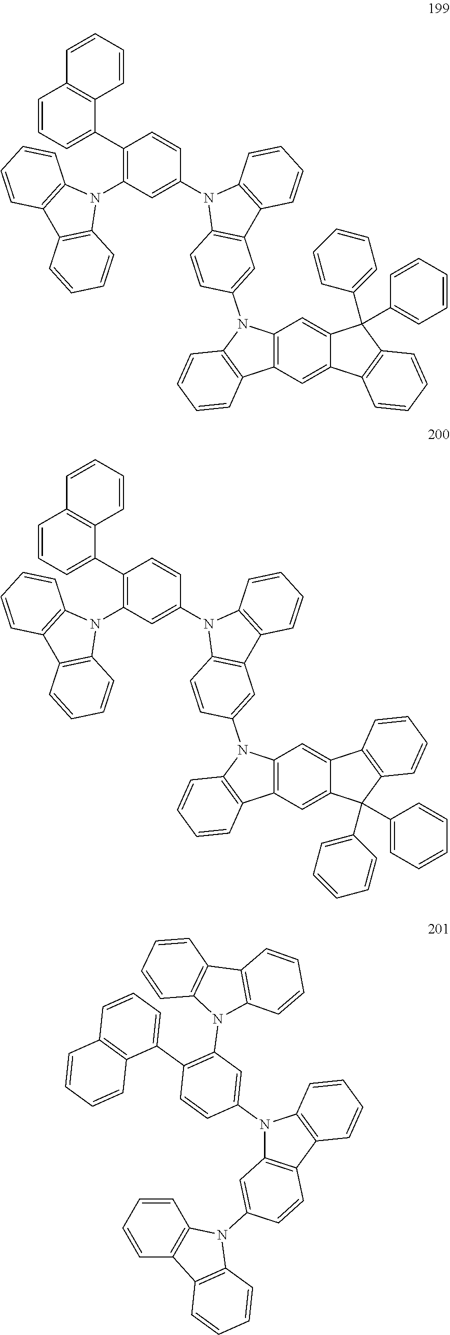 [SMILES: c1ccc(C2(c3ccccc3)c3ccccc3-c3cc4c5ccccc5n(-c5ccc6c(c5)c5ccccc5n6-c5ccc(-c6cccc7ccccc67)c(-n6c7ccccc7c7ccccc76)c5)c4cc32)cc1 ; c1ccc(C2(c3ccccc3)c3ccccc3-c3cc4c(cc32)c2ccccc2n4-c2ccc3c(c2)c2ccccc2n3-c2ccc(-c3cccc4ccccc34)c(-n3c4ccccc4c4ccccc43)c2)cc1 ; c1ccc2c(-c3ccc(-n4c5ccccc5c5ccc(-n6c7ccccc7c7ccccc76)cc54)cc3-n3c4ccccc4c4ccccc43)cccc2c1]

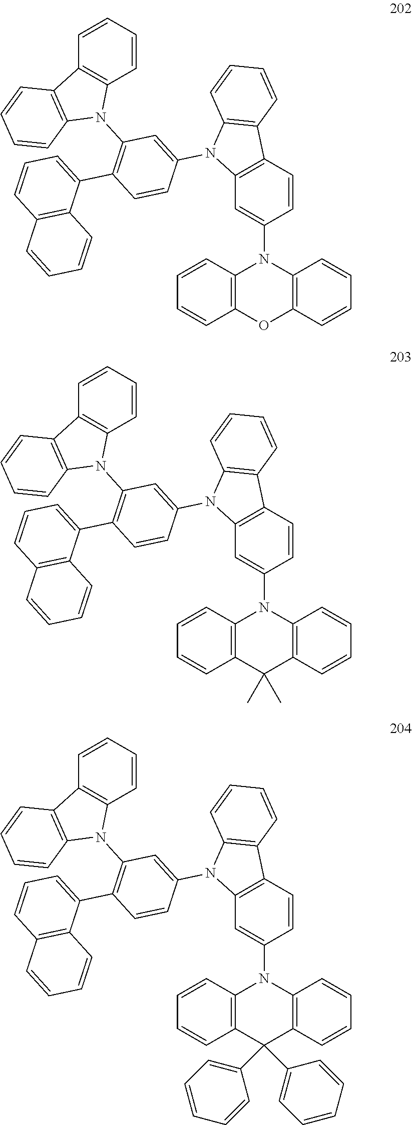 [SMILES: c1ccc2c(c1)Oc1ccccc1N2c1ccc2c3ccccc3n(-c3ccc(-c4cccc5ccccc45)c(-n4c5ccccc5c5ccccc54)c3)c2c1 ; CC1(C)c2ccccc2N(c2ccc3c4ccccc4n(-c4ccc(-c5cccc6ccccc56)c(-n5c6ccccc6c6ccccc65)c4)c3c2)c2ccccc21 ; c1ccc(C2(c3ccccc3)c3ccccc3N(c3ccc4c5ccccc5n(-c5ccc(-c6cccc7ccccc67)c(-n6c7ccccc7c7ccccc76)c5)c4c3)c3ccccc32)cc1]

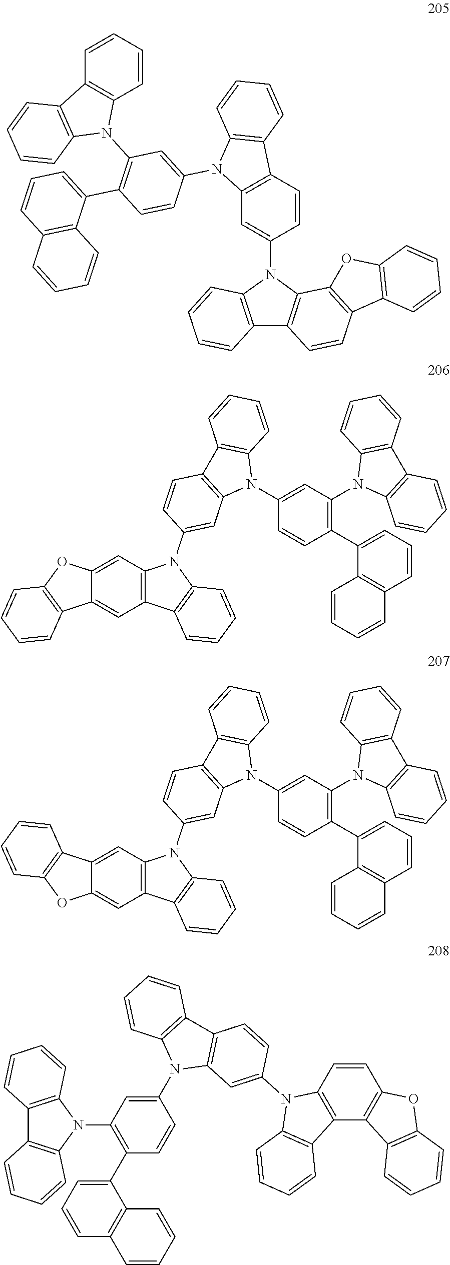 [SMILES: c1ccc2c(-c3ccc(-n4c5ccccc5c5ccc(-n6c7ccccc7c7ccc8c9ccccc9oc8c76)cc54)cc3-n3c4ccccc4c4ccccc43)cccc2c1 ; c1ccc2c(-c3ccc(-n4c5ccccc5c5ccc(-n6c7ccccc7c7cc8c(cc76)oc6ccccc68)cc54)cc3-n3c4ccccc4c4ccccc43)cccc2c1 ; c1ccc2c(-c3ccc(-n4c5ccccc5c5ccc(-n6c7ccccc7c7cc8oc9ccccc9c8cc76)cc54)cc3-n3c4ccccc4c4ccccc43)cccc2c1 ; c1ccc2c(-c3ccc(-n4c5ccccc5c5ccc(-n6c7ccccc7c7c8c(ccc76)oc6ccccc68)cc54)cc3-n3c4ccccc4c4ccccc43)cccc2c1]

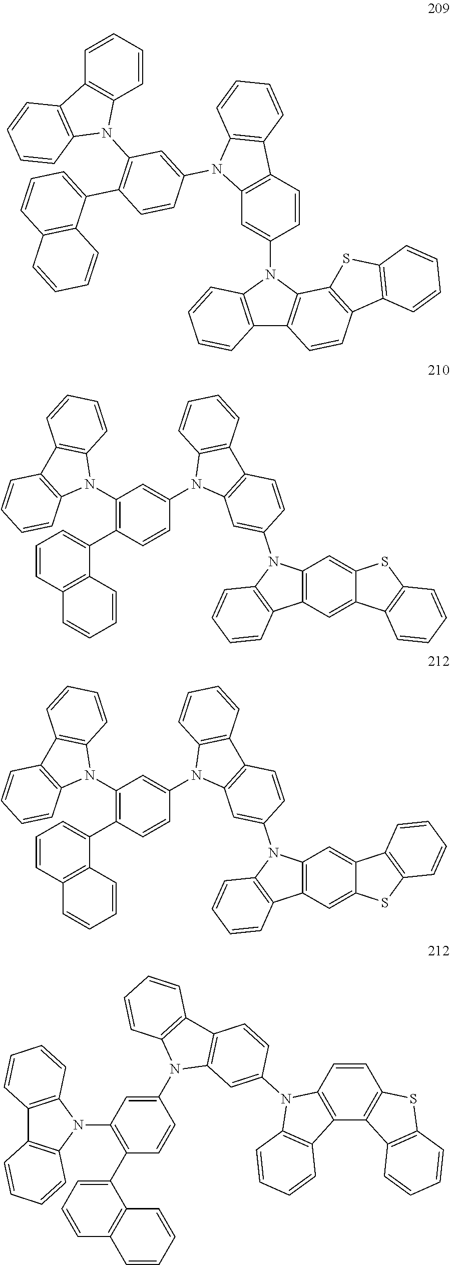 [SMILES: c1ccc2c(-c3ccc(-n4c5ccccc5c5ccc(-n6c7ccccc7c7ccc8c9ccccc9sc8c76)cc54)cc3-n3c4ccccc4c4ccccc43)cccc2c1 ; c1ccc2c(-c3ccc(-n4c5ccccc5c5ccc(-n6c7ccccc7c7cc8c(cc76)sc6ccccc68)cc54)cc3-n3c4ccccc4c4ccccc43)cccc2c1 ; c1ccc2c(-c3ccc(-n4c5ccccc5c5ccc(-n6c7ccccc7c7cc8sc9ccccc9c8cc76)cc54)cc3-n3c4ccccc4c4ccccc43)cccc2c1 ; c1ccc2c(-c3ccc(-n4c5ccccc5c5ccc(-n6c7ccccc7c7c8c(ccc76)sc6ccccc68)cc54)cc3-n3c4ccccc4c4ccccc43)cccc2c1]

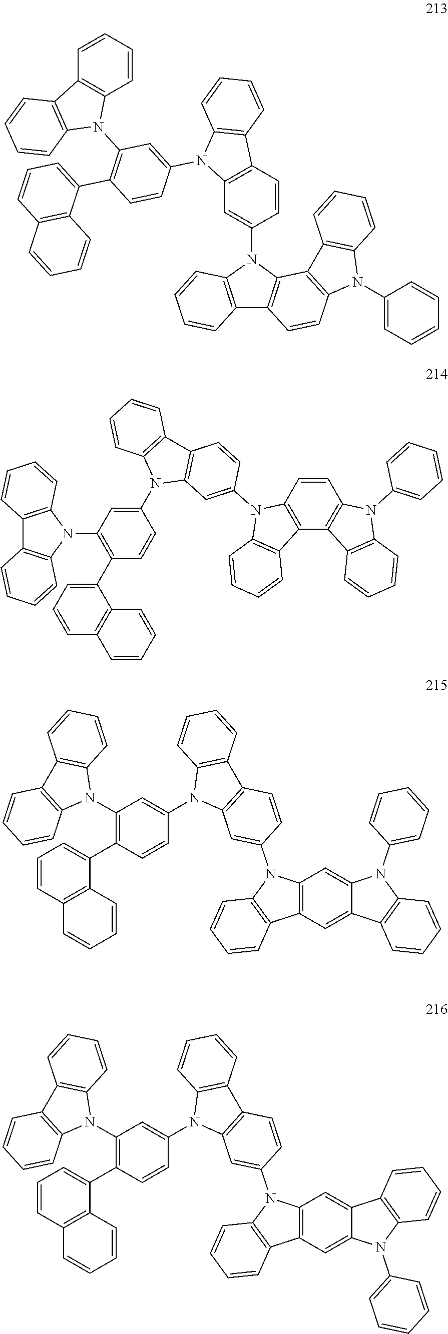 [SMILES: c1ccc(-n2c3ccccc3c3c2ccc2c4ccccc4n(-c4ccc5c6ccccc6n(-c6ccc(-c7cccc8ccccc78)c(-n7c8ccccc8c8ccccc87)c6)c5c4)c23)cc1 ; c1ccc(-n2c3ccccc3c3c4c5ccccc5n(-c5ccc6c7ccccc7n(-c7ccc(-c8cccc9ccccc89)c(-n8c9ccccc9c9ccccc98)c7)c6c5)c4ccc32)cc1 ; c1ccc(-n2c3ccccc3c3cc4c5ccccc5n(-c5ccc6c7ccccc7n(-c7ccc(-c8cccc9ccccc89)c(-n8c9ccccc9c9ccccc98)c7)c6c5)c4cc32)cc1 ; c1ccc(-n2c3ccccc3c3cc4c(cc32)c2ccccc2n4-c2ccc3c4ccccc4n(-c4ccc(-c5cccc6ccccc56)c(-n5c6ccccc6c6ccccc65)c4)c3c2)cc1]

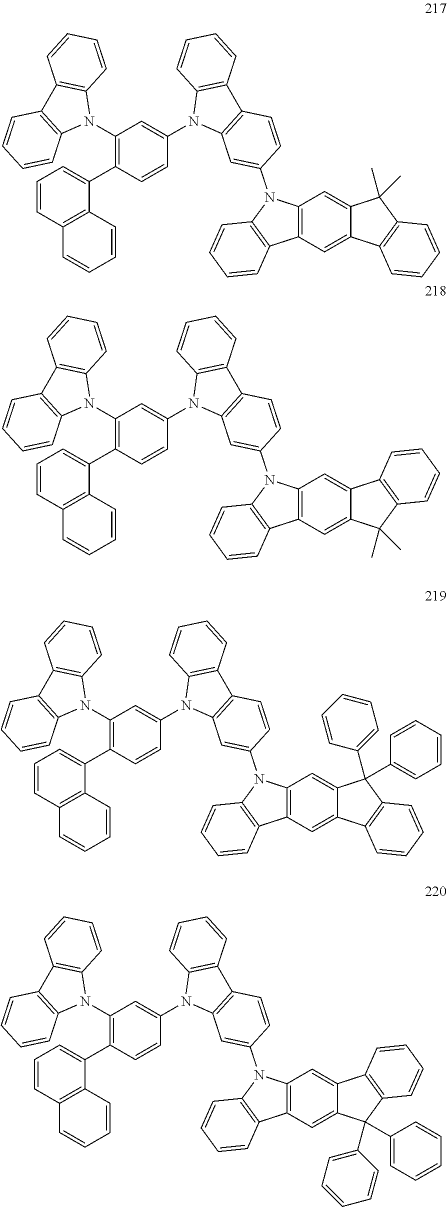 [SMILES: CC1(C)c2ccccc2-c2cc3c4ccccc4n(-c4ccc5c6ccccc6n(-c6ccc(-c7cccc8ccccc78)c(-n7c8ccccc8c8ccccc87)c6)c5c4)c3cc21 ; CC1(C)c2ccccc2-c2cc3c(cc21)c1ccccc1n3-c1ccc2c3ccccc3n(-c3ccc(-c4cccc5ccccc45)c(-n4c5ccccc5c5ccccc54)c3)c2c1 ; c1ccc(C2(c3ccccc3)c3ccccc3-c3cc4c5ccccc5n(-c5ccc6c7ccccc7n(-c7ccc(-c8cccc9ccccc89)c(-n8c9ccccc9c9ccccc98)c7)c6c5)c4cc32)cc1 ; c1ccc(C2(c3ccccc3)c3ccccc3-c3cc4c(cc32)c2ccccc2n4-c2ccc3c4ccccc4n(-c4ccc(-c5cccc6ccccc56)c(-n5c6ccccc6c6ccccc65)c4)c3c2)cc1]

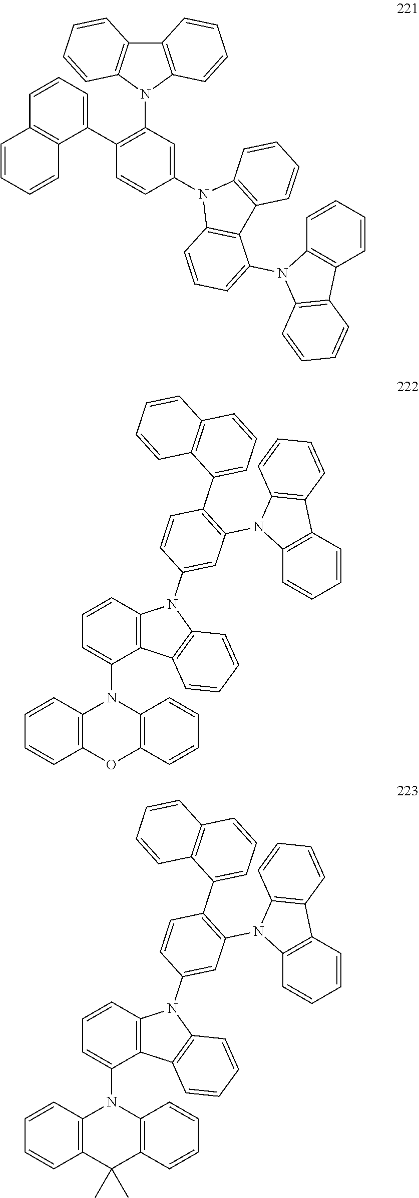

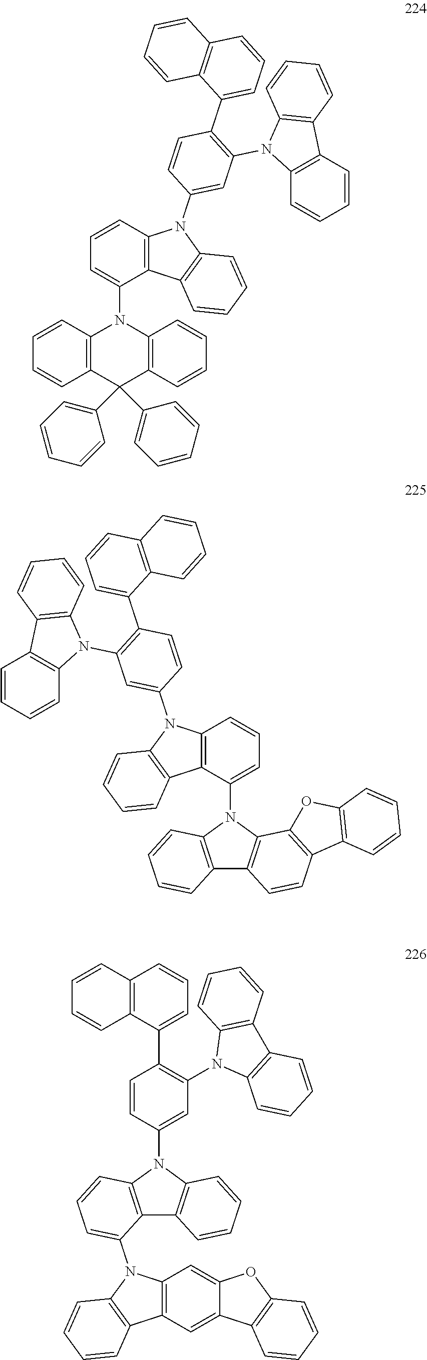 [SMILES: c1ccc(C2(c3ccccc3)c3ccccc3N(c3cccc4c3c3ccccc3n4-c3ccc(-c4cccc5ccccc45)c(-n4c5ccccc5c5ccccc54)c3)c3ccccc32)cc1 ; c1ccc2c(-c3ccc(-n4c5ccccc5c5c(-n6c7ccccc7c7ccc8c9ccccc9oc8c76)cccc54)cc3-n3c4ccccc4c4ccccc43)cccc2c1 ; c1ccc2c(-c3ccc(-n4c5ccccc5c5c(-n6c7ccccc7c7cc8c(cc76)oc6ccccc68)cccc54)cc3-n3c4ccccc4c4ccccc43)cccc2c1]

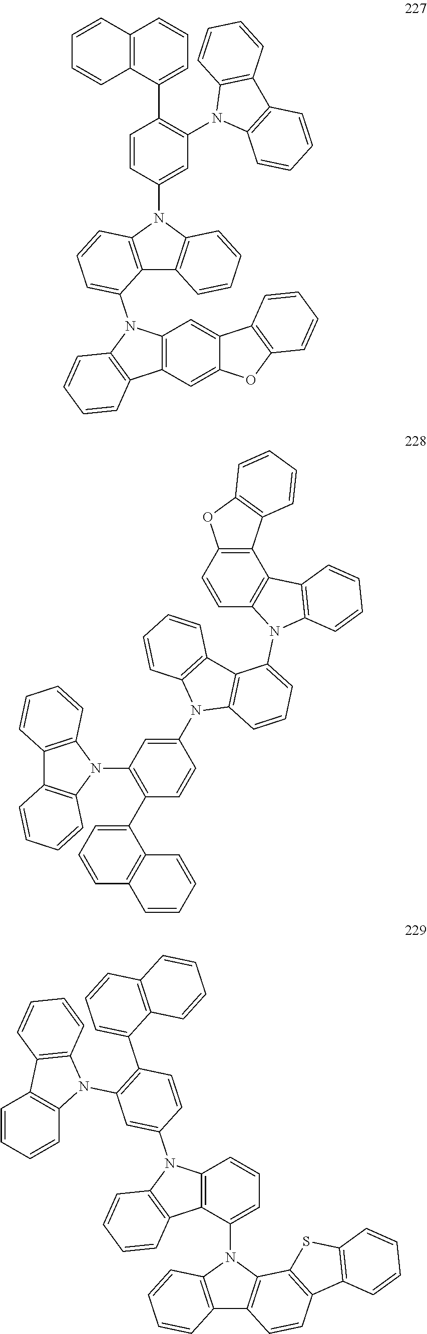

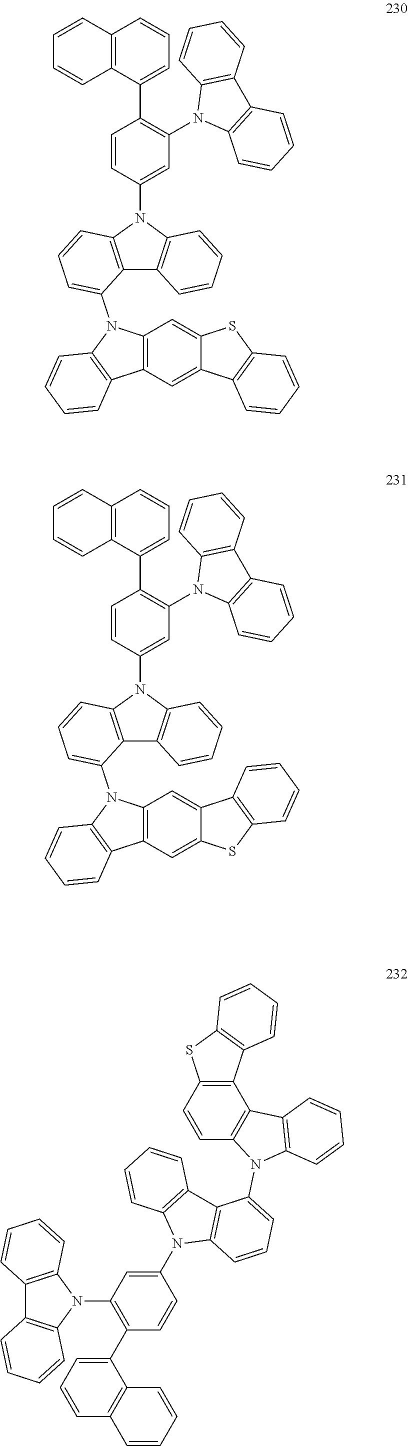 [SMILES: c1ccc2c(-c3ccc(-n4c5ccccc5c5c(-n6c7ccccc7c7cc8c(cc76)sc6ccccc68)cccc54)cc3-n3c4ccccc4c4ccccc43)cccc2c1 ; c1ccc2c(-c3ccc(-n4c5ccccc5c5c(-n6c7ccccc7c7cc8sc9ccccc9c8cc76)cccc54)cc3-n3c4ccccc4c4ccccc43)cccc2c1 ; c1ccc2c(-c3ccc(-n4c5ccccc5c5c(-n6c7ccccc7c7c8c(ccc76)sc6ccccc68)cccc54)cc3-n3c4ccccc4c4ccccc43)cccc2c1]

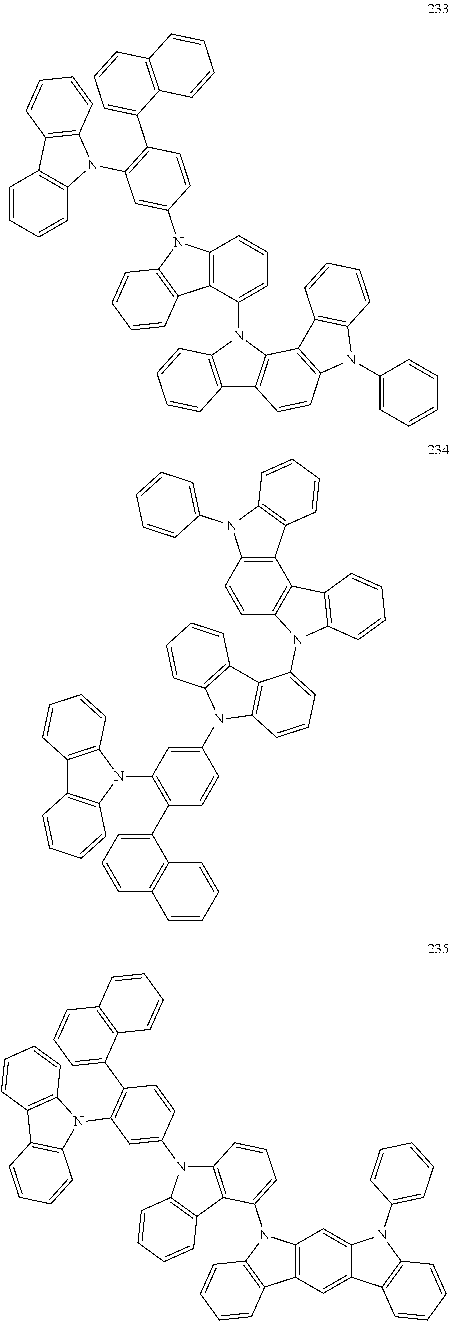 [SMILES: c1ccc(-n2c3ccccc3c3c2ccc2c4ccccc4n(-c4cccc5c4c4ccccc4n5-c4ccc(-c5cccc6ccccc56)c(-n5c6ccccc6c6ccccc65)c4)c23)cc1 ; c1ccc(-n2c3ccccc3c3c4c5ccccc5n(-c5cccc6c5c5ccccc5n6-c5ccc(-c6cccc7ccccc67)c(-n6c7ccccc7c7ccccc76)c5)c4ccc32)cc1 ; c1ccc(-n2c3ccccc3c3cc4c5ccccc5n(-c5cccc6c5c5ccccc5n6-c5ccc(-c6cccc7ccccc67)c(-n6c7ccccc7c7ccccc76)c5)c4cc32)cc1]

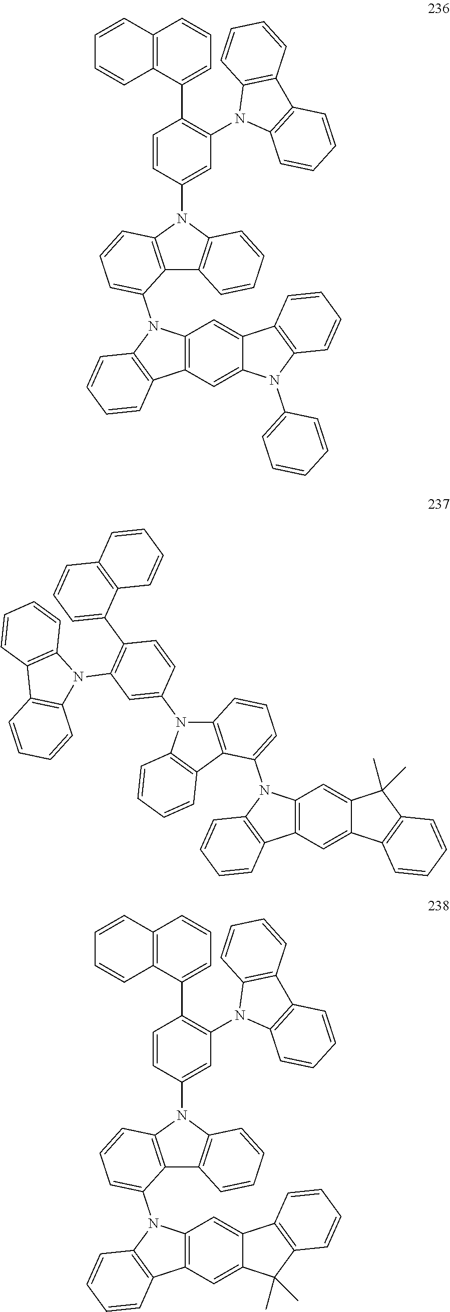 [SMILES: c1ccc(-n2c3ccccc3c3cc4c(cc32)c2ccccc2n4-c2cccc3c2c2ccccc2n3-c2ccc(-c3cccc4ccccc34)c(-n3c4ccccc4c4ccccc43)c2)cc1 ; CC1(C)c2ccccc2-c2cc3c4ccccc4n(-c4cccc5c4c4ccccc4n5-c4ccc(-c5cccc6ccccc56)c(-n5c6ccccc6c6ccccc65)c4)c3cc21 ; CC1(C)c2ccccc2-c2cc3c(cc21)c1ccccc1n3-c1cccc2c1c1ccccc1n2-c1ccc(-c2cccc3ccccc23)c(-n2c3ccccc3c3ccccc32)c1]

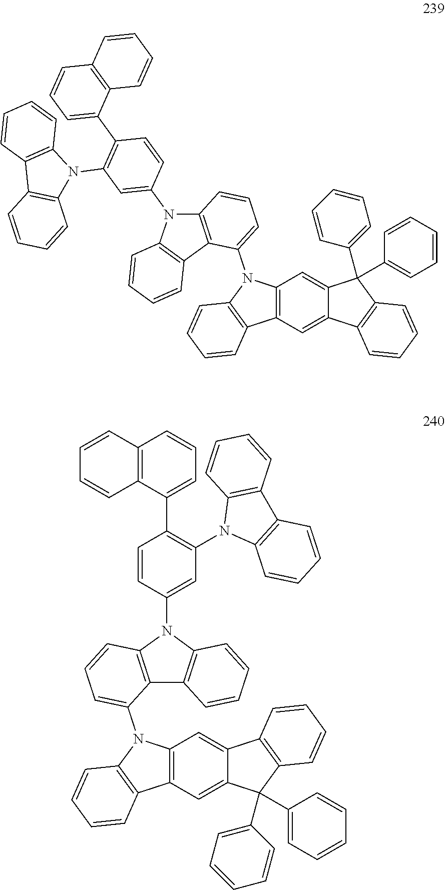

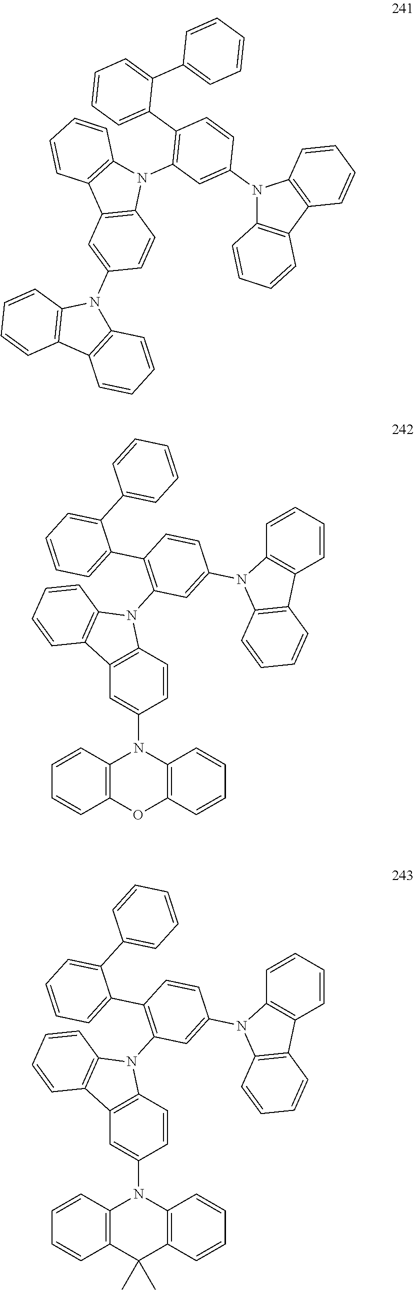

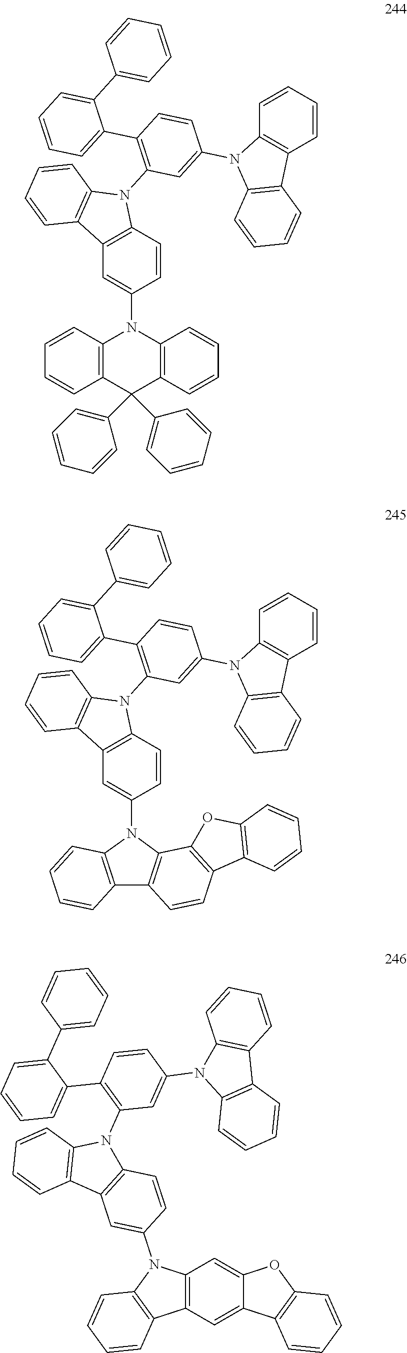 [SMILES: c1ccc(-c2ccccc2-c2ccc(-n3c4ccccc4c4ccccc43)cc2-n2c3ccccc3c3cc(N4c5ccccc5C(c5ccccc5)(c5ccccc5)c5ccccc54)ccc32)cc1 ; c1ccc(-c2ccccc2-c2ccc(-n3c4ccccc4c4ccccc43)cc2-n2c3ccccc3c3cc(-n4c5ccccc5c5ccc6c7ccccc7oc6c54)ccc32)cc1 ; c1ccc(-c2ccccc2-c2ccc(-n3c4ccccc4c4ccccc43)cc2-n2c3ccccc3c3cc(-n4c5ccccc5c5cc6c(cc54)oc4ccccc46)ccc32)cc1]

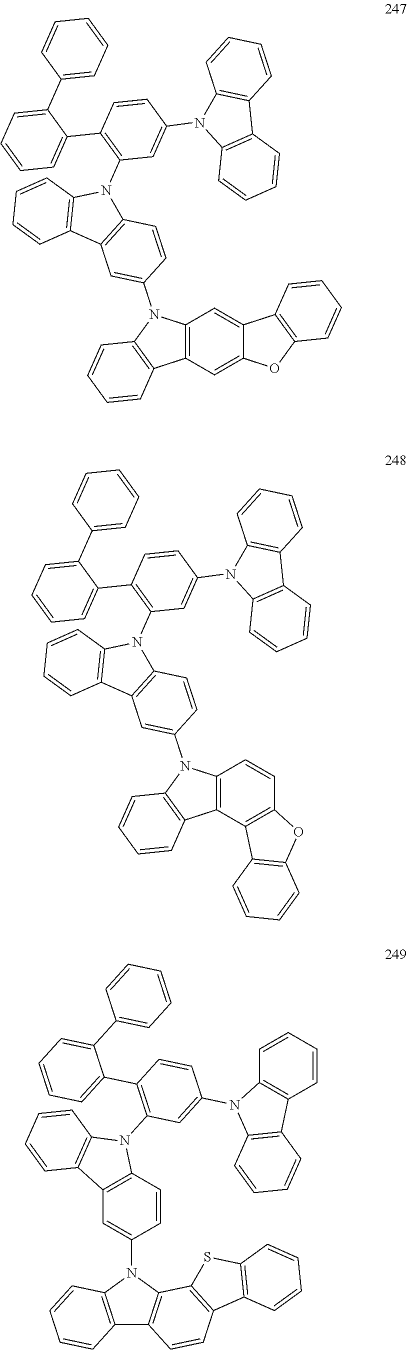 [SMILES: c1ccc(-c2ccccc2-c2ccc(-n3c4ccccc4c4ccccc43)cc2-n2c3ccccc3c3cc(-n4c5ccccc5c5cc6oc7ccccc7c6cc54)ccc32)cc1 ; c1ccc(-c2ccccc2-c2ccc(-n3c4ccccc4c4ccccc43)cc2-n2c3ccccc3c3cc(-n4c5ccccc5c5c6c(ccc54)oc4ccccc46)ccc32)cc1 ; c1ccc(-c2ccccc2-c2ccc(-n3c4ccccc4c4ccccc43)cc2-n2c3ccccc3c3cc(-n4c5ccccc5c5ccc6c7ccccc7sc6c54)ccc32)cc1]

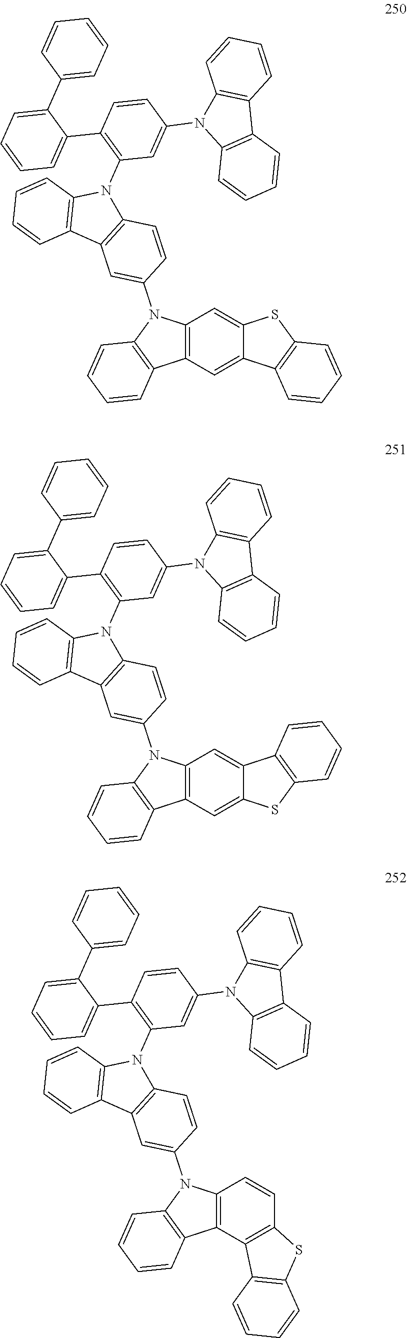

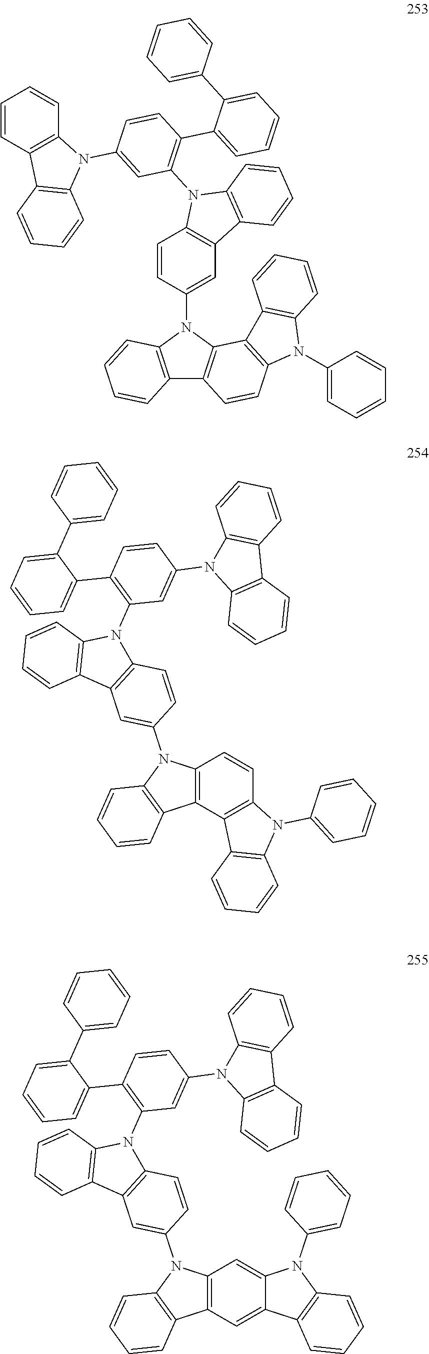 [SMILES: c1ccc(-c2ccccc2-c2ccc(-n3c4ccccc4c4ccccc43)cc2-n2c3ccccc3c3cc(-n4c5ccccc5c5ccc6c(c7ccccc7n6-c6ccccc6)c54)ccc32)cc1 ; c1ccc(-c2ccccc2-c2ccc(-n3c4ccccc4c4ccccc43)cc2-n2c3ccccc3c3cc(-n4c5ccccc5c5c6c7ccccc7n(-c7ccccc7)c6ccc54)ccc32)cc1 ; c1ccc(-c2ccccc2-c2ccc(-n3c4ccccc4c4ccccc43)cc2-n2c3ccccc3c3cc(-n4c5ccccc5c5cc6c7ccccc7n(-c7ccccc7)c6cc54)ccc32)cc1]

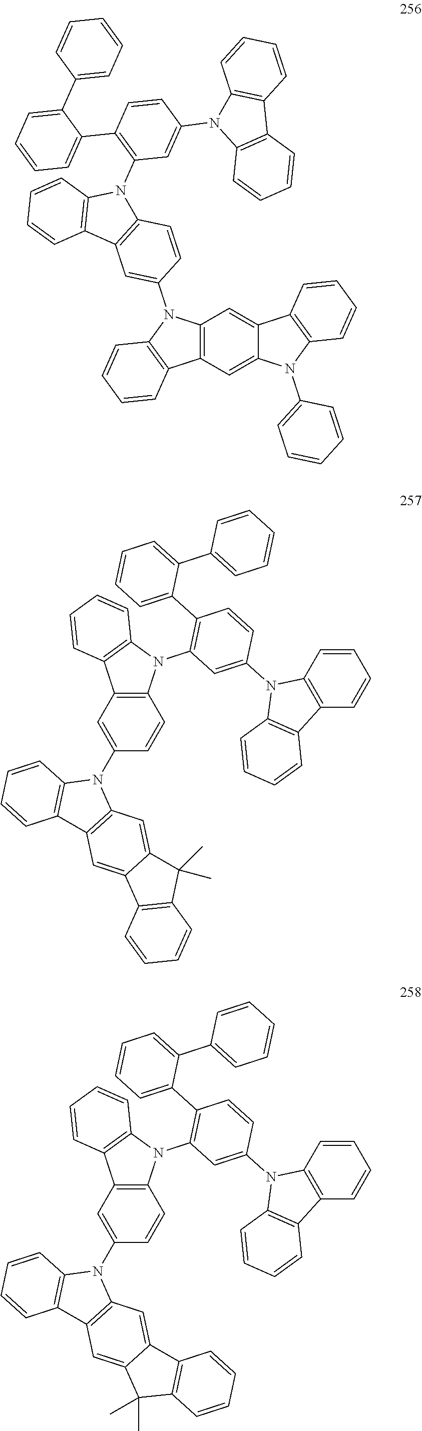 [SMILES: c1ccc(-c2ccccc2-c2ccc(-n3c4ccccc4c4ccccc43)cc2-n2c3ccccc3c3cc(-n4c5ccccc5c5cc6c(cc54)c4ccccc4n6-c4ccccc4)ccc32)cc1 ; CC1(C)c2ccccc2-c2cc3c4ccccc4n(-c4ccc5c(c4)c4ccccc4n5-c4cc(-n5c6ccccc6c6ccccc65)ccc4-c4ccccc4-c4ccccc4)c3cc21 ; CC1(C)c2ccccc2-c2cc3c(cc21)c1ccccc1n3-c1ccc2c(c1)c1ccccc1n2-c1cc(-n2c3ccccc3c3ccccc32)ccc1-c1ccccc1-c1ccccc1]

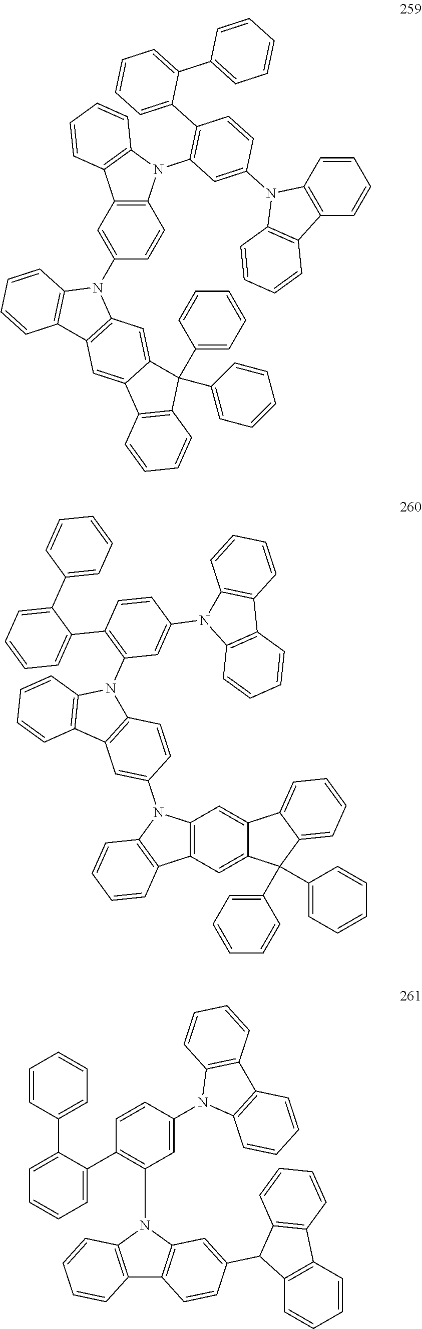 [SMILES: c1ccc(-c2ccccc2-c2ccc(-n3c4ccccc4c4ccccc43)cc2-n2c3ccccc3c3cc(-n4c5ccccc5c5cc6c(cc54)C(c4ccccc4)(c4ccccc4)c4ccccc4-6)ccc32)cc1 ; c1ccc(-c2ccccc2-c2ccc(-n3c4ccccc4c4ccccc43)cc2-n2c3ccccc3c3cc(-n4c5ccccc5c5cc6c(cc54)-c4ccccc4C6(c4ccccc4)c4ccccc4)ccc32)cc1 ; c1ccc(-c2ccccc2-c2ccc(-n3c4ccccc4c4ccccc43)cc2-n2c3ccccc3c3ccc(C4c5ccccc5-c5ccccc54)cc32)cc1]

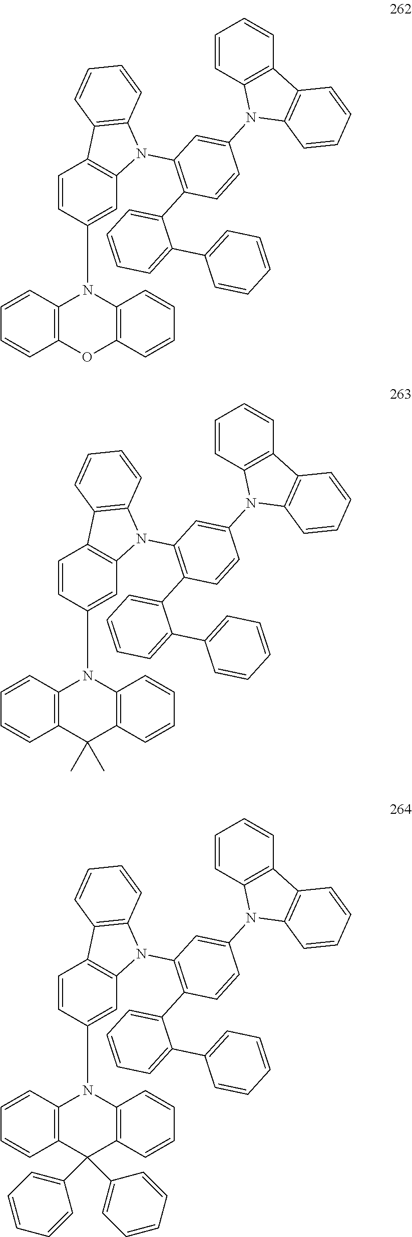 [SMILES: c1ccc(-c2ccccc2-c2ccc(-n3c4ccccc4c4ccccc43)cc2-n2c3ccccc3c3ccc(N4c5ccccc5Oc5ccccc54)cc32)cc1 ; CC1(C)c2ccccc2N(c2ccc3c4ccccc4n(-c4cc(-n5c6ccccc6c6ccccc65)ccc4-c4ccccc4-c4ccccc4)c3c2)c2ccccc21 ; c1ccc(-c2ccccc2-c2ccc(-n3c4ccccc4c4ccccc43)cc2-n2c3ccccc3c3ccc(N4c5ccccc5C(c5ccccc5)(c5ccccc5)c5ccccc54)cc32)cc1]

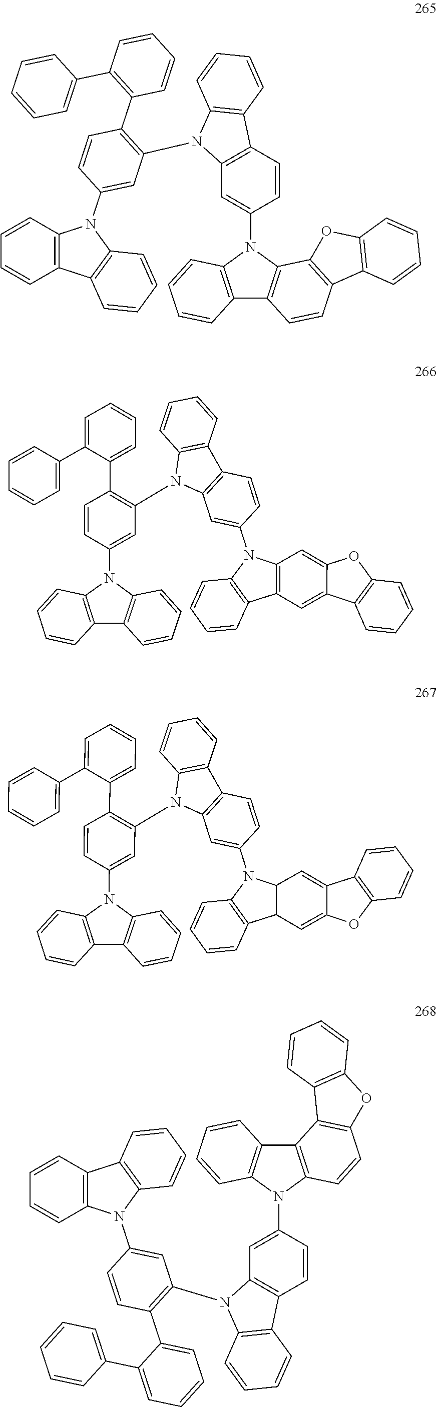 [SMILES: c1ccc(-c2ccccc2-c2ccc(-n3c4ccccc4c4ccccc43)cc2-n2c3ccccc3c3ccc(-n4c5ccccc5c5ccc6c7ccccc7oc6c54)cc32)cc1 ; c1ccc(-c2ccccc2-c2ccc(-n3c4ccccc4c4ccccc43)cc2-n2c3ccccc3c3ccc(-n4c5ccccc5c5cc6c(cc54)oc4ccccc46)cc32)cc1 ; C1=c2oc3ccccc3c2=CC2C1c1ccccc1N2c1ccc2c3ccccc3n(-c3cc(-n4c5ccccc5c5ccccc54)ccc3-c3ccccc3-c3ccccc3)c2c1 ; c1ccc(-c2ccccc2-c2ccc(-n3c4ccccc4c4ccccc43)cc2-n2c3ccccc3c3ccc(-n4c5ccccc5c5c6c(ccc54)oc4ccccc46)cc32)cc1]

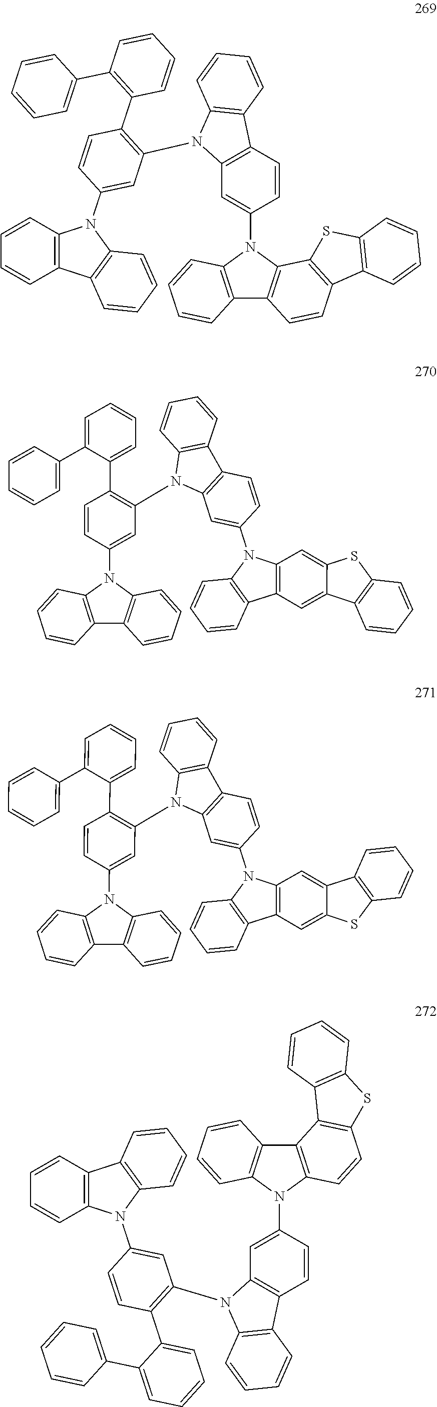 [SMILES: c1ccc(-c2ccccc2-c2ccc(-n3c4ccccc4c4ccccc43)cc2-n2c3ccccc3c3ccc(-n4c5ccccc5c5ccc6c7ccccc7sc6c54)cc32)cc1 ; c1ccc(-c2ccccc2-c2ccc(-n3c4ccccc4c4ccccc43)cc2-n2c3ccccc3c3ccc(-n4c5ccccc5c5cc6c(cc54)sc4ccccc46)cc32)cc1 ; c1ccc(-c2ccccc2-c2ccc(-n3c4ccccc4c4ccccc43)cc2-n2c3ccccc3c3ccc(-n4c5ccccc5c5cc6sc7ccccc7c6cc54)cc32)cc1 ; c1ccc(-c2ccccc2-c2ccc(-n3c4ccccc4c4ccccc43)cc2-n2c3ccccc3c3ccc(-n4c5ccccc5c5c6c(ccc54)sc4ccccc46)cc32)cc1]

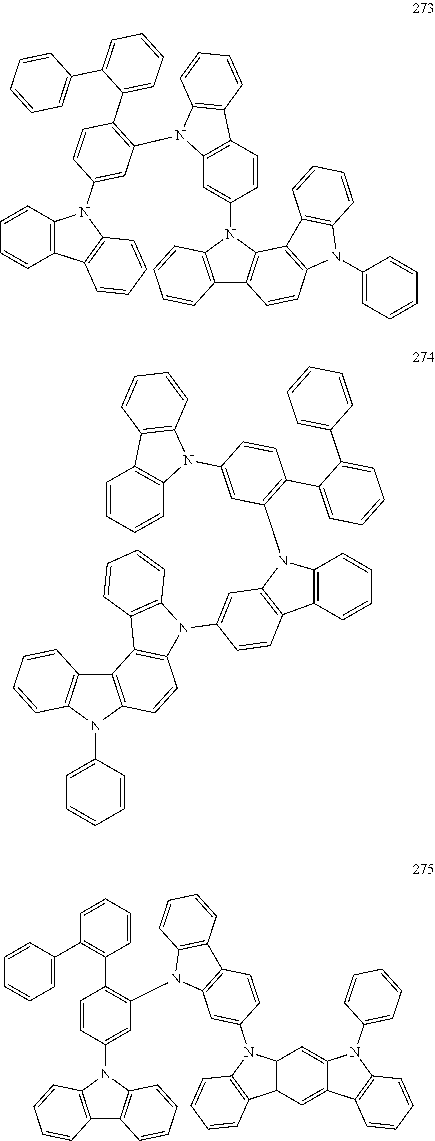 [SMILES: c1ccc(-c2ccccc2-c2ccc(-n3c4ccccc4c4ccccc43)cc2-n2c3ccccc3c3ccc(-n4c5ccccc5c5ccc6c(c7ccccc7n6-c6ccccc6)c54)cc32)cc1 ; c1ccc(-c2ccccc2-c2ccc(-n3c4ccccc4c4ccccc43)cc2-n2c3ccccc3c3ccc(-n4c5ccccc5c5c6c7ccccc7n(-c7ccccc7)c6ccc54)cc32)cc1 ; C1=c2c(n(-c3ccccc3)c3ccccc23)=CC2C1c1ccccc1N2c1ccc2c3ccccc3n(-c3cc(-n4c5ccccc5c5ccccc54)ccc3-c3ccccc3-c3ccccc3)c2c1]

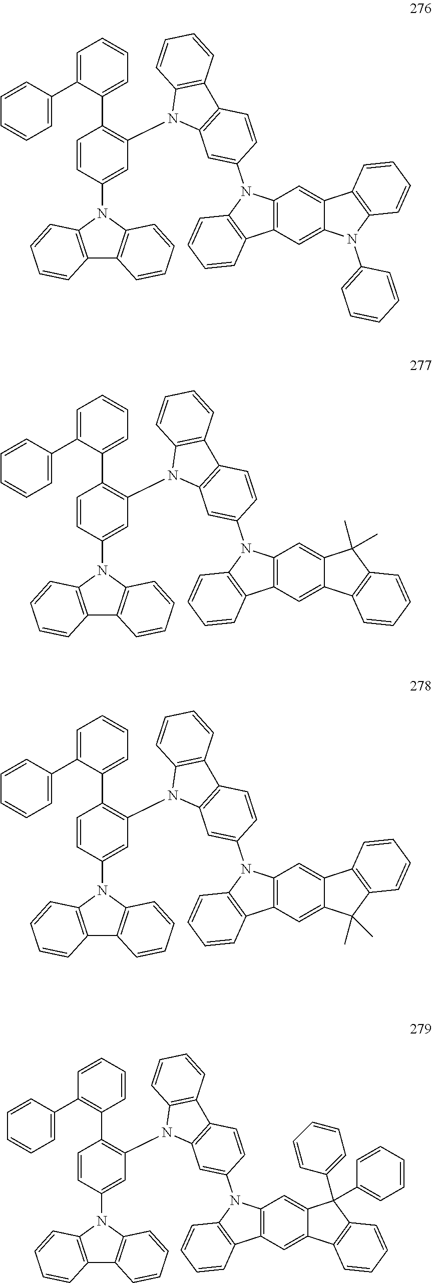 [SMILES: c1ccc(-c2ccccc2-c2ccc(-n3c4ccccc4c4ccccc43)cc2-n2c3ccccc3c3ccc(-n4c5ccccc5c5cc6c(cc54)c4ccccc4n6-c4ccccc4)cc32)cc1 ; CC1(C)c2ccccc2-c2cc3c4ccccc4n(-c4ccc5c6ccccc6n(-c6cc(-n7c8ccccc8c8ccccc87)ccc6-c6ccccc6-c6ccccc6)c5c4)c3cc21 ; CC1(C)c2ccccc2-c2cc3c(cc21)c1ccccc1n3-c1ccc2c3ccccc3n(-c3cc(-n4c5ccccc5c5ccccc54)ccc3-c3ccccc3-c3ccccc3)c2c1 ; c1ccc(-c2ccccc2-c2ccc(-n3c4ccccc4c4ccccc43)cc2-n2c3ccccc3c3ccc(-n4c5ccccc5c5cc6c(cc54)C(c4ccccc4)(c4ccccc4)c4ccccc4-6)cc32)cc1]

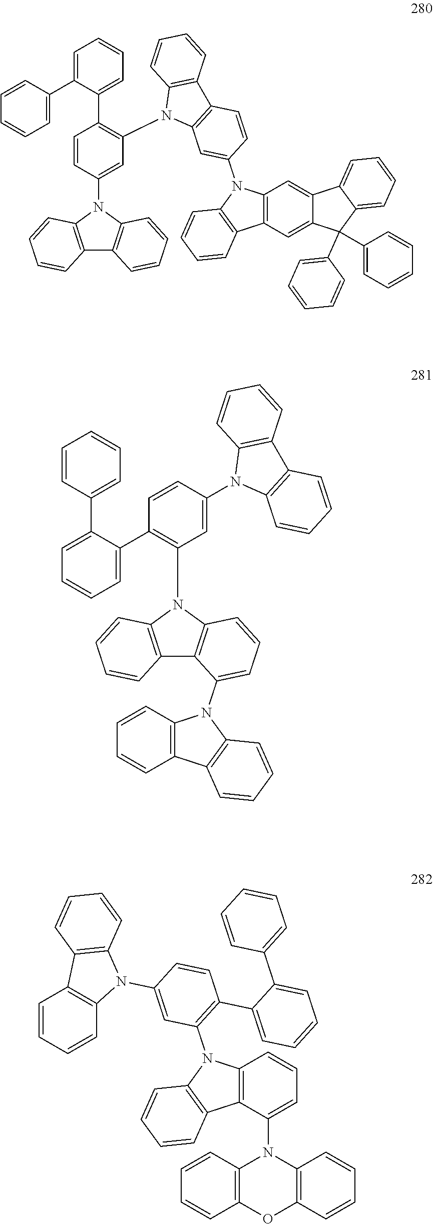 [SMILES: c1ccc(-c2ccccc2-c2ccc(-n3c4ccccc4c4ccccc43)cc2-n2c3ccccc3c3ccc(-n4c5ccccc5c5cc6c(cc54)-c4ccccc4C6(c4ccccc4)c4ccccc4)cc32)cc1 ; c1ccc(-c2ccccc2-c2ccc(-n3c4ccccc4c4ccccc43)cc2-n2c3ccccc3c3c(-n4c5ccccc5c5ccccc54)cccc32)cc1 ; c1ccc(-c2ccccc2-c2ccc(-n3c4ccccc4c4ccccc43)cc2-n2c3ccccc3c3c(N4c5ccccc5Oc5ccccc54)cccc32)cc1]

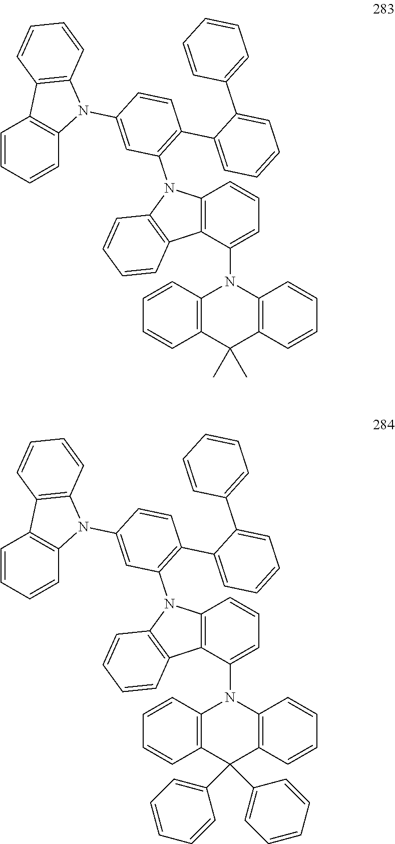 [SMILES: CC1(C)c2ccccc2N(c2cccc3c2c2ccccc2n3-c2cc(-n3c4ccccc4c4ccccc43)ccc2-c2ccccc2-c2ccccc2)c2ccccc21 ; c1ccc(-c2ccccc2-c2ccc(-n3c4ccccc4c4ccccc43)cc2-n2c3ccccc3c3c(N4c5ccccc5C(c5ccccc5)(c5ccccc5)c5ccccc54)cccc32)cc1]

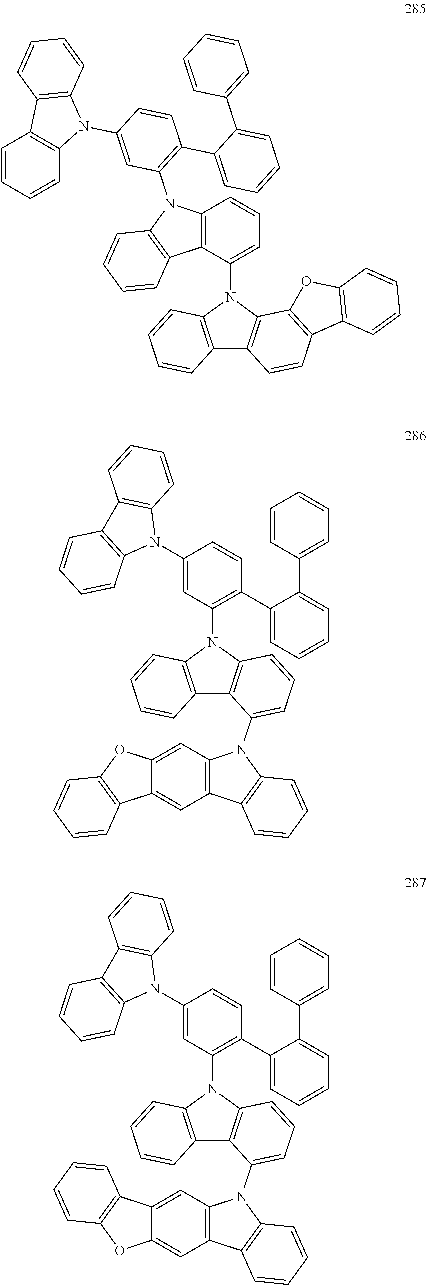 [SMILES: c1ccc(-c2ccccc2-c2ccc(-n3c4ccccc4c4ccccc43)cc2-n2c3ccccc3c3c(-n4c5ccccc5c5ccc6c7ccccc7oc6c54)cccc32)cc1 ; c1ccc(-c2ccccc2-c2ccc(-n3c4ccccc4c4ccccc43)cc2-n2c3ccccc3c3c(-n4c5ccccc5c5cc6c(cc54)oc4ccccc46)cccc32)cc1 ; c1ccc(-c2ccccc2-c2ccc(-n3c4ccccc4c4ccccc43)cc2-n2c3ccccc3c3c(-n4c5ccccc5c5cc6oc7ccccc7c6cc54)cccc32)cc1]

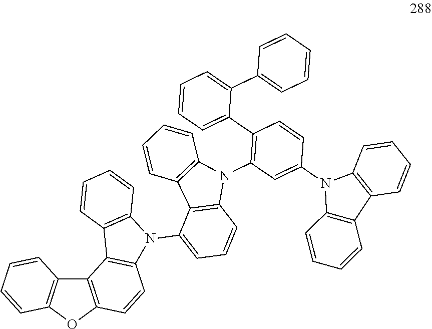 [SMILES: c1ccc(-c2ccccc2-c2ccc(-n3c4ccccc4c4ccccc43)cc2-n2c3ccccc3c3c(-n4c5ccccc5c5c6c(ccc54)oc4ccccc46)cccc32)cc1]

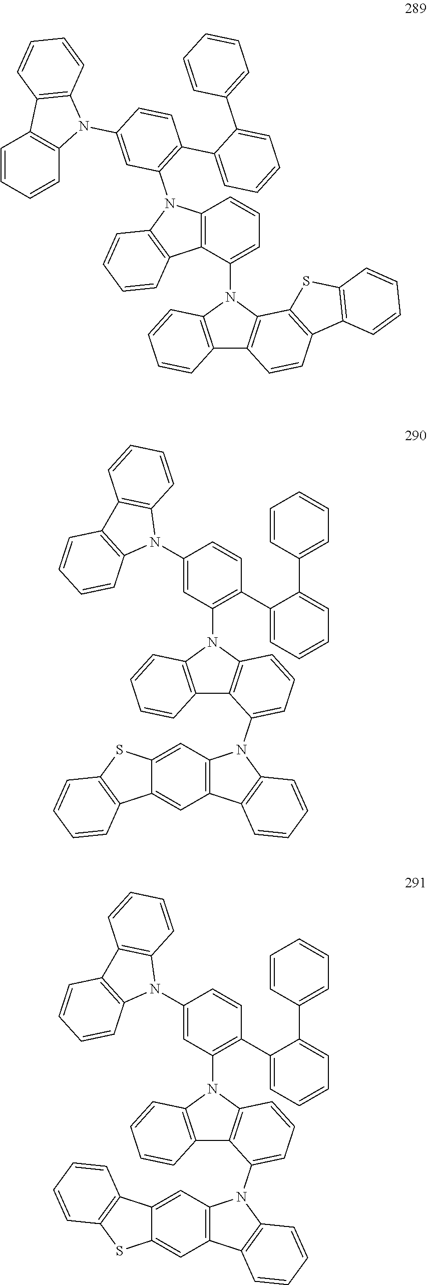 [SMILES: c1ccc(-c2ccccc2-c2ccc(-n3c4ccccc4c4ccccc43)cc2-n2c3ccccc3c3c(-n4c5ccccc5c5ccc6c7ccccc7sc6c54)cccc32)cc1 ; c1ccc(-c2ccccc2-c2ccc(-n3c4ccccc4c4ccccc43)cc2-n2c3ccccc3c3c(-n4c5ccccc5c5cc6c(cc54)sc4ccccc46)cccc32)cc1 ; c1ccc(-c2ccccc2-c2ccc(-n3c4ccccc4c4ccccc43)cc2-n2c3ccccc3c3c(-n4c5ccccc5c5cc6sc7ccccc7c6cc54)cccc32)cc1]

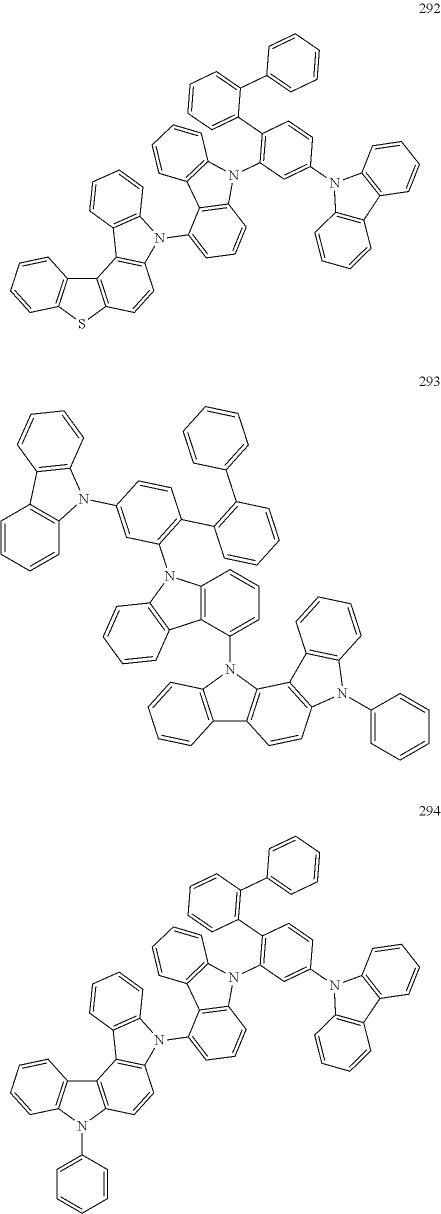 [SMILES: c1ccc(-c2ccccc2-c2ccc(-n3c4ccccc4c4ccccc43)cc2-n2c3ccccc3c3c(-n4c5ccccc5c5c6c(ccc54)sc4ccccc46)cccc32)cc1 ; c1ccc(-c2ccccc2-c2ccc(-n3c4ccccc4c4ccccc43)cc2-n2c3ccccc3c3c(-n4c5ccccc5c5ccc6c(c7ccccc7n6-c6ccccc6)c54)cccc32)cc1 ; c1ccc(-c2ccccc2-c2ccc(-n3c4ccccc4c4ccccc43)cc2-n2c3ccccc3c3c(-n4c5ccccc5c5c6c7ccccc7n(-c7ccccc7)c6ccc54)cccc32)cc1]

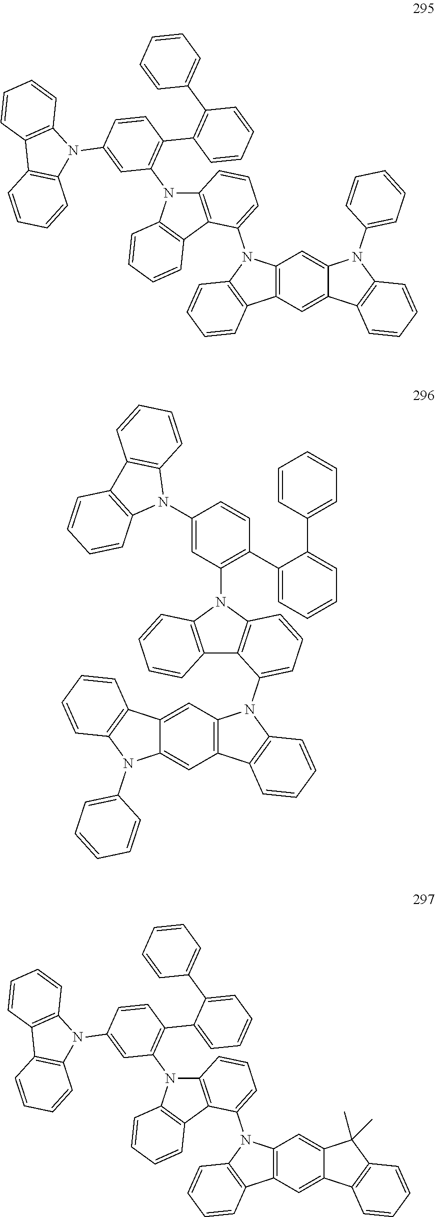 [SMILES: c1ccc(-c2ccccc2-c2ccc(-n3c4ccccc4c4ccccc43)cc2-n2c3ccccc3c3c(-n4c5ccccc5c5cc6c7ccccc7n(-c7ccccc7)c6cc54)cccc32)cc1 ; c1ccc(-c2ccccc2-c2ccc(-n3c4ccccc4c4ccccc43)cc2-n2c3ccccc3c3c(-n4c5ccccc5c5cc6c(cc54)c4ccccc4n6-c4ccccc4)cccc32)cc1 ; CC1(C)c2ccccc2-c2cc3c4ccccc4n(-c4cccc5c4c4ccccc4n5-c4cc(-n5c6ccccc6c6ccccc65)ccc4-c4ccccc4-c4ccccc4)c3cc21]

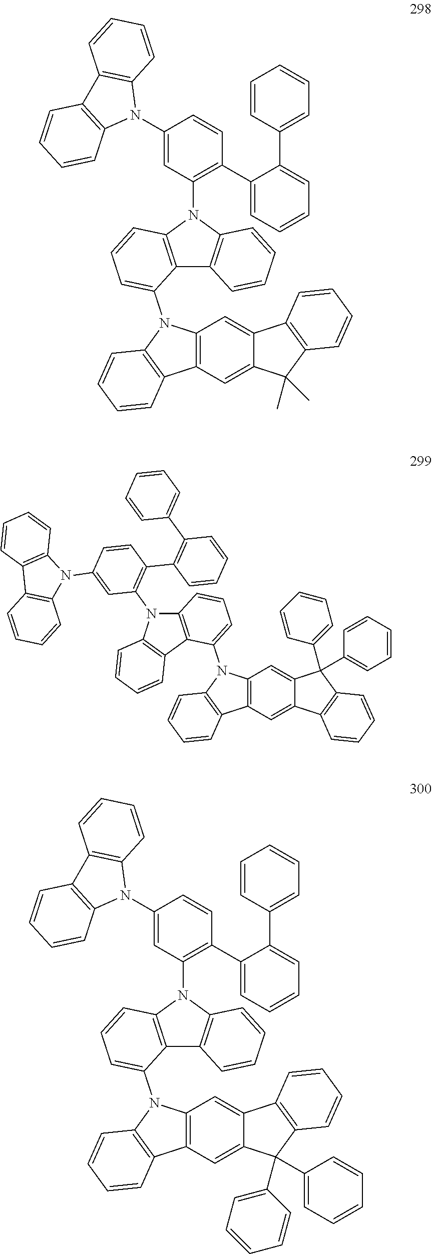 [SMILES: CC1(C)c2ccccc2-c2cc3c(cc21)c1ccccc1n3-c1cccc2c1c1ccccc1n2-c1cc(-n2c3ccccc3c3ccccc32)ccc1-c1ccccc1-c1ccccc1 ; c1ccc(-c2ccccc2-c2ccc(-n3c4ccccc4c4ccccc43)cc2-n2c3ccccc3c3c(-n4c5ccccc5c5cc6c(cc54)C(c4ccccc4)(c4ccccc4)c4ccccc4-6)cccc32)cc1 ; c1ccc(-c2ccccc2-c2ccc(-n3c4ccccc4c4ccccc43)cc2-n2c3ccccc3c3c(-n4c5ccccc5c5cc6c(cc54)-c4ccccc4C6(c4ccccc4)c4ccccc4)cccc32)cc1]

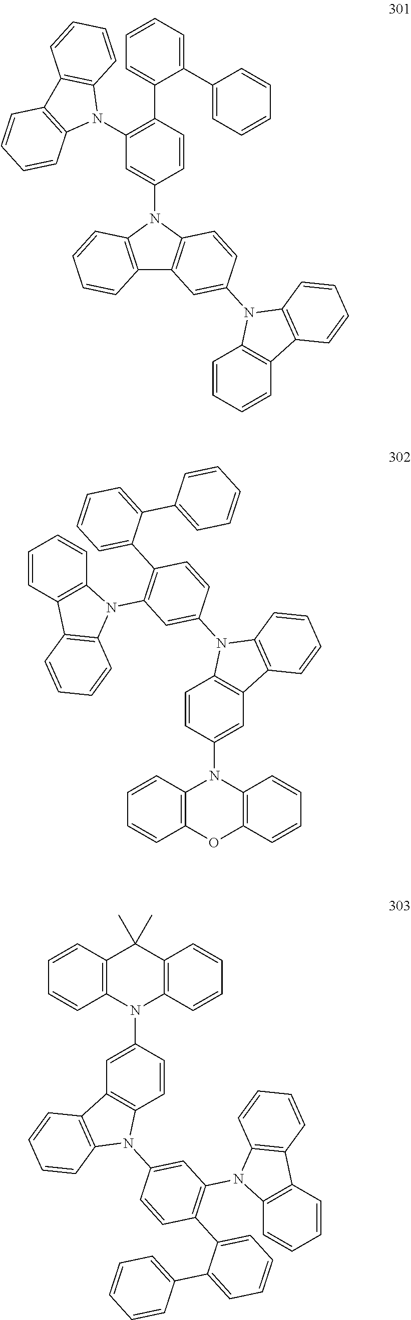 [SMILES: c1ccc(-c2ccccc2-c2ccc(-n3c4ccccc4c4cc(-n5c6ccccc6c6ccccc65)ccc43)cc2-n2c3ccccc3c3ccccc32)cc1 ; c1ccc(-c2ccccc2-c2ccc(-n3c4ccccc4c4cc(N5c6ccccc6Oc6ccccc65)ccc43)cc2-n2c3ccccc3c3ccccc32)cc1 ; CC1(C)c2ccccc2N(c2ccc3c(c2)c2ccccc2n3-c2ccc(-c3ccccc3-c3ccccc3)c(-n3c4ccccc4c4ccccc43)c2)c2ccccc21]

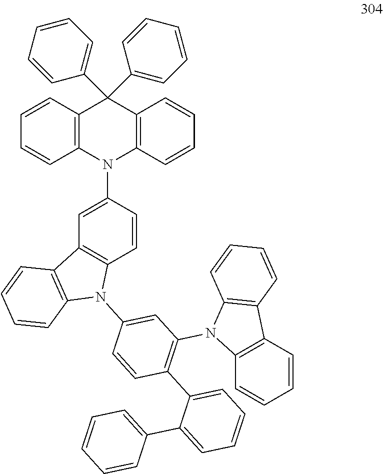 [SMILES: c1ccc(-c2ccccc2-c2ccc(-n3c4ccccc4c4cc(N5c6ccccc6C(c6ccccc6)(c6ccccc6)c6ccccc65)ccc43)cc2-n2c3ccccc3c3ccccc32)cc1]

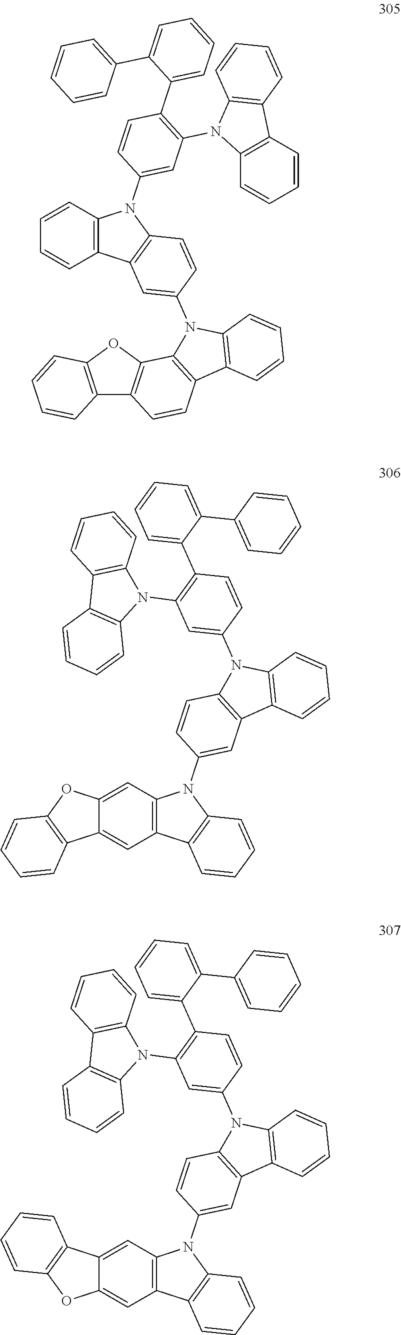 [SMILES: c1ccc(-c2ccccc2-c2ccc(-n3c4ccccc4c4cc(-n5c6ccccc6c6ccc7c8ccccc8oc7c65)ccc43)cc2-n2c3ccccc3c3ccccc32)cc1 ; c1ccc(-c2ccccc2-c2ccc(-n3c4ccccc4c4cc(-n5c6ccccc6c6cc7c(cc65)oc5ccccc57)ccc43)cc2-n2c3ccccc3c3ccccc32)cc1 ; c1ccc(-c2ccccc2-c2ccc(-n3c4ccccc4c4cc(-n5c6ccccc6c6cc7oc8ccccc8c7cc65)ccc43)cc2-n2c3ccccc3c3ccccc32)cc1]

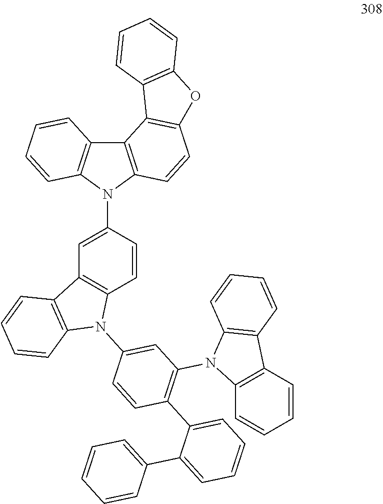 [SMILES: c1ccc(-c2ccccc2-c2ccc(-n3c4ccccc4c4cc(-n5c6ccccc6c6c7c(ccc65)oc5ccccc57)ccc43)cc2-n2c3ccccc3c3ccccc32)cc1]

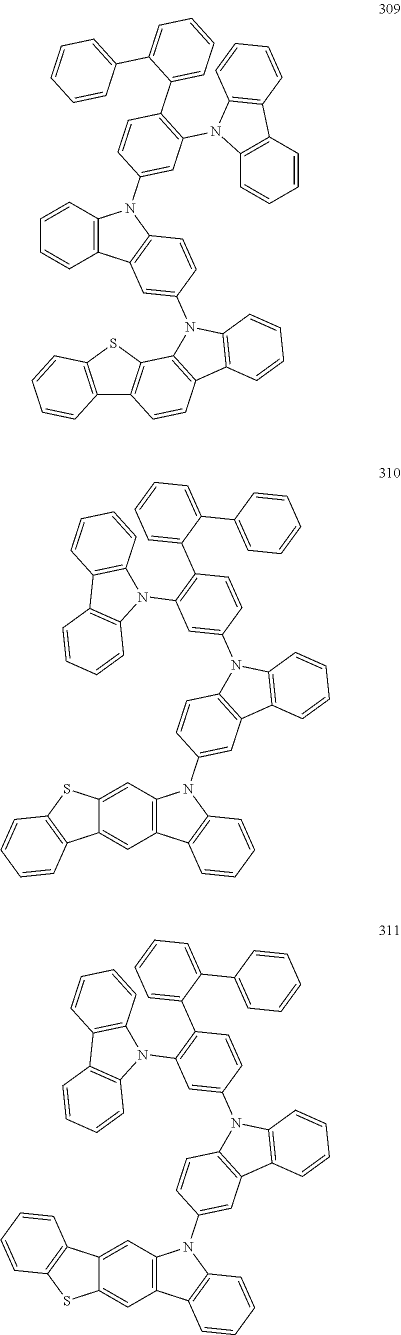 [SMILES: c1ccc(-c2ccccc2-c2ccc(-n3c4ccccc4c4cc(-n5c6ccccc6c6ccc7c8ccccc8sc7c65)ccc43)cc2-n2c3ccccc3c3ccccc32)cc1 ; c1ccc(-c2ccccc2-c2ccc(-n3c4ccccc4c4cc(-n5c6ccccc6c6cc7c(cc65)sc5ccccc57)ccc43)cc2-n2c3ccccc3c3ccccc32)cc1 ; c1ccc(-c2ccccc2-c2ccc(-n3c4ccccc4c4cc(-n5c6ccccc6c6cc7sc8ccccc8c7cc65)ccc43)cc2-n2c3ccccc3c3ccccc32)cc1]

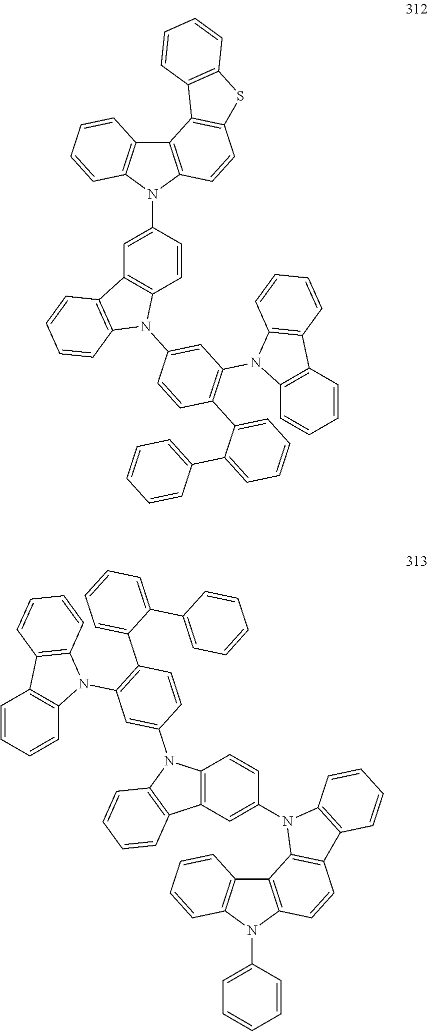 [SMILES: c1ccc(-c2ccccc2-c2ccc(-n3c4ccccc4c4cc(-n5c6ccccc6c6c7c(ccc65)sc5ccccc57)ccc43)cc2-n2c3ccccc3c3ccccc32)cc1 ; c1ccc(-c2ccccc2-c2ccc(-n3c4ccccc4c4cc(-n5c6ccccc6c6ccc7c(c8ccccc8n7-c7ccccc7)c65)ccc43)cc2-n2c3ccccc3c3ccccc32)cc1]

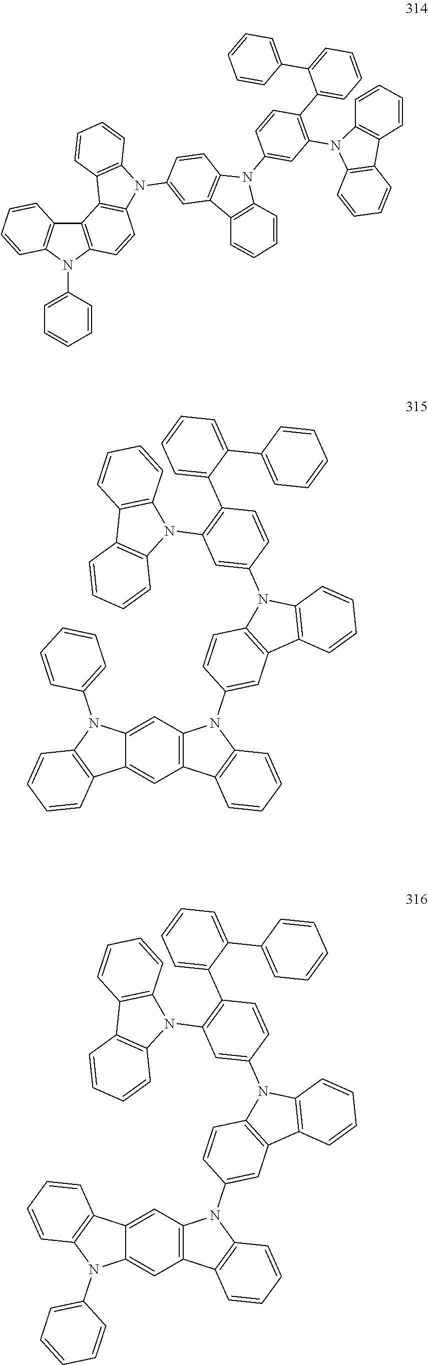 [SMILES: c1ccc(-c2ccccc2-c2ccc(-n3c4ccccc4c4cc(-n5c6ccccc6c6c7c8ccccc8n(-c8ccccc8)c7ccc65)ccc43)cc2-n2c3ccccc3c3ccccc32)cc1 ; c1ccc(-c2ccccc2-c2ccc(-n3c4ccccc4c4cc(-n5c6ccccc6c6cc7c8ccccc8n(-c8ccccc8)c7cc65)ccc43)cc2-n2c3ccccc3c3ccccc32)cc1 ; c1ccc(-c2ccccc2-c2ccc(-n3c4ccccc4c4cc(-n5c6ccccc6c6cc7c(cc65)c5ccccc5n7-c5ccccc5)ccc43)cc2-n2c3ccccc3c3ccccc32)cc1]

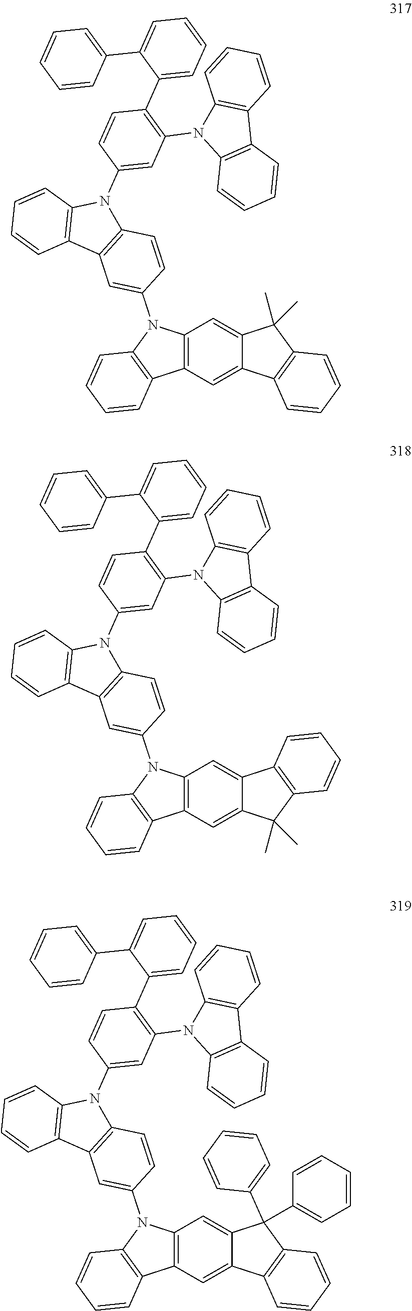 [SMILES: CC1(C)c2ccccc2-c2cc3c4ccccc4n(-c4ccc5c(c4)c4ccccc4n5-c4ccc(-c5ccccc5-c5ccccc5)c(-n5c6ccccc6c6ccccc65)c4)c3cc21 ; CC1(C)c2ccccc2-c2cc3c(cc21)c1ccccc1n3-c1ccc2c(c1)c1ccccc1n2-c1ccc(-c2ccccc2-c2ccccc2)c(-n2c3ccccc3c3ccccc32)c1 ; c1ccc(-c2ccccc2-c2ccc(-n3c4ccccc4c4cc(-n5c6ccccc6c6cc7c(cc65)C(c5ccccc5)(c5ccccc5)c5ccccc5-7)ccc43)cc2-n2c3ccccc3c3ccccc32)cc1]

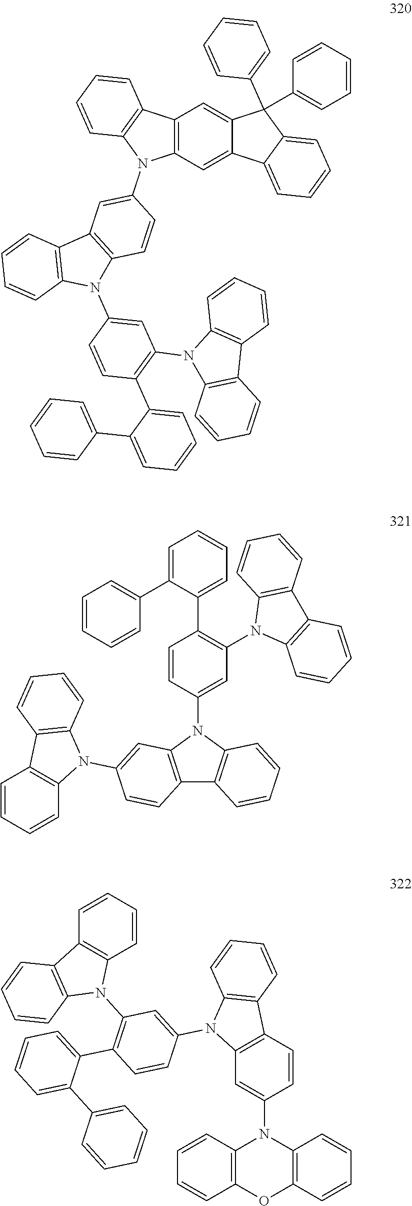 [SMILES: c1ccc(-c2ccccc2-c2ccc(-n3c4ccccc4c4cc(-n5c6ccccc6c6cc7c(cc65)-c5ccccc5C7(c5ccccc5)c5ccccc5)ccc43)cc2-n2c3ccccc3c3ccccc32)cc1 ; c1ccc(-c2ccccc2-c2ccc(-n3c4ccccc4c4ccc(-n5c6ccccc6c6ccccc65)cc43)cc2-n2c3ccccc3c3ccccc32)cc1 ; c1ccc(-c2ccccc2-c2ccc(-n3c4ccccc4c4ccc(N5c6ccccc6Oc6ccccc65)cc43)cc2-n2c3ccccc3c3ccccc32)cc1]

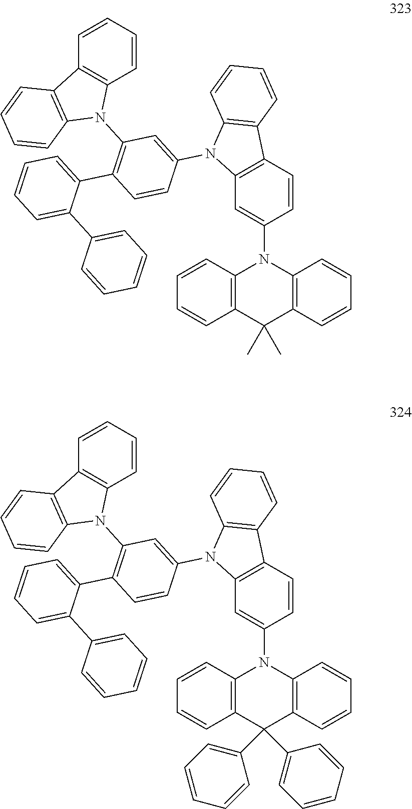 [SMILES: CC1(C)c2ccccc2N(c2ccc3c4ccccc4n(-c4ccc(-c5ccccc5-c5ccccc5)c(-n5c6ccccc6c6ccccc65)c4)c3c2)c2ccccc21 ; c1ccc(-c2ccccc2-c2ccc(-n3c4ccccc4c4ccc(N5c6ccccc6C(c6ccccc6)(c6ccccc6)c6ccccc65)cc43)cc2-n2c3ccccc3c3ccccc32)cc1]

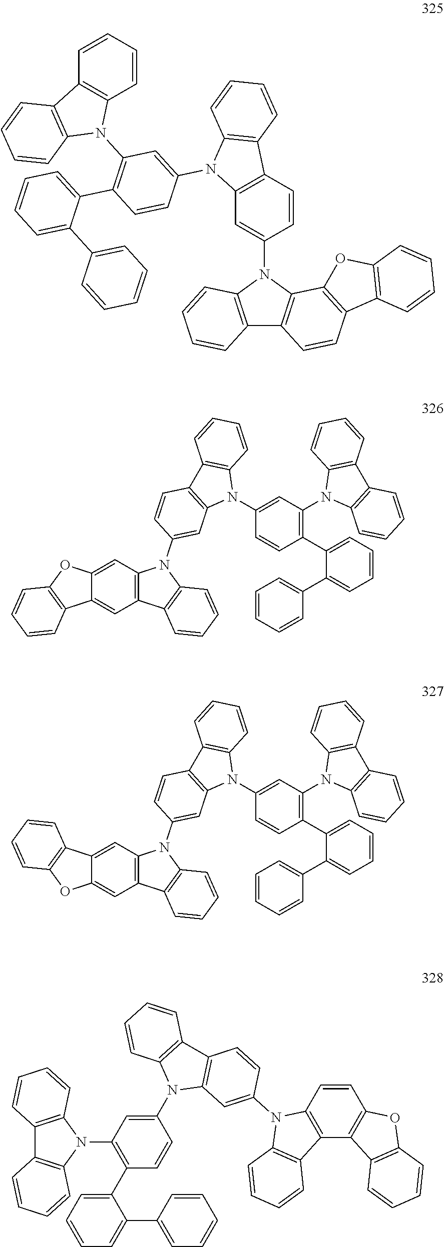 [SMILES: c1ccc(-c2ccccc2-c2ccc(-n3c4ccccc4c4ccc(-n5c6ccccc6c6ccc7c8ccccc8oc7c65)cc43)cc2-n2c3ccccc3c3ccccc32)cc1 ; c1ccc(-c2ccccc2-c2ccc(-n3c4ccccc4c4ccc(-n5c6ccccc6c6cc7c(cc65)oc5ccccc57)cc43)cc2-n2c3ccccc3c3ccccc32)cc1 ; c1ccc(-c2ccccc2-c2ccc(-n3c4ccccc4c4ccc(-n5c6ccccc6c6cc7oc8ccccc8c7cc65)cc43)cc2-n2c3ccccc3c3ccccc32)cc1 ; c1ccc(-c2ccccc2-c2ccc(-n3c4ccccc4c4ccc(-n5c6ccccc6c6c7c(ccc65)oc5ccccc57)cc43)cc2-n2c3ccccc3c3ccccc32)cc1]

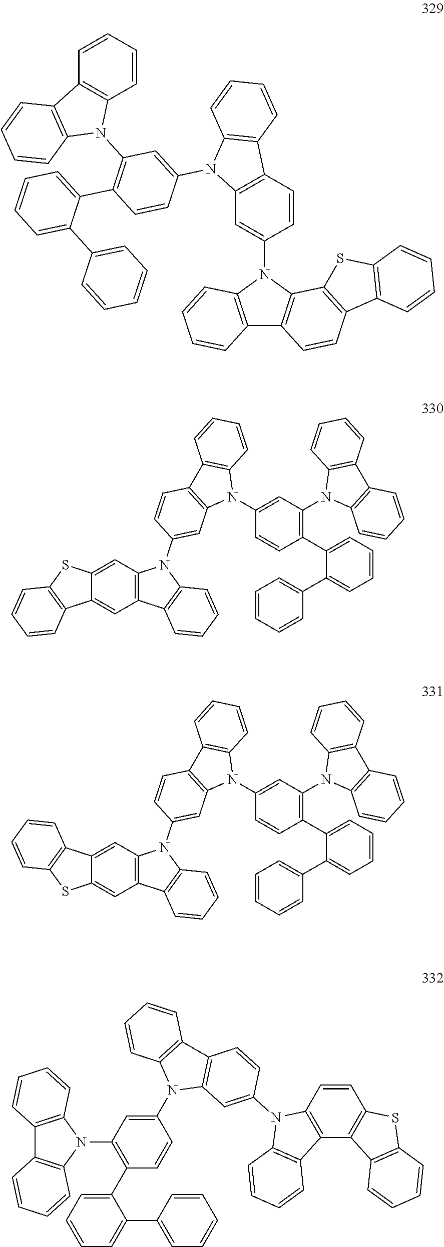 [SMILES: c1ccc(-c2ccccc2-c2ccc(-n3c4ccccc4c4ccc(-n5c6ccccc6c6ccc7c8ccccc8sc7c65)cc43)cc2-n2c3ccccc3c3ccccc32)cc1 ; c1ccc(-c2ccccc2-c2ccc(-n3c4ccccc4c4ccc(-n5c6ccccc6c6cc7c(cc65)sc5ccccc57)cc43)cc2-n2c3ccccc3c3ccccc32)cc1 ; c1ccc(-c2ccccc2-c2ccc(-n3c4ccccc4c4ccc(-n5c6ccccc6c6cc7sc8ccccc8c7cc65)cc43)cc2-n2c3ccccc3c3ccccc32)cc1 ; c1ccc(-c2ccccc2-c2ccc(-n3c4ccccc4c4ccc(-n5c6ccccc6c6c7c(ccc65)sc5ccccc57)cc43)cc2-n2c3ccccc3c3ccccc32)cc1]

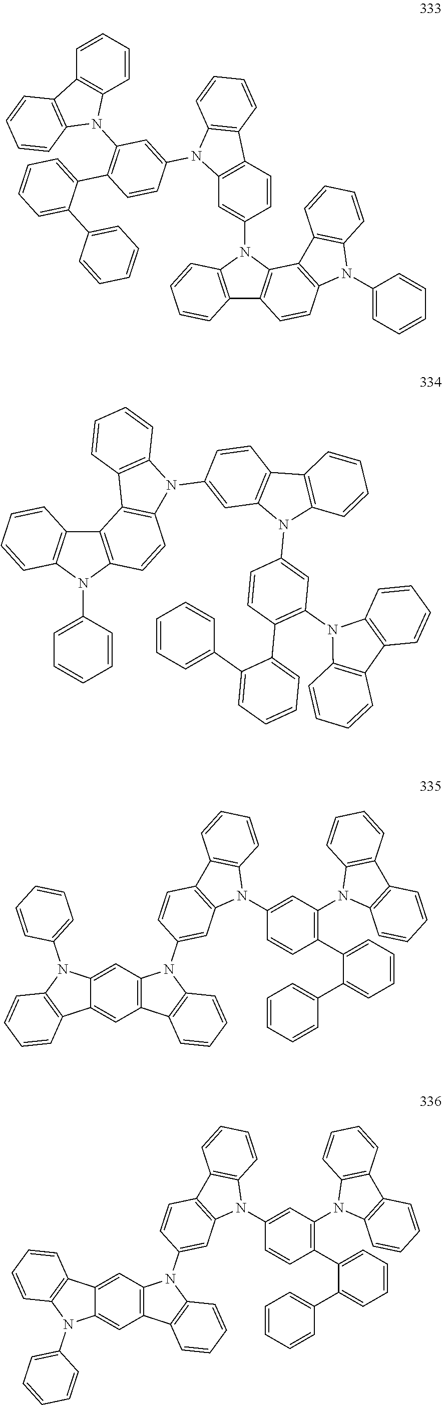 [SMILES: c1ccc(-c2ccccc2-c2ccc(-n3c4ccccc4c4ccc(-n5c6ccccc6c6ccc7c(c8ccccc8n7-c7ccccc7)c65)cc43)cc2-n2c3ccccc3c3ccccc32)cc1 ; c1ccc(-c2ccccc2-c2ccc(-n3c4ccccc4c4ccc(-n5c6ccccc6c6c7c8ccccc8n(-c8ccccc8)c7ccc65)cc43)cc2-n2c3ccccc3c3ccccc32)cc1 ; c1ccc(-c2ccccc2-c2ccc(-n3c4ccccc4c4ccc(-n5c6ccccc6c6cc7c8ccccc8n(-c8ccccc8)c7cc65)cc43)cc2-n2c3ccccc3c3ccccc32)cc1 ; c1ccc(-c2ccccc2-c2ccc(-n3c4ccccc4c4ccc(-n5c6ccccc6c6cc7c(cc65)c5ccccc5n7-c5ccccc5)cc43)cc2-n2c3ccccc3c3ccccc32)cc1]

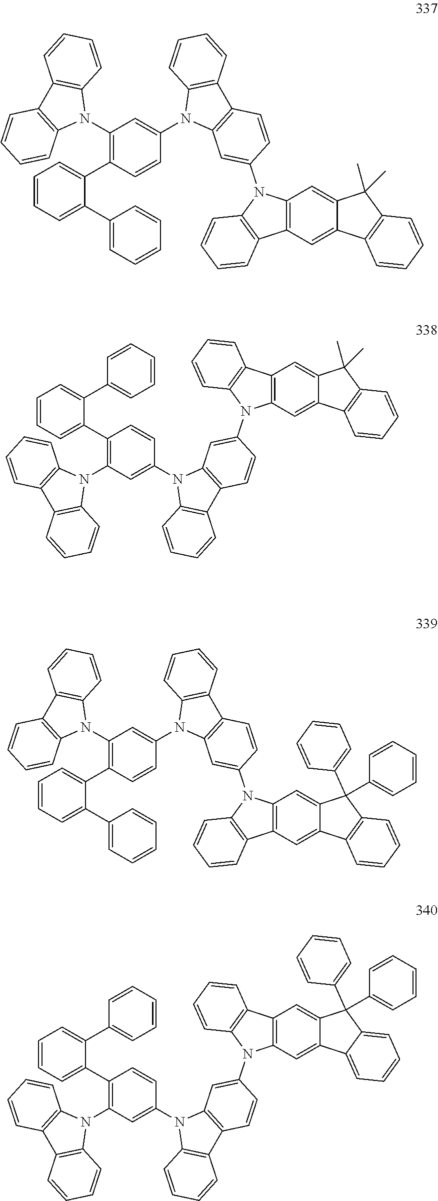 [SMILES: CC1(C)c2ccccc2-c2cc3c4ccccc4n(-c4ccc5c6ccccc6n(-c6ccc(-c7ccccc7-c7ccccc7)c(-n7c8ccccc8c8ccccc87)c6)c5c4)c3cc21 ; CC1(C)c2ccccc2-c2cc3c(cc21)c1ccccc1n3-c1ccc2c3ccccc3n(-c3ccc(-c4ccccc4-c4ccccc4)c(-n4c5ccccc5c5ccccc54)c3)c2c1 ; c1ccc(-c2ccccc2-c2ccc(-n3c4ccccc4c4ccc(-n5c6ccccc6c6cc7c(cc65)C(c5ccccc5)(c5ccccc5)c5ccccc5-7)cc43)cc2-n2c3ccccc3c3ccccc32)cc1 ; c1ccc(-c2ccccc2-c2ccc(-n3c4ccccc4c4ccc(-n5c6ccccc6c6cc7c(cc65)-c5ccccc5C7(c5ccccc5)c5ccccc5)cc43)cc2-n2c3ccccc3c3ccccc32)cc1]

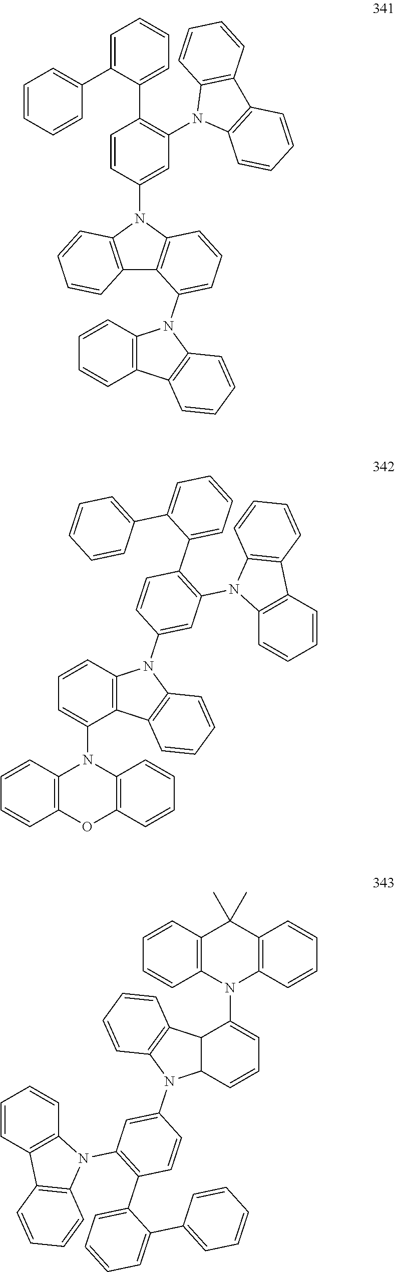 [SMILES: c1ccc(-c2ccccc2-c2ccc(-n3c4ccccc4c4c(-n5c6ccccc6c6ccccc65)cccc43)cc2-n2c3ccccc3c3ccccc32)cc1 ; c1ccc(-c2ccccc2-c2ccc(-n3c4ccccc4c4c(N5c6ccccc6Oc6ccccc65)cccc43)cc2-n2c3ccccc3c3ccccc32)cc1 ; CC1(C)c2ccccc2N(C2=CC=CC3C2c2ccccc2N3c2ccc(-c3ccccc3-c3ccccc3)c(-n3c4ccccc4c4ccccc43)c2)c2ccccc21]

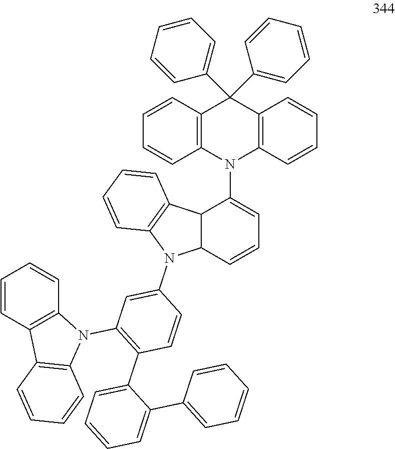 [SMILES: C1=CC2C(C(N3c4ccccc4C(c4ccccc4)(c4ccccc4)c4ccccc43)=C1)c1ccccc1N2c1ccc(-c2ccccc2-c2ccccc2)c(-n2c3ccccc3c3ccccc32)c1]

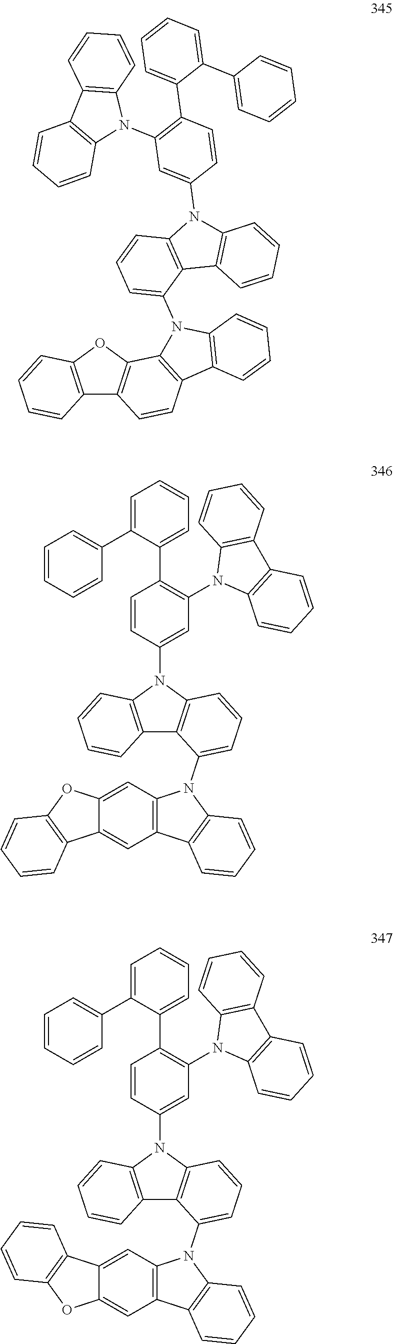 [SMILES: c1ccc(-c2ccccc2-c2ccc(-n3c4ccccc4c4c(-n5c6ccccc6c6ccc7c8ccccc8oc7c65)cccc43)cc2-n2c3ccccc3c3ccccc32)cc1 ; c1ccc(-c2ccccc2-c2ccc(-n3c4ccccc4c4c(-n5c6ccccc6c6cc7c(cc65)oc5ccccc57)cccc43)cc2-n2c3ccccc3c3ccccc32)cc1 ; c1ccc(-c2ccccc2-c2ccc(-n3c4ccccc4c4c(-n5c6ccccc6c6cc7oc8ccccc8c7cc65)cccc43)cc2-n2c3ccccc3c3ccccc32)cc1]

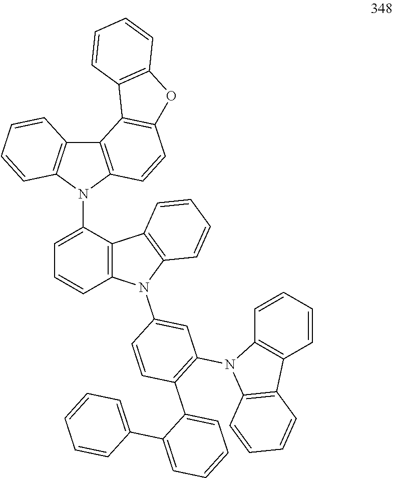 [SMILES: c1ccc(-c2ccccc2-c2ccc(-n3c4ccccc4c4c(-n5c6ccccc6c6c7c(ccc65)oc5ccccc57)cccc43)cc2-n2c3ccccc3c3ccccc32)cc1]

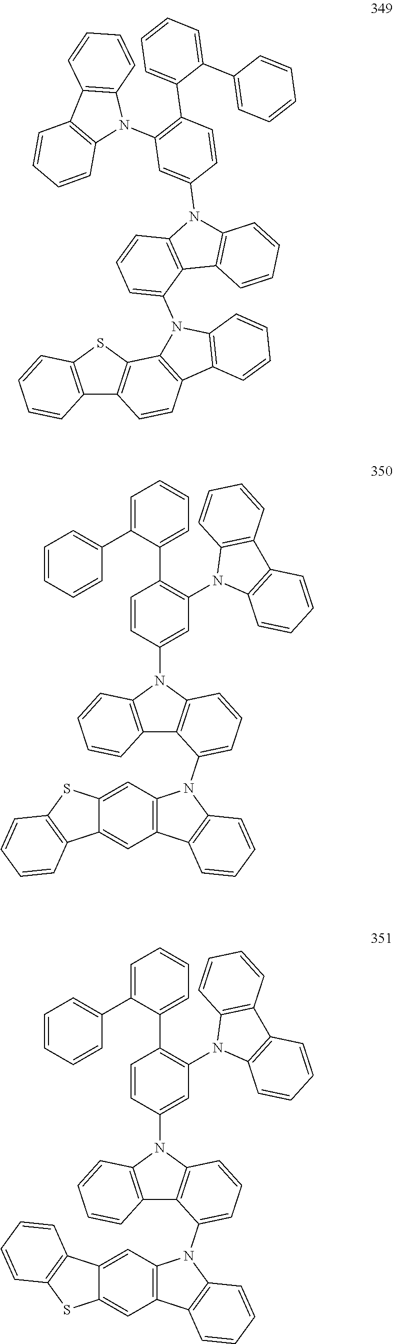 [SMILES: c1ccc(-c2ccccc2-c2ccc(-n3c4ccccc4c4c(-n5c6ccccc6c6ccc7c8ccccc8sc7c65)cccc43)cc2-n2c3ccccc3c3ccccc32)cc1 ; c1ccc(-c2ccccc2-c2ccc(-n3c4ccccc4c4c(-n5c6ccccc6c6cc7c(cc65)sc5ccccc57)cccc43)cc2-n2c3ccccc3c3ccccc32)cc1 ; c1ccc(-c2ccccc2-c2ccc(-n3c4ccccc4c4c(-n5c6ccccc6c6cc7sc8ccccc8c7cc65)cccc43)cc2-n2c3ccccc3c3ccccc32)cc1]

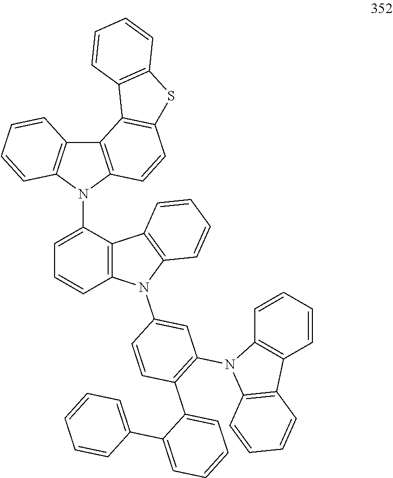 [SMILES: c1ccc(-c2ccccc2-c2ccc(-n3c4ccccc4c4c(-n5c6ccccc6c6c7c(ccc65)sc5ccccc57)cccc43)cc2-n2c3ccccc3c3ccccc32)cc1]

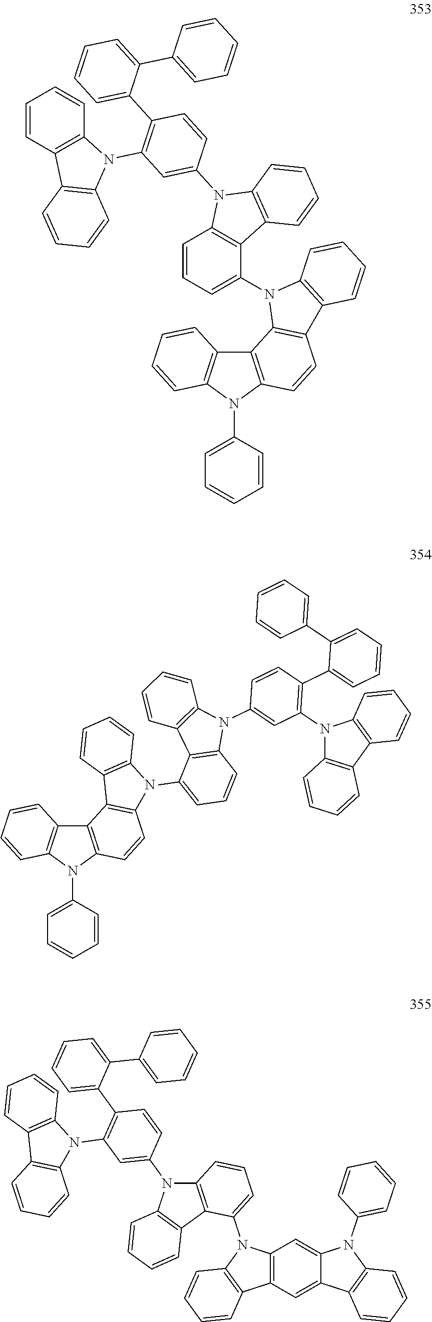 [SMILES: c1ccc(-c2ccccc2-c2ccc(-n3c4ccccc4c4c(-n5c6ccccc6c6ccc7c(c8ccccc8n7-c7ccccc7)c65)cccc43)cc2-n2c3ccccc3c3ccccc32)cc1 ; c1ccc(-c2ccccc2-c2ccc(-n3c4ccccc4c4c(-n5c6ccccc6c6c7c8ccccc8n(-c8ccccc8)c7ccc65)cccc43)cc2-n2c3ccccc3c3ccccc32)cc1 ; c1ccc(-c2ccccc2-c2ccc(-n3c4ccccc4c4c(-n5c6ccccc6c6cc7c8ccccc8n(-c8ccccc8)c7cc65)cccc43)cc2-n2c3ccccc3c3ccccc32)cc1]

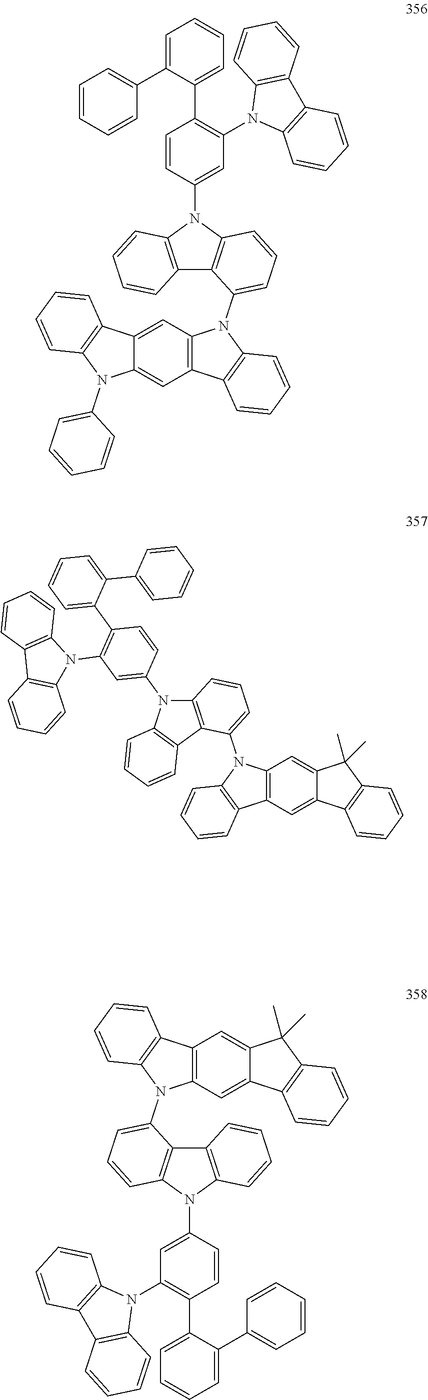 [SMILES: c1ccc(-c2ccccc2-c2ccc(-n3c4ccccc4c4c(-n5c6ccccc6c6cc7c(cc65)c5ccccc5n7-c5ccccc5)cccc43)cc2-n2c3ccccc3c3ccccc32)cc1 ; CC1(C)c2ccccc2-c2cc3c4ccccc4n(-c4cccc5c4c4ccccc4n5-c4ccc(-c5ccccc5-c5ccccc5)c(-n5c6ccccc6c6ccccc65)c4)c3cc21 ; CC1(C)c2ccccc2-c2cc3c(cc21)c1ccccc1n3-c1cccc2c1c1ccccc1n2-c1ccc(-c2ccccc2-c2ccccc2)c(-n2c3ccccc3c3ccccc32)c1]

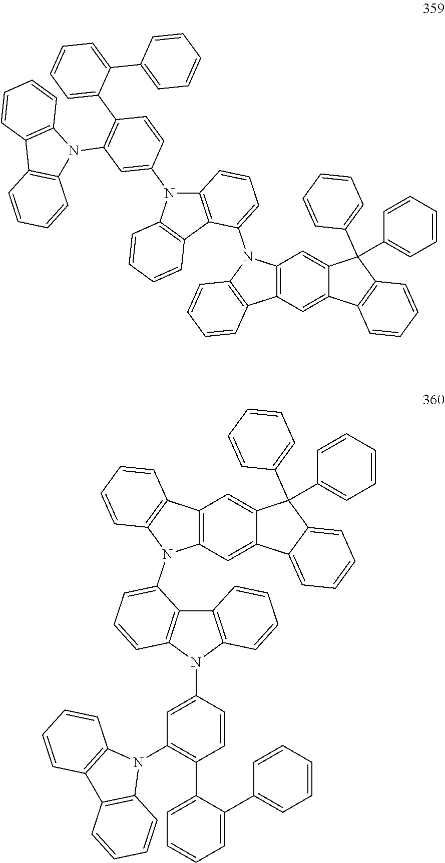 [SMILES: c1ccc(-c2ccccc2-c2ccc(-n3c4ccccc4c4c(-n5c6ccccc6c6cc7c(cc65)C(c5ccccc5)(c5ccccc5)c5ccccc5-7)cccc43)cc2-n2c3ccccc3c3ccccc32)cc1 ; c1ccc(-c2ccccc2-c2ccc(-n3c4ccccc4c4c(-n5c6ccccc6c6cc7c(cc65)-c5ccccc5C7(c5ccccc5)c5ccccc5)cccc43)cc2-n2c3ccccc3c3ccccc32)cc1]

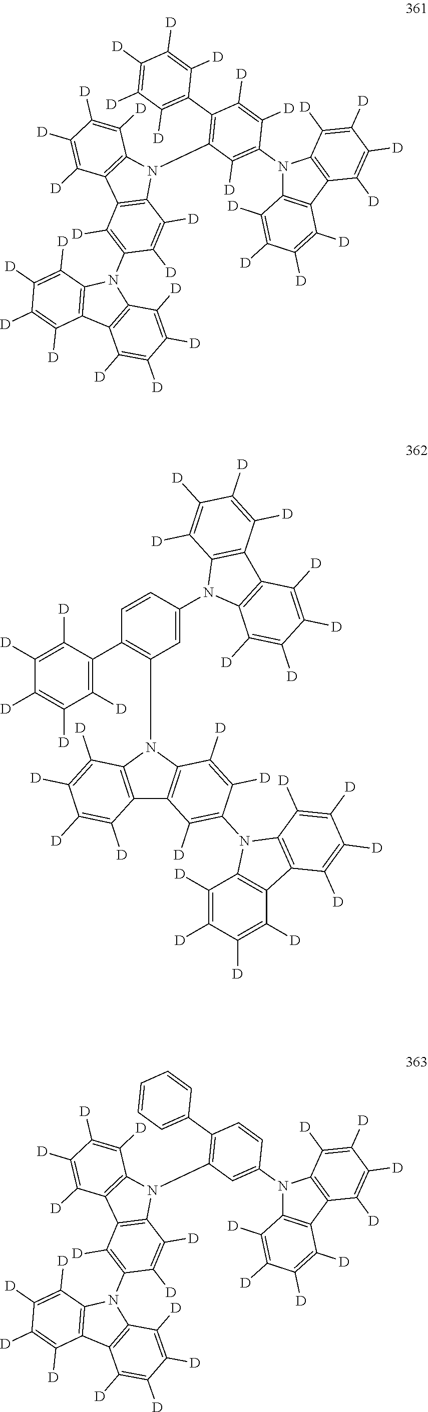

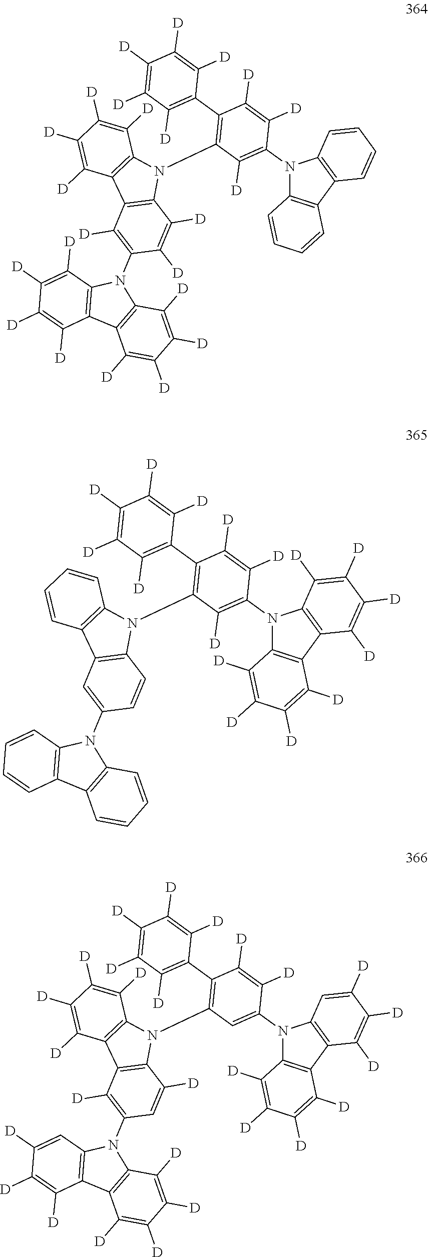

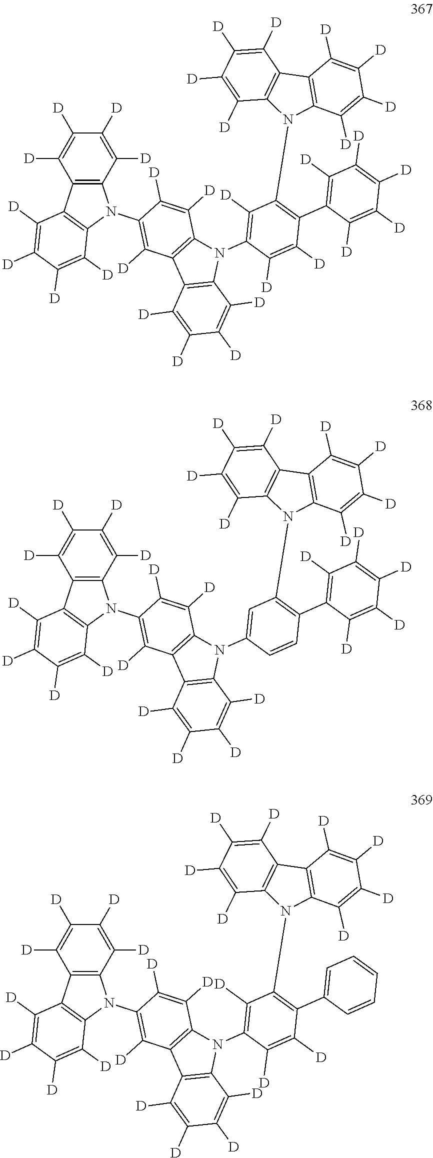

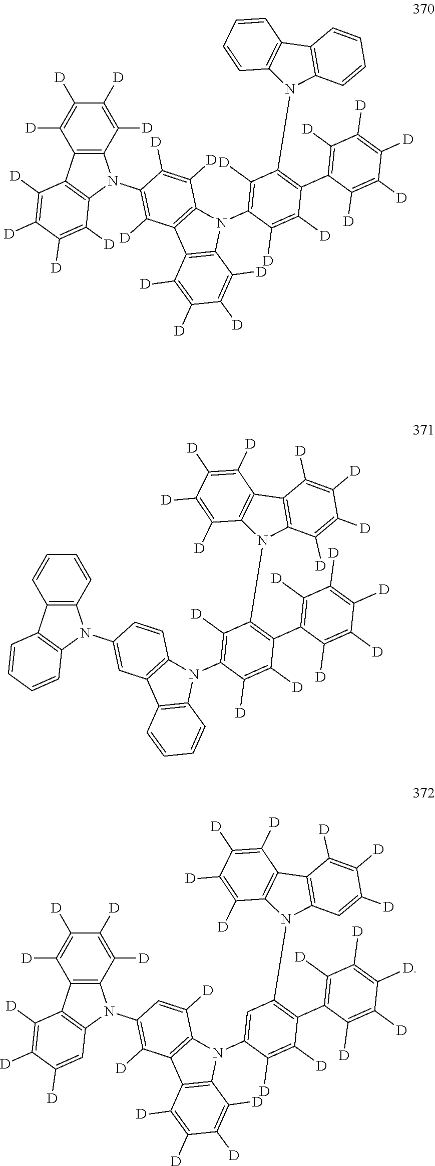

9. An organic light-emitting device comprising:
a first electrode;
a second electrode provided opposite to the first electrode; and
one or more organic material layers provided between the first electrode and the second electrode,
wherein one or more layers of the organic material layers include the heterocyclic compound of claim 1.

10. The organic light-emitting device of claim 9, wherein the organic material layer includes a light emitting layer, and the light emitting layer includes the heterocyclic compound.

11. The organic light-emitting device of claim 9, wherein the organic material layer includes a light emitting layer, the light emitting layer includes a host material, and the host material includes the heterocyclic compound.

12. The organic light-emitting device of claim 9, further comprising one, or two or more layers selected from the group consisting of a hole injection layer, a hole blocking layer, an electron injection layer, an electron transport layer and a hole blocking layer.
